(19) United States
(12) Patent Application Publication
(10) Pub. No.: US 2026/0151494 A1
(43) Pub. Date: Jun. 4, 2026

(54) CD71 BINDING FIBRONECTIN TYPE III DOMAINS

(71) Applicant: Aro Biotherapeutics Company, Philadelphia, PA (US)

(72) Inventors: YAO XIN, Philadelphia, PA (US); Zhanna Druzina, Philadelphia, PA (US); Karyn T. O'Neil, Philadelphia, PA (US)

(21) Appl. No.: 19/122,605

(22) PCT Filed: Oct. 19, 2023
(86) PCT No.: PCT/US2023/077333

§ 371 Date: Apr. 18, 2025

Related U.S. Application Data
(60) Provisional application No. 63/380,112, filed on Oct. 19, 2022.
(60) Provisional application No. 63/505,898, filed on Jun. 2, 2023.

(51) Int. Cl. A61K 47/64; C07K 14/78; G01N 33/575

(52) U.S. Cl. CPC A61K 47/6435; C07K 14/78; G01N 33/5759; G01N 2333/70582; G01N 2333/78

(57) ABSTRACT

The present disclosure relates to polypeptides, such as fibronectin type III (FN3) domains that can bind CD71, their conjugates, isolated nucleotides encoding the molecules, vectors, host-cells, as well as methods of making and using the same.

### CROSS-REFERENCE TO RELATED APPLICATIONS

The present application is a national phase application under 35 U.S.C. § 371 of International Application No. PCT/US2023/077333, filed Oct. 19, 2023, which claims priority to U.S. Provisional Application No. 63/380,112, filed Oct. 19, 2022, and U.S. Provisional Application No. 63/505,898, filed Jun. 2, 2023, each of which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Dec. 27, 2023, is named “ROO-031WO_SL.XML” and is 1,625,016 bytes in size.

### FIELD

The present embodiments relate to fibronectin type III domains (FN3) that specifically bind cluster of differentiation 71 (CD71) and methods of making and using the molecules.

### BACKGROUND

CD71, also known as transferrin receptor 1, is a transmembrane protein that is essential for iron transport into cells. It is highly expressed on many tumor types and at the blood brain barrier, and has thus become an important target for drug delivery. Following binding to iron-loaded transferrin, CD71 is rapidly endocytosed and efficiently recycled back to the cell surface. Studies with anti-CD71 antibody drug conjugates suggest that targeting CD71 can improve specificity and selectivity of drug delivery and widen the therapeutic index. In addition, studies using anti-CD71 monoclonal antibodies indicate that binding affinity can play an important role in enabling tissue-specific delivery, including smooth or skeletal muscle delivery, and blood brain barrier transcytosis. Antibodies with high affinity for CD71 are rapidly internalized and alter normal receptor trafficking so that instead of recycling, the receptor is targeted to the lysosome for degradation. In contrast, antibodies with low affinity for CD71 allow for receptor recycling and higher brain exposure.

While antibodies or antibody fragments are the most widely used class of therapeutic proteins when high affinity and specificity for a target molecule are desired, non-antibody proteins can be engineered to also bind such targets. These “alternative scaffold” proteins have advantages over traditional antibodies due to their small size, lack of disulfide bonds, high stability, ability to be expressed in prokaryotic hosts, easy purification, and they are easily conjugated to drugs/toxins, penetrate efficiently into tissues and are readily formatted into multispecific binders.

One such alternative scaffold is the immunoglobulin (Ig) fold. This fold is found in the variable regions of antibodies, as well as thousands of non-antibody proteins. It has been shown that one such Ig protein, the tenth fibronectin type III (FN3) repeat from human fibronectin, can tolerate a number of mutations in surface exposed loops while retaining the overall Ig-fold structure. Thus, what is needed is a FN3 domain that can specifically bind to CD71, and methods of using such molecules for novel therapeutics that enable intracellular access via receptor mediated internalization of CD71.

### SUMMARY

In some embodiments, FN3 domains (e.g. polypeptides) that specifically bind CD71 protein are provided. In some embodiments, the FN3 domains are isolated. In some embodiments, the FN3 domains are recombinant. In some embodiments, the FN3 domains are non-naturally occurring.

In some embodiments, the CD71-binding FN3 domain comprises an amino acid sequence that is at least 70%, 75%, 80%, 85%, 90%, or 95% identical to an amino acid sequence selected from the group consisting of SEQ ID NOs: 100-209, 211-301, 303-317, 319-552, and 972-976. In some embodiments, the CD71-binding FN3 domain comprises an amino acid sequence selected from the group consisting of SEQ ID NOs: 100-209, 211-301, 303-317, 319-552, and 972-976. In some embodiments, the CD71-binding FN3 domain comprises two of SEQ ID NOs: 100-209, 211-301, 303-317, 319-552, and 972-976. In some embodiments, the CD71-binding FN3 domain binds to human CD71 at a site on CD71 that does not compete with transferrin binding to CD71.

In some embodiments, the CD71-binding FN3 domain is conjugated to a detectable label, an oligonucleotide, a therapeutic agent, or any combination thereof. In some embodiments, the CD71-binding FN3 domain is coupled to a half-life extending moiety. In some embodiments, the half-life extending moiety is an albumin binding molecule, a polyethylene glycol (PEG), albumin, albumin variant, at least a portion of an Fc region of an immunoglobulin.

In some embodiments, an isolated polynucleotide encoding the CD71-binding FN3 domain is provided. In some embodiments, a vector comprising the isolated polynucleotide is provided. In some embodiments, a host cell comprising the vector is provided. In some embodiments, a method of producing a polypeptide that binds CD71 is provided, the method comprising culturing the isolated host cell under conditions that the polypeptide is expressed, and purifying the polypeptide.

In some embodiments, a method of treating cancer in a subject in need thereof is provided, the method comprising administering to the subject a polypeptide provided for herein, such as SEQ ID NOs: 100-209, 211-301, 303-317, 319-552, and 972-976, with a therapeutic agent.

In some embodiments, a method of treating cancer in a subject in need thereof is provided, the method comprising administering to the subject a polypeptide described herein, such as SEQ ID NOs: 100-209, 211-301, 303-317, 319-552, and 972-976, conjugated with an antiviral agent, an immune system modulating agent, or an nucleic acid molecule.

In some embodiments, a method of detecting CD71-expressing cancer cells in a tumor tissue is provided, the method comprising a) obtaining a sample of the tumor tissue from a subject; and, b) detecting whether CD71 is expressed in the tumor tissue by contacting the sample of the tumor tissue with a polypeptide comprising the amino acid sequence of one of SEQ ID NOs: 100-209, 211-301, 303-317, 319-552, and 972-976, and detecting the binding between CD71 and the polypeptide.

In some embodiments, a method of isolating CD71 expressing cells is provided, the method comprising a) obtaining a sample from a subject; b)contacting the sample with the polypeptide comprising the amino acid sequence of one of SEQ ID NOs: 100-209, 211-301, 303-317, 319-552, and 972-976, and c) isolating the cells bound to the polypeptide.

In some embodiments, a method of detecting CD71-expressing cancer cells in a tumor tissue is provided, the method comprising a) conjugating the peptide comprising the amino acid sequence of one of SEQ ID NOs: 100-209, 211-301, 303-317, 319-552, and 972-976 to a detectable label to form a conjugate; b) administering the conjugate to a subject; and c) visualizing the CD71 expressing cancer cells to which the conjugate is bound.

In some embodiments, a method of delivering an agent of interest to a CD71 positive cell is provided, the method comprising contacting a cell with the agent of interest coupled to a FN3 domain that binds to CD71 as provided herein.

In some embodiments, a method of identifying a FN3 protein that binds to CD71 at a site that does not compete or inhibit transferrin binding to CD71 is provided, the method comprising: a) contacting CD71 in the presence of transferrin or an agent that binds to the CD71 transferrin binding site with a test FN3 protein; and b) identifying a test FN3 protein that binds to CD71 in the presence of transferrin or an agent that binds to the CD71 transferrin binding site.

In some embodiments, a composition is provided having a formula of

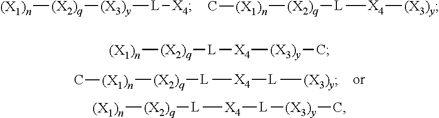

wherein X₁ is a first FN3 domain; X₂ is second FN3 domain; X₃ is a third FN3 domain or half-life extender molecule; L is a linker; and X₄ is a nucleic acid molecule, such as a siRNA molecule provided herein, C is a polymer, such as PEG, albumin binding protein, or an aliphatic chain that binds to serum proteins; wherein n, q, and y are each independently 0 or 1. In some embodiments, the first, second, or third FN3 domain has an amino acid sequence as provided herein. In some embodiments, X₄ is a siRNA molecule provided for herein.

In some embodiments, a composition is provided having a formula A₁-B₁, wherein A₁ has a formula of (C)ₙ-(L₁)ₜ-Xₛ and B₁ has a formula of X_{AS}-(L₂)_{q}-(F₁)_{y}, wherein C is a polymer, such as PEG, albumin binding protein, or an aliphatic chain that binds to serum proteins; L₁ and L₂ are each, independently, a linker; X_{S} is a 5′ to 3′ oligonucleotide sense strand of a double stranded siRNA molecule; X_{AS} is a 3′ to 5′ oligonucleotide antisense strand of a double stranded siRNA molecule; F₁ is a polypeptide comprising at least one FN3 domain; wherein n, t, q, and y are each independently 0 or 1; wherein X_{S} and X_{AS} form a double stranded oligonucleotide molecule.

In some embodiments, a composition is provided having a formula A₁-B₁, wherein A₁ has a formula of (F₁)ₙ-(L₁)ₜ-Xₛ and B₁ has a formula of X_{AS}-(L₂)_{q}-(C)_{y}, wherein: C is a polymer, such as PEG, albumin binding protein, or an aliphatic chain that binds to serum proteins; L₁ and L₂ are each, independently, a linker; X_{S} is a 5′ to 3′ oligonucleotide sense strand of a double stranded siRNA molecule; X_{AS} is a 3′ to 5′ oligonucleotide antisense strand of a double stranded siRNA molecule; F₁ is a polypeptide comprising at least one FN3 domain; wherein n, t, q, and y are each independently 0 or 1; wherein X_{S} and X_{AS} form a double stranded oligonucleotide molecule.

In some embodiments, a method of treating immunological diseases in a subject in need thereof is provided, the method comprising administering to the subject any composition provided herein.

In some embodiments, a method of reducing the expression of a target gene in a cell is provided, the method comprising contacting the immune cell with any composition provided herein.

In some embodiments, a method of delivering a siRNA molecule to a cell in a subject is provided, the method comprising administering to the subject a pharmaceutical composition comprising any composition provided herein.

### BRIEF DESCRIPTION OF THE DRAWINGS

FIGS. 1A and 1B depict the binding kinetics to both human (FIG. 1A) and cynomolgus monkey (FIG. 1B) CD71 extracellular domain (ECD) of various CD71-binding FN3 domains.

FIGS. 2A and 2B depict CTG assays to determine cell internalization of two different groups of selected CD71-binding FN3 domains. FIG. 2A depicts the results of the first group, while FIG. 2B depicts the results of the second group.

FIGS. 3A and 3B depict luciferase reporter assays to determine gene delivery and expression with two different groups of selected CD71-binding FN3 domains conjugated to KRAS2. FIG. 3A depicts the results from the first group, while FIG. 3B depicts the results of the second group.

FIGS. 4A and 4B depict relative mRNA expression from SkBr3 breast cancer cells contacted with selected CD71-binding FN3 domains conjugated to an AHSA1 siRNA. FIG. 4A depicts all results by concentration, while FIG. 4B depicts each construct separately.

FIGS. 5A and 5B depict relative mRNA expression from primary cynomolgus dermal fibroblasts contacted with selected CD71-binding FN3 domains conjugated to an AHSA1 siRNA. FIG. 5A depicts all results by concentration, while FIG. 5B depicts each construct separately.

FIG. 6 depicts mRNA expression from SkBr3 breast cancer cells contacted with a second group of CD71-binding FN3 domains conjugated to an AHSA1 siRNA.

### DETAILED DESCRIPTION OF THE DISCLOSURE

As used in this specification and the appended claims, the singular forms “a,” “an,” and “the” include plural referents unless the content clearly dictates otherwise. Thus, for example, reference to “a cell” includes a combination of two or more cells, and the like. “Fibronectin type III (FN3) domain” (FN3 domain) refers to a domain occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. No. 8,278,419. Individual FN3 domains are referred to by domain number and protein name, e.g., the 3^{rd} FN3 domain of tenascin (TN3), or the 10 FN3 domain of fibronectin (FN10).

The term “capture agent” refers to substances that bind to a particular type of cells and enable the isolation of that cell from other cells. Exemplary capture agents are magnetic beads, ferrofluids, encapsulating reagents, molecules that bind the particular cell type and the like.

“Sample” refers to a collection of similar fluids, cells, or tissues isolated from a subject, as well as fluids, cells, or tissues present within a subject. Exemplary samples are tissue biopsies, fine needle aspirations, surgically resected tissue, organ cultures, cell cultures and biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium and lavage fluids and the like.

“Substituting” or “substituted” or ‘mutating” or “mutated” refers to altering, deleting of inserting one or more amino acids or nucleotides in a polypeptide or polynucleotide sequence to generate a variant of that sequence.

“Variant” refers to a polypeptide or a polynucleotide that differs from a reference polypeptide or a reference polynucleotide by one or more modifications for example, substitutions, insertions or deletions.

“Specifically binds” or “specific binding” refers to the ability of a FN3 domain to bind to its target, such as CD71, with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Alternatively, “specific binding” refers to the ability of a FN3 domain to bind to its target (e.g. CD71) at least 5-fold above a negative control in standard solution ELISA assay. In some embodiments, a negative control is an FN3 domain that does not bind CD71. In some embodiment, an FN3 domain that specifically binds CD71 may have cross-reactivity to other related antigens, for example to the same predetermined antigen from other species (homologs), such as Macaca fascicularis (cynomolgous monkey, cyno) or Pan troglodytes (chimpanzee).

“Library” refers to a collection of variants. The library may be composed of polypeptide or polynucleotide variants.

“Stability” refers to the ability of a molecule to maintain a folded state under physiological conditions such that it retains at least one of its normal functional activities, for example, binding to a predetermined antigen such as CD71.

“Tencon” refers to the synthetic fibronectin type III (FN3) domain having the consensus sequence:

(SEQ ID NO: 1)
LPAPKNLVVSEVTEDSLRLSWTAPDAAFDSFLIQYQESE
KVGEAINLTVPGSERSYDLTGLKPGTEYTVSIYGVKGGH
RSNPLSAEFTT

as described in U.S. Pat. Publ. No. 2010/0216708.

“CD71” refers to human CD71 protein having the amino acid sequence of SEQ ID NOs: 3 or 4. In some embodiments, SEQ ID NO: 3 is full length human CD71 protein. In some embodiments, SEQ ID NO: 4 is the extracellular domain of human CD71.

A “cancer cell” or a “tumor cell” refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific markers levels, invasiveness, tumor growth or suppression in suitable animal hosts such as nude mice, and the like, in vitro, in vivo, and ex vivo (Freshney, Culture of Animal Cells: A Manual of Basic Technique (3rd ed. 1994)).

An “immune cell” refers to the cells of the immune system categorized as lymphocytes (T-cells, B-cells and NK cells), neutrophils, or monocytes/macrophages. Immune cells also include dendritic cells. A “dendritic cell” refers to a type of antigen-presenting cell (APC) that form an important role in the adaptive immune system. The main function of dendritic cells is to present antigens to T lymphocytes, and to secrete cytokines that may further modulate the immune response directly or indirectly. Dendritic cells have the capacity to induce a primary immune response in the inactive or resting naïve T lymphocytes.

“Vector” refers to a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

“Expression vector” refers to a vector that can be utilized in a biological system or in a reconstituted biological system to direct the translation of a polypeptide encoded by a polynucleotide sequence present in the expression vector.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than about 50 amino acids may be referred to as “peptides”.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Subject” includes any human or nonhuman animal. “Nonhuman animal” includes all vertebrates, e.g., mammals and non-mammals, such as nonhuman primates, sheep, dogs, cats, horses, cows chickens, amphibians, reptiles, etc. Except when noted, the terms “patient” or “subject” are used interchangeably.

“Isolated” refers to a homogenous population of molecules (such as synthetic polynucleotides or a polypeptide such as FN3 domains) which have been substantially separated and/or purified away from other components of the system the molecules are produced in, such as a recombinant cell, as well as a protein that has been subjected to at least one purification or isolation step. “Isolated FN3 domain” refers to an FN3 domain that is substantially free of other cellular material and/or chemicals and encompasses FN3 domains that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

### Compositions

In some embodiments, FN3 proteins comprising a polypeptide that binds CD71 are provided. In some embodiments, the polypeptide comprises a FN3 domain that binds to CD71. In some embodiments, the polypeptide comprises an amino acid sequence of SEQ ID NOs: 100-209, 211-301, 303-317, 319-552, and 972-976. In some embodiments, the polypeptide that binds CD71 comprises an amino acid sequence of SEQ ID NOs: 100-209, 211-301, 303-317, 319-552, and 972-976. The sequence of CD71 protein that the polypeptides can bind to can be, for example, SEQ ID NO: 3 or SEQ ID NO: 4. In some embodiments, the FN3 domain that binds to CD71 specifically binds to CD71.

In some embodiments, the FN3 domain that binds CD71 is based on Tencon sequence of SEQ ID NO: 1 or Tencon27 sequence of SEQ ID NO: 2 (LPAPKNLVVSRVTEDSARLSWTAPDAAFDSFLIQYQESEKVGEAIVLTVPGSERSYD LTGLKPGTEYTVSIYGVKGGHRSNPLSAIFTT), optionally having substitutions at residues positions 11, 14, 17, 37, 46, 73, or 86 (residue numbering corresponding to SEQ ID NO: 2).

In some embodiments, an isolated FN3 domain that binds CD71 comprises the amino acid sequence of SEQ ID NOs: 100-209, 211-301, 303-317, 319-552, and 972-976.

In some embodiments, proteins comprising a polypeptide comprising an amino acid sequence of SEQ ID NO: 90. SEQ ID NO: 90 is a consensus sequence based on the sequences of SEQ ID NO: 91, SEQ ID NO: 92, SEQ ID NO: 93, and SEQ ID NO: 94. The sequence of SEQ ID NO: 90 is

MLPAPKNLVVSRVTEDSARLSWTAPDAAFDSFX₁IX₂YX₃
EX₄X₅X₆X₇GEAIX₈LX₉VPGSERSYDLTGLKPGTEYX₁₀
VX₁₁IX₁₂X₁₃VKGGX₁₄X₁₅SX₁₆PLX₁₇AX₁₈FTT

wherein X₈, X₉, X₁₇, and X₁₈ are each, independently, any amino acid other than methionine or proline, and
X₁ is selected from D, F, Y, or H,
X₂ is selected from Y, G, A, or V,
X₃ is selected from I, T, L, A, or H,
X₄ is selected from S, Y or P,
X₅ is selected from Y, G, Q, or R,
X₆ is selected from G or P,
X₇ is selected from A, Y, P, D, or S,
X₁₀ is selected from W, N, S, or E,
X₁₁ is selected from L, Y, or G,
X₁₂ is selected from D, Q, H, or V,
X₁₃ is selected from G or S,
X₁₄ is selected from R, G, F, L, or D,
X₁₅ is selected from W, S, P, or L, and
X₁₆ is selected from T, V, M, or S.

In some embodiments:
X₁ is selected from D, F, Y, or H,
X₂ is selected from G, A, or V,
X₃ is selected from T, L, A, or H,
X₄ is selected from Y or P,
X₅ is selected from G, Q, or R,
X₆ is selected from G or P,
X₇ is selected from Y, P, D, or S,
X₁₀ is selected from W, N, S, or E,
X₁₁ is selected from L, Y, or G,
X₁₂ is selected from Q, H, or V,
X₁₃ is selected from G or S,
X₁₄ is selected from G, F, L, or D,
X₁₅ is selected from S, P, or L, and
X₁₆ is selected from V, M, or S.

In some embodiments, X₁, X₂, X₃, X₄, X₅, X₆, X₇, X₁₀, X₁₁, X₁₂, X₁₃, X₁₄, X₁₅, and X₁₁ are as shown in the sequence of SEQ ID NO: 91. In some embodiments, X₁, X₂, X₃, X₄, X₅, X₆, X₇, X₁₀, X₁₁, X₁₂, X₁₃, X₁₄, X₁₅, and X₁₁ are as shown in the sequence of SEQ ID NO: 92. In some embodiments, X₁, X₂, X₃, X₄, X₅, X₆, X₇, X₁₀, X₁₁, X₁₂, X₁₃, X₁₄, X₁₅, and X₁₁ are as shown in the sequence of SEQ ID NO: 93. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 94.

In some embodiments, X₈, X₉, X₁₇, and X₁₈ is, independently, alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, not alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, alanine. In some embodiments, X₈, X₉, X₁₇, and X₁₈ is, independently, arginine. In some embodiments, X₈, X₉, X₁₇, and X₁₈ is, independently asparagine. In some embodiments, X₈, X₉, X₁₇, and X₁₈ is, independently, aspartic acid. In some embodiments, X₈, X₉, X₁₇, and X₁₈ is, independently, cysteine. In some embodiments, X₈, X₉, X₁₇, and X₁₈ is, independently, glutamine. In some embodiments, X₈, X₉, X₁₇, and X₁₈ is, independently, glutamic acid. In some embodiments, X₈, X₉, X₁₇, and X₁₈ is, independently, glycine. In some embodiments, X₈, X₉, X₁₇, and X₁₈ is, independently, histidine. In some embodiments, X₈, X₉, X₁₇, and X₁₈ is, independently, isoleucine. In some embodiments, X₈, X₉, X₁₇, and X₁₈ is, independently, leucine. In some embodiments, X₈, X₉, X₁₇, and X₁₈ is, independently, lysine. In some embodiments, X₈, X₉, X₁₇, and X₁₈ is, independently, phenylalanine. In some embodiments, X₈, X₉, X₁₇, and X₁₈ is, independently serine. In some embodiments, X₈, X₉, X₁₇, and X₁₈ is, independently, threonine. In some embodiments, X₈, X₉, X₁₇, and X₁₈ is, independently, tryptophan. In some embodiments, X₈, X₉, X₁₇, and X₁₈ is, independently, tyrosine. In some embodiments, X₈, X₉, X₁₇, and X₁₈ is, independently valine.

In some embodiments, the sequence is set forth as shown in in the sequence of SEQ ID NO: 91, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 92, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 93, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 94, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, provided herein are proteins comprising a polypeptide comprising an amino acid sequence that is at least 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the sequence of SEQ ID NO: 90. In some embodiments, the polypeptide is at least 85%, 86%, 87%, 88%, 89%, 90%, 91%, 92%, 93%, 94%, 95%, 96%, 97%, 98% or 99% identical to the sequence of SEQ ID NO: 90. In some embodiments, the polypeptide is at least 90%, 91%, 92%, 93%, 94%, 95%, 96%, 97%, 98% or 99% identical to the sequence of SEQ ID NO: 90. In some embodiments, the polypeptide is at least 95%, 96%, 97%, 98% or 99% identical to the sequence of SEQ ID NO: 90. In some embodiments, the polypeptide is at least 70%, 75%, 80%, 85%, or 90% identical to the sequence of SEQ ID NO: 90.

Sequences SEQ ID NOs: 91-94 are listed in Table 1 below.

TABLE 1

| SEQ ID | |
|---|---|
| NO: | SEQUENCE |
| 91 | MLPAPKNLVVSRVTEDSARLSWTAPDAAFDS |
| | FYIAYAEPRPDGEAIVLTVPGSERSYDLTGL |
| | KPGTEYSVLIHGVKGGLLSSPLSAIFTT |
| 92 | MLPAPKNLVVSRVTEDSARLSWTAPDAAFDS |
| | FFIGYLEPQPPGEAIVLTVPGSERSYDLTGL |
| | KPGTEYNVTIQGVKGGFPSMPLSAIFTT |
| 93 | MLPAPKNLVVSRVTEDSARLSWTAPDAAFDS |
| | FHIVYHEPRPSGEAIVLTVPGSERSYDLTGL |
| | KPGTEYEVGIVSVKGGDLSVPLSAIFTT |
| 94 | MLPAPKNLVVSRVTEDSARLSWTAPDAAFDS |
| | FDIGYTEYGGYGEAIVLTVPGSERSYDLTGL |
| | KPGTEYWVLIQGVKGGGSSVPLSAIFTT |

Percent identity can be determined using the default parameters to align two sequences using BlastP available through the NCBI website.

The polypeptides provided herein can be part of a larger polypeptide and can be referred to as a domain. The homology or identity between two domains in different polypeptides is based on the domains that are similar as opposed to the overall polypeptide.

For example, if a polypeptide comprises a polypeptide comprising a FN3 domain comprising SEQ ID NO: 100 and said domain is conjugated to a scFV antibody, another protein that has a domain that is similar but not identical to SEQ ID NO: 100 can be at least 90% o identical even if the scFV shares no homology. Thus, the 00 identity can be based on the domain or on the entire length of the polypeptide. Methods of determining % identity are provided for herein or are known to one of skill in the art.

As provided herein, in some embodiments, the FN3 domain that binds to CD71 binds to human mature CD71 or the human mature CD71 extracellular domain. In some embodiments, the human mature CD71 is SEQ ID NO: 3, and the human mature CD71 extracellular binding domain is SEQ ID NO: 4, each of which is provided below in Table 2.

TABLE 2
CD71 Sequences

| SEQ | |
|---|---|
| ID | |
| NO | SEQUENCE |
| 3 | MMDQARSAFSNLEGGEPLSYTRESLARQVDGDNSHVEMKLAVDEE |
| | ENADNNTKANVTKPKRCSGSICYGTIAVIVFFLIGEMIGYLGYCK |
| | GVEPKTECERLAGTESPVREEPGEDFPAARRLYWDDLKRKLSEKL |
| | DSTDFTGTIKLLNENSYVPREAGSQKDENLALYVENQFREFKLSK |
| | VWRDQHFVKIQVKDSAQNSVIIVDKNGRLVYLVENPGGYVAYSKA |
| | ATVTGKLVHANFGTKKDFEDLYTPVNGSIVIVRAGKITFAEKVAN |
| | AESLNAIGVLIYMDQTKFPIVNAELSFFGHAHLGTGDPYTPGFPS |
| | ENHTQFPPSRSSGLPNIPVQTISRAAAEKLFGNMEGDCPSDWKTD |
| | STCRMVTSESKNVKLTVSNVLKEIKILNIFGVIKGFVEPDHYVVV |
| | GAQRDAWGPGAAKSGVGTALLLKLAQMFSDMVLKDGFQPSRSIIF |
| | ASWSAGDFGSVGATEWLEGYLSSLHLKAFTYINLDKAVLGTSNFK |
| | VSASPLLYTLIEKTMQNVKHPVTGQFLYQDSNWASKVEKLTLDNA |
| | AFPFLAYSGIPAVSFCFCEDTDYPYLGTTMDTYKELIERIPELNK |
| | VARAAAEVAGQFVIKLTHDVELNLDYERYNSQLLSFVRDLNQYRA |
| | DIKEMGLSLQWLYSARGDFFRATSRLTTDFGNAEKTDRFVMKKLN |
| | DRVMRVEYHELSPYVSPKESPFRHVFWGSGSHTLPALLENLKLRK |
| | QNNGAFNETLERNQLALATWTIQGAANALSGDVWDIDNEF |
| 4 | CKGVEPKTECERLAGTESPVREEPGEDFPAARRLYWDDLKRKLSE |
| | KLDSTDFTGTIKLLNENSYVPREAGSQKDENLALYVENQFREFKL |
| | SKVWRDQHFVKIQVKDSAQNSVIIVDKNGRLVYLVENPGGYVAYS |
| | KAATVTGKLVHANFGTKKDFEDLYTPVNGSIVIVRAGKITFAEKV |
| | ANAESLNAIGVLIYMDQTKFPIVNAELSFFGHAHLGTGDPYTPGF |
| | PSFNHTQFPPSRSSGLPNIPVQTISRAAAEKLFGNMEGDCPSDWK |
| | TDSTCRMVTSESKNVKLTVSNVLKEIKILNIFGVIKGFVEPDHYV |
| | VVGAQRDAWGPGAAKSGVGTALLLKLAQMESDMVLKDGFQPSRSI |
| | IFASWSAGDEGSVGATEWLEGYLSSLHLKAFTYINLDKAVLGTSN |
| | EKVSASPLLYTLIEKTMQNVKHPVTGQFLYQDSNWASKVEKLTLD |
| | NAAFPFLAYSGIPAVSFCFCEDTDYPYLGTTMDTYKELIERIPEL |
| | NKVARAAAEVAGQFVIKLTHDVELNLDYERYNSQLLSFVRDLNQY |
| | RADIKEMGLSLQWLYSARGDFFRATSRLTTDEGNAEKTDRFVMKK |
| | LNDRVMRVEYHFLSPYVSPKESPFRHVFWGSGSHTLPALLENLKL |
| | RKQNNGAFNETLFRNQLALATWTIQGAANALSGDVWDIDNEF |

As provided herein, the FN3 domains can bind to the CD71 protein. Also provided, even if not explicitly stated, is that the domains can also specifically bind to the CD71 protein. Thus, for example, a FN3 domain that binds to CD71 would also encompass a FN3 domain protein that specifically binds to CD71. These molecules can be used, for example, in therapeutic and diagnostic applications and in imaging. In some embodiments, polynucleotides encoding the FN3 domains disclosed herein or complementary nucleic acids thereof, vectors, host cells, and methods of making and using them are provided. In some embodiments, an isolated FN3 domain that binds or specifically binds CD71 is provided.

In some embodiments, the FN3 domain may bind CD71 with a dissociation constant (K_{D}) of less than about 1×10⁻⁷ M, for example less than about 1×10⁻⁸ M, less than about 1×10⁻⁹ M, less than about 1×10⁻¹⁰ M, less than about 1×10⁻¹¹ M, less than about 1×10⁻¹² M, or less than about 1×10⁻¹³ M as determined by surface plasmon resonance or the Kinexa method, as practiced by those of skill in the art. The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are made with standardized solutions of protein scaffold and antigen, and a standardized buffer, such as the buffers described herein.

In some embodiments, the FN3 domain may bind CD71 at least 5-fold above the signal obtained for a negative control in a standard solution ELISA assay.

In some embodiments, the FN3 domain that binds or specifically binds CD71 comprises an initiator methionine (Met) linked to the N-terminus of the molecule. In some embodiments, the FN3 domain that binds or specifically binds CD71 comprises a cysteine (Cys) linked to a C-terminus of the FN3 domain. The addition of the N-terminal Met and/or the C-terminal Cys may facilitate expression and/or conjugation to extend half-life and to provide other functions of molecules.

The FN3 domain can also contain cysteine substitutions, such as those that are described in U.S. Pat. No. 10,196,446, which is hereby incorporated by reference in its entirety. Briefly, in some embodiments, the polypeptides provided herein can comprise at least one cysteine substitution at a position selected from the group consisting of residues 6, 8, 10, 11, 14, 15, 16, 20, 30, 34, 38, 40, 41, 45, 47, 48, 53, 54, 59, 60, 62, 64, 70, 88, 89, 90, 91, and 93 of the FN3 domain based on SEQ ID NO: 1 or SEQ ID NO: 1 of U.S. Pat. No. 10,196,446 (LPAPKNLVVSEVTEDSLRLSWTAPDAAFDSFLIQYQESEKVGEAINLTVPGSERSYD LTGLKPGTEYTVSIYGVKGGHRSNPLSAEFTT (SEQ ID NO: 1)) which is hereby incorporated by reference in its entirety, and the equivalent positions in related FN3 domains.

In some embodiments, the substitution is at residue 6. In some embodiments, the substitution is at residue 8. In some embodiments, the substitution is at residue 10. In some embodiments, the substitution is at residue 11. In some embodiments, the substitution is at residue 14. In some embodiments, the substitution is at residue 15. In some embodiments, the substitution is at residue 16. In some embodiments, the substitution is at residue 20. In some embodiments, the substitution is at residue 30. In some embodiments, the substitution is at residue 34. In some embodiments, the substitution is at residue 38. In some embodiments, the substitution is at residue 40. In some embodiments, the substitution is at residue 41. In some embodiments, the substitution is at residue 45. In some embodiments, the substitution is at residue 47. In some embodiments, the substitution is at residue 48. In some embodiments, the substitution is at residue 53. In some embodiments, the substitution is at residue 54. In some embodiments, the substitution is at residue 59. In some embodiments, the substitution is at residue 60. In some embodiments, the substitution is at residue 62. In some embodiments, the substitution is at residue 64. In some embodiments, the substitution is at residue 70. In some embodiments, the substitution is at residue 88. In some embodiments, the substitution is at residue 89. In some embodiments, the substitution is at residue 90. In some embodiments, the substitution is at residue 91. In some embodiments, the substitution is at residue 93.

A cysteine substitution at a position in the domain or protein comprises a replacement of the existing amino acid residue with a cysteine residue. In some embodiments, instead of a substitution a cysteine is inserted into the sequence adjacent to the positions listed above. Other examples of cysteine modifications can be found in, for example, U.S. Patent Application Publication No. 20170362301, which is hereby incorporated by reference in its entirety. The alignment of the sequences can be performed using BlastP using the default parameters at, for example, the NCBI website.

In some embodiments, a cysteine residue is inserted at any position in the domain or protein.

In some embodiments, the FN3 domain that binds CD71 is internalized into a cell. In some embodiments, internalization of the FN3 domain may facilitate delivery of a detectable label or therapeutic into a cell. In some embodiments, internalization of the FN3 domain may facilitate delivery of a cytotoxic agent into a cell. The cytotoxic agent can act as a therapeutic agent. In some embodiments, internalization of the FN3 domain may facilitate the delivery of any detectable label, therapeutic, and/or cytotoxic agent disclosed herein into a cell. In some embodiments, internalization of the FN3 domain may facilitate delivery of a oligonucleotide or siRNA molecule into a cell. In some embodiments, the cell is a tumor cell. In some embodiments, the cell is a liver cell. In some embodiments, the cell is a muscle cell. In some embodiments, the cell is an immune cell. In some embodiments, the cell is a cell of the central nervous system. In some embodiments, the cell is a heart cell. In some embodiments, the therapeutic is a siRNA molecule as provided for herein. The FN3 domains that bind CD71 conjugated to a detectable label can be used to evaluate expression of CD71 on samples such as tumor tissue in vivo or in vitro. The FN3 domains that bind CD71 conjugated to a detectable label can be used to evaluate expression of CD71 on samples blood, immune cells, or muscle cells in vivo or in vitro.

In some embodiments, an isolated FN3 domain that binds CD71 comprises the amino acid sequence of SEQ ID NOs: 100-209, 211-301, 303-317, 319-552, and 972-976.

In some embodiments, an isolated FN3 domain that binds CD71 comprises the amino acid sequence of SEQ ID NO: 100. In some embodiments, an isolated FN3 domain that binds CD71 comprises the amino acid sequence of SEQ ID NO: 101. In some embodiments, an isolated FN3 domain that binds CD71 comprises the amino acid sequence of SEQ ID NO: 102. In some embodiments, an isolated FN3 domain that binds CD71 comprises the amino acid sequence of SEQ ID NO: 103. In some embodiments, an isolated FN3 domain that binds CD71 comprises the amino acid sequence of SEQ ID NO: 104. In some embodiments, an isolated FN3 domain that binds CD71 comprises the amino acid sequence of SEQ ID NO: 105. In some embodiments, an isolated FN3 domain that binds CD71 comprises the amino acid sequence of SEQ ID NO: 106. In some embodiments, an isolated FN3 domain that binds CD71 comprises the amino acid sequence of SEQ ID NO: 107. In some embodiments, an isolated FN3 domain that binds CD71 comprises the amino acid sequence of SEQ ID NO: 108. In some embodiments, an isolated FN3 domain that binds CD71 comprises the amino acid sequence of SEQ ID NO: 109. In some embodiments, an isolated FN3 domain that binds CD71 comprises the amino acid sequence of SEQ ID NO: 110. In some embodiments, an isolated FN3 domain that binds CD71 comprises the amino acid sequence of SEQ ID NO: 111. In some embodiments, an isolated FN3 domain that binds CD71 comprises the amino acid sequence of SEQ ID NO: 112. In some embodiments, an isolated FN3 domain that binds CD71 comprises the amino acid sequence of SEQ ID NO: 113. In some embodiments, an isolated FN3 domain that binds CD71 comprises the amino acid sequence of SEQ ID NO: 114. In some embodiments, an isolated FN3 domain that binds CD71 comprises the amino acid sequence of SEQ ID NO: 115. In some embodiments, an isolated FN3 domain that binds CD71 comprises the amino acid sequence of SEQ ID NO: 116. In some embodiments, an isolated FN3 domain that binds CD71 comprises the amino acid sequence of SEQ ID NO: 117. In some embodiments, an isolated FN3 domain that binds CD71 comprises the amino acid sequence of SEQ ID NO: 118. In some embodiments, an isolated FN3 domain that binds CD71 comprises the amino acid sequence of SEQ ID NO: 119. In some embodiments, an isolated FN3 domain that binds CD71 comprises the amino acid sequence of SEQ ID NO: 120. In some embodiments, an isolated FN3 domain that binds CD71 comprises the amino acid sequence of SEQ ID NO: 121. In some embodiments, an isolated FN3 domain that binds CD71 comprises the amino acid sequence of SEQ ID NO: 122. In some embodiments, an isolated FN3 domain that binds CD71 comprises the amino acid sequence of SEQ ID NO: 123. In some embodiments, an isolated FN3 domain that binds CD71 comprises the amino acid sequence of SEQ ID NO: 124. In some embodiments, an isolated FN3 domain that binds CD71 comprises the amino acid sequence of SEQ ID NO: 125. In some embodiments, an isolated FN3 domain that binds CD71 comprises the amino acid sequence of SEQ ID NO: 126. In some embodiments, an isolated FN3 domain that binds CD71 comprises the amino acid sequence of SEQ ID NO: 127. In some embodiments, an isolated FN3 domain that binds CD71 comprises the amino acid sequence of SEQ ID NO: 128. In some embodiments, an isolated FN3 domain that binds CD71 comprises the amino acid sequence of SEQ ID NO: 129. In some embodiments, an isolated FN3 domain that binds CD71 comprises the amino acid sequence of SEQ ID NO: 130. In some embodiments, an isolated FN3 domain that binds CD71 comprises the amino acid sequence of SEQ ID NO: 131. In some embodiments, an isolated FN3 domain that binds CD71 comprises the amino acid sequence of SEQ ID NO: 132. In some embodiments, an isolated FN3 domain that binds CD71 comprises the amino acid sequence of SEQ ID NO: 133. In some embodiments, an isolated FN3 domain that binds CD71 comprises the amino acid sequence of SEQ ID NO: 134. In some embodiments, an isolated FN3 domain that binds CD71 comprises the amino acid sequence of SEQ ID NO: 135. In some embodiments, an isolated FN3 domain that binds CD71 comprises the amino acid sequence of SEQ ID NO: 136. In some embodiments, an isolated FN3 domain that binds CD71 comprises the amino acid sequence of SEQ ID NO: 137. In some embodiments, an isolated FN3 domain that binds CD71 comprises the amino acid sequence of SEQ ID NO: 138. In some embodiments, an isolated FN3 domain that binds CD71 comprises the amino acid sequence of SEQ ID NO: 139. In some embodiments, an isolated FN3 domain that binds CD71 comprises the amino acid sequence of SEQ ID NO: 140. In some embodiments, an isolated FN3 domain that binds CD71 comprises the amino acid sequence of SEQ ID NO: 141. In some embodiments, an isolated FN3 domain that binds CD71 comprises the amino acid sequence of SEQ ID NO: 142. In some embodiments, an isolated FN3 domain that binds CD71 comprises the amino acid sequence of SEQ ID NO: 143. In some embodiments, an isolated FN3 domain that binds CD71 comprises the amino acid sequence of SEQ ID NO: 144. In some embodiments, an isolated FN3 domain that binds CD71 comprises the amino acid sequence of SEQ ID NO: 145. In some embodiments, an isolated FN3 domain that binds CD71 comprises the amino acid sequence of SEQ ID NO: 146. In some embodiments, an isolated FN3 domain that binds CD71 comprises the amino acid sequence of SEQ ID NO: 147. In some embodiments, an isolated FN3 domain that binds CD71 comprises the amino acid sequence of SEQ ID NO: 148. In some embodiments, an isolated FN3 domain that binds CD71 comprises the amino acid sequence of SEQ ID NO: 149. In some embodiments, an isolated FN3 domain that binds CD71 comprises the amino acid sequence of SEQ ID NO: 150. In some embodiments, an isolated FN3 domain that binds CD71 comprises the amino acid sequence of SEQ ID NO: 151. In some embodiments, an isolated FN3 domain that binds CD71 comprises the amino acid sequence of SEQ ID NO: 152. In some embodiments, an isolated FN3 domain that binds CD71 comprises the amino acid sequence of SEQ ID NO: 153. In some embodiments, an isolated FN3 domain that binds CD71 comprises the amino acid sequence of SEQ ID NO: 154. In some embodiments, an isolated FN3 domain that binds CD71 comprises the amino acid sequence of SEQ ID NO: 155. In some embodiments, an isolated FN3 domain that binds CD71 comprises the amino acid sequence of SEQ ID NO: 156. In some embodiments, an isolated FN3 domain that binds CD71 comprises the amino acid sequence of SEQ ID NO: 157. In some embodiments, an isolated FN3 domain that binds CD71 comprises the amino acid sequence of SEQ ID NO: 158. In some embodiments, an isolated FN3 domain that binds CD71 comprises the amino acid sequence of SEQ ID NO: 159. In some embodiments, an isolated FN3 domain that binds CD71 comprises the amino acid sequence of SEQ ID NO: 160. In some embodiments, an isolated FN3 domain that binds CD71 comprises the amino acid sequence of SEQ ID NO: 161. In some embodiments, an isolated FN3 domain that binds CD71 comprises the amino acid sequence of SEQ ID NO: 162. In some embodiments, an isolated FN3 domain that binds CD71 comprises the amino acid sequence of SEQ ID NO: 163. In some embodiments, an isolated FN3 domain that binds CD71 comprises the amino acid sequence of SEQ ID NO: 164. In some embodiments, an isolated FN3 domain that binds CD71 comprises the amino acid sequence of SEQ ID NO: 165. In some embodiments, an isolated FN3 domain that binds CD71 comprises the amino acid sequence of SEQ ID NO: 166. In some embodiments, an isolated FN3 domain that binds CD71 comprises the amino acid sequence of SEQ ID NO: 167. In some embodiments, an isolated FN3 domain that binds CD71 comprises the amino acid sequence of SEQ ID NO: 168. In some embodiments, an isolated FN3 domain that binds CD71 comprises the amino acid sequence of SEQ ID NO: 169. In some embodiments, an isolated FN3 domain that binds CD71 comprises the amino acid sequence of SEQ ID NO: 170. In some embodiments, an isolated FN3 domain that binds CD71 comprises the amino acid sequence of SEQ ID NO: 171. In some embodiments, an isolated FN3 domain that binds CD71 comprises the amino acid sequence of SEQ ID NO: 172. In some embodiments, an isolated FN3 domain that binds CD71 comprises the amino acid sequence of SEQ ID NO: 173. In some embodiments, an isolated FN3 domain that binds CD71 comprises the amino acid sequence of SEQ ID NO: 174. In some embodiments, an isolated FN3 domain that binds CD71 comprises the amino acid sequence of SEQ ID NO: 175. In some embodiments, an isolated FN3 domain that binds CD71 comprises the amino acid sequence of SEQ ID NO: 176. In some embodiments, an isolated FN3 domain that binds CD71 comprises the amino acid sequence of SEQ ID NO: 177. In some embodiments, an isolated FN3 domain that binds CD71 comprises the amino acid sequence of SEQ ID NO: 178. In some embodiments, an isolated FN3 domain that binds CD71 comprises the amino acid sequence of SEQ ID NO: 179. In some embodiments, an isolated FN3 domain that binds CD71 comprises the amino acid sequence of SEQ ID NO: 180. In some embodiments, an isolated FN3 domain that binds CD71 comprises the amino acid sequence of SEQ ID NO: 181. In some embodiments, an isolated FN3 domain that binds CD71 comprises the amino acid sequence of SEQ ID NO: 182. In some embodiments, an isolated FN3 domain that binds CD71 comprises the amino acid sequence of SEQ ID NO: 183. In some embodiments, an isolated FN3 domain that binds CD71 comprises the amino acid sequence of SEQ ID NO: 184. In some embodiments, an isolated FN3 domain that binds CD71 comprises the amino acid sequence of SEQ ID NO: 185. In some embodiments, an isolated FN3 domain that binds CD71 comprises the amino acid sequence of SEQ ID NO: 186. In some embodiments, an isolated FN3 domain that binds CD71 comprises the amino acid sequence of SEQ ID NO: 187. In some embodiments, an isolated FN3 domain that binds CD71 comprises the amino acid sequence of SEQ ID NO: 188. In some embodiments, an isolated FN3 domain that binds CD71 comprises the amino acid sequence of SEQ ID NO: 189. In some embodiments, an isolated FN3 domain that binds CD71 comprises the amino acid sequence of SEQ ID NO: 190. In some embodiments, an isolated FN3 domain that binds CD71 comprises the amino acid sequence of SEQ ID NO: 191. In some embodiments, an isolated FN3 domain that binds CD71 comprises the amino acid sequence of SEQ ID NO: 192. In some embodiments, an isolated FN3 domain that binds CD71 comprises the amino acid sequence of SEQ ID NO: 193. In some embodiments, an isolated FN3 domain that binds CD71 comprises the amino acid sequence of SEQ ID NO: 194. In some embodiments, an isolated FN3 domain that binds CD71 comprises the amino acid sequence of SEQ ID NO: 195. In some embodiments, an isolated FN3 domain that binds CD71 comprises the amino acid sequence of SEQ ID NO: 196. In some embodiments, an isolated FN3 domain that binds CD71 comprises the amino acid sequence of SEQ ID NO: 197. In some embodiments, an isolated FN3 domain that binds CD71 comprises the amino acid sequence of SEQ ID NO: 198. In some embodiments, an isolated FN3 domain that binds CD71 comprises the amino acid sequence of SEQ ID NO: 199. In some embodiments, an isolated FN3 domain that binds CD71 comprises the amino acid sequence of SEQ ID NO: 200. In some embodiments, an isolated FN3 domain that binds CD71 comprises the amino acid sequence of SEQ ID NO: 201. In some embodiments, an isolated FN3 domain that binds CD71 comprises the amino acid sequence of SEQ ID NO: 202. In some embodiments, an isolated FN3 domain that binds CD71 comprises the amino acid sequence of SEQ ID NO: 203. In some embodiments, an isolated FN3 domain that binds CD71 comprises the amino acid sequence of SEQ ID NO: 204. In some embodiments, an isolated FN3 domain that binds CD71 comprises the amino acid sequence of SEQ ID NO: 205. In some embodiments, an isolated FN3 domain that binds CD71 comprises the amino acid sequence of SEQ ID NO: 206. In some embodiments, an isolated FN3 domain that binds CD71 comprises the amino acid sequence of SEQ ID NO: 207. In some embodiments, an isolated FN3 domain that binds CD71 comprises the amino acid sequence of SEQ ID NO: 208. In some embodiments, an isolated FN3 domain that binds CD71 comprises the amino acid sequence of SEQ ID NO: 209. In some embodiments, an isolated FN3 domain that binds CD71 comprises the amino acid sequence of SEQ ID NO: 210. In some embodiments, an isolated FN3 domain that binds CD71 comprises the amino acid sequence of SEQ ID NO: 211. In some embodiments, an isolated FN3 domain that binds CD71 comprises the amino acid sequence of SEQ ID NO: 212. In some embodiments, an isolated FN3 domain that binds CD71 comprises the amino acid sequence of SEQ ID NO: 213. In some embodiments, an isolated FN3 domain that binds CD71 comprises the amino acid sequence of SEQ ID NO: 214. In some embodiments, an isolated FN3 domain that binds CD71 comprises the amino acid sequence of SEQ ID NO: 215. In some embodiments, an isolated FN3 domain that binds CD71 comprises the amino acid sequence of SEQ ID NO: 216. In some embodiments, an isolated FN3 domain that binds CD71 comprises the amino acid sequence of SEQ ID NO: 217. In some embodiments, an isolated FN3 domain that binds CD71 comprises the amino acid sequence of SEQ ID NO: 218. In some embodiments, an isolated FN3 domain that binds CD71 comprises the amino acid sequence of SEQ ID NO: 219. In some embodiments, an isolated FN3 domain that binds CD71 comprises the amino acid sequence of SEQ ID NO: 220. In some embodiments, an isolated FN3 domain that binds CD71 comprises the amino acid sequence of SEQ ID NO: 221. In some embodiments, an isolated FN3 domain that binds CD71 comprises the amino acid sequence of SEQ ID NO: 222. In some embodiments, an isolated FN3 domain that binds CD71 comprises the amino acid sequence of SEQ ID NO: 223. In some embodiments, an isolated FN3 domain that binds CD71 comprises the amino acid sequence of SEQ ID NO: 224. In some embodiments, an isolated FN3 domain that binds CD71 comprises the amino acid sequence of SEQ ID NO: 225. In some embodiments, an isolated FN3 domain that binds CD71 comprises the amino acid sequence of SEQ ID NO: 226. In some embodiments, an isolated FN3 domain that binds CD71 comprises the amino acid sequence of SEQ ID NO: 227. In some embodiments, an isolated FN3 domain that binds CD71 comprises the amino acid sequence of SEQ ID NO: 228. In some embodiments, an isolated FN3 domain that binds CD71 comprises the amino acid sequence of SEQ ID NO: 229. In some embodiments, an isolated FN3 domain that binds CD71 comprises the amino acid sequence of SEQ ID NO: 230. In some embodiments, an isolated FN3 domain that binds CD71 comprises the amino acid sequence of SEQ ID NO: 231. In some embodiments, an isolated FN3 domain that binds CD71 comprises the amino acid sequence of SEQ ID NO: 232. In some embodiments, an isolated FN3 domain that binds CD71 comprises the amino acid sequence of SEQ ID NO: 233. In some embodiments, an isolated FN3 domain that binds CD71 comprises the amino acid sequence of SEQ ID NO: 234. In some embodiments, an isolated FN3 domain that binds CD71 comprises the amino acid sequence of SEQ ID NO: 235. In some embodiments, an isolated FN3 domain that binds CD71 comprises the amino acid sequence of SEQ ID NO: 236. In some embodiments, an isolated FN3 domain that binds CD71 comprises the amino acid sequence of SEQ ID NO: 237. In some embodiments, an isolated FN3 domain that binds CD71 comprises the amino acid sequence of SEQ ID NO: 238. In some embodiments, an isolated FN3 domain that binds CD71 comprises the amino acid sequence of SEQ ID NO: 239. In some embodiments, an isolated FN3 domain that binds CD71 comprises the amino acid sequence of SEQ ID NO: 240. In some embodiments, an isolated FN3 domain that binds CD71 comprises the amino acid sequence of SEQ ID NO: 241. In some embodiments, an isolated FN3 domain that binds CD71 comprises the amino acid sequence of SEQ ID NO: 242. In some embodiments, an isolated FN3 domain that binds CD71 comprises the amino acid sequence of SEQ ID NO: 243. In some embodiments, an isolated FN3 domain that binds CD71 comprises the amino acid sequence of SEQ ID NO: 244. In some embodiments, an isolated FN3 domain that binds CD71 comprises the amino acid sequence of SEQ ID NO: 245. In some embodiments, an isolated FN3 domain that binds CD71 comprises the amino acid sequence of SEQ ID NO: 246. In some embodiments, an isolated FN3 domain that binds CD71 comprises the amino acid sequence of SEQ ID NO: 247. In some embodiments, an isolated FN3 domain that binds CD71 comprises the amino acid sequence of SEQ ID NO: 248. In some embodiments, an isolated FN3 domain that binds CD71 comprises the amino acid sequence of SEQ ID NO: 249. In some embodiments, an isolated FN3 domain that binds CD71 comprises the amino acid sequence of SEQ ID NO: 250. In some embodiments, an isolated FN3 domain that binds CD71 comprises the amino acid sequence of SEQ ID NO: 251. In some embodiments, an isolated FN3 domain that binds CD71 comprises the amino acid sequence of SEQ ID NO: 252. In some embodiments, an isolated FN3 domain that binds CD71 comprises the amino acid sequence of SEQ ID NO: 253. In some embodiments, an isolated FN3 domain that binds CD71 comprises the amino acid sequence of SEQ ID NO: 254. In some embodiments, an isolated FN3 domain that binds CD71 comprises the amino acid sequence of SEQ ID NO: 255. In some embodiments, an isolated FN3 domain that binds CD71 comprises the amino acid sequence of SEQ ID NO: 256. In some embodiments, an isolated FN3 domain that binds CD71 comprises the amino acid sequence of SEQ ID NO: 257. In some embodiments, an isolated FN3 domain that binds CD71 comprises the amino acid sequence of SEQ ID NO: 258. In some embodiments, an isolated FN3 domain that binds CD71 comprises the amino acid sequence of SEQ ID NO: 259. In some embodiments, an isolated FN3 domain that binds CD71 comprises the amino acid sequence of SEQ ID NO: 260. In some embodiments, an isolated FN3 domain that binds CD71 comprises the amino acid sequence of SEQ ID NO: 261. In some embodiments, an isolated FN3 domain that binds CD71 comprises the amino acid sequence of SEQ ID NO: 262. In some embodiments, an isolated FN3 domain that binds CD71 comprises the amino acid sequence of SEQ ID NO: 263. In some embodiments, an isolated FN3 domain that binds CD71 comprises the amino acid sequence of SEQ ID NO: 264. In some embodiments, an isolated FN3 domain that binds CD71 comprises the amino acid sequence of SEQ ID NO: 265. In some embodiments, an isolated FN3 domain that binds CD71 comprises the amino acid sequence of SEQ ID NO: 266. In some embodiments, an isolated FN3 domain that binds CD71 comprises the amino acid sequence of SEQ ID NO: 267. In some embodiments, an isolated FN3 domain that binds CD71 comprises the amino acid sequence of SEQ ID NO: 268. In some embodiments, an isolated FN3 domain that binds CD71 comprises the amino acid sequence of SEQ ID NO: 269. In some embodiments, an isolated FN3 domain that binds CD71 comprises the amino acid sequence of SEQ ID NO: 270. In some embodiments, an isolated FN3 domain that binds CD71 comprises the amino acid sequence of SEQ ID NO: 271. In some embodiments, an isolated FN3 domain that binds CD71 comprises the amino acid sequence of SEQ ID NO: 272. In some embodiments, an isolated FN3 domain that binds CD71 comprises the amino acid sequence of SEQ ID NO: 273. In some embodiments, an isolated FN3 domain that binds CD71 comprises the amino acid sequence of SEQ ID NO: 274. In some embodiments, an isolated FN3 domain that binds CD71 comprises the amino acid sequence of SEQ ID NO: 275. In some embodiments, an isolated FN3 domain that binds CD71 comprises the amino acid sequence of SEQ ID NO: 276. In some embodiments, an isolated FN3 domain that binds CD71 comprises the amino acid sequence of SEQ ID NO: 277. In some embodiments, an isolated FN3 domain that binds CD71 comprises the amino acid sequence of SEQ ID NO: 278. In some embodiments, an isolated FN3 domain that binds CD71 comprises the amino acid sequence of SEQ ID NO: 279. In some embodiments, an isolated FN3 domain that binds CD71 comprises the amino acid sequence of SEQ ID NO: 280. In some embodiments, an isolated FN3 domain that binds CD71 comprises the amino acid sequence of SEQ ID NO: 281. In some embodiments, an isolated FN3 domain that binds CD71 comprises the amino acid sequence of SEQ ID NO: 282. In some embodiments, an isolated FN3 domain that binds CD71 comprises the amino acid sequence of SEQ ID NO: 283. In some embodiments, an isolated FN3 domain that binds CD71 comprises the amino acid sequence of SEQ ID NO: 284. In some embodiments, an isolated FN3 domain that binds CD71 comprises the amino acid sequence of SEQ ID NO: 285. In some embodiments, an isolated FN3 domain that binds CD71 comprises the amino acid sequence of SEQ ID NO: 286. In some embodiments, an isolated FN3 domain that binds CD71 comprises the amino acid sequence of SEQ ID NO: 287. In some embodiments, an isolated FN3 domain that binds CD71 comprises the amino acid sequence of SEQ ID NO: 288. In some embodiments, an isolated FN3 domain that binds CD71 comprises the amino acid sequence of SEQ ID NO: 289. In some embodiments, an isolated FN3 domain that binds CD71 comprises the amino acid sequence of SEQ ID NO: 290. In some embodiments, an isolated FN3 domain that binds CD71 comprises the amino acid sequence of SEQ ID NO: 291. In some embodiments, an isolated FN3 domain that binds CD71 comprises the amino acid sequence of SEQ ID NO: 292. In some embodiments, an isolated FN3 domain that binds CD71 comprises the amino acid sequence of SEQ ID NO: 293. In some embodiments, an isolated FN3 domain that binds CD71 comprises the amino acid sequence of SEQ ID NO: 294. In some embodiments, an isolated FN3 domain that binds CD71 comprises the amino acid sequence of SEQ ID NO: 295. In some embodiments, an isolated FN3 domain that binds CD71 comprises the amino acid sequence of SEQ ID NO: 296. In some embodiments, an isolated FN3 domain that binds CD71 comprises the amino acid sequence of SEQ ID NO: 297. In some embodiments, an isolated FN3 domain that binds CD71 comprises the amino acid sequence of SEQ ID NO: 298. In some embodiments, an isolated FN3 domain that binds CD71 comprises the amino acid sequence of SEQ ID NO: 299. In some embodiments, an isolated FN3 domain that binds CD71 comprises the amino acid sequence of SEQ ID NO: 300. In some embodiments, an isolated FN3 domain that binds CD71 comprises the amino acid sequence of SEQ ID NO: 301. In some embodiments, an isolated FN3 domain that binds CD71 comprises the amino acid sequence of SEQ ID NO: 302. In some embodiments, an isolated FN3 domain that binds CD71 comprises the amino acid sequence of SEQ ID NO: 303. In some embodiments, an isolated FN3 domain that binds CD71 comprises the amino acid sequence of SEQ ID NO: 304. In some embodiments, an isolated FN3 domain that binds CD71 comprises the amino acid sequence of SEQ ID NO: 305. In some embodiments, an isolated FN3 domain that binds CD71 comprises the amino acid sequence of SEQ ID NO: 306. In some embodiments, an isolated FN3 domain that binds CD71 comprises the amino acid sequence of SEQ ID NO: 307. In some embodiments, an isolated FN3 domain that binds CD71 comprises the amino acid sequence of SEQ ID NO: 308. In some embodiments, an isolated FN3 domain that binds CD71 comprises the amino acid sequence of SEQ ID NO: 309. In some embodiments, an isolated FN3 domain that binds CD71 comprises the amino acid sequence of SEQ ID NO: 310. In some embodiments, an isolated FN3 domain that binds CD71 comprises the amino acid sequence of SEQ ID NO: 311. In some embodiments, an isolated FN3 domain that binds CD71 comprises the amino acid sequence of SEQ ID NO: 312. In some embodiments, an isolated FN3 domain that binds CD71 comprises the amino acid sequence of SEQ ID NO: 313. In some embodiments, an isolated FN3 domain that binds CD71 comprises the amino acid sequence of SEQ ID NO: 314. In some embodiments, an isolated FN3 domain that binds CD71 comprises the amino acid sequence of SEQ ID NO: 315. In some embodiments, an isolated FN3 domain that binds CD71 comprises the amino acid sequence of SEQ ID NO: 316. In some embodiments, an isolated FN3 domain that binds CD71 comprises the amino acid sequence of SEQ ID NO: 317. In some embodiments, an isolated FN3 domain that binds CD71 comprises the amino acid sequence of SEQ ID NO: 318. In some embodiments, an isolated FN3 domain that binds CD71 comprises the amino acid sequence of SEQ ID NO: 319. In some embodiments, an isolated FN3 domain that binds CD71 comprises the amino acid sequence of SEQ ID NO: 320. In some embodiments, an isolated FN3 domain that binds CD71 comprises the amino acid sequence of SEQ ID NO: 321. In some embodiments, an isolated FN3 domain that binds CD71 comprises the amino acid sequence of SEQ ID NO: 322. In some embodiments, an isolated FN3 domain that binds CD71 comprises the amino acid sequence of SEQ ID NO: 323. In some embodiments, an isolated FN3 domain that binds CD71 comprises the amino acid sequence of SEQ ID NO: 324. In some embodiments, an isolated FN3 domain that binds CD71 comprises the amino acid sequence of SEQ ID NO: 325. In some embodiments, an isolated FN3 domain that binds CD71 comprises the amino acid sequence of SEQ ID NO: 326. In some embodiments, an isolated FN3 domain that binds CD71 comprises the amino acid sequence of SEQ ID NO: 327. In some embodiments, an isolated FN3 domain that binds CD71 comprises the amino acid sequence of SEQ ID NO: 328. In some embodiments, an isolated FN3 domain that binds CD71 comprises the amino acid sequence of SEQ ID NO: 329. In some embodiments, an isolated FN3 domain that binds CD71 comprises the amino acid sequence of SEQ ID NO: 330. In some embodiments, an isolated FN3 domain that binds CD71 comprises the amino acid sequence of SEQ ID NO: 331. In some embodiments, an isolated FN3 domain that binds CD71 comprises the amino acid sequence of SEQ ID NO: 332. In some embodiments, an isolated FN3 domain that binds CD71 comprises the amino acid sequence of SEQ ID NO: 333. In some embodiments, an isolated FN3 domain that binds CD71 comprises the amino acid sequence of SEQ ID NO: 334. In some embodiments, an isolated FN3 domain that binds CD71 comprises the amino acid sequence of SEQ ID NO: 335. In some embodiments, an isolated FN3 domain that binds CD71 comprises the amino acid sequence of SEQ ID NO: 336. In some embodiments, an isolated FN3 domain that binds CD71 comprises the amino acid sequence of SEQ ID NO: 337. In some embodiments, an isolated FN3 domain that binds CD71 comprises the amino acid sequence of SEQ ID NO: 338. In some embodiments, an isolated FN3 domain that binds CD71 comprises the amino acid sequence of SEQ ID NO: 339. In some embodiments, an isolated FN3 domain that binds CD71 comprises the amino acid sequence of SEQ ID NO: 340. In some embodiments, an isolated FN3 domain that binds CD71 comprises the amino acid sequence of SEQ ID NO: 341. In some embodiments, an isolated FN3 domain that binds CD71 comprises the amino acid sequence of SEQ ID NO: 342. In some embodiments, an isolated FN3 domain that binds CD71 comprises the amino acid sequence of SEQ ID NO: 343. In some embodiments, an isolated FN3 domain that binds CD71 comprises the amino acid sequence of SEQ ID NO: 344. In some embodiments, an isolated FN3 domain that binds CD71 comprises the amino acid sequence of SEQ ID NO: 345. In some embodiments, an isolated FN3 domain that binds CD71 comprises the amino acid sequence of SEQ ID NO: 346. In some embodiments, an isolated FN3 domain that binds CD71 comprises the amino acid sequence of SEQ ID NO: 347. In some embodiments, an isolated FN3 domain that binds CD71 comprises the amino acid sequence of SEQ ID NO: 348. In some embodiments, an isolated FN3 domain that binds CD71 comprises the amino acid sequence of SEQ ID NO: 349. In some embodiments, an isolated FN3 domain that binds CD71 comprises the amino acid sequence of SEQ ID NO: 350. In some embodiments, an isolated FN3 domain that binds CD71 comprises the amino acid sequence of SEQ ID NO: 351. In some embodiments, an isolated FN3 domain that binds CD71 comprises the amino acid sequence of SEQ ID NO: 352. In some embodiments, an isolated FN3 domain that binds CD71 comprises the amino acid sequence of SEQ ID NO: 353. In some embodiments, an isolated FN3 domain that binds CD71 comprises the amino acid sequence of SEQ ID NO: 354. In some embodiments, an isolated FN3 domain that binds CD71 comprises the amino acid sequence of SEQ ID NO: 355. In some embodiments, an isolated FN3 domain that binds CD71 comprises the amino acid sequence of SEQ ID NO: 356. In some embodiments, an isolated FN3 domain that binds CD71 comprises the amino acid sequence of SEQ ID NO: 357. In some embodiments, an isolated FN3 domain that binds CD71 comprises the amino acid sequence of SEQ ID NO: 358. In some embodiments, an isolated FN3 domain that binds CD71 comprises the amino acid sequence of SEQ ID NO: 359. In some embodiments, an isolated FN3 domain that binds CD71 comprises the amino acid sequence of SEQ ID NO: 360. In some embodiments, an isolated FN3 domain that binds CD71 comprises the amino acid sequence of SEQ ID NO: 361. In some embodiments, an isolated FN3 domain that binds CD71 comprises the amino acid sequence of SEQ ID NO: 362. In some embodiments, an isolated FN3 domain that binds CD71 comprises the amino acid sequence of SEQ ID NO: 363. In some embodiments, an isolated FN3 domain that binds CD71 comprises the amino acid sequence of SEQ ID NO: 364. In some embodiments, an isolated FN3 domain that binds CD71 comprises the amino acid sequence of SEQ ID NO: 365. In some embodiments, an isolated FN3 domain that binds CD71 comprises the amino acid sequence of SEQ ID NO: 366. In some embodiments, an isolated FN3 domain that binds CD71 comprises the amino acid sequence of SEQ ID NO: 367. In some embodiments, an isolated FN3 domain that binds CD71 comprises the amino acid sequence of SEQ ID NO: 368. In some embodiments, an isolated FN3 domain that binds CD71 comprises the amino acid sequence of SEQ ID NO: 369. In some embodiments, an isolated FN3 domain that binds CD71 comprises the amino acid sequence of SEQ ID NO: 370. In some embodiments, an isolated FN3 domain that binds CD71 comprises the amino acid sequence of SEQ ID NO: 371. In some embodiments, an isolated FN3 domain that binds CD71 comprises the amino acid sequence of SEQ ID NO: 372. In some embodiments, an isolated FN3 domain that binds CD71 comprises the amino acid sequence of SEQ ID NO: 373. In some embodiments, an isolated FN3 domain that binds CD71 comprises the amino acid sequence of SEQ ID NO: 374. In some embodiments, an isolated FN3 domain that binds CD71 comprises the amino acid sequence of SEQ ID NO: 375. In some embodiments, an isolated FN3 domain that binds CD71 comprises the amino acid sequence of SEQ ID NO: 376. In some embodiments, an isolated FN3 domain that binds CD71 comprises the amino acid sequence of SEQ ID NO: 377. In some embodiments, an isolated FN3 domain that binds CD71 comprises the amino acid sequence of SEQ ID NO: 378. In some embodiments, an isolated FN3 domain that binds CD71 comprises the amino acid sequence of SEQ ID NO: 379. In some embodiments, an isolated FN3 domain that binds CD71 comprises the amino acid sequence of SEQ ID NO: 380. In some embodiments, an isolated FN3 domain that binds CD71 comprises the amino acid sequence of SEQ ID NO: 381. In some embodiments, an isolated FN3 domain that binds CD71 comprises the amino acid sequence of SEQ ID NO: 382. In some embodiments, an isolated FN3 domain that binds CD71 comprises the amino acid sequence of SEQ ID NO: 383. In some embodiments, an isolated FN3 domain that binds CD71 comprises the amino acid sequence of SEQ ID NO: 384. In some embodiments, an isolated FN3 domain that binds CD71 comprises the amino acid sequence of SEQ ID NO: 385. In some embodiments, an isolated FN3 domain that binds CD71 comprises the amino acid sequence of SEQ ID NO: 386. In some embodiments, an isolated FN3 domain that binds CD71 comprises the amino acid sequence of SEQ ID NO: 387. In some embodiments, an isolated FN3 domain that binds CD71 comprises the amino acid sequence of SEQ ID NO: 388. In some embodiments, an isolated FN3 domain that binds CD71 comprises the amino acid sequence of SEQ ID NO: 389. In some embodiments, an isolated FN3 domain that binds CD71 comprises the amino acid sequence of SEQ ID NO: 390. In some embodiments, an isolated FN3 domain that binds CD71 comprises the amino acid sequence of SEQ ID NO: 391. In some embodiments, an isolated FN3 domain that binds CD71 comprises the amino acid sequence of SEQ ID NO: 392. In some embodiments, an isolated FN3 domain that binds CD71 comprises the amino acid sequence of SEQ ID NO: 393. In some embodiments, an isolated FN3 domain that binds CD71 comprises the amino acid sequence of SEQ ID NO: 394. In some embodiments, an isolated FN3 domain that binds CD71 comprises the amino acid sequence of SEQ ID NO: 395. In some embodiments, an isolated FN3 domain that binds CD71 comprises the amino acid sequence of SEQ ID NO: 396. In some embodiments, an isolated FN3 domain that binds CD71 comprises the amino acid sequence of SEQ ID NO: 397. In some embodiments, an isolated FN3 domain that binds CD71 comprises the amino acid sequence of SEQ ID NO: 398. In some embodiments, an isolated FN3 domain that binds CD71 comprises the amino acid sequence of SEQ ID NO: 399. In some embodiments, an isolated FN3 domain that binds CD71 comprises the amino acid sequence of SEQ ID NO: 400. In some embodiments, an isolated FN3 domain that binds CD71 comprises the amino acid sequence of SEQ ID NO: 401. In some embodiments, an isolated FN3 domain that binds CD71 comprises the amino acid sequence of SEQ ID NO: 402. In some embodiments, an isolated FN3 domain that binds CD71 comprises the amino acid sequence of SEQ ID NO: 403. In some embodiments, an isolated FN3 domain that binds CD71 comprises the amino acid sequence of SEQ ID NO: 404. In some embodiments, an isolated FN3 domain that binds CD71 comprises the amino acid sequence of SEQ ID NO: 405. In some embodiments, an isolated FN3 domain that binds CD71 comprises the amino acid sequence of SEQ ID NO: 406. In some embodiments, an isolated FN3 domain that binds CD71 comprises the amino acid sequence of SEQ ID NO: 407. In some embodiments, an isolated FN3 domain that binds CD71 comprises the amino acid sequence of SEQ ID NO: 408. In some embodiments, an isolated FN3 domain that binds CD71 comprises the amino acid sequence of SEQ ID NO: 409. In some embodiments, an isolated FN3 domain that binds CD71 comprises the amino acid sequence of SEQ ID NO: 410. In some embodiments, an isolated FN3 domain that binds CD71 comprises the amino acid sequence of SEQ ID NO: 411. In some embodiments, an isolated FN3 domain that binds CD71 comprises the amino acid sequence of SEQ ID NO: 412. In some embodiments, an isolated FN3 domain that binds CD71 comprises the amino acid sequence of SEQ ID NO: 413. In some embodiments, an isolated FN3 domain that binds CD71 comprises the amino acid sequence of SEQ ID NO: 414. In some embodiments, an isolated FN3 domain that binds CD71 comprises the amino acid sequence of SEQ ID NO: 415. In some embodiments, an isolated FN3 domain that binds CD71 comprises the amino acid sequence of SEQ ID NO: 416. In some embodiments, an isolated FN3 domain that binds CD71 comprises the amino acid sequence of SEQ ID NO: 417. In some embodiments, an isolated FN3 domain that binds CD71 comprises the amino acid sequence of SEQ ID NO: 418. In some embodiments, an isolated FN3 domain that binds CD71 comprises the amino acid sequence of SEQ ID NO: 419. In some embodiments, an isolated FN3 domain that binds CD71 comprises the amino acid sequence of SEQ ID NO: 420. In some embodiments, an isolated FN3 domain that binds CD71 comprises the amino acid sequence of SEQ ID NO: 421. In some embodiments, an isolated FN3 domain that binds CD71 comprises the amino acid sequence of SEQ ID NO: 422. In some embodiments, an isolated FN3 domain that binds CD71 comprises the amino acid sequence of SEQ ID NO: 423. In some embodiments, an isolated FN3 domain that binds CD71 comprises the amino acid sequence of SEQ ID NO: 424. In some embodiments, an isolated FN3 domain that binds CD71 comprises the amino acid sequence of SEQ ID NO: 425. In some embodiments, an isolated FN3 domain that binds CD71 comprises the amino acid sequence of SEQ ID NO: 426. In some embodiments, an isolated FN3 domain that binds CD71 comprises the amino acid sequence of SEQ ID NO: 427. In some embodiments, an isolated FN3 domain that binds CD71 comprises the amino acid sequence of SEQ ID NO: 428. In some embodiments, an isolated FN3 domain that binds CD71 comprises the amino acid sequence of SEQ ID NO: 429. In some embodiments, an isolated FN3 domain that binds CD71 comprises the amino acid sequence of SEQ ID NO: 430. In some embodiments, an isolated FN3 domain that binds CD71 comprises the amino acid sequence of SEQ ID NO: 431. In some embodiments, an isolated FN3 domain that binds CD71 comprises the amino acid sequence of SEQ ID NO: 432. In some embodiments, an isolated FN3 domain that binds CD71 comprises the amino acid sequence of SEQ ID NO: 433. In some embodiments, an isolated FN3 domain that binds CD71 comprises the amino acid sequence of SEQ ID NO: 434. In some embodiments, an isolated FN3 domain that binds CD71 comprises the amino acid sequence of SEQ ID NO: 435. In some embodiments, an isolated FN3 domain that binds CD71 comprises the amino acid sequence of SEQ ID NO: 436. In some embodiments, an isolated FN3 domain that binds CD71 comprises the amino acid sequence of SEQ ID NO: 437. In some embodiments, an isolated FN3 domain that binds CD71 comprises the amino acid sequence of SEQ ID NO: 438. In some embodiments, an isolated FN3 domain that binds CD71 comprises the amino acid sequence of SEQ ID NO: 439. In some embodiments, an isolated FN3 domain that binds CD71 comprises the amino acid sequence of SEQ ID NO: 440. In some embodiments, an isolated FN3 domain that binds CD71 comprises the amino acid sequence of SEQ ID NO: 441. In some embodiments, an isolated FN3 domain that binds CD71 comprises the amino acid sequence of SEQ ID NO: 442. In some embodiments, an isolated FN3 domain that binds CD71 comprises the amino acid sequence of SEQ ID NO: 443. In some embodiments, an isolated FN3 domain that binds CD71 comprises the amino acid sequence of SEQ ID NO: 444. In some embodiments, an isolated FN3 domain that binds CD71 comprises the amino acid sequence of SEQ ID NO: 445. In some embodiments, an isolated FN3 domain that binds CD71 comprises the amino acid sequence of SEQ ID NO: 446. In some embodiments, an isolated FN3 domain that binds CD71 comprises the amino acid sequence of SEQ ID NO: 447. In some embodiments, an isolated FN3 domain that binds CD71 comprises the amino acid sequence of SEQ ID NO: 448. In some embodiments, an isolated FN3 domain that binds CD71 comprises the amino acid sequence of SEQ ID NO: 449. In some embodiments, an isolated FN3 domain that binds CD71 comprises the amino acid sequence of SEQ ID NO: 450. In some embodiments, an isolated FN3 domain that binds CD71 comprises the amino acid sequence of SEQ ID NO: 451. In some embodiments, an isolated FN3 domain that binds CD71 comprises the amino acid sequence of SEQ ID NO: 452. In some embodiments, an isolated FN3 domain that binds CD71 comprises the amino acid sequence of SEQ ID NO: 453. In some embodiments, an isolated FN3 domain that binds CD71 comprises the amino acid sequence of SEQ ID NO: 454. In some embodiments, an isolated FN3 domain that binds CD71 comprises the amino acid sequence of SEQ ID NO: 455. In some embodiments, an isolated FN3 domain that binds CD71 comprises the amino acid sequence of SEQ ID NO: 456. In some embodiments, an isolated FN3 domain that binds CD71 comprises the amino acid sequence of SEQ ID NO: 457. In some embodiments, an isolated FN3 domain that binds CD71 comprises the amino acid sequence of SEQ ID NO: 458. In some embodiments, an isolated FN3 domain that binds CD71 comprises the amino acid sequence of SEQ ID NO: 459. In some embodiments, an isolated FN3 domain that binds CD71 comprises the amino acid sequence of SEQ ID NO: 460. In some embodiments, an isolated FN3 domain that binds CD71 comprises the amino acid sequence of SEQ ID NO: 461. In some embodiments, an isolated FN3 domain that binds CD71 comprises the amino acid sequence of SEQ ID NO: 462. In some embodiments, an isolated FN3 domain that binds CD71 comprises the amino acid sequence of SEQ ID NO: 463. In some embodiments, an isolated FN3 domain that binds CD71 comprises the amino acid sequence of SEQ ID NO: 464. In some embodiments, an isolated FN3 domain that binds CD71 comprises the amino acid sequence of SEQ ID NO: 465. In some embodiments, an isolated FN3 domain that binds CD71 comprises the amino acid sequence of SEQ ID NO: 466. In some embodiments, an isolated FN3 domain that binds CD71 comprises the amino acid sequence of SEQ ID NO: 467. In some embodiments, an isolated FN3 domain that binds CD71 comprises the amino acid sequence of SEQ ID NO: 468. In some embodiments, an isolated FN3 domain that binds CD71 comprises the amino acid sequence of SEQ ID NO: 469. In some embodiments, an isolated FN3 domain that binds CD71 comprises the amino acid sequence of SEQ ID NO: 470. In some embodiments, an isolated FN3 domain that binds CD71 comprises the amino acid sequence of SEQ ID NO: 471. In some embodiments, an isolated FN3 domain that binds CD71 comprises the amino acid sequence of SEQ ID NO: 472. In some embodiments, an isolated FN3 domain that binds CD71 comprises the amino acid sequence of SEQ ID NO: 473. In some embodiments, an isolated FN3 domain that binds CD71 comprises the amino acid sequence of SEQ ID NO: 474. In some embodiments, an isolated FN3 domain that binds CD71 comprises the amino acid sequence of SEQ ID NO: 475. In some embodiments, an isolated FN3 domain that binds CD71 comprises the amino acid sequence of SEQ ID NO: 476. In some embodiments, an isolated FN3 domain that binds CD71 comprises the amino acid sequence of SEQ ID NO: 477. In some embodiments, an isolated FN3 domain that binds CD71 comprises the amino acid sequence of SEQ ID NO: 478. In some embodiments, an isolated FN3 domain that binds CD71 comprises the amino acid sequence of SEQ ID NO: 479. In some embodiments, an isolated FN3 domain that binds CD71 comprises the amino acid sequence of SEQ ID NO: 480. In some embodiments, an isolated FN3 domain that binds CD71 comprises the amino acid sequence of SEQ ID NO: 481. In some embodiments, an isolated FN3 domain that binds CD71 comprises the amino acid sequence of SEQ ID NO: 482. In some embodiments, an isolated FN3 domain that binds CD71 comprises the amino acid sequence of SEQ ID NO: 483. In some embodiments, an isolated FN3 domain that binds CD71 comprises the amino acid sequence of SEQ ID NO: 484. In some embodiments, an isolated FN3 domain that binds CD71 comprises the amino acid sequence of SEQ ID NO: 485. In some embodiments, an isolated FN3 domain that binds CD71 comprises the amino acid sequence of SEQ ID NO: 486. In some embodiments, an isolated FN3 domain that binds CD71 comprises the amino acid sequence of SEQ ID NO: 487. In some embodiments, an isolated FN3 domain that binds CD71 comprises the amino acid sequence of SEQ ID NO: 488. In some embodiments, an isolated FN3 domain that binds CD71 comprises the amino acid sequence of SEQ ID NO: 489. In some embodiments, an isolated FN3 domain that binds CD71 comprises the amino acid sequence of SEQ ID NO: 490. In some embodiments, an isolated FN3 domain that binds CD71 comprises the amino acid sequence of SEQ ID NO: 491. In some embodiments, an isolated FN3 domain that binds CD71 comprises the amino acid sequence of SEQ ID NO: 492. In some embodiments, an isolated FN3 domain that binds CD71 comprises the amino acid sequence of SEQ ID NO: 493. In some embodiments, an isolated FN3 domain that binds CD71 comprises the amino acid sequence of SEQ ID NO: 494. In some embodiments, an isolated FN3 domain that binds CD71 comprises the amino acid sequence of SEQ ID NO: 495. In some embodiments, an isolated FN3 domain that binds CD71 comprises the amino acid sequence of SEQ ID NO: 496. In some embodiments, an isolated FN3 domain that binds CD71 comprises the amino acid sequence of SEQ ID NO: 497. In some embodiments, an isolated FN3 domain that binds CD71 comprises the amino acid sequence of SEQ ID NO: 498. In some embodiments, an isolated FN3 domain that binds CD71 comprises the amino acid sequence of SEQ ID NO: 499. In some embodiments, an isolated FN3 domain that binds CD71 comprises the amino acid sequence of SEQ ID NO: 500. In some embodiments, an isolated FN3 domain that binds CD71 comprises the amino acid sequence of SEQ ID NO: 501. In some embodiments, an isolated FN3 domain that binds CD71 comprises the amino acid sequence of SEQ ID NO: 502. In some embodiments, an isolated FN3 domain that binds CD71 comprises the amino acid sequence of SEQ ID NO: 503. In some embodiments, an isolated FN3 domain that binds CD71 comprises the amino acid sequence of SEQ ID NO: 504. In some embodiments, an isolated FN3 domain that binds CD71 comprises the amino acid sequence of SEQ ID NO: 505. In some embodiments, an isolated FN3 domain that binds CD71 comprises the amino acid sequence of SEQ ID NO: 506. In some embodiments, an isolated FN3 domain that binds CD71 comprises the amino acid sequence of SEQ ID NO: 507. In some embodiments, an isolated FN3 domain that binds CD71 comprises the amino acid sequence of SEQ ID NO: 508. In some embodiments, an isolated FN3 domain that binds CD71 comprises the amino acid sequence of SEQ ID NO: 509. In some embodiments, an isolated FN3 domain that binds CD71 comprises the amino acid sequence of SEQ ID NO: 510. In some embodiments, an isolated FN3 domain that binds CD71 comprises the amino acid sequence of SEQ ID NO: 511. In some embodiments, an isolated FN3 domain that binds CD71 comprises the amino acid sequence of SEQ ID NO: 512. In some embodiments, an isolated FN3 domain that binds CD71 comprises the amino acid sequence of SEQ ID NO: 513. In some embodiments, an isolated FN3 domain that binds CD71 comprises the amino acid sequence of SEQ ID NO: 514. In some embodiments, an isolated FN3 domain that binds CD71 comprises the amino acid sequence of SEQ ID NO: 515. In some embodiments, an isolated FN3 domain that binds CD71 comprises the amino acid sequence of SEQ ID NO: 516. In some embodiments, an isolated FN3 domain that binds CD71 comprises the amino acid sequence of SEQ ID NO: 517. In some embodiments, an isolated FN3 domain that binds CD71 comprises the amino acid sequence of SEQ ID NO: 518. In some embodiments, an isolated FN3 domain that binds CD71 comprises the amino acid sequence of SEQ ID NO: 519. In some embodiments, an isolated FN3 domain that binds CD71 comprises the amino acid sequence of SEQ ID NO: 520. In some embodiments, an isolated FN3 domain that binds CD71 comprises the amino acid sequence of SEQ ID NO: 521. In some embodiments, an isolated FN3 domain that binds CD71 comprises the amino acid sequence of SEQ ID NO: 522. In some embodiments, an isolated FN3 domain that binds CD71 comprises the amino acid sequence of SEQ ID NO: 523. In some embodiments, an isolated FN3 domain that binds CD71 comprises the amino acid sequence of SEQ ID NO: 524. In some embodiments, an isolated FN3 domain that binds CD71 comprises the amino acid sequence of SEQ ID NO: 525. In some embodiments, an isolated FN3 domain that binds CD71 comprises the amino acid sequence of SEQ ID NO: 526. In some embodiments, an isolated FN3 domain that binds CD71 comprises the amino acid sequence of SEQ ID NO: 527. In some embodiments, an isolated FN3 domain that binds CD71 comprises the amino acid sequence of SEQ ID NO: 528. In some embodiments, an isolated FN3 domain that binds CD71 comprises the amino acid sequence of SEQ ID NO: 529. In some embodiments, an isolated FN3 domain that binds CD71 comprises the amino acid sequence of SEQ ID NO: 530. In some embodiments, an isolated FN3 domain that binds CD71 comprises the amino acid sequence of SEQ ID NO: 531. In some embodiments, an isolated FN3 domain that binds CD71 comprises the amino acid sequence of SEQ ID NO: 532. In some embodiments, an isolated FN3 domain that binds CD71 comprises the amino acid sequence of SEQ ID NO: 533. In some embodiments, an isolated FN3 domain that binds CD71 comprises the amino acid sequence of SEQ ID NO: 534. In some embodiments, an isolated FN3 domain that binds CD71 comprises the amino acid sequence of SEQ ID NO: 535. In some embodiments, an isolated FN3 domain that binds CD71 comprises the amino acid sequence of SEQ ID NO: 536. In some embodiments, an isolated FN3 domain that binds CD71 comprises the amino acid sequence of SEQ ID NO: 537. In some embodiments, an isolated FN3 domain that binds CD71 comprises the amino acid sequence of SEQ ID NO: 538. In some embodiments, an isolated FN3 domain that binds CD71 comprises the amino acid sequence of SEQ ID NO: 539. In some embodiments, an isolated FN3 domain that binds CD71 comprises the amino acid sequence of SEQ ID NO: 540. In some embodiments, an isolated FN3 domain that binds CD71 comprises the amino acid sequence of SEQ ID NO: 541. In some embodiments, an isolated FN3 domain that binds CD71 comprises the amino acid sequence of SEQ ID NO: 542. In some embodiments, an isolated FN3 domain that binds CD71 comprises the amino acid sequence of SEQ ID NO: 543. In some embodiments, an isolated FN3 domain that binds CD71 comprises the amino acid sequence of SEQ ID NO: 544. In some embodiments, an isolated FN3 domain that binds CD71 comprises the amino acid sequence of SEQ ID NO: 545. In some embodiments, an isolated FN3 domain that binds CD71 comprises the amino acid sequence of SEQ ID NO: 546. In some embodiments, an isolated FN3 domain that binds CD71 comprises the amino acid sequence of SEQ ID NO: 547. In some embodiments, an isolated FN3 domain that binds CD71 comprises the amino acid sequence of SEQ ID NO: 548. In some embodiments, an isolated FN3 domain that binds CD71 comprises the amino acid sequence of SEQ ID NO: 549. In some embodiments, an isolated FN3 domain that binds CD71 comprises the amino acid sequence of SEQ ID NO: 550. In some embodiments, an isolated FN3 domain that binds CD71 comprises the amino acid sequence of SEQ ID NO: 551. In some embodiments, an isolated FN3 domain that binds CD71 comprises the amino acid sequence of SEQ ID NO: 552. In some embodiments, an isolated FN3 domain that binds CD71 comprises the amino acid sequence of SEQ ID NO: 972. In some embodiments, an isolated FN3 domain that binds CD71 comprises the amino acid sequence of SEQ ID NO: 973. In some embodiments, an isolated FN3 domain that binds CD71 comprises the amino acid sequence of SEQ ID NO: 974. In some embodiments, an isolated FN3 domain that binds CD71 comprises the amino acid sequence of SEQ ID NO: 975. In some embodiments, an isolated FN3 domain that binds CD71 comprises the amino acid sequence of SEQ ID NO: 976.

In some embodiments, the isolated FN3 domain that binds CD71 comprises an initiator methionine (Met) linked to the N-terminus of the molecule. In some embodiments, the polypeptide does not comprise a N-terminal methionine.

In some embodiments, the isolated FN3 domain that binds CD71 comprises an amino acid sequence that is 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to one of the amino acid sequences of SEQ ID NOs: 100-209, 211-301, 303-317, 319-552, and 972-976. Percent identity can be determined using the default parameters to align two sequences using BlastP available through the NCBI website. The sequences of the FN3 domains that bind to CD71 can be found, for example, in Table 3. These sequences are illustrated with a N-terminal methionine. The sequence of the domain can also be utilized without the N-terminal methionine. Simply for the avoidance of duplicating almost identical sequences, a table of such sequences is not being provided, but one of skill in the art could immediately envisage the sequences provided for herein without the N-terminal methionine and the disclosure should be understood and construed to include such sequences.

TABLE 3
CD71-binding FN3 Domains

| SEQ ID | |
|---|---|
| NO | SEQUENCE |
| 100 | MLPAPKNLVVSRVTEDSARLSWTAPDAAFDSFYIAYAEPRPDGEAILLLVPGS |
| | ERSYDLTGLKPGTEYSVLIHGVKGGLLSSPLVASFTT |
| 101 | MLPAPKNLVVSRVTEDSARLSWTAPDAAFDSFGIGYWERRWYGEAIVLTVPGS |
| | ERSYDLTGLKPGTEYEVTIRGVKGGGYSGPLSAIFTT |
| 102 | MLPAPKNLVVSRVTEDSARLSWTAPDAAFDSFDIGYTEYGGYGEAIILQVPGS |
| | ERSYDLTGLKPGTEYWVLIQGVKGGGSSVPLVAYFTT |
| 103 | MLPAPKNLVVSRVTEDSARLSWTAPDAAFDSFYIAYAEPRPDGEAIILGVPGS |
| | ERSYDLTGLKPGTEYSVLIHGVKGGLLSSPLSAYSTT |
| 104 | MLPAPKNLVVSRVTEDSARLSWTAPDAAFDSFFIGYLEPQPPGEAIHLIVPGS |
| | ERSYDLTGQKPGTEYNVTIQGVKGGFPSDPLVASFTT |
| 105 | MLPAPKNLVVSRVTEDSARLSWTAPDAAFDSFHIVYHEPRPSGEAIWLWVPGS |
| | ERSYDLTGLKPGTEYEVGIVSVKGGDLSVPLSAIFTT |
| 106 | MLPAPKNLVVSRVTEDSARLSWTAPDAVEDSFYIAYAEPRPDGEAIGLYVPGS |
| | ERSYDLTGLKPGTEYSVLIHGVKGGLLSSPLTASFTT |
| 107 | MLPAPKNLVVSRVTEDSARLSWTAPDAAFDSFFIGYLEPQPPGEAIGLQVPGS |
| | ERSYDLTGLKPGTEYNVTIQGVKGGFPSMPLSAIFTT |
| 108 | MLPAPKNLVVSRVTEDSARLSWTAPDAAFDSFYIAYAEPRPDGEAILLQVPGS |
| | ERSYDLTGLKPGTEYSVLIHGVKGGLLSSPLVAKFTT |
| 109 | MLPAPKNLVVSRVTEDSARLSWTAPDAAFDSFFIGYLEPQPPGEAIILHVPGS |
| | ERSYDLTGLKPGTEYNVTIQGVKGGFPSLPLNANFTT |
| 110 | MLPAPKNLVVSRVTEDSARLSWTAPDAAFDSFHIVYHEPRPSGEAIYLEVPGS |
| | ERSYDLTGLKPGTEYEVGIVSVKGGDLSVPLRAHFTT |
| 111 | MLPAPKNLVVSRVTEDSARLSWTAPDAAFDSFFIGYLEPQPPGEAISLWVPGS |
| | ERSYDLTGLKPGTEYNVTIQGVKGGFPSIPLWAIFTT |
| 112 | MLPAPKNLVVSRVTEDSARLSWTAPDAAFDSFFIGYLEPQPPGEAIDLWVPGS |
| | ERSYDLTGLKPGTEYNVTIQGVKGGFPSMPLSAIFTT |
| 113 | MLPAPKNLVVSRVTEDSARLSWTAPDAAFDSFFIGYLEPQPPGEAIYLLVPGS |
| | ERSYDLTGLKPGTEYNVTIQGVKGGFPSDPLSAIFTT |
| 114 | MLPAPKNLVVSRVTEDSARLSWTAPDAAFDSFHIVYHEPRPSGEAIWLFVPGS |
| | ERSYDLTGLKPGTEYEVGIVSVKGGDLSVPLSAIFTT |
| 115 | MLPAPKNLVVSRVTEDSARLSWTAPDAAFDSFFIGYLEPQPPGEAIDLYVPGS |
| | ERSYDLTGLKPGIEYNVTIQGVKGGFPSLPLQAHFTT |
| 116 | MLPAPKNLVVSRVTEDSARLSWTAPDAAFDSFYIAYAEPRPDGEAILLLVPGS |
| | ERSYDLTGLKPGTEYSVLIHGVKGGLLSSPLIAGFTT |
| 117 | MLPAPKNLVVSRVTEDSARLSWTAPDAAFDSFFIGYLEPQPPGEAIWLLVPGS |
| | ERSYDLTGLKPGTEYNVTIQGVKGGFPSTPLSAIFTT |
| 118 | MLPAPKNLVVSRVTEDSARLSWTAPDAAFDSFYIAYAEPRPDGEAITLWVPGS |
| | ERSYDLTGLKPGTEYSVTIHGVKGGLLSSPLSAIFTT |
| 119 | MSLPAPKNLVVSRVTEDSARLSWTAPDAAFDSFFIGYLEPQPPGEAIELYVPG |
| | SERSYDLTGLKPGTEYNVTIQGVKGGFPSDPLVAFFTT |
| 120 | MSLPAPKNLVVSRVTEDSARLSWTAPDAAFDSFEIAYQELSLWGEAIVLTVPG |
| | SERSYDLTGLKPGTEYWVQIGGVKGGEWSAPLSAIFTT |
| 121 | MSLPAPKNLVVSRVTEDSARLSWTAPDAAFDSFFIGYLEPQPPGEAITLYVPG |
| | SERSYDLTGLKPGTEYNVTIQGVKGGFPSAPLSAIFTT |
| 122 | MSLPAPKNLVVSRVTEDSARPSWTAPDAAFDSFEIGYFENLYLGEAIVLTVPG |
| | SERSYDLTGLKPGTEYYVHIGGVKGGIWSSPLSAYFTT |
| 123 | MSLPAPKNLVVSRVTEDSARLSWTAPDAAFDSFAIRYWELRATGEAIPLSVPG |
| | SERSYDLTGLKPGTEYHVAISGVKGGKSSYPLRASFTT |
| 124 | MSLPAPKNLVVSRVIEDSARLSWTAPDAAFDSFEIGYFENLYLGEAIVLTVPG |
| | SERSYDLTGLKPGTEYWVQIGGVKGGNWSAPLSAIFTT |
| 125 | MSLPAPKNLVVSRVTEDSARLSWTAPDAAFDSFYIAYAEPRPDGEAIILLVPG |
| | SERSYDLTGLKPGTEYSVLIHGVKGGLLSSPLHAHFTT |
| 126 | MSLPAPKNLVVSRVTEDSARLSWTAPDAVEDSFEIAYQELSLWGEAIVLTVPG |
| | SERSYDLTGLKPGTEYYVHIGGVKGGIWSSPLSAYFTT |
| 127 | MSLPAPKNLVVSRVTEDSARLSWTAPDAAFDSFFIGYLEPQPPGEAITLLVPG |
| | SERSYDLTGLKPGTEYNVTIQGVKGGFPSGPLQASFTT |
| 128 | MSSLPAPKNLVVSRVTEDSARLSWTAPDAAFDSFEIGYFENLYLGEAIVLTVP |
| | GSERSYDLTGLKPGTEYWVQIGGVKGGNWSAPLSAIFTT |
| 129 | MGGSLPAPKNLVVSRVTEDSARLSWTAPDAAFDSFEIGYFENLYLGEAIVLTV |
| | PGSERSYDLTGLKPGTEYWVQIGGVKGGNWSAPLSAIFTT |
| 130 | MSLPAPKNLVVSRVTEDSARLSWTAPDAAFDSFEIAYFERTWFGEAIVLTVPG |
| | SERSYDLTGLKPGTEYWVQIGGVKGGDWSAPLSAIFTT |
| 131 | MSLPAPKNLVVSRVTEDSARLSWTAPDAAFDSFEIAYFENDWAGEAIVLTVPG |
| | SERSYDLTGLKPGTEYVVFIGGVKGGDFSPPLSAIFTT |
| 132 | MSLPAPKNLVVSRVTEDSARLSWTAPDAAFDSFEIGYFENLYLGEAIVLTVPG |
| | SERSYDLTGLKPGTEYWVQIGGVKGVNWSAPLSAIFTT |
| 133 | MSLPAPKNLVVSRVTEDSARLSWTAPDAAFDSFKIKYAEYDRYGEAIALFVPG |
| | SERSYDLTGLKPGTEYFVHIDGVKGGTDSQPLVASFTT |
| 134 | MSLPAPKNLVVSRVTEDSARLSWTAPDAAFDSFDINYWENESGGEAIALFVPG |
| | SERSYDLTGLKPGTEYLVTIAGVKGGWWSKPLYATFTT |
| 135 | MSLPAPKNLVVSRVTEDSARLSWTAPDAAFDSFFIGYLEPQSPGEAIALYVPG |
| | SERSYDLTGLKPGTEYNVTIQGVKGGFPSEPLIANFTT |
| 136 | MSLPAPKNLVVSRVTEDSARLSWTAPDAAFDSFEIDYVEFIFTGEAIGLIVPG |
| | SERSYDLTGLKPGTEYWVTIAGVKGGEWSTPLQAFLTT |
| 137 | MSLPAPKNLVVSRVTEDSARLSWTAPDAAFDSFFIGYLEPQPPGEAITLYVPG |
| | SERSYDLTGLKPGTEYNVTIQGVKGGFPSDPLVAHFTT |
| 138 | MSLPAPKNLVVSRVTEDSARLSWTAPDAAFDSFFIGYLEPQPPGEVIHLFVPG |
| | SERSYDLTGLKPGTEYNVTIQGVKGGFPSMPLSAIFTT |
| 139 | MSLPAPKNLVVSRVTEDSARLSWTAPDAAFDSFEIGYFENLYLGEAIVLTVPG |
| | SERSYDLTGLKPGTEYWVQIGGVKGGAYSTPLSAIFTT |
| 140 | MSLPAPKNLVVSRVTEDSARLSWTAPDAAFDSFFIGYLEPQPPGEAIHLYVPG |
| | SERSYDLTGLKPGTEYNVTIQGVKGGFPSMPLSAIFTT |
| 141 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAYFTT |
| 142 | MSLPAPKNLVVSRVTEDSARLSWTASDAAFDSFHIWYFEKANEGEAIPLVVPG |
| | SERSYDLTGLKPGTEYDVDIGGVKGGAWSIPLGARFTT |
| 143 | MSLPAPKNLVVSRVTEDSARLSWTAPDAAFDSFAIQYVEVIGSGEAIELIVPG |
| | SERSYDLTGLKPGTEYHVYIDGVKGGKDSKPLYAGFTT |
| 144 | MSLPAPKNLVVSRVTEDSARLSWTAPDAAFDSFEIGYFENLYLGEAIVLTVPG |
| | SERSYDLTSLKPGTEYWVQIGGVKGGNWSAPLSATFTT |
| 145 | MSLPAPKNLVVSRVTEDSARLSWTAPDAAFDSFFIGYLEPQPPGEAIALLVPG |
| | SERSYDLTGLKPGTEYNVTIQGVKGGFPSHPLSAKFTT |
| 146 | MSLPAPKNLVVSRVTEDSARLSWTAPDAAFDSEDIGYFENTYEGEAIVLTVPG |
| | SERSYDLTGLKPGTEYVVWIGGVKGGSYSSPLSAIFTT |
| 147 | MSLPAPKNLVVSRVTEDSARLSWTAPDAAFDSFFIGYLEPQPPGEAIELFVPG |
| | SERSYDLTGLKPGTEYNVTIQGVKGGFPSGPLIASFTT |
| 148 | MSLPAPKNLVVSRVTEDSARLSWTAPDAAFDSFEIVYQENSAYGEAIVLTVPG |
| | SERSYDLTGLKPGTEYWVQIGGVKGGEWSAPLSAIFTT |
| 149 | MSLPAPKNLVVSRVTEDSARLSWTAPDAAFDSFDIGYFELRYYGEAIVLTVPG |
| | SERSYDLTGLKPGTEYWVSIGGVKGGTYSAPLSAIFTT |
| 150 | MSLPAPKNLVVSRVTEDSARLSWTAPDAAFDSFDIGYFENSYAGEAIVLTVPG |
| | SERSYDLTGLKPGTEYYVSIAGVKGGRESPPLSAIFTT |
| 151 | MSLPAPKNLVVSRITEDSARLSWTAPDAAFDSFEIAYFETCRTGEAIVLTVPG |
| | SERSYDLTGLKPGTEYRVWIGGVKGGVWSRPLSAIVTT |
| 152 | MSLPAPKNLVVSRVTEDSARLSWTAPDAAFDSFDIGYFEATYYGEAIVLTVPG |
| | SERSYDLTGLKPGTEYWVQIGGVKGGNWSAPLSAIFTT |
| 153 | MSLPAPKNLVVSRVTEDSARLSWTAPDAAFDSFYIAYAEPRPDGEAIVLAVPG |
| | SERSYDLTGLKPGTEYSVLIHGVKGGLLSSPLGADETT |
| 154 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 155 | MSLPAPENLVVSRVTEDSARLSWTAPDAAFDSFEIGYFENLYLGEAIVLTVPG |
| | SERSYDLTGLKPGTEYWVQIGGVKGGVWSRPLSAIFTT |
| 156 | MSLPAPKNLVVSRVTEDSARLSWTAPDAAFDSFEIGYFEMSWTGEAIVLTVPG |
| | SERSYDLTGLKPGTEYVVFIGGVKGGNWSAPLSAIFTT |
| 157 | MSLPAPKNLVVSRVTEDSARLSWTAPDAAFDSFEIGYGEFTYYGEAIVLTVPG |
| | SERSYDLTGLKPGTEYWVQIGGVKGGNWSNPLSAIFTT |
| 158 | MSLPAPKNLVVSRVTEDSARLSWTAPDAAFDSFEIAYFERTWFGEAIVLTVPG |
| | SERSYDLTGLKPGTEYWVQIGGVKGGDWSKPLSAIFTT |
| 159 | MSLPAPKNLVVSRVTEDSARLSWTAPDAAFDSFEIVYQENSAYGEAIVLTVPG |
| | SERSYDLTGLKPGTEYWVQIGGVKGGNWSAPLSAISTT |
| 160 | MSLPAPKNLVVSRVTEDSARLSWTAPDAAFDSFEIGYFENLYLGEAIVLTVPG |
| | SERSYDLTGLKPGTEYWVQIGGVKGGNWSVPLSAIFTT |
| 161 | MSLPAPKNLVVSRVTEDSARLSWTAPDAAFDSFFIGYLEPQPPGEAIVLTVPG |
| | SERSYDLTGLKPGTEYNVTIQGVKGGFPSEPLVAHFTT |
| 162 | MSLPAPKNLVVSRVTEDSARLSWTALDAAFDSFEISYWEHTENGEAIILFVPG |
| | SERSYDLTGLKPGTEYYVTIGGVKGGAWSPPLWAEFTT |
| 163 | MSLPAPKNLVVSRVTEDSARLSWTAPDAAFDSFFIGYLEPQPPGEVIHLYVPG |
| | SERSYDLTGLKPGTEYVVFIGGVKGGVWSVPLSAIFTT |
| 164 | MSLPAPKNLVVSRVTEDSARLSWTAPDAAFDSFFIGYLEPQPPGEAIHLYVPG |
| | SERSYDLTGLKPGTEYWVQIGGVKGGNWSAPLSAIFTT |
| 165 | MSLPAPKNLVVSRVTEDSARLSWTAPDAAFDSFYIAYAEPRPDGEAILLEVPG |
| | SERSYDLAGLKPGTEYSVLIHGVKGGLLSSPLGAEFTT |
| 166 | MSLPAPKNLVVSRVTEDSARLSWTAPDAAFDSFEIGYFENLYLGEAIVLTVPG |
| | SERSYDLTGLKPGTEYWVQIGGIKGGNWSAPLSAIFTT |
| 167 | MSLPAPKNLVVSRVTEDSARLSWTAPDAAFDSFFIGYLEPQPPGEAIVLTVPG |
| | SERSYDLTGLKPGTEYNVTIQGVKGGFPSVPLSAAFTT |
| 168 | MSLPAPKNLVVSRVTEDSARLSWTAPDAAFDSFIIGYLEPQPPDEAIALYVPG |
| | SERSYDLTGLKPGTEYNVTIQGVKGGFPSLPLVATFTT |
| 169 | MSLPAPKNLVVSRVTEDSARLSWTAPDAAFDSFEIGYFENLYLGEAIVLTVPG |
| | SERSYDLTGLKPGTEYWVQIGGVKGGNWSAPLSATFTT |
| 170 | MSLPAPKNLVVSRVTEDSARLSWTAPDAAFDSFEIGYFENLYLGEAIVLTVPG |
| | SERSYDLTGLKPGTEYWVQIGGVKGGNWSTPLSAIFTT |
| 171 | MSLPAPKNLVVSRVTEDSARLSWTAPDAAFDSFEIGYFENLYLGEAIVLTVPG |
| | SERSYDLTGLKPGTEYWVQIGGVKGGEWSSPLSAIFTT |
| 172 | MSRPAPKNLVVSRVTEDSARLSWTAPGAAFDSFEIAYFERTWFGEAIVLTVPS |
| | SERSYDLTGLKPGTEYWVQIGGVKGGDWSKPLSAIFTT |
| 173 | MSLPAPKNLVVSRVTEDSARLSWTAPDAAFDSFEIAYQELSLWGEAIVLTVPG |
| | SERSYDLTGLKPGTEYYVHIGGVKGGIWSSPLVAHFTT |
| 174 | MSLPAPKNLVVSRVTEDSARLSWTAPNAAFDSFEIGYFENLYLGEAIVLTVPG |
| | SERSYDLTGLKPGTEYWVQIGGVKGGNWSAPLSAIFTT |
| 175 | MSLPAPKNLVVSRVTEDSARLSWTAPDAAFDSFEIAYQELSLWGEAIVLTVPG |
| | SERSYDLTGLKPGTEYVVFIGGVKGGVWSVPLSAIFTT |
| 176 | MSLPAPKNLVVSRVTEDSARLSWTAPDAAFDSFFIGYLEPQPPGEAIHLFVPG |
| | SERSYDLTGLKPGTEYWVQIGGVKGGNWSAPLSAIFTT |
| 177 | MSLPAPKNLVVSRVTEDSARLSWTAPDAAFDSFEIGYFENLYLGEAIVLTVPG |
| | SERSYDLTSLKPGTEYWVQIGGVKGGNWSAPLSAIFTT |
| 178 | MGSLPAPKNLVVSRVTEDSARLSWTAPDAAFDSFFIGYLEPQPPGEAISLYVP |
| | GSERSYDLTGLKPGTEYNVTIQGVKGGFPSDPLVAHFTT |
| 179 | MSLPAPKNLVVSRVTEDSARLSWTAPDAAFDSFFIGYLEPQPPGEAITLLVPG |
| | SERSYDLTGLKPGTEYNVTIQGVKGGFPSHPLVATTTT |
| 180 | MSLPAPKNLVVSRVTEDSARLSWTAPDAAFDSFFIGYLEPQPPGEAISLYVPG |
| | SERSYDLTGLKPGTEYNVTIQGVKGGFPSEPLIANFTT |
| 181 | MSLPAPKNLVVSRVTEDSARLSWTAPDAAFDSFEIGYFENDYFGEAIVLTVPG |
| | SERSYDLTGLKPGTEYWVQIGGVKGGNWSAPLSAIFTT |
| 182 | MSLPAPENLVVSRVTEDSARLSWTAPDAAFDSFEIGYWENAYKGEAIVLTVPG |
| | SERSYDLTGLKPGTEYVVFIGGVKGGVWSVPLSVIFTT |
| 183 | MSLPAPKNLVISRVTEDSARLSWTAPDAAFDSFEIVYAEPVRFGEAIVLTVPG |
| | SERSYDLTGLKPGTEYWVQIGGVKGGEWSSPLSAIFTT |
| 184 | MSLPAPKNLVVSRVTEDSARPSWTAPDAAFDSFEIAYFERTWFGEAIVLTVPG |
| | SERSYDLTGLKPGTEYWVQIGGVKGGDWSKPLSAIFTT |
| 185 | MGSLPAPKNLVVSRVTEDSARLSWTAPDAAFDSFEISYWEHTENGEAIILFVP |
| | GSERSYDLTGLKPGTEYWVQIGGVKGGNWSAPLSAIFTT |
| 186 | MSLPAPKNLVVSRVTEDSARLSWTAPDAAFDSFEIGYFENPYLGEAIVLTVPG |
| | SERSYDLTGLKPGTEYWVQIGGVKGGNWSAPLSAIFTT |
| 187 | MSLPAPKNLVVSRVTEDSARLSWTAPDAAFDSFEIGYWENAYKGEAIVLTVPG |
| | SERSYDLTGLKPGTEYRVWIGGVKGGVWSRPLSAIFTT |
| 188 | MSLPAPKNLVVSRVTEDSARLSWTAPDAAFDSFEIGYWENAYKGEAIVLTVPG |
| | SERSYDLTGLKPGTEYVVFIGGVKGGHGSQPLSAIFTT |
| 189 | MSLPAPKNLVVSRVTEDSARLSWTAPDAAFDSFEIGYFEHTYYGEAIVLTVPG |
| | SERSYDLTGLKPGTEYWVQIGGVKGGNWSAPLSAIFTT |
| 190 | MSLPAPKNLVISRVTEDSARLSWTAPDAAFDSFEIGYFENLYLGEAIVLTVPG |
| | SERSYDLTGLKPGTEYWVQIGGVKGGNWSAPLSAISTT |
| 191 | MSLPAPKNLVVSRVTEDSARLSWTAPDAAFDSFEIGYFENLYLGEAIVLTVPG |
| | SERSYDLAGLKPGTEYWVQIGGVKGGNWSAPLSAIFTT |
| 192 | MSLPAPKNLVVSRVTEDSARLSWTAPDAAFDSFEIGYFENLYLGEAIVLTVPG |
| | SERSYDLTGLKPGTEYWVQIGGVKGGWGSNPLSAIFTT |
| 193 | MSLPAPKNLVVSRVTEDSARLSWTAPDAAFDSFEIGYFENLYLGEAIVLTVPG |
| | SERSYDLTGLKPGTEYWVQIGGVKGGNWSEPLSAIFTT |
| 194 | MSLPAPKNLVVSRVTEDSARLSWTAPDAAFDSFEIGYFENLYLGEAIVLTVPG |
| | SERSYDLTGLKPGTEYPVWIGGVKGGTWSVPLSTIFTT |
| 195 | MSLPAPKNLIVSRVTEDSARLSWTAPDAAFDSFEIGYFENLYLGEAIVLTVPG |
| | SERSYDLTGLKPGTEYWVQIGGVKGSNWSAPLSAIFTT |
| 196 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 197 | MSLPAPKNLVVSRVTEDSARLSWTAPDAAFDSFFIGYLEPQPPGEAISLLVPG |
| | SERSYDLTGLKPGTEYNVTIQGVKGGFPSEPLHAIFTT |
| 198 | MSLPAPKNLVVSRVTEDSARLSWTAPDAAFDSFLIGYTEYSVYGEAIVLTVPG |
| | SERSYDLTGLKPGTEYTVWIMGVKGGIKSTPLSAISTT |
| 199 | MSLPAPKNLVVSRVTEDSARLSWTAPDAAFDSFEIGYFENLYLGEAIVLTVPG |
| | YERSYDLTGLKPGTEYWVQIGGVKGGNWSAPLSAIFTT |
| 200 | MSLPAPKNLVVSRVTEDSARLSWTAPDAAFDSFEIGYFENLYLGEAIVLTVPG |
| | SERSYDLTGLKPGTEYWVQIGGVKGSNWSAPLSAIFTT |
| 201 | MSLPAPKNLVVSRVTEDSARLSWTAPDAAFDSFEIAYFEGTFHGEAIVLFVPG |
| | SERSYDLTGLKPGTEYAVWIGGVKGGDYSRPLSAAFTT |
| 202 | MSLPAPKNLVVSRVTEDSARLSWTAPDAAFDSFEIGYFENLYLGEAIVLTVPG |
| | SERSYDLTGLKPGTEYYVHIGGVKGGIWSSPLSAIFTT |
| 203 | MSLPAPKNLVVSRVTEDFARLSWTAPDAAFDSFEIAYWEQSYTGEAIVLTVPG |
| | SERSYDLTGLKPGTEYFVHIGGVKGGVWSTPLSAIFTT |
| 204 | MSLPAPKNLVVSRVTEDSARLSWTAPDAAFDSFLIQYDEWPTYGEAIVLTVPG |
| | SERSYDLTGLKPGTEYLVEIVGVKGGNLSGPLSAIFTT |
| 205 | MSLPAPKNLVVSRVTEDSARLSWTAPDAAFDSFEIGYFENLYLGEAIHLFVPG |
| | SERSYDLTGLKPGTEYNVTIQGVKGGFPSEPLIANFTT |
| 206 | MSLPAPKNLVASRVTEDSARLSWTAPDAAFDSFEIGYFENLYLGEAIVLTVPG |
| | SERSYDLTGLKPGTEYWVQIGGVKGGNWSAPLSAIFTT |
| 207 | MSLPAPKNLVVSRVTEDSARLSWTAPDAAFDSFEIGYWENAYKGEAIVLTVPG |
| | SERSYDLTGLKPGTEYVVFIGGVKGGVWSVPLSAISTT |
| 208 | MSLPAPKNLVVSRVTEDSARLSWTAPDAAFDSFFIGYFENLYLGEAIVLTVPG |
| | SERSYDLTGLKPGTEYWVQIGGVKGGNWSAPLSAIFTT |
| 209 | MSLPAPKNLVVSRVTEDSARLSWTAPDAAFDSFEIAYFERTWFGEAIVLTVPG |
| | SERSYDLTGLKPGTEYWVQIGGVKGGDWSRPLSAIFTT |
| 211 | MSLPAPKNLVVSRVTEDSARLSWTAPDAAFDSFEIVYAEPVRFGEAIVLTVPG |
| | SERSYDLTGLKPGTEYWVQIGGVKGGEWSSLLSAIFTT |
| 212 | MSLPAPKNLVVSRVTEDSARLSWTAPDAAFDSFEIGYFENLYLGEAIVLTVPG |
| | SERYYDLTGLKPGTEYWVQIGGVKGGNWSAPLSAIFTT |
| 213 | MSLPAPKNLVVSRVTEDSARLSWTAPDAAFDSFEIGYFENLYLGEAIVLTVPG |
| | SERSYDLTGLKPGTEYWVQIGGAKGGNWSAPLSAIFTT |
| 214 | MSLPAPKNLVVSRVTEDSARLSWTAPDAAFDSFEIDYGEYSQAGEAIGLLVPG |
| | SERSYDLTGLKPGTEYWVQIGGVKGGNWSAPLSAIFTT |
| 215 | MSLPAPKNLVVSRVTEDSARLSWTAPDAAFDSLEIGYFENLYLGEAIVLTVPG |
| | SERSYDLTGLKPGTEYWVQIGGVKGGNWSAPLSAIFTT |
| 216 | MSLPAPKNLVVSRVTEDSARLSWTAPDAAFDSFEIAYFEQTYTGEAIVLTVPG |
| | SERSYDLTGLKPGTEYWVQIGGVKGGTWSAPLSAISTT |
| 217 | MSLPAPKNLVVSRVTEDSARLSWTAPDAAFDSFEIAYFEQTYTGEAIVLTVPG |
| | SERSYDLTGLKPGTEYWVAIGGVKGGLWSLPLSAIFTT |
| 218 | MSLPAPKNLVVSRVTEDSARLSWTAPDAAFDSFEIAYHEDDFYGEAIALLVPG |
| | SERSYDLTGLKPGTEYIVHIGGVKGGFFSSPLYAWFTT |
| 219 | MSLPAPKNLVVSRVTEDSARLSWTAPDAAFDSFAIMYGERGNPGEAIVLTVPG |
| | SERSYDLTGLKPGTEYAVWIYGVKGGNYSYPLSAIFTT |
| 220 | MSLPAPKNLVVSRVTEDSARLSWTAPDAAFDSFEIAYWEQSYTGEAIVLTVPG |
| | SERSYDLTGLKPGTEYWVQIGGVKGGNWSAPLSAIFTT |
| 221 | MSLPAPKNLVVSRVTEDSARLSWTAPDAAFDSFFIGYLEPRPPGEAIHLYVPG |
| | SERSYDLTGLKPGTEYNITIQGVKGGFPSIPLIASFTT |
| 222 | MSLPAPKNLVVSRVTEDSARLSWTAPDAAFDSFFIGYLEPQPPGEAITLYVPG |
| | SERSYDLTGLKPGTEYNVTIQGVKGGFPSGPLIASFTT |
| 223 | MSLPAPKNLVVSRVTEDSARLSWTAPDAAFDSFEIAYFERTWFGEAIVLTVPG |
| | SERSYDLTGLKPGTEYWVAIGGVKGGLWSLPLSVIFTT |
| 224 | MSLPAPKNLVVSRVTEDSARLSWTAPDAAFDSFEIGYWENAYKGEAIVLTVPG |
| | SERSYDLTGLKPGTEYVVFIGGVKGGGWSGPLSAIFTT |
| 225 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 226 | MSLPAPKNLVISRVTEDSARLSWTAPDAAFDSFEIGYFENLYLGEAIVLTVPG |
| | SERSYDLTGLKPGTEYWVAIGGVKGGLWSLPLSAIFTT |
| 227 | MSLPAPKNLVVSRVTEDSARLSWTAPDAAFDSFEIAYAELSTGEAIVLTVPGS |
| | ERSYDLTGLKPGTEYYVHIGGVKGGSWSIPLSAIFTT |
| 228 | MSLPAPKNLVVSRVTEDSARLSWTAPDAAFDSFEIGYWENAYKGEAIVLTVPG |
| | SERSYDLTGLKPGTEYVVFIGGVKGGVWSKPLSVIFTT |
| 229 | MSLPAPKNLVVSRVTEDSARLSWTAPDAAFDSESIMYAEQKVNGEAIVLTVPG |
| | SERSYDLTGLKPGTEYLVLIWGVKGGGRSLPLSAIFTT |
| 230 | MSLPAPKNLIVSRVTEDSARLSWTAPDAAFDSLEIGYFENLYLGEAIVLTVPG |
| | SERSYDLTGLKPGTEYWVQIGGVKGGNWSAPLSAIFTT |
| 231 | MSLPAPKNLVVSRVTEDSARLSWTAPDAAFDSFFIGYLEPQPPGEAIHLFVPG |
| | SERSYDLTGLKPGTEYNVTIQGVKGGFPSEPLIANSTT |
| 232 | MSLPAPKNLVVSRVTEDSARLSWTAPDAAFDSFEISYWEHTENGEAIILFVPG |
| | SERSYDLTGLKPGTEYVVFIGGVKGGVWSVPLSAIFTT |
| 233 | MSLPAPKNLVVSRVTEDSARLSWTAPDAAFDSFFIGYLEPQPPGEAIHLSVPG |
| | SERSYDLTGLKPGTEYWVQIGGVKGGNWSAPLSAIFTT |
| 234 | MSLPAPKNLVVSRVTEDSARLSWTAPDAAFDSFFIGYLEPQPPGEAIALYVPG |
| | SERSYDLTGLKPGTEYNVTIQGVKGGFPSEPLVAHFTT |
| 235 | MSLPAPKNLVVSRVTEDSARLSWTAPDAAFDSFEIVYAEPVRFGEAIVLTVPG |
| | SERSYDLTGLKPGTEYWVQIGGVKGGNWSAPLSAIFTT |
| 236 | MSLPAPKNLVVSRVTEDSARLSWTEPDAAFDSFEIGYFENLYLGEAIVLTVPG |
| | SERSYDLTGLKPGTEYWVQIGGVKGGNWSAPLSAIFTT |
| 237 | MSLPAPKNLVVSRVTEDSARLSWTAPDAAFDSFEIAYFERTWFGEAIVLTVPG |
| | SERSYDLTGLKPGTEYWVQIGGVKGGDWSKPLSAIFTT |
| 238 | MSLPAPKNLVVSRVTEDSARLSWTAPDAAFDSFEIGYFENDYFGEAIVLTVPG |
| | SERSYDLTGLKPGTEYVVFIGGVKGGKWSAPLSAIFTT |
| 239 | MSLPAPKNLVVSRVTEDSARLSWTAPDAAFDSFEIVYQENSAYGEAIVLTVPG |
| | SERSYDLTGLKPGTEYWVQIGGVKGGNWSAPLSAIFTT |
| 240 | MSLPAPKNLVVSRVTEDSARLSWTAPDAAFDSFHIWYFEKANEGEAIPLVVPG |
| | SERSYDLTGLKPGTEYDVDIGGVKGGAWSIPLGARFTT |
| 241 | MSLPAPKNLVVSRVTEDSARLSWTAPDAAFDSEMIAYDEFIVWGEAVVLTVPG |
| | SERSYDLTGLKPGTEYLVEILGVKGGTISGPLSAIFTT |
| 242 | MSLPAPKNLVVSRVTEDSARLSWTAPDAAFDSFEIGYFENLYLGEAIVLTVPG |
| | SERSYDLTGLKPGTKYWVQIGGVKGGNWSAPLSAIFTT |
| 243 | MSLPAPKNLVVSRVTEDSARLSWTAPDAAFDSFFIGYLEPQPPGEAIHLYVPG |
| | SERSYDLTGLKPGTEYNVTIQGVKGGEWSAPLSAIFTT |
| 244 | MSLPAPKNLVVSRVTEDSARLSWTAPDAAFDSFEIGYFENLYLGEAIVLTVPG |
| | SERSYDLTGLKPGTEYVVFIGGVKGGEYSIPLSAIFTT |
| 245 | MSLPAPKNLVVSRVTEDSARLSWTAPDAAFDSFEISYWEHTENGEAIILFVPG |
| | SERSYDLTGLKPGTEYYVTIGGVKGGAWSPPLWAHFTT |
| 246 | MSLPAPKNLVVSRVTEDSARLSWTAPDAAFDSFEIAYQELSLWGEAIVLTVPG |
| | SERSYDLTGLKPGTEYYVHIGGVKGGIWSSPLSAYFTT |
| 247 | MSLPAPKNLVVSRVTEDSARLSWTAPDAAFDSFEIGYFENLYLGEAIVLTVPG |
| | SERSYDLTGLKPGTEYWVQIGGVKGGEWSAPLSAIFTT |
| 248 | MSLPAPKNLVVSRVTEDSARLSWTAPDAAFDSFEIGYFENLYLGEAIVLTVPG |
| | SERSYDLTGLKPGTEYVVFIGGVKGGDFSPPLSAIFTT |
| 249 | MSLPAPKNLVVSRVTEDSARLSWTAPDAAFDSFEIGYWENAYRGEAIVLTVPG |
| | SERSYDLTGLKPGTEYVVFIGGVKGGVWSVPLSAIFTT |
| 250 | MSLPAPKNLVVSRVTEDSARLSWTAPDAAFDSFEIGYFENLYLGEAIVLTVPG |
| | SERSYDLTGLKPGTEYWVQIGGVNGGNWSAPLSAIFTT |
| 251 | MSLPAPKNLVVSRVTEDSARLSWTAPDAAFDSFEIGYFENLYLGEAIVLTVPG |
| | SERSYDLTGLKPGTEYWVQIGGVKGGNWSAPLVATFTT |
| 252 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 253 | MSLPAPKNLVVSRVTEDSARLSWTAPDAAFDSFDINYWENESGGEAIALFVPG |
| | SERSYDLTGLKPGTEYWVSIGGVKGGRFSEPLYARFTT |
| 254 | MSLPAPKNLVVSRVTEDSARLSWTAPDAAFDSFEIGYFENLYLGEAIVLWVPG |
| | SERSYDLTGLKPGTEYWVQIGGVKGGNWSAPLSAIFTT |
| 255 | MSLPAPKNLVVSRVTEDSARLSWTTPDAAFDSFEIAYFEIAWLGEAIVLTVPG |
| | SERSYDLTGLKPGTEYWVQIGGVKGGAYSTPLSAIFTT |
| 256 | MSLPVPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 257 | MSLPAPKNLVVSHVTEDSARLSWTAPDAAFDSFEIGYWENAYKGEAIVLTVPG |
| | SERSYDLTGLKPGTEYVVFIGGVKGGVWSVPLSAIFTT |
| 258 | MSLPAPKNLVVSRVTEDSARLSWTAPDAAFDSFFIGYLEPQPPGEAIVLGVPG |
| | SERSYDLTGLKPGTEYNVTIQGVKGGFPSDPLVASFTT |
| 259 | MSLPAPKNLVVSRVTEDSARLSWTAPDAAFDSFYIAYAEPRPDGEAILLEVPG |
| | SERSYDLTGLKPGTEYSVLIHGVKGGLLSSPLSATFTT |
| 260 | MSLPAPKNLVVSRITEDSARLSWTAPDAAFDSFEIGYFENLYLGEAIVLTVPG |
| | SERSYDLTGLKPGTEYWVQIGGVKGGNWSAPLSAIFTT |
| 26 | MSLPAPKNLVVSRVTEDSARLSWTAPDAAFDSFYIAYAEPRPDGEAILLWVPG |
| | SERSYDLTGLKPGTEYSVLIHGVKGGLLSSPLHAYFTT |
| 262 | MSLPAPKNLVVSRVTEDSARLSWTAPDAAFDSFFIGYLEPQPPGEVIHLFVPG |
| | SERSYDLTGLKPGTEYNVTIQGVKGGFPSDPLVASFTT |
| 263 | MSLPAPKNLVVSRVTEDSARLSWTAPDAAFDSFEIGYFENLYLGEAIVLTVPG |
| | SERSYDLTGLKPGTEYWVQIGGVKGGNWSSPLSAIFTT |
| 264 | MSLPAPKNLVVSRVTEDSARLSWTAPDAAFDSFFIGYLEPQPPGEAIALYVPG |
| | SERSYDLTGLKPGTEYNVTIQGVKGGFPSEPLVVHFTT |
| 265 | MSLPAPKNLVVSRVTEDSARLSWTAPDAAFDSFFIGYLEPQPPGEAISLYVPG |
| | SERSYDLTGLKPGTEYNVTIQGVKGGFPSDPLVAHFTT |
| 266 | MSLPAPKNLVVSRVTEDSARLSWTAPDAAFDSFFIGYLEPQPPGEAIYLLVPG |
| | SERSYDLTGLKPGTEYNVTIQGVKGGFPSLPLVATFTT |
| 267 | MSLPAPKNLVVSRVTEDSARLSWTAPDAAFDSFEISYWEHTENGEAIILFVPG |
| | SERSYDLTGLKPGTEYYVTIGGVKGGAWSPPLWAEFTT |
| 268 | MSLPAPKNLVVSRVTEDSARLSWTAPDAAFDSFEIGYFENLYLGEAIVLTVPG |
| | SERSYDPTGLKPGTEYWVQIGGVKGGNWSAPLSAIFTT |
| 269 | MSLPAPKNLVVSRVTEDSARLSWTAPDAAFDSFEIGYFENDYFGEAIVLTVPG |
| | SERSYDLTGLKPGTEYVVFIGGVKGGNWSAPLSAIFTT |
| 270 | MSLPAPKNLVVSRVTEDSARLSWTAPDAAFDSFFIGYLEPQPPGEAIHLYVPG |
| | SERSYDLTGLKPGTEYNVTIQGVKGGFPSEPLIANSTT |
| 271 | MSLPAPKNLVVSRITEDSARLSWTAPDAAFDSFEIGYWENAYKGEAIVLTVPG |
| | SERSYDLTGLKPGTEYVVFIGGVKGGVWSVPLSAIFTT |
| 272 | MSLPAPKNLVVSRVTEDSARLSWTAPDAAFDSFEIAYGEAAYDGEAIALLVPG |
| | SERSYDLTGLKPGTEYWVQIGGVKGGNWSAPLSAIFTT |
| 273 | MLPAPKNLVVSRVTEDSARLSWTAPDAAFDSFEISYWEHTENGEAIILFVPGS |
| | ERSYDLTGLKPGTEYYVTIGGVKGGAWSPPLWAEFTT |
| 274 | MSLPAPKNLVVSRVTEDSARLSWTAPDAAFDSFEIVYWEQVGVGEAIVLTVPG |
| | SERSYDLTGLKPGTEYWVQIGGVKGGGFSEPLSAIFTT |
| 275 | MSLPAPKNLVVSRVTEDSARLSWTAPDAAFDSFEIGYFENLYLGEAIVLTVPG |
| | SERSYDLTGLKPGTEYWVQIGGVKGGNWSAPLVAEFTT |
| 276 | MSLPAPKNLVVSRVTEDSARLSWTAPDAAFDSFEIAYFELDYVGEAIVLTVPG |
| | SERSYDLTGLKPGTEYWVQIGGVKGGTWSGPLSAIFTI |
| 277 | MSLPAPKNLVVSRVTEDSARLSWTAPDAAFDSFEIGYFELTYYGEAIVLTVPG |
| | SERSYDLTGLKPGTEYWVQIGGVKGGGFSEPLSAIFTT |
| 278 | MSLPAPKNLVVSRVTEDSARLSWTAPDAAFDSFEIGYFENLYLGEAIVLTVPG |
| | SERSHDLTGLKPGTEYWVQIGGVKGGNWSAPLSAIFTT |
| 279 | MSLPAPKNLVVSRVTEDSARLSWTAPDAAFDSFFIGYLEPQPPGEAIHLYVPG |
| | SERSYDLTGLKPGTEYNVTIQGVKGGNWSAPLSAIFTT |
| 280 | MSLPAPKNLVVSRVTEDSARLSWTALDAAFDSFEIGYFENLYLGEAIVLTVPG |
| | SERSYDLTGLKPGTEYWVQIGGVKGGNWSAPLSAIFTT |
| 281 | MSLPAPKNLVVSRVTEDSARLSWTAPDAAFDSFFIGYLEPQPPGEAIALYVPG |
| | SERSYDLTGLKPGTEYNVTIQGVKGGFPSEPLIANFTT |
| 282 | MSLPAPKNLVVSRVTEDSARLSWTAPDAAFDSFDIGYFELTWYGEAIVLTVPG |
| | SERSYDLTGLKPGTEYWVSIGGVKGGRESEPLYARFTT |
| 283 | MSLPAPKNLVVSRVTEDSARLSWTAPDAAFDSFEIAYQELSLWGEAIVLTVPG |
| | SERSYDLTGLKPGTEYYVHIGGVKGGIWSSPLSAYSTT |
| 284 | MSLPAPKNLVVSRVTEDSARLSWTAPDAAFDSFEIGYWENAYKGEAIVLTVPG |
| | SERSYDLTGLKPGTEYMVFIGGVKGGVWSVPLSAIFTT |
| 285 | MSLPAPKNLVVSRVTEDSARLSWTATDAAFDSFEIGYWENAYKGEAIVLTVPG |
| | SERSYDLTGLKPGTEYVVFIGGVKGGVWSVPLSAIFTT |
| 286 | MSLPAPKNLVVSRVTEDSARLSWTAPDAAFDSFEIVYAEPVRFGEAIVLTVPG |
| | SERSYDLTGLKPGTEYWVQIGGVKGGEWSSPLSAIFTT |
| 287 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 288 | MSLPAPKNLVVSRITEDSARLSWTAPDAAFDSFEIGYFENLYLGEAIVLTVPG |
| | SERSYDLTGLKPGTEYWVQIGGVKGGNWSAPLSAIFTT |
| 289 | MSLPAPKNLVVSRVTEDSARLSWTAPDAAFDSFEIAYFEIAWLGEAIVLTVPG |
| | SERSYDLTGLKPGTEYWVQIGGVKGGAYSTPLSAIFTT |
| 290 | MSLPAPKNLVVSRVTEDSARLSWTAPDAAFDSFFIGYLEPQPPGEAIHLYVPG |
| | SERSYDLTGLKPGTEYNVTIQGVKGGFPSEPLVATFTT |
| 291 | MSLPAPKNLVVSRITEDSARLSWTAPDAAFDSFEIGYFENLYLGEAIVLTVPG |
| | SERSYDLTGLKPGTEYWVQIGGVKGGNWSAPLSAIFTT |
| 292 | MSLPAPKNLVVSRVTEDSARLSWTAPDAAFDSFEIGYWENAYKGEAIVLTVPG |
| | SERSYDLTGLKPGTEYVVFIGGVKGGVWSVSLSAIFTT |
| 293 | MSLPAPKNLVVSRVTEDSARLSWTAPDAAFDSFFIGYLEPQPPGEAIDLYVPG |
| | SERSYDLTGLKPGTEYNVTIQGVKGGFPSHPLVATFTT |
| 294 | MSLPAPKNLVVSRVTEDSARLSWTAPDAAFDSFFIGYLEPQPLGEAIHLFVPG |
| | SERSYDLTGLKPGTEYNVTIQGVKGGFPSEPLIASFTT |
| 295 | MSLPAPKNLVVSRVTEDSARLSWTAPDAAFDSFFIGYLEPQPPGEAIDLYVPG |
| | SERSYDLTGLKPGTEYNVTIQGVKGGFPSEPLVATFTT |
| 296 | MSLPAPKNLVVSRVTEDSARLSWTAPDAAFDSFDIGYFELTWYGEAIVLTVPG |
| | SERSYDLTGLKPGTEYWVSIGGVKGGIWSSPLSAIFTT |
| 297 | MSLPAPKNLVVSRVTEDSARLSWTAPDAAFDSFEISYFENTWYGEAIVLTVPG |
| | SERSYDLTGLKPGTEYSVRIFGVKGGNESFPLSAIFTT |
| 298 | MSLPAPKNLVVSRVTEDSARLSWTAPDAAFDSFEIGYFENLYLGEAIVLTVPG |
| | SERSYDLTGLKPGTEYWVQIGGVKGDNWSAPLSAIFTT |
| 299 | MSLPAPKNLVVSRVTEDSARLSWTAPDAAFDSFEIAYFENLYLGEAIVLTVPG |
| | SERSYDLTGLKPGTEYWVQIGGVKGGNWSAPLSAIFTT |
| 300 | MSLPAPKNLVVSRVTEDSARLSWTAPDAAFDSFEIGYFEATPNGEAIVLTVPG |
| | SERSYDLTGLKPGTEYKVFIGGVKGGRWSKPLSAIFTT |
| 301 | MSLPAPKNLVVSRVTEDSARLSWTTPDAAFDSFEIGYFENLYLGEAIVLTVPG |
| | SERSYDLTGLKPGTEYWVQIGGVKGGNWSAPLSAIFTT |
| 303 | MSLPAPKNLVVSRVTEDSARLSWTAPDAAFDSFWIEYWEGWKSGEAIVLTVPG |
| | SERSYDLTGLKPGTEYRVHIWGVKGGIVSWPLSAIFTT |
| 304 | MSLPAPKNLVVSRVTEDSARLSWTAPDAAFDSFEIGYFENLYLGEAIVLTVPG |
| | SERSYDLTGLKPGTEYVVFIGGVKGGVWSVPLSAIFTT |
| 305 | MSLPAPKNLVVSRVTEDSARLSWTAPDAAFDSFEIAYFEQTYTGEAIVLTVPG |
| | SERSYDLTGLKPGTEYWVQIGGVKGGNWSAPLSAIFTT |
| 306 | MSLPAPKNLVVSRVTEDSARLSWTAPDAAFDSFEIGHFENLYLGEAIVLTVPG |
| | SERSYDLTGLKPGIEYWVQIGGVKGGVWSVSLSAIFTT |
| 307 | MSLPAPKNLVVSRVTEDSARLSWTAPDAAFNSFEIGYFENLYLGEAIVLTVPG |
| | SERSYDLTGLKPGTEYWVQIGGVKGGTFSAPLSAIFTT |
| 308 | MSLPAPKNLVVSRVTEDSARMSWTTPDAAFDSFEIGYFENLYLGEAIVLTVPG |
| | SERSYDLTGLKPGTEYWVQIGGVKGGNWSAPLSAIFTT |
| 309 | MSLPAPENLVVSRVTEDSARLSWTAPDAAFDSFEIGYFENLYLGEAIVLTVPG |
| | SERSYDLTGLKPGTEYWVQIGGVKGGNWSAPLSAIFTT |
| 310 | MSLPAPKNLVVSRVTEDSARLSWTAPDAAFDSFEIDYGEYSQAGEAIGLLVPG |
| | SERSYDLTGLKPGTEYAVWIGGVKGGFFSTPLEADFTT |
| 311 | MSLPAPKNLVVSRVTEDSARLSWTAPDAAFDSFEIGYWENAYKGEAIVLTVPG |
| | SERSYDLTGLKPGTEYVVFIGGVKGGSWSNPLSAIFTT |
| 312 | MSLPAPKNLVVSRVTEDSARLSWTAPDAAFDSFEIGYFENLYLGEAIVLTIPG |
| | SERSYDLTGLKPGTEYWVQIGGVKGGNWSAPLSAIFTT |
| 313 | MSLPAPKNLVVSRVTEDSARLSWTAPDAAFDSFEIAYFENDWAGEAIVLTIPG |
| | SERSYDLTGLKPGTEYWVQIGGVKGGNWSAPLSAIFTT |
| 314 | MGSLPAPKNLVVSRVTEDSARLSWTAPDAAFDSFEIGYFENLYLGEAIVLTVP |
| | GSERSYDLTGLKPGTEYWVQIGGVKGGNWSAPLSAIFTT |
| 315 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAISTT |
| 316 | MSLPAPKNLVISRVTEDSARLSWTAPDAAFDSFEIGYFENLYLGEAIVLTVPG |
| | SERSYDLTGLKPGTEYWVQIGGVKGGNWSAPLSAIFTT |
| 317 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 319 | MSLPAPKNLVVSRVTEDSARLSWTAPGAAFDSFEIGYFENLYLGEAIVLTVPG |
| | SERSYDLTGLKPGTEYWVQIGGVKGGVWSVPLSAIFTT |
| 320 | MSLPVPKNLVVSRVTEDSARLSWTAPDAAFDSFEIGYWENAYKGEAIVLTVPG |
| | SERSYDLTGLKPGTEYWVQIGGVKGGNWSAPLSAIFTT |
| 321 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIFTT |
| 322 | MSLPAPKNLVVSRVTEDSARLSWTAPDAAFDSFEIGYFEITRYGEAIILFVPG |
| | SERSYDLTGLKPGTEYWVQIGGVKGGNWSAPLSAIFTT |
| 323 | MSLPAPKNLVVSRVTEDSARLSWTAPDAAFDSFEIGYFENLYLGEAIVLTVPG |
| | SERSYDLTGLKPGTEYWVQIGGVKGGNWSGPLSAIFTT |
| 324 | MSLPAPKNLVVSRITEDSARLSWTAPDAAFDSFEIGYFENLYLGEAIVLTVPG |
| | SERSYDLTGLKPGTEYWVQIGGVKGGNWSAPLSAISTT |
| 325 | MSLPAPKNLVVSRVTEDSARLSWTAPDAAFDSFEIGYFENLYLGEAIVLTVPG |
| | SERSYDLTGLKPGTEYWVQIGGVKGGNWSAPLSEIFTT |
| 326 | MSLPAPKNLVVSRVTEDSARLSWTAPDAAFDSFEIGYWENAYKGEAIVLTVPG |
| | SERSYDLTGLKPGTEYWVQIGGVKGGVWSTPLSAIFTT |
| 327 | MSLPAPKNLVVSRVTEDSARLSWTAPDAAFDSFEIGYWENAYKGEAIVLTVPG |
| | SERSYDLTGLKPGTEYWVQIGGVKGGNWSAPLSAIFTT |
| 328 | MSLPAPKNLVVSRVTEDSARLSWTAPDAAFDSFEIGYFGNLYLGEAIVLTVPG |
| | SERSYDLTGLKPGTEYWVQIGGVKGGNWSAPLSAIFTT |
| 329 | MSLPAPKNLVVSRVTEDSARLSWTAPDAAFDSFDIGYFEATYYGEAIVLTVPG |
| | SERSYDLTGLKPGTEYRVWIGGVKGGYYSNPLSAIFTT |
| 330 | MSLPAPKNLVVSRVTEDSARLSWTAPDAAFDSFEIGYFENLYLGEAIVLTVPG |
| | SERSYDLTGLKPGTEYWVQIGGVKGGNWSAPLSAIVTT |
| 331 | MSLPAPKNLVVSRVTEDSARLSWTAPDAAFDSFFIGYLEPQPPGEAIHLYVPG |
| | SERSYDLTGLKPGTEYNVTIQGVKVGFPSEPLIANFTT |
| 332 | MSLPAPKNLVVSRVTEDSARLSWTAPDAAFDSFEIGYWEGPGGGEAIILVVPG |
| | SERSYDLTGLKPGTEYYVQIGGVKGGDWSTPLWATFTT |
| 333 | MSLPAPENLVVSRVTEDSARLSWTAPDAAFDSFFIGYLEPQPPGEAIALYVPG |
| | SERSYDLTGLKPGTEYNVTIQGVKGGFPSLPLVATFTT |
| 334 | MSLPAPKNLVVSRVTEDSARLSWTAPDAAFDSFEIAYQELSLWGEAIVLTVPG |
| | SERSYDLTGLKPGTEYWVQIGGVKGGIWSSPLVASFTT |
| 335 | MSLPAPKNLVVSRVTEDSARLSWTAPDAAFDSFFIGYLEPQPPGEAIDLYVPG |
| | SERSYDLTGLKPGTEYNVTIQGVKGGFPSLPLVATFTT |
| 336 | MSLPAPKNLVVSRVTEDSARLSWTAPDAAFDSFNIMYHEHYDNGEAIVLTVPG |
| | SERSYDLTGLKPGTEYSVVINGVKGGPHSAPLSAIFTT |
| 337 | MSLPAPKNLVVSRVTEDSARLSWTAPDAAFDSFFIGYLEPQPPGEAIHLYVPG |
| | SERSYDLTGLKPGTEYNVTIQGVKGGFPSIPLIASFTT |
| 338 | MSLPAPKNLVVSRVTEDSARLSWTAPDAAFDSFEIGYFENLYLGEAIVLTVPG |
| | SERSYDLTGLKPGTEYCVYICGVKGGRDSMPLSAIFTT |
| 339 | MSLPAPKNLVVSRVTEDSARLSWTAPDAAFDSFEIGYWEMSYYGEAIVLTVPG |
| | SERSYDLTGLKPGTEYGVSIGGVKGGRWSLPLSAIFTT |
| 340 | MSLPAPKNLVVSRVTEDSSRLSWTAPDAAFDSFEIGYFENLYLGEAIVLTVPG |
| | SERSYDLTGLKPGTEYWVQIGGVKGGNWSAPLSAIFTT |
| 341 | MSLPAPKNLVVSRVTEDSARLSWTAPDAAFDSFFIGYLEPQPPGEAIELYVPG |
| | SERSYDLTGLKPGTEYNVTIQGVKGGFPSLPLVAIFTT |
| 342 | MSLPAPKNLVVSRVTEDSARLSWTAPDAAFDSFFIGYLEPQPPGEAIVLTVPG |
| | SERSYDLTGLKPGTEYNVTIQGVKGGFPSEPLIANFTT |
| 343 | MSLPAPKNLVVSRVTEDSARLSWTAPDAAFDSFFIGYLEPQPPGEAIALWVPG |
| | SERSYDLTGLKPGTEYNVTIQGVKGGFPSEPLVATFTT |
| 344 | MSLPAPKNLFVSRVTEDSARLSWTAPDAAFDSFEIGYWENAYKGEAIVLTVPG |
| | SERSYDLTGLKPGTEYWVQIGGVKGGNWSAPLSAIFTT |
| 345 | MSLPAPKNLVVSRVTEDSAHLSWTAPDAAFDSFEIGYFENDYFGEAIVLTVPG |
| | SERSYDLTGLKPGTEYVVFIGGVKGGKWSAPLSAIFTT |
| 346 | MLPAPKNLVVSRVTEDSARLSWTAPDAAFDSFFIGYLEPQPPGEAISLFVPGS |
| | ERSYDLTGLKPGTEYNVTIQGVKGGFPSDPLNATENT |
| 347 | MLPAPKNLVVSRVTEDSARLSWTAPDAAFDSFFIGYLEPQPPGEAIGLFVPGS |
| | ERSYDLTGLKPGTEYNVTIQGVKGGFPSEPLKAAFTT |
| 348 | MLPAPKNLVVSRVTEDSARLSWTAPDAAFDSFEISYFENTWYGEAIVLTVPGS |
| | ERSYDLTGLKPGTEYVVWIGGVKGGLWSKPLSAIFTT |
| 349 | MLPAPKNLVVSRVTEDSARLSWTAPDAAFDSFFIGYLEPQPPGEAIELYVPGS |
| | ERSYDLTGLKPGTEYNVTIQGVKGGFPSEPLIAHFTT |
| 350 | MLPAPKNLVVSRVTEDSARLSWTAPDAAFDSFFIGYLEPQPPGEAISLFVPGS |
| | ERSYDLTGLKPGTEYNVTIQGVKGGFPSMPLVAQFTT |
| 351 | MLPAPKNLVVSRVTEDSARLSWTAPDAAFDSFFIGYLEPQPPGEAITLAVPGS |
| | ERSYDLTGLKPGTEYNVTIQGVKGGFPSNPLLAAFTT |
| 352 | MLPAPKNLVVSRVTEDSARLSWTAPDAAFDSFEIAYQELSLWGEAIVLTVPGS |
| | ERSYDLTGLKPGTEYYVHIGGVKGGIWSSPLSAIFTT |
| 353 | MLPAPKNLVVSRVTEDSARLSWTAPDAAFDSFFIGYLEPQPPGEAIGLFVPGS |
| | ERSYDLTGLKPGTEYNVTIQGVKGGFPSNPLEARFTT |
| 354 | MLPAPKNLVVSRVTEDSARLSWTAPDAAFDSFFIGYLEPQPPGEAIQLWVPGS |
| | ERSYDLTGLKPGTEYNVTIQGVKGGFPSLPLVATFTT |
| 355 | MLPAPKNLVVSRVTEDSARLSWTAPDAAFDSFFIGYLEPQPPGEAIHLYVPGS |
| | ERSYDLTGLKPGTEYNVTIQGVKGGFPSHPLYADFTT |
| 356 | MLPAPKNLVVSRITEDSARLSWTAPDAAFDSFFIGYLEPQPPGEAITLLVPAS |
| | ERSYDLTGLKPGTEYNVTIQGVKGGFPSHPLQAQFTT |
| 357 | MLPAPKNLVVSRVTEDSARLSWTAPDAAFDSFFIGYLEPQPPGEAIGLFVPGS |
| | ERSYDLTGLKPGTEYNVTIQGVKGGFPSDPLVAYFTT |
| 358 | MLPAPKNLVVSRVTEDSARLSWTAPDAAFDSFEIGYWENAYKGEAIVLTVPGS |
| | ERSYDLTGLKPGTEYVVFIGGVKGGVWSVPLSAIFTT |
| 359 | MLPAPKNLVVSRVTEDSARLSWTAPDAAFDSFLIPYAETSPSGEAIVLTVPGS |
| | ERSYDLTGLKPGTEYSVLIHGVKGGDYSSPLSAIFTT |
| 360 | MLPAPKNLVVSRVTEDSARLSWTAPDAAFDSFFIGYLEPQPPGEAISLYVPGS |
| | ERSYDLTGLKPGTEYNVTIQGVKGGFPSHPLLAVFTT |
| 361 | MLPAPKNLVVSRVTEDSARLSWTAPDAAFDSFFIGYLEPQPPGEAIQLFVPGS |
| | ERSYDLTGLKPGTEYNVTIQGVKGGFPSIPLSALFTT |
| 362 | MLPAPKNLVVSRVTEDSARLSWTAPDAAFDSFEIAYWENNENGEAIILIVPGS |
| | ERSYDLTGLKPGTEYVVFISGVKGGTWSYPLVAQFTT |
| 363 | MLPAPKNLVVSRVTEDSARLSWTAPDAAFDSFFIGYLEPQPPGEAIDLWVPGS |
| | ERSYDLTGLKPGTEYNVTIQGVKGGFPSLPLSAIFTT |
| 364 | MLPAPNNLVVSRVTEDSARLSWTAPDAAFDSFFIGYLEPQPPGEAIHLFVPGS |
| | ERSYDLTGLKPGTEYNVTIQGVKGGFPSGPLHATFTT |
| 365 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKFTT |
| 366 | MLPAPKNLVVSRVTEDSARLSWTAPDAAFDSFFIGYLEPQPPGEAIDLWVPGS |
| | ERSYDLTGLKPGTEYNVTIQGVKGGFPSNPLKAQFTT |
| 367 | MLPAPKNLVVSRVTEDSARLSWTAPDAAFDSFYIAYAEPRPDGEAILLVVPGS |
| | ERSYDLTGLKPGTEYSVLIHGVKGGLLSSPLVAFFTT |
| 368 | MLPAPKNLVVSRVTEDSARLSWTAPDAAFDSFFIGYLEPQPPGEAITLTVPGS |
| | ERSYDLTGLKPGTEYNVTIQGVKGGFPSGPLIASFTT |
| 369 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKSTT |
| 370 | MLPAPKNLVVSRVTEDSARLSWTAPDAAFDSFYIAYAEPRPDGEAIVLVVPGS |
| | ERSYDLTGLKPGTEYSVLIHGVKGGLLSSPLSAHFTT |
| 371 | MLPAPKNLVVSRVTEDSARLSWTAPDAAFDSFDIDYVEFGWTGEAIALLVPGS |
| | ERSYDLTGLKPGTEYWVWIGGVKGGDYSPPLNAYFTT |
| 372 | MLPAPKNLVVSRVTEDSARLSWTAPDAAFDSFFIGYLEPQPPGEAINLLVPGS |
| | ERSYDLTGLKPGTEYNVTIQGVKGGFPSLPLLAEFTT |
| 373 | MLPAPKNLVVSRVTEDSARLSWTAPDAAFDSFFIGYLEPQPPGEAISLFVPGS |
| | ERSYDLTGLKPGTEYNVTIQGVKGGFPSDPLVASFTT |
| 374 | MLPAPKNLVVSRVTEDSARLSWTAPDAAFDSFYIAYAEPRPDGEAIQLSVPGS |
| | ERSYDLTGLKPGTEYSVLIHGVKGGLLSSPLHASFTT |
| 375 | MLPAPKNLVVSRVTEDSARLSWTAPDAAFDSFFIGYLEPQPPGEAIDLTVPGS |
| | ERSYDLTGLKPGTEYNVTIQGVKGGFPSMPLSAIFTT |
| 376 | MLPAPKNLVVSRVTEDSARLSWTAPDAAFDSFFIGYLEPQPPGEAIALLVPGS |
| | ERSYDLTGLKPGTEYNVTIQGVKGGFPSNPLEAKFTT |
| 377 | MLPAPKNLVVSRVTEDSARLSWTAPDAAFDSFFIGYLEPQPPGEAIQLWVPGS |
| | ERSYDLTGLKPGTEYNVTIQGVKGGFPSTPLQALFTT |
| 378 | MLPAPKNLVVSRVTEDSARLSWTAPDAAFDSFFIGYLEPQPPGEAIDLWVPGS |
| | ERSYDLTGLKPGTEYNVTIQGVKGGFPSTPLIATVTT |
| 379 | MLPAPKNLVVSRVTEDSARLSWTAPDAAFDSFYIEYWEGYRSGEAIVLTVPGS |
| | ERSYDLTGLKPGTEYRVHIWGVKGGAVSYPLSAIFTT |
| 380 | MLPAPKNLVVSRVTEDSARLSWTAPDAAFDSFFIGYLEPQPPGEAIGLFVPGS |
| | ERSYDLTGLKPGTEYNVTIQGVKGGFPSMPLSAIFTT |
| 381 | MLPAPKNLVVSRVTEDSARLSWTAPDAAFDSFEIAYQELSLWGEAIVLTVPGS |
| | ERSYDLTGLKPGTEYYVHIGGVKGGIWSRPLSAIFTT |
| 382 | MLPAPKNLVVSRVTEDSARLSWTAPDAAFDSFFIGYLEPQPPGEAIELYVPGS |
| | ERSYDLTGLKPGTEYNVTIQGVKGGFPSDPLEADETT |
| 383 | MLPAPKNLVVSRVTEDSARLSWTAPDAAFDSFFIGYLEPQPPGEAIDLWVPGS |
| | ERSYDLTGLKPGTEYNVTIQGVKGGFPSLPLDAVFTT |
| 384 | MLPAPKNLVVSRVTEDSARLSWTAPDAAFDSFEIDYAENRHHGEAIALLVPGS |
| | ERSYDLTGLKPGTEYIVFIGGVKGGRWSQPLVASFTT |
| 385 | MLPAPKNLVVSRVTEDSARLSWTAPDAAFDSFFIGYLEPQPPGEAIDLYVPGS |
| | ERSYDLTGLKPGTEYNVTIQGVKGGFPSHPLQAHFTT |
| 386 | MLPAPKNLVVSRVTEDSARLSWTAPDAAFDSFYIAYAEPRPDGEAITLLVPGS |
| | ERSYDLTGLKPGTEYSVLIHGVKGGLLSSPLSAIFTT |
| 387 | MLPAPKNLVVSRVTEDSARLSWTAPDAAFDSFFIGYLEPQPPGEAIHLVVPGS |
| | ERSYDLTGLKPGTEYNVTIQGVKGGFPSDPLYAWFTT |
| 388 | MLPAPKNLVVSRVTEDSARLSWTAPDAAFDSFEIAYFEYCLNGEAIVLTVPGS |
| | ERSYDLTGLKPGTEYWVQIGGVKGGTWSWPLSAIFTT |
| 389 | MLPAPKNLVVSRVTEDSARLSWTAPDAAFDSFFIGYLEPQPPGEAIHLLVPGS |
| | ERSYDLTGLKPGTEYNVTIQGVKGGFPSMPLSAIVTT |
| 390 | MLPAPKNLVVSRVTEDSARLSWTAPDAAFDSFEIGYFERAWFGEAIVLTVPGS |
| | ERSYDLTGLKPGTEYAVFIGGVKGGSYSYPLSAIFTT |
| 391 | MLPAPKNLIVSRVTEDSARLSWTAPDAAFDSFFIGYLEPQPPGEAISLYVPGS |
| | ERSYDLTGLKPGTEYNVTIQGVKGGFPSDPLQAIFTT |
| 392 | MLPAPKNLVVSRVTEDSARLSWTAPDAAFDSFFIGYLEPQPPGEAIHLGVPGS |
| | ERSYDLTGLKPGTEYNVTIQGVKGGFPSLPLVATFTT |
| 393 | MLPAPKNLVVSRVTEDSARLSWTAPDAAFDSFFIGYLEPQPPGEAIGLFVPGS |
| | ERSYDLTGLKPGTEYNVTIQGVKGGFPSLPLQAHFTT |
| 394 | MLPAPKNLVVSRVTEDSARLSWTAPDAAFDSFFIGYLEPQPPGEAIYLLVPGS |
| | ERSYDLTGLKPGTEYNVTIQGVKGGFPSTPLEASFTT |
| 395 | MLPAPKNLVVSRVTEDSARLSWTAPDTAFDSFFIGYLEPQPPGEAIGLFVPGS |
| | ERSYDLTGLKPGTEYNVTIQGVKGGFPSDPLSAFFTT |
| 396 | MLPAPKNLVVSRVTEDSARLSWTAPDAAFDSFFIGYLEPQPPGEAIYLTVPGS |
| | ERSYDLTGLKPGTEYNVTIQGVKGGFPSMPLSAIFTT |
| 397 | MLPAPKNLVVSRVTEDSARLSWTAPDAAFDSFIILYGEAQYDGEAIVLTVPGS |
| | ERSYDLTGLKPGTEYPVDIYGVKGGPYSWPLSAIFTT |
| 398 | MLPAPKNLVVSRVTEDSARLSWTAPDAAFDSFFIGYLEPQPPGEAIYLTVPGS |
| | ERSYDLTGLKPGTEYNVTIQGVKGGFPSKPLTANFTT |
| 399 | MLPAPKNLVVSRVTEDSARLSWTAPDAAFDSFFIGYLEPQPPGEAITLYVPGS |
| | ERSYDLTGLKPGTEYNVTIQGVKGGFPSTPLTATFTT |
| 400 | MLPAPKNLVVSRVTEDSARLSWTAPDAAFDSFFIGYLEPQPPGEAIHLFVPGS |
| | ERSYDLTGLKPGTEYNVTIQGVKGGFPSGPLHATFTT |
| 401 | MLPAPKNLVVSRVTEDSARLSWTAPDAAFDSFFIGYLEPQPPGEAIALWVPGS |
| | ERSYDLTGLKPGTEYNITIQGVKGGFPSMPLVANFTT |
| 402 | MLPAPKNLVVSRVTEDSARLSWTAPDAAFDSFGIYYWEYTGGEAIVLTVPGSE |
| | RSYDLTGLKPGTEYVVRILGVKGGAYSTPLSAIFTT |
| 403 | MLPAPKNLVVSRVTEDSARLSWTAPDAAFDSFYIAYAEPRPDGEAIVLDVPGS |
| | ERSYDLTGLKPGTEYSVLIHGVKGGLLSSPLVADFTT |
| 404 | MLPAPKNLVVSRVTEDSARLSWTAPDAAFDSFFIGYLEPQPPGEAIHLLVPGS |
| | ERSYDLTGLKPGTEYNVTIQGVKGGFPSEPLLAIVTT |
| 405 | MLPAPKNLVVSRVTEDSARLSWTAPDAAFDSFFIGYLEPQPPGEAIHLWVPGS |
| | ERSYDLTGLKPGTEYNVTIQGVKGGFPSLPLSAFFTT |
| 406 | MLPAPKNLVVSRVTEDSARLSWTAPDAAFDSFFIDYDESLDSGEAIVLTVPGS |
| | ERSYDLTGLKPGTEYNVTIQGVKGGFPSSPLEAAFTT |
| 407 | MLPAPKNLVVSRVTEDSARLSWTAPDAAFDSFEIAYFETCRTGEAIVLTVPGS |
| | ERSYDLTGLKPGTEYRVWIGGVKGGVWSRPLSAIFTT |
| 408 | MLPAPKNLVVSRVTEDSARLSWTAPDAAFDSFFIGYLEPQPPGEAIGLWVPGS |
| | ERSYDLTGLKPGTEYNVTIQGVKGGFPSMPLSAIFTT |
| 409 | MLPAPKNLVVSRVTEDSARLSWTAPDAAFDSFFIGYLEPQPPGEAIHLFVPGS |
| | ERSYDLTGLKPGTEYNVTIQGVKGGFPSNPLSAQFTT |
| 410 | MLPAPKNLVVSRVTEDSARLSWTAPDAAFDSFFIGYLEPQPPGEAIALWVPGS |
| | ERSYDLTGLKPGTEYNVTIQGVKGGFPSQPLHAHFTT |
| 411 | MLPAPKNLVVSRVTEDSARLSWTAPDAAFDSFFIGYLEPQPPGEAISLFVPGS |
| | ERSYDLTGLKPGTEYNVTIQGVKGGFPSVPLSATFTT |
| 412 | MLPAPKNLVVSRVTEDSARLSWTAPDAAFDSFFIGYLEPQPPGEAIGLLVPGS |
| | ERSYDLTGLKPGTEYNVTIQGVKGGFPSHPLLAVETT |
| 413 | MLPAPKNLVVSRVTEDSARLSWTAPDAAFDSFFIGYLEPQPPGEAIYLRVPGS |
| | ERSYDLTGLKPGTEYNVTIQGVKGGFPSAPLHAHFTT |
| 414 | MLPAPKNLVVSRVTEDSARLSWTAPDAAFDSFFIGYLEPQPPGEAIHLYVPGS |
| | ERSYDLTGLKPGTEYNVTIQGVKGGFPSHPLIANFTT |
| 415 | MLPAPKNLVVSRVTEDSARLSWTAPDAAFDSFFIGYLEPQPPGEAIHLQVPGS |
| | ERSYDLTGLKPGTEYNVTIQGVKGGFPSMPLSAIFTT |
| 416 | MLPAPKNLVVSRVTEDSARLSWTAPDAAFDSFFIGYLEPQPPGEAIALYVPGS |
| | ERSYDLTGLKPGTEYNVTIQGVKGGFPSLPLVATFTT |
| 417 | MLPAPKNLVVSRVTEDSARLSWTAPDAAFDSFFIGYLEPQPPGEAIQLFVPGS |
| | ERSYDLTGLKPGTEYNVTIQGVKGGFPSDPLVASFTT |
| 418 | MLPAPKNLVVSRVTEDSARLSWTAPDAAFDSFEIAYFENSYYGEAIVLTVPGS |
| | ERSYDLTGLKPGTEYAVYIGGVKGGSWSNPLSAISTT |
| 419 | MLPAPKNLVVSRVTEDSARLSWTAPDAAFDSFFIGYLEPQPPGEAIALLVPGS |
| | ERSYDLTGLKPGTEYNVTIQGVKGGFPSSPLNAYFTT |
| 420 | MLPAPKNLVVSRVTEDSARLSWTAPDAAFDSFFIGYLEPQPPGEAIHLQVPGS |
| | ERSYDLTGLKPGTEYNVTIHGVKGGIPSMPLSAKFTT |
| 421 | MLPAPKNLVVSRVTEDSARLSWTAPDAAFDSFFIGYLEPQPPGEAIDLWVPGS |
| | ERSYDLTGLKPGTEYNVTIQGVKGGFPSMPLEAVETT |
| 422 | MLPAPKNLVVSRVTEDSARLSWTAPDAAFDSFFIGYLEPQPPGEAISLLVPGS |
| | ERSYDLTGLKPGTEYNVTIQGVKGGFPSHPLDASFTT |
| 423 | MLPAPKNLVVSRVTEDSARLSWTAPDAAFDSFFIGYLEPQPPGEAIALYVPGS |
| | ERSYDLTGLKPGTEYNVTIQGVKGGFPSEPLVATFTT |
| 424 | MLPAPKNLVVSRVTEDSARLSWTAPDAAFDSFFIGYLEPQPPGEAIHLIVPGS |
| | ERSYDLTGLKPGTEYNVTIQGVKGGFPSDPLDASFTT |
| 425 | MLPAPKNLVVSRVTEDSARLSWTAPDAAFDSFFIGYLEPQPPGEAIGLLVPGS |
| | ERSYDLTGLKPGTEYNVTIQGVKGGFPSNPLSAIFTT |
| 426 | MLPAPKNLVVSRVTEDSARLSWTAPDAAFDSFFIGYLEPQPPGEAIFLLVPGS |
| | ERSYDLTGLKPGTEYNVTIQGVKGGFPSMPLSAIFTT |
| 427 | MLPAPKNLVVSRVTEDSARLSWTAPDAAFDSFFIGYLEPQPPGEAIHLRVPGS |
| | ERSYDLTGLKPGTEYNVTIQGVKGGFPSDPLIAVFTT |
| 428 | MLPAPKNLVVSRVTEDSARLSWTAPDAAFDSFFIGYLEPQPPGEAISLYVPGS |
| | ERSYDLTGLKPGTEYNVTIQGVKGGFPSNPLIAQFTT |
| 429 | MLPAPKNLVVSRVTEDSARLSWTAPDAAFDSFEIGYFERSFYGEAIILFVPGS |
| | ERSYDLTGLKPGTEYAVWIGGVKGGVWSRPLSAIFTT |
| 430 | MLPAPKNLVVSRVTEDSARLSWTAPDAAFDSFFIGYLEPQPPGEAIDLWVPGS |
| | ERSYDLTGLKPGTEYNVTIQGVKGGFPSYPLSASFTT |
| 431 | MLPAPKNLVVSRVTEDSARLSWTAPDAAFDSFFIGYLEPQPPGEAIVLFVPGS |
| | ERSYDLTGLKPGTEYNVTIQGVKGGFPSNPLSAISTT |
| 432 | MLPAPKNLVVSRVTEDSARLSWTAPDAAFDSFFIGYLEPQPPGEAIALYVPGS |
| | ERSYDLTGLKPGTEYNVTIQGVKGGFPSGPLEAYFTT |
| 433 | MLPAPKNLVVSRVTEDSARLSWTAPDAAFDSFFIGYLEPQPPGEAIALYVPGS |
| | ERSYDLTGLKPGTEYNVTIQGVKGGFPSLPLEANFTT |
| 434 | MLPAPKNLVVSRVTEDSARLSWTAPDAAFDSFFIGYLEPQPPGEAIGLQVPGS |
| | ERSYDLTGLKPGTEYNVTIQGVKGGFPSDPLQAIFTT |
| 435 | MLPAPKNLVVSRVTEDSARLSWTAPDAAFDSFFIGYLEPQPPGEAIALWVPGS |
| | ERSYDLTGLKPGTEYNVTIQGVKGGFPSHPLAAVETT |
| 436 | MLPAPKNLVVSRVTEDSARLSWTAPDAAFDSFFIGYLEPQPPGEAIHLLVPGS |
| | ERSYDLTGLKPGTEYNVTIQGVKGGFPSEPLWAKFTT |
| 437 | MLPAPKNLVVSRVTEDSARLSWTAPDAAFDSFFIGYLEPQPPGEAISLWVPGS |
| | ERSYDLTGLKPGTEYNVTIQGVKGGFPSTPLSAKFTT |
| 438 | MLPAPKNLVVSRVTEDSARLSWTAPDAAFDSFFIGYLEPQPPGEAIDLWVPGS |
| | ERSYDLTGLKPGTEYNVTIQGVKGGFPSYPLLAIFTT |
| 439 | MLPAPKNLVVSRVTEDSARLSWTAPDAAFDSFFIGYLEPQPPGEAIHLFVPGS |
| | ERSYDLTGLKPGTEYNVTIQGVKGGFPSVPLNAYFTT |
| 440 | MLPAPKNLVVSRVTEDSARLSWTAPDAAFDSFEIVYAEWTQHGEAIVLTVPGS |
| | ERSYDLTGLKPGTEYYVHIGGVKGGKWSPPLYAIFTT |
| 441 | MLPAPKNLVVSRVTEDSARLSWTAPDAAFDSFFIGYLEPQPPGEAILLWVPGS |
| | ERSYDLTGLKPGTEYNVTIQGVKGGFPSDPLIANFTT |
| 442 | MLPAPKNLVVSRVTEDSARLSWTAPDAAFDSFEIWYAEWRQVGEAIVLTVPGS |
| | ERSYDLTGLKPGTEYNVDIHGVKGGKVSWPLSAISTT |
| 443 | MLPAPKNLVVSRVTEDSARLSWTAPDAAFDSFFIGYLEPQPPGEAIELYVPGS |
| | ERSYDLTGLKPGTEYNVTIQGVKGGFPSDPLHAAFTT |
| 444 | MLPAPKNLVVSRVTEDSARLSWTAPDAAFDSFEIDYVEFIFTGEAIGLIVPGS |
| | ERSYDLTGLKPGTKYWVTIAGVKGGEWSTPLQAFFTT |
| 445 | MLPAPKNLVVSRVTEDSARLSWTAPDAAFDSFFIGYLEPQPPGEAIHLFVPGS |
| | ERSYDLTGLKPGTEYNVTIQGVKGGFPSDPLVASFTT |
| 446 | MLPAPKNLVVSRVTEDSARLSWTAPDAAFDSFFIGYLEPQPPGEAIQLFVPGS |
| | ERSYDLTGLKPGTEYNVTIQGVKGGFPSLPLIAYFTT |
| 447 | MLPAPKNLVVSRVTEDSARLSWTAPDAAFDSFFIGYLEPQPPGEAIYLYVPGS |
| | ERSYDLTGLKPGTEYNVTIQGVKGGFPSYPLTAQFTT |
| 448 | MLPAPKNLVVSRVTEDSARLSWTAPDAAFDSFFIGYLEPQPPGEAIDLYVPGS |
| | ERSYDLTGLKPGTEYNVTIQGVKGGFPSMPLYARFTT |
| 449 | MLPAPKNLVVSRVTEDSARLSWTAPDAAFDSFYIAYQELSLWGEAIVLTVPGS |
| | ERSYDLTGLKPGTEYNVTIQGVKGGFPSLPLAAQFTT |
| 450 | MLPAPKNLVVSRVTEDSARLSWTAPDAAFDSFFIGYLEPQPPGEAISLFVPGS |
| | ERSYDLTGLKPGTEYNVTIQGVKGGFPSEPLAAKFTT |
| 451 | MLPAPKNLVVSRVTEDSARLSWTAPDAAFDSFFIGYLEPQPPGEAIGLLVPGS |
| | ERSYDLTGLKPGTEYNVTIQGVKGGFPSIPLVADFTT |
| 452 | MLPAPKNLVVSRVTEDSARLSWTAPDAAFDSFFIGYLEPQPPGEAIHLQVPGS |
| | ERSYDLTGLKPGTEYNVTIQGVKGGFPSIPLVAQFTT |
| 453 | MLPAPKNLVVSRVTEDSARLSWTAQDAAFDSFFIGYLEPQPPGEAIALLVPGS |
| | ERSYDLTGLKPGTEYNVTIQGVKGGFPSHPLSASFTT |
| 454 | MLPAPKNLVVSRVTEDSARLSWTAPDAAFDSFFIGYLEPQPPGEAINLTVPGS |
| | ERSYDLTGLKPGTEYNVTIQGVKGGFPSYPLQASFTT |
| 455 | MLPAPKNLVVSRVTEDSARLSWTAPDAAFDSFTISYPEEAKHGEAIVLTVPGS |
| | ERSYDLTGLKPGTEYGVPINGVKGGVSSLPLSAIFTT |
| 456 | MLPAPKNLVVSRVTEDSARLSWTAPDAAFDSFFIGYLEPQPPGEAIALYVPGS |
| | ERSYDLTGLKPGTEYNVTIQGVKGGFPSHPLQAHFTT |
| 457 | MLPAPKNLVVSRVTEDSARLSWTAPDAAFDSFFIGYLEPQPPGEAIVLIVPGS |
| | ERSYDLTGLKPGTEYNVTIQGVKGGFPSAPLVATFTT |
| 458 | MLPAPKNLVVSRVTEDSARLSWTAPDAAFDSELDLLPSNWDIAGEAIVLTVPG |
| | SERSYDLTGLKPGTEYHVNILGVKGGKESLPLVANFTT |
| 459 | MLPAPKNLVVSRVTEDSARLSWTAPDAAFDSFFIRYLEPQPPGEAIHLSVPGS |
| | ERSYDLTGLKPGTEYNVTIQGVKGGFPSMPLYAIFTT |
| 460 | MLPAPKNLVVSRVTEDSARLSWTAPDAAFDSFFIGYLEPQPPGEAISLYVPGS |
| | ERSYDQTGLKPGTEYNVTIQGVKGGFPSDPLSARFTT |
| 461 | MLPAPKNLVVSRVTEDSARLSWTAPDAAFDSFFIGYLEPQPPGEAIGLFVPGS |
| | ERSYDLTGLKPGTEYNVTIQGVKGGFPSMPLSPIFTT |
| 462 | MLPAPKNLVVSRVTEDSARLSWTAPDAAFDSFFIGYLEPQPPGEAIHLQVPGS |
| | ERSYDLTGLKPGTEYNVTIQGVKGGFPSVPLHARFTT |
| 463 | MLPAPKNLVVSRVTEDSARLSWTAPDAAFDSFLILYIEQDHRGEAIVLTVPGS |
| | ERSYDLTGLKPGTEYWVHITGVKGGYYSAPLSAIFTT |
| 464 | MLPAPKNLVVSRVTEDSARLSWTAPDAAFDSFFIGYLEPQPPGEAIQLFVPGS |
| | ERSYDLTGLKPGTEYNVTIQGVKGGFPSTPLVASFTT |
| 465 | MLPAPKNLVVSRVTEDSARLSWTAPDAAFDSFFIGYLEPQPPGEAIDLYVPGS |
| | ERSYDLTGLKPGTEYNVTIQGVKGGFPSEPLWADFTT |
| 466 | MLPAPKNLVVSRVTEDSARLSWTAPDAAFDSFYIAYAEPRPDGEAIWLLVPGS |
| | ERSYDLTGLKPGTEYSVLIHGVKGGLLSSPLSADFTT |
| 467 | MLPAPKNLVVSRVTEDSARLSWTAPDAAFDSFFIGYLEPQPPGEAIQLLVPGS |
| | ERSYDLTGLKPGTEYNVTIQGVKGGFPSDPLVAYSTT |
| 468 | MLPAPKNLVVSRVTEDSARLSWTAPDAAFDSFLIQYDEEGVWGEAIVLTVPGS |
| | ERSYDLTGLKPGTEYSVGIGGVKGGWSSVPLSAIFTT |
| 469 | MLPAPKNLVVSRVTEDSARLSWTAPDAAFDSFFIGYLEPQPPGEAIHLYVPGS |
| | ERSYDLTGLKPGTEYNVTIQGVKGGFPSLPLVATFTT |
| 470 | MLPAPKNLVVSRVTEDSARLSWTAPDAAFDSFFIGYLEPQPPGEAITLLVPGS |
| | ERSYDLTGLKPGTEYNVTIQGVKGGFPSMPLSAIFTT |
| 471 | MLPAPKNLVVSRVTEDSARLSWTAPDAAFDSFTIWYYESPTGGEAIVLTVPGS |
| | ERSYDLTGLKPGTEYMVFIQGVKGGCFSTPLYAIFTT |
| 472 | MLPAPKNLVVSRVTEDSARLSWTAPDAAFDSFFIGYLEPQPPGEAIELYVPGS |
| | ERSYDLTGLKPGTEYNVTIQGVKGGFPSLPLVATFTT |
| 473 | MLPAPKNLVVSRVTEDSARLSWTAPDAAFDSFFIGYLEPQPPGEAIDLWVPGS |
| | ERSYDLTGLKPGTEYNVTIQGVKGGFPSLPLSASFTT |
| 474 | MLPAPKNLVVSRVTEDSARLSWTAPDAAFDSFFIGYLEPQPPGEAIGLAVPGS |
| | ERSYDLTGLKPGTEYNVTIQGVKGGFPSEPLIADFTT |
| 475 | MLPAPKNLVVSRVTEDSARLSWTAPDAAFDSFFIGYLEPQPPGEAITLYVPGS |
| | ERSYDLTGLKPGTEYNVTIQGVKGGFPSIPLSAEFTT |
| 476 | MLPAPKNLVVSRVTEDSARLSWTAPDAAFDSFFIGYLEPQPPGEAIHLQVPGS |
| | ERSYDLTGLKPGTEYNVTIQGVKGGFPSIPLIAVETT |
| 477 | MLPAPKNLVVSRVTEDSARLSWTAPDAAFDSFEIAYFETCRTGEAIVLTVPGS |
| | ERSYDLTGLKPGTEYGVSIYGVKGGAHSGPLSAIFTT |
| 478 | MLPAPKNLVVSRVTEDSARLSWTAPDAAFDSFFIGYLEPQPPGEAIHLYVPGS |
| | ERSYDLTGLKPGTEYNVTIQGVKGGFPSVPLIAKFTT |
| 479 | MLPAPKNLVVSRVTEDSARLSWTAPDAAFDSFFIGYLEPQPPGEAINLWVPGS |
| | ERSYDLTGLKPGTEYNVTIQGVKGGFPSTPLFAGFTT |
| 480 | MLPAPKNLVVSRVTEDSARLSWTAPDAAFDSFEIDYAEDIREGEAIALVVPGS |
| | ERSYDLTGLKPGTEYWVSIGGVKGGTWSRPLFAPFTT |
| 481 | MLPAPKNLVVSRVTEDSARLSWTAPDAAFDSFFIGYLEPQPPGEAITLYVPGS |
| | ERSYDMTGLKPGTEYNVTIQGVKGGFPSLPLQAHFTT |
| 482 | MLPAPKNLIVSRVTEDSARLSWTAPDAAFDSFFIGYLEPQPPGEAIHLQVPGS |
| | ERSYDLTGLKPGTEYNVTIQGVKGGFPSNPLHAEFTT |
| 483 | MLPAPKNLVVSRVTEDSARLSWTAPDAAFDSFFIGYLEPQPPGEAITLVVPGS |
| | ERSYDLTGLKPGTEYNVTIQGVKGGFPSDPLVASFTT |
| 484 | MLPAPKNLVVSRVTEDSARLSWTAPDAAFDSFEIAYQELSLWGEAIVLTVPGS |
| | ERSYDLTGLKPGTEYYVHIGGVKGGIWSSPLSAISTT |
| 485 | MLPAPKNLVVSRVTEDSARLSWTAPDAAFDSFFIGYLEPQPPGEAIHLWVPGS |
| | ERSYDLTGLKPGTEYNVTIQGVKGGFPSLPLLAKFTT |
| 486 | MLPAPENLVVSRVTEDSARLSWTALDAAFDSFFIGYLEPQPPGEAISLLVPGS |
| | ERSYDLTGLKPGTEYNVTIQGVKGGIASEPLSAAFTT |
| 487 | MLPAPKNLVVSRVTEDSARLSWTAPDAAFDSFFIGYLEPQPPGEAIGLRVPGS |
| | ERSYDLTGLKPGTEYNVTIQGVKGGFPSEPLIADFTT |
| 488 | MLPAPKNLVVSRVTEDSARLSWTAPDAAFDSFIIQYGEYLRWGEAIVLTVPGS |
| | ERSYDLTGLKPGTEYQVEIYGVKGGPLSKPLSAIFTT |
| 489 | MLAPKNLVVSRVTEDSARLSWTAPDAAFDSFFIGYLEPQPPGEAISLLVPGSE |
| | RSYDLTGLKPGTEYNVTIQGVKGGFPSSPLLAGFTT |
| 490 | MLPAPKNLVVSRVTEDSARLSWTAPDAAFDSFFIGYLEPQPPGEAISLQVPGS |
| | ERSYDLTGLKPGTEYNVTIQGVKGGFPSAPLVANFTT |
| 491 | MLPAPKNLVVSRVTEDSARLSWTAPDAAFDSFFIGYLEPQPSGEAIHLIVPGS |
| | ERSYDLTGLKPGTEYNVTIQGVKGGFPSDPLVASFTT |
| 492 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKFTT |
| 493 | MLPAPKNLVVSRVTEDSARLSWTAPDAAFDSFFIGYLEPQPPGEAILLAVPGS |
| | ERSYDLTGLKPGTEYNVTIQGVKGGFPSLPLVAHFTT |
| 494 | MLPAPKNLVVSRVTEDSARLSWTTPDAAFDSFFIGYLEPQPPGEAISLYVPGS |
| | ERSYDLTGLKPGTEYNVTIQGVKGGFPSMPLSAIFTT |
| 495 | MLPAPKNLVVSRVTEDSARLSWTAPDAAFDSFFINYREDKWIGEAIVLTVPGS |
| | ERSYDLTGLKPGTEYSVPIDGVKGGAASPPLSAIFTT |
| 496 | MLPAPKNLVVSRVTEDSARLSWTAPDAAFDSFFIGYLEPQPPGEAIILWVPGS |
| | ERSYDLTGLKPGTEYNVTIQGVKGGFPSVPLIAQFTT |
| 497 | MLPAPKNLVVSRVTEDSARLSWTAPDAAFDSFFIGYLEPQPPGEAITLLVPGS |
| | ERSYDLTGLKPGTEYNVTIQGVKGGFPSKPLYAYFTT |
| 498 | MLPAPKNLVVSRVTEDSARLSWTAPDAAFDSFFIGYLEPQPPGEAIILWVPGS |
| | ERSYDLTGLKPGTEYNVTIQGVKGGFPSIPLIAVFTT |
| 499 | MLPAPKNLVVSRVTEDSARLSWTAPDAAFDSFFIGYLEPQPPGEAISLYVPGS |
| | ERSYDLTGLKPGTEYNVTIQGVKGGFPSSPLFADETT |
| 500 | MLPAPKNLVVSRVTEDSARLSWTAPDAAFDSFFIGYLEPQPPGEAIDLLVPGS |
| | ERSYDLTGLKPGTEYNVTIQGVKGGFPSMPLSAIFTT |
| 501 | MLPAPKNLVVSRVTEDSARLSWTAPDAAFDSFFIGYLEPQPPGEAISLLVPGS |
| | ERSYDLTGLKPGTEYNVTIQGVKGGFPSVPLSAIFTT |
| 502 | MLPAPKNLVVSRVTEDSARLSWTAPDAAFDSFFIGYLEPQPPGEAIGLHVPGS |
| | ERSYDLTGLKPGTEYNVTIQGVKGGFPSIPLEAKFTT |
| 503 | MLPAPKNLVVSRVTEDSARLSWTAPDAAFDSFEISYFETSWHGEAIVLTVLGS |
| | ERSYDLTGLKPGTEYRVYIGGVKGGSWSQPLSAIFTT |
| 504 | MLPAPKNLVVSRVTEDSARLSWTAPDAAFDSFFIGYLEPQPPGEAIGLVVPGS |
| | ERSYDLTGLKPGTEYNVTIQGVKGGFPSHPLGAIFTT |
| 505 | MLPAPKNLVVSRVTEDSARLSWTALDAAFDSFFIGYLEPQPPGEAIHLFVPGS |
| | ERSYDLTGLKPGTEYNVTIQGVKGGFPSEPLIANFTT |
| 506 | MLPAPKNLVVSRVTEDSARLSWTAPDAAFDSFEIGYWENAYKGEAIVLTVPGS |
| | ERSYDLTGLKPGTEYVVFIGGVKGGVWSVPLSAISTT |
| 507 | MLPAPKNLVVSRVTEDSARLSWTAPDAAFDSFFIGYLEPQPPGEAIHLLVPGS |
| | ERSYDLTGLKPGTEYNVTIQGVKGGFPSMPLSAIFTT |
| 508 | MLPAPKNLVVSRVTEDSARLSWTAPDAAFDSFFIGYLEPQPPGEAITLSVPGS |
| | ERSYDLTGLKPGTEYNVTIQGVKGGFPSEPLIAIFTT |
| 509 | MLPAPKNLVVSRVTEDSARLSWTAPDAAFDSFEIGYWENAYKGEAIVLTVPGS |
| | ERSYDLTGLKPGTEYEVFIGGVKGGVWSVPLSAIFTT |
| 510 | MLPAPKNLVVSRVTEDSARLSWTAPDAAFDSFFIAYGESPESGEAIVLTVPGS |
| | ERSYDLTGLKPGTEYLVWIAGVKGGYYSDPLSAIFTT |
| 511 | MLPAPKNLVVSRVTEDSARLSWTAPDAAFDSFFIGYLEPQPPGEAIDLFVPGS |
| | ERSYDLTGLKPGTEYNVTIQGVKGGFPSLPLVASFTT |
| 512 | MLPAPKNLVVSRVTEDSARLSWTAPDAAFDSFFIGYLEPQPPGEAIDLWVPGS |
| | ERSYDLTGLKPGTEYNVTIQGVKGGFPSDPLDAHFTT |
| 513 | MLPAPKNLVVSRVTEDSARLSWTAPDAAFDSFFIGYLEPQPPGEAISLYVPGS |
| | ERSYDLTGLKPGTEYNVTIQGVKGGFPSLPLAAQFTT |
| 514 | MLPAPKNLVVSRVTEDSARLSWTAPDAAFDSFFIGYLEPQPPGEAIHLFVPGS |
| | ERSYDLTGLKPGTEYNVTIQGVKGGFPSEPLIANFTT |
| 515 | MLPAPKNLVVSRVTEDSARLSWTAPDAAFDSFFIGYLEPQPPGEAIALWVPGS |
| | ERSYDLTGLKPGTEYNVTIQGVKGGFPSTPLIAQFTT |
| 516 | MLPAPKNLVVSRVTEDSARLSWTAPDAAFDSFFIGYLEPQPPGEAISLWVPGS |
| | ERSYDLTGLKPGTEYNVTIQGVKGGFPSSPLVAYFTT |
| 517 | MLPAPKNLVVSRVTEDSARLSWTAPDAAFDSFFIGYLEPQPPGEAIALVVPGS |
| | ERSYDLTGLKPGTEYNVTIQGVKGGFPSMPLFAYFTT |
| 518 | MLPAPKNLVVSRVTEDSARLSWTAPDAAFDSFFIGYLEPQPPGEAISLWVPGS |
| | ERSYDLTGLKPGTEYNVTIQGVKGGFPSDPLGAHFTT |
| 519 | MLPAPKNLVVSRVTEDSARLSWTAPDAAFDSFPIAYPEHLPPGEAIVLTVPGS |
| | ERSYDLTGLKPGTEYPVNIRGVKGGFVSFPLSAIFTT |
| 520 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAKFTT |
| 521 | MLPAPKNLVVSRVTEDSARLSWTAPDAAFDSFFIGYLEPQPPGEAIVLWVPGS |
| | ERSYDLTGLKPGTEYNVTIQGVKGGFPSHPLVAHFTT |
| 522 | MLPAPKNLVVSRVTEDSARLSWTAPDAAFDSFFIGYLEPQPPGEAITLYVPGS |
| | ERSYDLNGLKPGTEYNVTIQGVKGGFPSMPLSAIFTT |
| 523 | MLPAPKNLVVSRVTEDSARLSWTAPDAAFDSFFIGYLEPQPPGEAIDLLVPGS |
| | ERSYDLTGLKPGTEYNVTIQGVKGGFPSEPLTAEFTT |
| 524 | MLPAPKNLVVSRVTEDSARLSWTAPDAAFDSFFIGYLEPQPPGEAIHLWVPGS |
| | ERSYDLTGLKPGTEYNVTIQGVKGGFPSMPLSAIFTT |
| 525 | MLPAPKNLVVSRVTEDSARLSWTAPDAAFDSFFIGYLEPQPPGEAIALYVPGS |
| | ERSYDLTGLKPGTEYNVTIQGVKGGFPSEPLVATFTT |
| 526 | MLPAPKNLVVSRVTEDSARLSWTAPDAAFDSFFIGYLEPQPPGEAISLIVPGS |
| | ERSYDLTGLKPGTEYNVTIQGVKGGFPSDPLIAIFTT |
| 527 | MLPAPKNLVVSRVTEDSARLSWTAPDAAFDSFFIGYLEPQPPGEAIHLYVPGS |
| | ERSYDLTGLKPGTEYNVTIQGVKGGFPSEPLIANFTT |
| 528 | MLPAPKNLVVSRVTEDSARLSWTAPDAAFDSFFIGYLEPQPPGEAITLLVPGS |
| | ERSYDLTGLKPGTEYNVTIQGVKGGFPSIPLIAIFTT |
| 529 | MLPAPKNLVVSRVTEDSARLSWTAPDAAFDSFFIGYLEPQPPGEAIDLYVPGS |
| | ERSYDLTGLKPGTEYNVTIQGVKGGFPSLPLQAHFTT |
| 530 | MLPAPKNLVVSRVTEDSARLSWTAPDAAFDSFFIGYLEPQPPGEAIDLLVPGS |
| | ERSYDLTGLKPGTEYNVTIQGVKGGFPSEPLFAHFTT |
| 531 | MLPAPKNLVVSRVTEDSARLSWTAPDAAFDSFEIGYFEYSYYGEAIVLTVPGS |
| | ERSYDLTGLKPGTEYRVYIGGVKGGGWSRPLSAIFTT |
| 532 | MLPAPKNLVVSRVTEDSARLSWTAPDAAFDSFDINYDELSGEGEAIALFVPGS |
| | ERSYDLTGLKPGTEYWVSIGGVKGGRESEPLYARFTT |
| 533 | MLPAPKNLVVSRVTEDSARLSWTAPDAAFDSFFIGYLEPQPPGEAINLFVPGS |
| | ERSYDLTGLKPGTEYNVTIQGVKGGFPSMPLSAIFTT |
| 534 | MLPAPKNLVVSRVTEDSARLSWTAPDAAFDSFFIGYLEPQPPGEAIHLLVPGS |
| | ERSYDLTGLKPGTEYNVTIQGVKGGFPSGPLQASFTT |
| 535 | MLPAPKNLVVSRVTEDSARLSWTAPDAAFDSFFIGYLEPQPPGEAIHLQVPGS |
| | ERSYDLTGLKPGTEYNVTIQGVKGGFPSDPLVAKFTT |
| 536 | MLPAPKNLVVSRVTEDSARLSWTAPDAAFDSFDIGYTETVSFGEAIVLTVPGS |
| | ERSYDLTGLKPGTEYIVKILGVKGGFASFPLSAIFTT |
| 537 | MLPAPKNLVVSRVTEDSARLSWTAPDAAFDSFEIGYFENLYLGEAIVLTVPGS |
| | ERSYDLTGLKPGTEYWVQIGGVKGGNWSAPLSAIFTT |
| 538 | MLPAPKNLVVSRVTEDSARLSWTAPDAAFDSFFIGYLEPQPPGEAIDLYVPGS |
| | ERSYDLTGLKPGTEYNVTIQGVKGGFPSHPLVAVFTT |
| 539 | MLPAPKNLVVSRVTEDSARLSWTAPDAAFDSFFIGYLEPQPPGEAIHLFVPGS |
| | ERSYDLTGLKPGTEYNVTIQGVKGGFPSDPLHAAFTT |
| 540 | MLPAPKNLVVSRVTEDSARLSWTAPDAAFDSFYIAYAEPRPDGEAIILVVPGS |
| | ERSYDLTGLKPGTEYSVLIHGVKGGLLSSPLDAGFTT |
| 541 | MLPAPKNLVVSRVTEDSARLSWTAPDAAFDSFFIGYLEPQPPGEAIDLWVPGS |
| | ERSYDLTGLKPGTEYNVTIQGVKGGFPSMPLDAYFTT |
| 542 | MLPAPKNLVVSRVTEDSARLSWTAPDAAFDSFFIGYLEPQPPGEAIELFVPGS |
| | ERSYDLTGLKPGTEYNVTIQGVKGGFPSMPLSAIFTT |
| 543 | MLPAPKNLVVSRVTEDSARLSWTAPDAAFDSFFIGYLEPQPPGEAISLYVPGS |
| | ERSYDLTGLKPGTEYNVTIQGVKGGFPSIPLVAVETT |
| 544 | MLPAPKNLVVSRVTEDSARLSWTAPDAAFDSFFIGYLEPQPPGEAIALLVPGS |
| | ERSYDLTGLKPGTEYNVTIQGVKGGFPSHPLSASFTT |
| 545 | MLPAPKNLVVSRVTEDSARLSWTAPDAAFDSFFIGYLEPQPPGEAISLVVPGS |
| | ERSYDLTGLKPGTEYNVTIQGVKGGFPSNPLIATFTT |
| 546 | MLPAPKNLVVSRVTEDSARLSWTAPDAAFDSFEIAYIEVNIQGEAIVLTVPGS |
| | ERSYDLTGLKPGTEYYVHIGGVKGGPSSSPLSAIFTT |
| 547 | MLPAPKNLVVSRVTEDSARLSWTAPDAAFDSFFIGYLEPQPPGEAIHLIVPGS |
| | ERSYDLTGLKPGTEYNVTIQGVKGGFPSDPLVASFTT |
| 548 | MLPAPKNLVVSRVTEDSARLSWTAPDAAFDSFFIGYLEPQPPGEAIYLFVPGS |
| | ERSYDLTGLKPGTEYNVTIQGVKGGFPSMPLSAIFTT |
| 549 | MLPAPKNLVVSRVTEDSARLSWTAPDAAFDSFFIGYLEPQPPGEAISLWVPGS |
| | ERSYDLTGLKPGTEYNVTIQGVKGGFPSMPLSAIFTT |
| 550 | MLPAPKNLVVSRVTEDSARLSWTAPDAAFDSFFIGYLEPQPPGEAIHLFVPGS |
| | ERSYDLTGLKPGTEYNVTIQGVKGGFPSIPLYARFTT |
| 551 | MLPAPKNLVVSRVTEDSARLSWTAPDAAFDSFEIAYFETCRTGEAIVLTVPGS |
| | ERSYDLTGLKPGTEYWVQIGGVKGGNWSAPLSAIFTT |
| 552 | MLPAPKNLVVSRVTEDSARLSWTAPDAAFDSFFIGYLEPQPPGEAIHLFVPGS |
| | ERSYDLTGLKPGTEYNVTIQGVKGGFPSMPLSAIFTT |
| 972 | MSLPAPKNLVVSRVTEDSARLSWTAPDAAFDSFFIGYLEPQPPGEAIALYVPG |
| | SERSYDLTGLKPGTEYNVTIQGAKGGFPSEPLVVHFTT |
| 973 | MSLPAPKNLVVSRVTEDSARLSWTAPDAAFDSFFIGYLEPQPPGEAIHLYVPG |
| | SERSYDLTGLKPGTEYNGTIQGVKVGFPSEPLIANFTT |
| 974 | MSLPAPKNLVVSRVTEDSARLSWTAPDAAFDSFFIGYLEPQPPGEAIALYVPG |
| | SERSYDLTGLKPGTEYNVTIQGVKGGFPSLPLVATFTT |
| 975 | MLPAPKNLVVSRVTEDSARLSWTAPDAAFDSFFIGYLEPQPPGEAIELYVPGS |
| | CRSYDLTGLKPGTEYNVTIQGVKGGFPSLPLVATFTT |
| 976 | MLPAPKNLVVSRVTEDSARLSWTAPDAAFDSFFIGYLEPQPPGEAIHLYVPGS |
| | CRSYDLTGLKPGTEYNVTIQGVKGGFPSVPLIAKFTT |

### Conjugates of the CD71-Binding FN3 Domains of the Disclosure

In some embodiments, an isolated FN3 domain that binds to CD71 is conjugated or linked to a heterologous molecule(s).

In some embodiments, the FN3 domain that binds to CD71 is conjugated or linked to another binding moiety. In some embodiments, the binding moiety is one or more FN3 domains. In some embodiments, the additional FN3 domains bind different targets other than CD71. This would enable the peptide to be multi-specific (e.g. bi-specific, tri-specific, etc. . . . ), such that it binds to CD71 and another target molecule. The dimers and multimers may be generated by linking monospecific, bi- or multispecific protein scaffolds, for example, by the inclusion of an amino acid linker, for example a linker containing poly-glycine, glycine and serine, or alanine and proline. In some embodiments, the linker can be a flexible linker. The linker can be a short peptide sequence, such as those described herein. For example, the linker can be a G/S or G/A linker and the like. As provided herein, the linker can be, for example, a linker as shown in Table 4.

TABLE 4
Linkers

| SEQ ID NO | Linker Sequence |
|---|---|
| 46 | (GS)₂ |
| 47 | (GGGS)₂ |
| 48 | (GGGGS)₁₋₅ |
| 49 | (GGGGS)₅ |
| 50 | (GGGGA)₁₋₅ |
| 51 | (AP)₁₋₂₀ |
| 52 | (AP)₂₋₂₀ |
| 53 | (AP)₂ |
| 54 | (AP)₅ |
| 55 | (AP)₁₀ |
| 56 | (AP)₂₀ |
| 57 | A(EAAAK)₅AAA |
| 58 | (EAAAK)₁₋₅ |
| 59 | EAAAKEAAAKEAAAKEAAAK |
| 60 | GGGGSGGGGSGGGGSGGGGS |
| 61 | APAPAPAPAP |
| 62 | EAAAK |

In some embodiments, the FN3 domain comprising two FN3 domains connected by a linker have the amino acid sequence of one of SEQ ID Nos: 46-62. The dimers and multimers may be linked to each other in a N-to C-direction. The use of naturally occurring as well as synthetic peptide linkers to connect polypeptides into novel linked fusion polypeptides is well known in the literature (Hallewell et al., J Biol Chem 264, 5260-5268, 1989; Alfthan et al., Protein Eng. 8, 725-731, 1995; Robinson & Sauer, Biochemistry 35, 109-116, 1996; U.S. Pat. No. 5,856,456). The linkers described in this paragraph may be also be used to link the domains provided in the formula provided herein and above.

The FN3 domains may also, in some embodiments, incorporate other subunits for example via covalent interaction. In some embodiments, the FN3 domains that further comprise a half-life extending moiety. Exemplary half-life extending moieties are albumin, albumin variants, albumin-binding proteins and/or domains, an aliphatic chain or chains that thing to serum proteins, transferrin and fragments and analogues thereof, and Fc regions. Amino acid sequences of the human Fc regions are well known, and include IgG1, IgG2, IgG3, IgG4, IgM, IgA and IgE Fc regions. In some embodiments, the FN3 domain binds to albumin, albumin variants, albumin-binding proteins and/or domains, and fragments and analogues thereof extending the half-life of the entire molecule.

In some embodiments, the albumin binding domain comprises the amino acid sequence of SEQ ID NOs: 5, 6, 7, 8, 9, 10, 11, 12, 13, 14, 15, 16, 17, 18, 19, 20, 21, 22, or 23, provided in Table 5 below. In some embodiments, the albumin binding domain (protein) is isolated. In some embodiments, the albumin binding domain comprises an amino acid sequence that is at least, or is, 85%, 86%, 87%, 88%, 89%, 90%, 91%, 92%, 93%, 94%, 95%, 96%, 97%, 98%, or 99% identical to the amino acid sequence of SEQ ID NO: 5, 6, 7, 8, 9, 10, 11, 12, 13, 14, 15, 16, 17, 18, 19, 20, 21, 22, or 23. In some embodiments, the albumin binding domain comprises an amino acid sequence that is at least, or is, 85%, 86%, 87%, 88%, 89%, 90%, 91%, 92%, 93%, 94%, 95%, 96%, 97%, 98%, or 99% identical to the amino acid sequence of SEQ ID NO: 5, 6, 7, 8, 9, 10, 11, 12, 13, 14, 15, 16, 17, 18, 19, 20, 21, 22, or 23 provided that the protein has a substitution that corresponds to position 10 of SEQ ID NO: 5, 6, 7, 8, 9, 10, 11, 12, 13, 14, 15, 16, 17, 18, 19, 20, 21, 22, or 23. In some embodiments, the substitution is A10V. In some embodiments, the substitution is A10G, A10L, A10I, A10T, or A10S. In some embodiments, the substitution at position 10 is any naturally occurring amino acid. In some embodiments, the isolated albumin binding domain comprises an amino acid sequence that has 1, 2, 3, 4, 5, 6, 7, 8, 9, 10, 11, 12, 13, or 14 substitutions when compared to the amino acid sequence of SEQ ID NOs: 5, 6, 7, 8, 9, 10, 11, 12, 13, 14, 15, 16, 17, 18, 19, 20, 21, 22, or 23. In some embodiments, the substitution is at a position that corresponds to position 10 of SEQ ID NOs: 5, 6, 7, 8, 9, 10, 11, 12, 13, 14, 15, 16, 17, 18, 19, 20, 21, 22, or 23. In some embodiments, FN3 domains provided comprises a cysteine residue in at least one residue position corresponding to residue positions 6, 11, 22, 25, 26, 52, 53, 61, 88 or positions 6, 8, 10, 11, 14, 15, 16, 20, 30, 34, 38, 40, 41, 45, 47, 48, 53, 54, 59, 60, 62, 64, 70, 88, 89, or 90 of SEQ ID NO: 5, 6, 7, 8, 9, 10, 11, 12, 13, 14, 15, 16, 17, 18, 19, 20, 21, 22, or 23, or at a C-terminus. Although the positions are listed in a series, each position can also be chosen individually. In some embodiments, the cysteine is at a position that corresponds to position 6, 53, or 88. In some embodiments, additional examples of albumin binding domains can be found in U.S. Pat. No. 10,925,932, which hereby incorporated by reference.

TABLE 5
Albumin-binding FN3 Domains

| SEQ ID | |
|---|---|
| NO: | SEQUENCE |
| 5 | MLPAPKNLVASRVTEDSARLSWTAPDAAFDSFNIAYWEPGIGGEAIWLRVPGS |
| | ERSYDLTGLKPGTEYKVWIHGVKGGASSPPLIARFTT |
| 6 | MLPAPKNLVVSRVTEDSARLSWTAPDAAFDSFNIAYWEPGIGGEAIWLRVPGS |
| | ERSYDLTGLKPGTEYKVWIHGVKGGASSPPLIARFTT |
| 7 | MLPAPKNLVVSRVTEDSARLSWTAPDAAFDSFAIAYWEPGIGGEAIWLRVPGS |
| | ERSYDLTGLKPGTEYKVWIHGVKGGASSPPLIARFTT |
| 8 | MLPAPKNLVVSRVTEDSARLSWTAPDAAFDSFNISYWEPGIGGEAIWLRVPGS |
| | ERSYDLTGLKPGTEYKVWIHGVKGGASSPPLIARFTT |
| 9 | MLPAPKNLVVSRVTEDSARLSWTAPDAAFDSFNIAYAEPGIGGEAIWLRVPGS |
| | RSYDLTGLKPGTEYKVWIHGVKGGASSPPLIARFTT |
| 10 | MLPAPKNLVVSRVTEDSARLSWTAPDAAFDSFNIAYWEAGIGGEAIWLRVPGS |
| | ERSYDLTGLKPGTEYKVWIHGVKGGASSPPLIARFTT |
| 11 | MLPAPKNLVVSRVTEDSARLSWTAPDAAFDSFNIAYWEPAIGGEAIWLRVPGS |
| | ERSYDLTGLKPGTEYKVWIHGVKGGASSPPLIARFTT |
| 12 | MLPAPKNLVVSRVTEDSARLSWTAPDAAFDSFNIAYWEPGAGGEAIWLRVPGS |
| | ERSYDLTGLKPGTEYKVWIHGVKGGASSPPLIARFTT |
| 13 | MLPAPKNLVVSRVTEDSARLSWTAPDAAFDSFNIAYWEPGIAGEAIWLRVPGS |
| | ERSYDLTGLKPGTEYKVWIHGVKGGASSPPLIARFTT |
| 14 | MLPAPKNLVVSRVTEDSARLSWTAPDAAFDSFNIAYWEPGIGGEAIALRVPGS |
| | ERSYDLTGLKPGTEYKVWIHGVKGGASSPPLIARFTT |
| 15 | MLPAPKNLVVSRVTEDSARLSWTAPDAAFDSFNIAYWEPGIGGEAIWLAVPGS |
| | ERSYDLTGLKPGTEYKVWIHGVKGGASSPPLIARFTT |
| 16 | MLPAPKNLVVSRVTEDSARLSWTAPDAAFDSFNIAYWEPGIGGEAIWLRVPGS |
| | ERSYDLTGLKPGTEYAVWIHGVKGGASSPPLIARFTT |
| 17 | MLPAPKNLVVSRVTEDSARLSWTAPDAAFDSFNIAYWEPGIGGEAIWLRVPGS |
| | ERSYDLTGLKPGTEYKVAIHGVKGGASSPPLIARFTT |
| 18 | MLPAPKNLVVSRVTEDSARLSWTAPDAAFDSFNIAYWEPGIGGEAIWLRVPGS |
| | ERSYDLTGLKPGTEYKVWIAGVKGGASSPPLIARFTT |
| 19 | MLPAPKNLVVSRVTEDSARLSWTAPDAAFDSFNIAYWEPGIGGEAIWLRVPGS |
| | ERSYDLTGLKPGTEYKVWIHGVKGGSSSPPLIARFTT |
| 20 | MLPAPKNLVVSRVTEDSARLSWTAPDAAFDSFNIAYWEPGIGGEAIWLRVPGS |
| | ERSYDLTGLKPGTEYKVWIHGVKGGAASPPLIARFTT |
| 21 | MLPAPKNLVVSRVTEDSARLSWTAPDAAFDSFNIAYWEPGIGGEAIWLRVPGS |
| | ERSYDLTGLKPGTEYKVWIHGVKGGASSAPLIARFTT |
| 22 | MLPAPKNLVVSRVTEDSARLSWTAPDAAFDSFNIAYWEPGIGGEAIWLRVPGS |
| | ERSYDLTGLKPGTEYKVWIHGVKGGASSPPLAARFTT |
| 23 | MLPAPKNLVVSRVTEDSARLSWTAPDAAFDSFNIAYWEPGIGGEAIWLRVPGS |
| | ERSYDLTGLKPGTEYKVWIHGVKGGASSPPLIAAFTT |

In some embodiments, other molecules linked to the FN3 domain can include Endoporter, INF-7, TAT, polyarginine, polylysine, or an amphipathic peptide. These moieties can be used in place of or in addition to other half-life extending moieties provided for herein. In some embodiments, other molecules linked to the FN3 domain are molecules that deliver the complex into the cell, the endosome, or the ER; said molecules are selected from those peptides listed in Table 6.

TABLE 6
Half-Life Extending Moieties

| SEQ ID | | |
|---|---|---|
| NO: | NAME | SEQUENCE |
| 24 | TAT | RKKRRQRRR |
| 25 | Penetratin | RQIKIWFQNRRMKWKK |
| 26 | Transportan | GWTLNSAGYLLGKINKALAALAKKIL |
| 27 | MAP | KLALKLALKALKAALKLA |
| 28 | Pep-1 | KETWWETWWTEWSQPKKKRKV |
| 29 | KDEL | KDEL |
| 30 | GALA | WEAALAEALAELAEHLAEALAEALEALAA |
| 31 | HA2 | GDIMGEWGNEIFGAIAGFLGC |
| 32 | Aurine 1.2 | GLFDIIKKIAESF |
| 33 | MPG | GALFLGWLGAAGSTMGAPKSKRKV |
| 34 | TP-10 | AGYLLGKINLKALAALAKKIL |
| 35 | EB-1 | LIRLWSHLIHIWFQNRRLKWKKK |
| 36 | HA2- | GLFGAIAGFIENGWEGMIDGRQIKIWFQNRRMKWKK |
| | Penetratin | |
| 37 | Endosomolytic | FFKKLAHALHLLALLALHLAHALKKA |
| 38 | Endosomolytic | LFEAIEGFIENGWGMIDGWYG |
| 39 | Endosomolytic | LFEAIEGFIENGWEGMIDGWYGRKKRRQRRR |
| 40 | Endosomolytic | IGAVLKVLTTGLPALISWIKRKRQQ |
| 41 | ER Targeting | MKLAVTLTLVTLALSSSSASA |
| 42 | ER Targeting | RLIEDICLPRWGCLWEDDKDEL |
| 43 | ER Targeting | MIRTLLLSTLVAGALSK |
| 44 | ER Targeting | ILSSLTVTQLLRRLHQWIK |
| 45 | ER Targeting | MIRTLLLSTLVAGALSKDEL |

All or a portion of an antibody constant region may be attached to the FN3 domain to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, down regulation of cell surface receptors (e.g., B cell receptor; BCR), and may be further modified by modifying residues in the Fc responsible for these activities (for review; see Strohl, Curr Opin Biotechnol. 20, 685-691, 2009).

Additional moieties may be incorporated into the FN3 domains such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules disclosed herein.

A PEG moiety may for example be added to the FN3 domain t by incorporating a cysteine residue to the C-terminus of the molecule, or engineering cysteines into residue positions that face away from the binding face of the molecule, and attaching a PEG group to the cysteine using well known methods.

FN3 domains incorporating additional moieties may be compared for functionality by several well-known assays. For example, altered properties due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating pharmacokinetic properties of the molecules disclosed herein in in vivo models.

In some embodiments, an FN3 domain that binds CD71 conjugated to a detectable label is provided. Detectable labels include compositions that when conjugated to the FN3 domains that bind CD71 renders CD71 detectable, via spectroscopic, photochemical, biochemical, immunochemical, or other chemical methods.

Exemplary detectable labels include, but are not limited to, radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, cintillants, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In some embodiments, the detectable label emits a signal as a result of being stimulated by an external stimulus, such as a magnetic or electric, or electromagnetic field.

Exemplary radioactive isotopes may be γ-emitting, Auger-emitting, β-emitting, an alpha-emitting or positron-emitting radioactive isotope. Exemplary radioactive isotopes include ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e., iodine) to 83 (i.e., bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn²⁺, Hg²⁺, Ni²⁺, Ni³⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The FN3 domains that specifically bind CD71 conjugated to a detectable label may be used, for example, as an imaging agent to evaluate tumor distribution, diagnosis for the presence of tumor cells and/or, recurrence of tumor.

In some embodiments, the peptide is conjugated to a lipid nanoparticle, which can be used, for example, for cell-specific targeting.

In some embodiments, an FN3 domain that binds CD71 conjugated to a therapeutic agent is provided. Non-limiting examples of therapeutic agents, such as, but not limited to, cytotoxic agents, are provided for herein.

In some embodiments, the therapeutic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate), an oligonucleotide, a RNA molecule, an siRNA molecule, an mi RNA molecule, an antisense oligonucleotide, or a DNA molecule.

The FN3 domains that bind CD71 conjugated to a therapeutic agent disclosed herein may be used in the targeted delivery of the therapeutic agent to CD71 expressing cells (e.g. tumor cells), and intracellular accumulation therein. Although not bound to any particular theory, this type of delivery can be helpful where systemic administration of these unconjugated agents may result in unacceptable levels of toxicity to normal cells.

In some embodiments, the therapeutic agent can elicit their cytotoxic and/or cytostatic effects by mechanisms such as, but not limited to, tubulin binding, DNA binding, topoisomerase inhibition, DNA cross linking, chelation, spliceosome inhibition, NAMPT inhibition, and HDAC inhibition.

In some embodiments, the therapeutic agent is a spliceosome inhibitor, a NAMPT inhibitor, or a HDAC inhibitor. In some embodiments, the agent is an immune system agonist, for example, TLR7,8,9, RIG-I (dsRNA), and STING (CpG) agonists. In some embodiments, the agent is daunomycin, doxorubicin, methotrexate, vindesine, bacterial toxins such as diphtheria toxin, ricin, geldanamycin, maytansinoids or calicheamicin.

In some embodiments, the therapeutic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, or the tricothecenes.

In some embodiments, the therapeutic agent is a radionuclide, such as ²¹²Bi, ¹³¹I, ¹³¹In, ⁹⁰Y or ¹⁸⁶Re.

In some embodiments, the therapeutic agent is dolastatin or dolastatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the FN3 domain through the N (amino) terminus or the C (carboxyl) terminus of the peptidic drug moiety (WO 02/088172), or via any cysteine engineered into the FN3 domain.

In some embodiments, therapeutic agent can be, for example, auristatins, camptothecins, duocarmycins, etoposides, maytansines and maytansinoids, taxanes, benzodiazepines or benzodiazepine containing drugs (e.g., pyrrolo[1,4]-benzodiazepines (PBDs), indolinobenzodiazepines, and oxazolidinobenzodiazepines) or vinca alkaloids.

### FN3 Domains Conjugated to Oligonucleotides

In some embodiments, the FN3 domain is conjugated to an oligonucleotide. For example, the oligonucleotide can be used for inhibiting the expression of a gene or mRNA transcript. The oligonucleotide can be a siRNA, miRNA, antisense oligonucleotide, and the like. Accordingly, in some embodiments, the FN3 domain can be conjugated to a polynucleotide, such as, but not limited to, a siRNA molecule, an antisense molecule, a RNA molecule, or a DNA molecule.

In some embodiments, the siRNA is a double-stranded RNAi (dsRNA) agent capable of inhibiting the expression of a target gene. The dsRNA agent comprises a sense strand (passenger strand) and an antisense strand (guide strand). In some embodiments, each strand of the dsRNA agent can range from 12-40 nucleotides in length. For example, each strand can be from 14-40 nucleotides in length, 17-37 nucleotides in length, 25-37 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

In some embodiments, the sense strand and antisense strand typically form a duplex dsRNA. The duplex region of a dsRNA agent may be from 12-40 nucleotide pairs in length. For example, the duplex region can be from 14-40 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotide pairs in length.

In some embodiments, the dsRNA comprises one or more overhang regions and/or capping groups of dsRNA agent at the 3′-end, or 5′-end or both ends of a strand. The overhang can be 1-10 nucleotides in length, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be other sequence. The first and second strands can also be joined, e.g., by additional bases to form a hairpin, or by other non-base linkers.

In some embodiments, the nucleotides in the overhang region of the dsRNA agent can each independently be a modified or unmodified nucleotide including, but not limited to 2′-sugar modified, such as, 2-F, 2′-Omethyl, 2′-O-(2-methoxyethyl), 2′-O-(2-methoxyethyl), 2′-O-(2-methoxyethyl), and any combinations thereof. For example, TT (UU) can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA agent may be phosphorylated. In some embodiments, the overhang region contains two nucleotides having a phosphorothioate, phosphorodithioate, phosphonate, phosphoramidate, or mesyl phosphoramidate between the two nucleotides, where the two nucleotides can be the same or different. In one embodiment, the overhang is present at the 3′-end of the sense strand, antisense strand or both strands. In one embodiment, this 3′-overhang is present in the antisense strand. In one embodiment, this 3′-overhang is present in the sense strand.

The dsRNA agent may comprise only a single overhang, which can strengthen the interference activity of the dsRNA, without affecting its overall stability. For example, the single-stranded overhang is located at the 3′-terminal end of the sense strand or, alternatively, at the 3′-terminal end of the antisense strand. The dsRNA may also have a blunt end, located at the 5′-end of the antisense strand (or the 3′-end of the sense strand) or vice versa. Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC. For example the single overhang comprises at least two, three, four, five, six, seven, eight, nine, or ten nucleotides in length.

In some embodiments, the dsRNA agent may also have two blunt ends, at both ends of the dsRNA duplex.

In some embodiments, every nucleotide in the sense strand and antisense strand of the dsRNA agent may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2 hydroxyl on the ribose sugar; wholesale replacement of the phosphate moiety with “dephospho” linkers; modification or replacement of a naturally occurring base; and replacement or modification of the ribose-phosphate backbone.

In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl (2′-OMe) modified instead of the ribosugar of the nucleobase, and modifications in the phosphate group, e.g., phosphorothioate, phosphorodithoate, phosphonate, phosphoramidate, or mesyl phosphoramidate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In one embodiment, each residue of the sense strand and antisense strand is independently modified with 2′-O-methyl or 2′-fluoro.

In some embodiments, at least two different modifications are typically present on the sense strand and antisense strand. Those two modifications may be the 2′-deoxy, 2′-O-methyl or 2′-fluoro modifications, acyclic nucleotides or others.

In one embodiment, the sense strand and antisense strand each comprises two differently modified nucleotides selected from 2′-fluoro, 2′-O-methyl or 2′-deoxy.

The dsRNA agent may further comprise at least one phosphorothioate, phosphorodithoate, phosphonate, phosphoramidate, mesyl phosphoramidate, or methylphosphonate internucleotide linkage. The phosphorothioate, phosphorodithoate, phosphonate, phosphoramidate, mesyl phosphoramidate, or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA agent comprises the phosphorothioate, phosphorodithoate, phosphonate, phosphoramidate, mesyl phosphoramidate, or methylphosphonate internucleotide linkage modification in the overhang region. For example, the overhang region comprises two nucleotides having a phosphorothioate, phosphorodithoate, phosphonate, phosphoramidate, mesyl phosphoramidate, or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate, phosphorodithoate, phosphonate, phosphoramidate, mesyl phosphoramidate, or methylphosphonate internucleotide linkage, and optionally, there may be additional phosphorothioate, phosphorodithoate, phosphonate, phosphoramidate, mesyl phosphoramidate, or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. In some embodiments, these terminal three nucleotides may be at the 3′-end of the antisense strand.

In some embodiments, the dsRNA composition is linked by a modified base or nucleoside analogue as described in U.S. Pat. No. 7,427,672, which is incorporated herein by reference. In some embodiments, the modified base or nucleoside analogue is referred to as the linker or L in formulas described herein.

In some embodiments, the modified base or nucleoside analogue has the structure as shown in Chemical Formula I and a salt thereof:

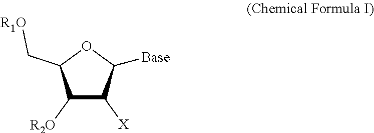

where Base represents an aromatic heterocyclic group or aromatic hydrocarbon ring group optionally having a substituent, R₁ and R₂ are identical or different, and each represent a hydrogen atom, a protective group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected with a protective group for nucleic acid synthesis, or —P(R₄)R₅ where R₄ and R₅ are identical or different, and each represent a hydroxyl group, a hydroxyl group protected with a protective group for nucleic acid synthesis, a mercapto group, a mercapto group protected with a protective group for nucleic acid synthesis, an amino group, an alkoxy group having 1 to 5 carbon atoms, an alkylthio group having 1 to 5 carbon atoms, a cyanoalkoxy group having 1 to 6 carbon atoms, or an amino group substituted by an alky group having 1 to 5 carbon atoms, and X denotes OMe or F.

In some embodiments, the modified base or nucleoside analogue has the structure as shown in Chemical Formula I and salts thereof, wherein Ri is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the modified base or nucleoside analogue has the structure as shown in Chemical Formula I and salts thereof, wherein R₁ is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the modified base or nucleoside analogue has the structure as shown in Chemical Formula I and salts thereof, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the modified base or nucleoside analogue has the structure as shown in Chemical Formula I and salts thereof, wherein R₂ is a hydrogen atom, an acetyl group, a benzoyl group, a methanesulfonyl group, a p-toluenesulfonyl group, a benzyl group, a p-methoxybenzyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the modified base or nucleoside analogue has the structure as shown in Chemical Formula I and salts thereof, wherein Base is a purin-9-yl group, a 2-oxopyrimidin-1-yl group, or a purin-9-yl group or a 2-oxopyrimidin-1-yl group having a substituent selected from the following a group: a group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the modified base or nucleoside analogue has the structure as shown in Chemical Formula I and salts thereof, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the modified base or nucleoside analogue has the structure as shown in Chemical Formula IB and a salt thereof

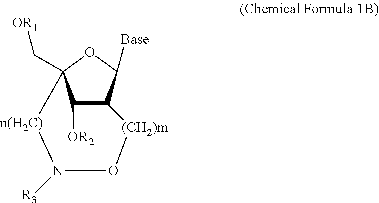

where Base represents an aromatic heterocyclic group or aromatic hydrocarbon ring group optionally having a substituent, R₁ and R₂ are identical or different, and each represent a hydrogen atom, a protective group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected with a protective group for nucleic acid synthesis, or —P(R₄)R₅ where R₄ and R₅ are identical or different, and each represent a hydroxyl group, a hydroxyl group protected with a protective group for nucleic acid synthesis, a mercapto group, a mercapto group protected with a protective group for nucleic acid synthesis, an amino group, an alkoxy group having 1 to 5 carbon atoms, an alkylthio group having 1 to 5 carbon atoms, a cyanoalkoxy group having 1 to 6 carbon atoms, or an amino group substituted by an alky group having 1 to 5 carbon atoms, R₃ represents a hydrogen atom, an alkyl group, an alkenyl group, a cycloalkyl group, an aryl group, an aralkyl group, an acyl group, a sulfonyl group, or a functional molecule unit substituent, and m denotes an integer of 0 to 2, and n denotes an integer of 0 to 3. In some embodiments, m and n are 0.

In some embodiments, the modified base or nucleoside analogue has the structure as shown in Chemical Formula IB and salts thereof, wherein Ri is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the modified base or nucleoside analogue has the structure as shown in Chemical Formula IB and salts thereof, wherein Ri is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the modified base or nucleoside analogue has the structure as shown in Chemical Formula IB and salts thereof, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the modified base or nucleoside analogue has the structure as shown in Chemical Formula IB and salts thereof, wherein R₂ is a hydrogen atom, an acetyl group, a benzoyl group, a methanesulfonyl group, a p-toluenesulfonyl group, a benzyl group, a p-methoxybenzyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the modified base or nucleoside analogue has the structure as shown in Chemical Formula IB and salts thereof, wherein R₃ is a hydrogen atom, a phenoxyacetyl group, an alkyl group having 1 to 5 carbon atoms, an alkenyl group having 1 to 5 carbon atoms, an aryl group having 6 to 14 carbon atoms, a methyl group substituted by one to three aryl groups, a lower aliphatic or aromatic sulfonyl group such as a methanesulfonyl group or a p-toluenesulfonyl group, an aliphatic acyl group having 1 to 5 carbon atoms such as an acetyl group, or an aromatic acyl group such as a benzoyl group.

In some embodiments, the modified base or nucleoside analogue has the structure as shown in Chemical Formula IB and salts thereof, wherein the functional molecule unit substituent as R₃ is a fluorescent or chemiluminescent labeling molecule, a nucleic acid incision activity functional group, or an intracellular or nuclear transfer signal peptide.

In some embodiments, the modified base or nucleoside analogue has the structure as shown in Chemical Formula IB and salts thereof, wherein Base is a purin-9-yl group, a 2-oxopyrimidin-1-yl group, or a purin-9-yl group or a 2-oxopyrimidin-1-yl group having a substituent selected from the following a group: a group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the modified base or nucleoside analogue has the structure as shown in Chemical Formula IB and salts thereof, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the modified base or nucleoside analogue has the structure as shown in Chemical Formula IB and salts thereof, wherein m is 0, and n is 1.

In some embodiments, the modified base or nucleoside analogue is a DNA oligonucleotide or RNA oligonucleotide analogue, containing one or two or more of one or more types of unit structures of nucleoside analogues having the structure as shown in Chemical Formula II, or a pharmacologically acceptable salt thereof, provided that a form of linking between respective nucleosides in the oligonucleotide analogue may contain one or two or more phosphorothioate bonds [—OP(O)(S⁻)O—], phosphorodithioate bonds [—O₂PS₂—], phosphonate bonds [—PO(OH)₂—], phosphoramidate bonds [—O═P(OH)₂—], or mesyl phosphoramidate bonds [—OP(O)(N)(SO₂)(CH₃)O—] aside from a phosphodiester bond [—OP(O₂⁻)O—] identical with that in a natural nucleic acid, and if two or more of one or more types of these structures are contained, Base may be identical or different between these structures:

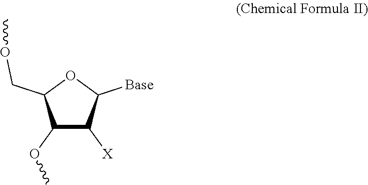

where Base represents an aromatic heterocyclic group or aromatic hydrocarbon ring group optionally having a substituent, and X denotes OMe or F.

In some embodiments, the oligonucleotide analogue or the pharmacologically acceptable salt thereof has the structure as shown in Chemical Formula II, wherein Base is a purin-9-yl group, a 2-oxopyrimidin-1-yl group, or a purin-9-yl group or a 2-oxopyrimidin-1-yl group having a substituent selected from the following α group: α group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the oligonucleotide analogue or the pharmacologically acceptable salt thereof has the structure as shown in Chemical Formula II, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl group having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the modified base or nucleoside analogue is a DNA oligonucleotide or RNA oligonucleotide analogue, containing one or two or more of one or more types of unit structures of nucleoside analogues having the structure as shown in Chemical Formula IIB, or a pharmacologically acceptable salt thereof, provided that a form of linking between respective nucleosides in the oligonucleotide analogue may contain one or two or more phosphorothioate bonds [—OP(O)(S⁻)O—], phosphorodithioate bonds [—O₂PS₂—], phosphonate bonds [—PO(OH)₂—], phosphoramidate bonds [—O═P(OH)₂—], or mesyl phosphoramidate bonds [—OP(O)(N)(SO₂)(CH₃)O—] aside from a phosphodiester bond [—OP(O₂⁻)O—] identical with that in a natural nucleic acid, and if two or more of one or more types of these structures are contained, Base may be identical or different between these structures:

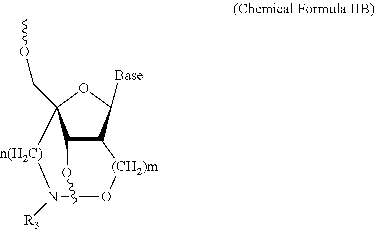

where Base represents an aromatic heterocyclic group or aromatic hydrocarbon ring group optionally having a substituent, R₃ represents a hydrogen atom, an alkyl group, an alkenyl group, a cycloalkyl group, an aryl group, an aralkyl group, an acyl group, a sulfonyl group, a silyl group, or a functional molecule unit substituent, and m denotes an integer of 0 to 2, and n denotes an integer of 0 to 3. In some embodiments, m and n are 0.

In some embodiments, the oligonucleotide analogue or the pharmacologically acceptable salt thereof has the structure as shown in Chemical Formula IIB, wherein Ri is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, or a silyl group.

In some embodiments, the oligonucleotide analogue or the pharmacologically acceptable salt thereof has the structure as shown in Chemical Formula IIB, wherein Ri is a hydrogen atom, an acetyl group, a benzoyl group, a methanesulfonyl group, a p-toluenesulfonyl group, a benzyl group, a p-methoxybenzyl group, a trityl group, a dimethoxytrityl group, a monomethoxytrityl group, or a tert-butyldiphenylsilyl group.

In some embodiments, the oligonucleotide analogue or the pharmacologically acceptable salt thereof has the structure as shown in Chemical Formula IIB, wherein R₂ is a hydrogen atom, an aliphatic acyl group, an aromatic acyl group, an aliphatic or aromatic sulfonyl group, a methyl group substituted by one to three aryl groups, a methyl group substituted by one to three aryl groups having an aryl ring substituted by a lower alkyl, lower alkoxy, halogen, or cyano group, a silyl group, a phosphoroamidite group, a phosphonyl group, a phosphate group, or a phosphate group protected with a protective group for nucleic acid synthesis.

In some embodiments, the oligonucleotide analogue or the pharmacologically acceptable salt thereof has the structure as shown in Chemical Formula IIB, wherein R₂ is a hydrogen atom, an acetyl group, a benzoyl group, a benzyl group, a p-methoxybenzyl group, a methanesulfonyl group, a p-toluenesulfonyl group, a tert-butyldiphenylsilyl group, —P(OC₂H₄CN)(N(i-Pr)₂), —P(OCH₃)(N(i-Pr)₂), a phosphonyl group, or a 2-chlorophenyl- or 4-chlorophenylphosphate group.

In some embodiments, the oligonucleotide analogue or the pharmacologically acceptable salt thereof has the structure as shown in Chemical Formula IIB, wherein R₃ is a hydrogen atom, a phenoxyacetyl group, an alkyl group having 1 to 5 carbon atoms, an alkenyl group having 1 to 5 carbon atoms, an aryl group having 6 to 14 carbon atoms, a methyl group substituted by one to three aryl groups, a lower aliphatic or aromatic sulfonyl group such as a methanesulfonyl group or a p-toluenesulfonyl group, an aliphatic acyl group having 1 to 5 carbon atoms such as an acetyl group, or an aromatic acyl group such as a benzoyl group.

In some embodiments, the oligonucleotide analogue or the pharmacologically acceptable salt thereof has the structure as shown in Chemical Formula IIB, wherein the functional molecule unit substituent as R₃ is a fluorescent or chemiluminescent labeling molecule, a nucleic acid incision activity functional group, or an intracellular or nuclear transfer signal peptide.

In some embodiments, the oligonucleotide analogue or the pharmacologically acceptable salt thereof has the structure as shown in Chemical Formula IIB, wherein Base is a purin-9-yl group, a 2-oxopyrimidin-1-yl group, or a purin-9-yl group or a 2-oxopyrimidin-1-yl group having a substituent selected from the following a group: a group: A hydroxyl group, a hydroxyl group protected with a protective group for nucleic acid synthesis, an alkoxy group having 1 to 5 carbon atoms, a mercapto group, a mercapto group protected with a protective group for nucleic acid synthesis, an alkylthio group having 1 to 5 carbon atoms, an amino group, an amino group protected with a protective group for nucleic acid synthesis, an amino group substituted by an alkyl group having 1 to 5 carbon atoms, an alkyl group having 1 to 5 carbon atoms, and a halogen atom.

In some embodiments, the oligonucleotide analogue or the pharmacologically acceptable salt thereof has the structure as shown in Chemical Formula IIB, wherein Base is 6-aminopurin-9-yl (i.e., adeninyl), 6-aminopurin-9-yl having the amino group protected with a protective group for nucleic acid synthesis, 2,6-diaminopurin-9-yl, 2-amino-6-chloropurin-9-yl, 2-amino-6-chloropurin-9-yl having the amino group protected with a protective group for nucleic acid synthesis, 2-amino-6-fluoropurin-9-yl, 2-amino-6-fluoropurin-9-yl having the amino group protected with a protective group for nucleic acid synthesis, 2-amino-6-bromopurin-9-yl, 2-amino-6-bromopurin-9-yl having the amino group protected with a protective group for nucleic acid synthesis, 2-amino-6-hydroxypurin-9-yl (i.e., guaninyl), 2-amino-6-hydroxypurin-9-yl having the amino group protected with a protective group for nucleic acid synthesis, 6-amino-2-methoxypurin-9-yl, 6-amino-2-chloropurin-9-yl, 6-amino-2-fluoropurin-9-yl, 2,6-dimethoxypurin-9-yl, 2,6-dichloropurin-9-yl, 6-mercaptopurin-9-yl, 2-oxo-4-amino-1,2-dihydropyrimidin-1-yl (i.e., cytosinyl), 2-oxo-4-amino-1,2-dihydropyrimidin-1-yl having the amino group protected with a protective group for nucleic acid synthesis, 2-oxo-4-amino-5-fluoro-1,2-dihydropyrimidin-1-yl, 2-oxo-4-amino-5-fluoro-1,2-dihydropyrimidin-1-yl group having the amino group protected with a protective group for nucleic acid synthesis, 4-amino-2-oxo-5-chloro-1,2-dihydropyrimidin-1-yl, 2-oxo-4-methoxy-1,2-dihydropyrimidin-1-yl, 2-oxo-4-mercapto-1,2-dihydropyrimidin-1-yl, 2-oxo-4-hydroxy-1,2-dihydropyrimidin-1-yl (i.e., uracinyl), 2-oxo-4-hydroxy-5-methyl-1,2-dihydropyrimidin-1-yl (i.e., thyminyl), 4-amino-5-methyl-2-oxo-1,2-dihydropyrimidin-1-yl (i.e., 5-methylcytosinyl), or 4-amino-5-methyl-2-oxo-1,2-dihydropyrimidin-1-yl having the amino group protected with a protective group for nucleic acid synthesis.

In some embodiments, the oligonucleotide analogue or the pharmacologically acceptable salt thereof has the structure as shown in Chemical Formula IIB, wherein m is 0, and nis 1.

In some embodiments, the dsRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS2), 5′-end vinylphosphonate (5′-VP), 5′-end methylphosphonate (MePhos), 5′-end mesyl phosphoramidate (5′MsPA), or 5′-deoxy-5′-C-malonyl. When the 5′-end phosphorus-containing group is 5′-end vinylphosphonate (5′-VP), the 5′-VP can be either 5′-E-VP isomer, such as trans-vinylphosphate or cis-vinylphosphate, or mixtures thereof. Representative structures of these modifications can be found in, for example, U.S. Pat. No. 10,233,448, which is hereby incorporated by reference in its entirety.

In some embodiments, nucleotide analogues or synthetic nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, methyl, ethyl, n-propyl, iso-propyl, n-butyl, iso-butyl, tert-butyl, C₁-C₁₀ chain lengths both linear and branched. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazine or hydroxylamino) group, an isocyanate or cyanate group, or a sulfur containing group (e.g., sulfoxide, sulfone, sulfide, and disulfide). In some instances, the alkyl moiety further comprises additional hetero atom such as O, S, N, Se and each of these hetero atoms can be further substituted with alky groups as described above. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. Exemplary chemical structures of a 2′-O-methyl modification of an adenosine molecule and 2′O-methoxyethyl modification of an uridine are illustrated below.

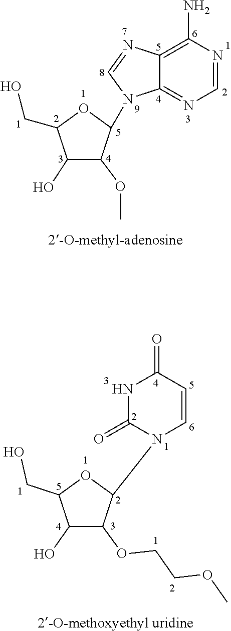 [SMILES: COC1C(O)C(CO)OC1n1cnc2c(N)ncnc21 ; COCCOC1C(O)C(CO)OC1n1ccc(=O)[nH]c1=O]

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

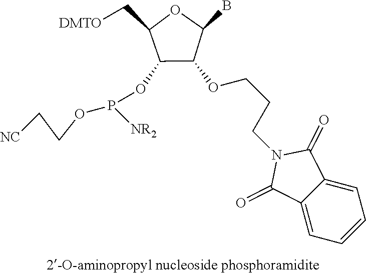

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

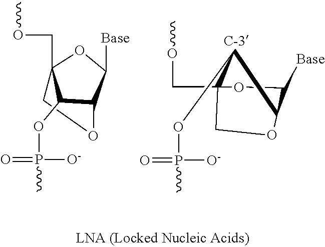

In some instances, the modification at the 2′ hydroxyl group comprises ethylene nucleic acids (ENA) such as for example 2′-4′-ethylene-bridged nucleic acid, which locks the sugar conformation into a C3′-endo sugar puckering conformation. ENA are part of the bridged nucleic acids class of modified nucleic acids that also comprises LNA. Exemplary chemical structures of the ENA and bridged nucleic acids are illustrated below.

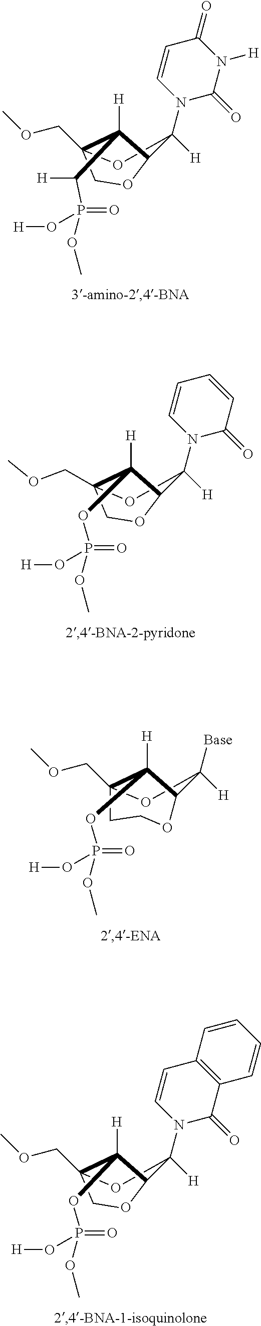

In some embodiments, additional modifications at the 2′ hydroxyl group include 2′-deoxy, 2′-deoxy-2′-fluoro, 2′-O-aminopropyl (2′-O-AP), 2-O-dimethylaminoethyl (2′-O-DMAOE), 2-O-dimethylaminopropyl (2′-O-DMAP), 2′-O-dimethylaminoethyloxyethyl (2′-O-DMAEOE), or 2′-O—N-methylacetamido (2′-O—NMA).

In some embodiments, nucleotide analogues comprise modified bases such as, but not limited to, 5-propynyluridine, 5-propynylcytidine, 6-methyladenine, 6-methylguanine, N, N,—dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. In some instances, the five-member ribose ring is substituted with a six member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

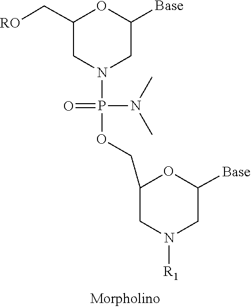

In some embodiments, peptide nucleic acid (PNA) does not contain sugar ring or phosphate linkage and the bases are attached and appropriately spaced by oligoglycine-like molecules, therefore, eliminating a backbone charge.

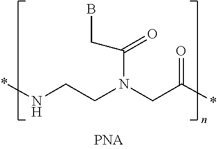

In some embodiments, one or more modifications optionally occur at the internucleotide linkage. In some instances, modified internucleotide linkage include, but is not limited to, phosphorothioates, mesyl phosphoramidate, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. Mesyl phosphoramidate antisense oligonucleotides (MsPA ASO) are antisense oligonucleotides comprising a mesyl phosphoramidate linkage.

In some instances, the modification is a methyl or thiol modification such as methylphosphonate, mesyl phosphoramidate, or thiolphosphonate modification. In some instances, a modified nucleotide includes, but is not limited to, 2′-fluoro N3-P5′-phosphoramidites.

In some instances, a modified nucleotide includes, but is not limited to, hexitol nucleic acid (or 1′, 5′-anhydrohexitol nucleic acids (HNA)).

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, the oligonucleotide molecule comprises one or more of the synthetic nucleotide analogues described herein. In some instances, the oligonucleotide molecule comprises 1, 2, 3, 4, 5, 6, 7, 8, 9, 10, 11, 12, 13, 14, 15, 16, 17, 18, 20, 25, or more of the synthetic nucleotide analogues described herein. In some embodiments, the synthetic nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide molecule comprises 1, 2, 3, 4, 5, 6, 7, 8, 9, 10, 11, 12, 13, 14, 15, 16, 17, 18, 20, 25, or more of the synthetic nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the oligonucleotide molecule comprises 1, 2, 3, 4, 5, 6, 7, 8, 9, 10, 11, 12, 13, 14, 15, 16, 17, 18, 20,25, or more of 2′-O-methoxyethyl (2′-O-MOE) modified nucleotides. In some instances, the oligonucleotide molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the oligonucleotide molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification. In some instances, the oligonucleotide molecule comprises 100% modification.

In some cases, the oligonucleotide molecule comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification.

In some cases, the oligonucleotide molecule comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification.

In some instances, the oligonucleotide molecule comprises at least one of: from about 10% to about 70% modification, from about 20% to about 70% modification, from about 30% to about 70% modification, from about 40% to about 70% modification, from about 50% to about 70% modification, and from about 60% to about 70% modification.

In some instances, the oligonucleotide molecule comprises at least one of: from about 10% to about 60% modification, from about 20% to about 60% modification, from about 30% to about 60% modification, from about 40% to about 60% modification, and from about 50% to about 60% modification.

In some cases, the oligonucleotide molecule comprises at least one of: from about 10% to about 50% modification, from about 20% to about 50% modification, from about 30% to about 50% modification, and from about 40% to about 50% modification.

In some cases, the oligonucleotide molecule comprises at least one of: from about 10% to about 40% modification, from about 20% to about 40% modification, and from about 30% to about 40% modification.

In some cases, the oligonucleotide molecule comprises at least one of: from about 10% to about 30% modification, and from about 20% to about 30% modification.

In some cases, the oligonucleotide molecule comprises from about 10% to about 20% modification.

In some cases, the oligonucleotide molecule comprises from about 15% to about 90%, from about 20% to about 80%, from about 30% to about 70%, or from about 40% to about 60% modifications.

In additional cases, the oligonucleotide molecule comprises at least about 15%, 20%, 30%, 40%, 50%, 60%, 70%, 80%, 90%, 95%, or 99% modifications.

In some embodiments, the oligonucleotide molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 modifications.

In some instances, the oligonucleotide molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 modified nucleotides.

In some instances, from about 5 to about 100% of the oligonucleotide molecule comprise the synthetic nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95% or 100% of the oligonucleotide molecule comprise the synthetic nucleotide analogues described herein. In some instances, about 5% of the oligonucleotide molecule comprises the synthetic nucleotide analogues described herein. In some instances, about 10% of the oligonucleotide molecule comprises the synthetic nucleotide analogues described herein. In some instances, about 15% of the oligonucleotide molecule comprises the synthetic nucleotide analogues described herein. In some instances, about 20% of the oligonucleotide molecule comprises the synthetic nucleotide analogues described herein. In some instances, about 25% of the oligonucleotide molecule comprises the synthetic nucleotide analogues described herein. In some instances, about 30% of the oligonucleotide molecule comprises the synthetic nucleotide analogues described herein. In some instances, about 35% of the oligonucleotide molecule comprises the synthetic nucleotide analogues described herein. In some instances, about 40% of the oligonucleotide molecule comprises the synthetic nucleotide analogues described herein. In some instances, about 45% of the oligonucleotide molecule comprises the synthetic nucleotide analogues described herein. In some instances, about 50% of the oligonucleotide molecule comprises the synthetic nucleotide analogues described herein. In some instances, about 55% of the oligonucleotide molecule comprises the synthetic nucleotide analogues described herein. In some instances, about 60% of the oligonucleotide molecule comprises the synthetic nucleotide analogues described herein. In some instances, about 65% of the oligonucleotide molecule comprises the synthetic nucleotide analogues described herein. In some instances, about 70% of the oligonucleotide molecule comprises the synthetic nucleotide analogues described herein. In some instances, about 75% of the oligonucleotide molecule comprises the synthetic nucleotide analogues described herein. In some instances, about 80% of the oligonucleotide molecule comprises the synthetic nucleotide analogues described herein. In some instances, about 85% of the oligonucleotide molecule comprises the synthetic nucleotide analogues described herein. In some instances, about 90% of the oligonucleotide molecule comprises the synthetic nucleotide analogues described herein. In some instances, about 95% of the oligonucleotide molecule comprises the synthetic nucleotide analogues described herein. In some instances, about 96% of the oligonucleotide molecule comprises the synthetic nucleotide analogues described herein. In some instances, about 97% of the oligonucleotide molecule comprises the synthetic nucleotide analogues described herein. In some instances, about 98% of the oligonucleotide molecule comprises the synthetic nucleotide analogues described herein. In some instances, about 99% of the oligonucleotide molecule comprises the synthetic nucleotide analogues described herein. In some instances, about 100% of the oligonucleotide molecule comprises the synthetic nucleotide analogues described herein. In some embodiments, the synthetic nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the oligonucleotide molecule comprises from about 1 to about 25 modifications in which the modification comprises an synthetic nucleotide analogues described herein. In some embodiments, the oligonucleotide molecule comprises about 1 modification in which the modification comprises a synthetic nucleotide analogue described herein. In some embodiments, the oligonucleotide molecule comprises about 2 modifications in which the modifications comprise a synthetic nucleotide analogue described herein. In some embodiments, the oligonucleotide molecule comprises about 3 modifications in which the modifications comprise a synthetic nucleotide analogue described herein. In some embodiments, the oligonucleotide molecule comprises about 4 modifications in which the modifications comprise a synthetic nucleotide analogue described herein. In some embodiments, the oligonucleotide molecule comprises about 5 modifications in which the modifications comprise a synthetic nucleotide analogue described herein. In some embodiments, the oligonucleotide molecule comprises about 6 modifications in which the modifications comprise a synthetic nucleotide analogue described herein. In some embodiments, the oligonucleotide molecule comprises about 7 modifications in which the modifications comprise a synthetic nucleotide analogue described herein. In some embodiments, the oligonucleotide molecule comprises about 8 modifications in which the modifications comprise a synthetic nucleotide analogue described herein. In some embodiments, the oligonucleotide molecule comprises about 9 modifications in which the modifications comprise a synthetic nucleotide analogue described herein. In some embodiments, the oligonucleotide molecule comprises about 10 modifications in which the modifications comprise a synthetic nucleotide analogue described herein. In some embodiments, the oligonucleotide molecule comprises about 11 modifications in which the modifications comprise a synthetic nucleotide analogue described herein. In some embodiments, the oligonucleotide molecule comprises about 12 modifications in which the modifications comprise a synthetic nucleotide analogue described herein. In some embodiments, the oligonucleotide molecule comprises about 13 modifications in which the modifications comprise a synthetic nucleotide analogue described herein. In some embodiments, the oligonucleotide molecule comprises about 14 modifications in which the modifications comprise a synthetic nucleotide analogue described herein. In some embodiments, the oligonucleotide molecule comprises about 15 modifications in which the modifications comprise a synthetic nucleotide analogue described herein. In some embodiments, the oligonucleotide molecule comprises about 16 modifications in which the modifications comprise a synthetic nucleotide analogue described herein. In some embodiments, the oligonucleotide molecule comprises about 17 modifications in which the modifications comprise a synthetic nucleotide analogue described herein. In some embodiments, the oligonucleotide molecule comprises about 18 modifications in which the modifications comprise a synthetic nucleotide analogue described herein. In some embodiments, the oligonucleotide molecule comprises about 19 modifications in which the modifications comprise a synthetic nucleotide analogue described herein. In some embodiments, the oligonucleotide molecule comprises about 20 modifications in which the modifications comprise a synthetic nucleotide analogue described herein. In some embodiments, the oligonucleotide molecule comprises about 21 modifications in which the modifications comprise a synthetic nucleotide analogue described herein. In some embodiments, the oligonucleotide molecule comprises about 22 modifications in which the modifications comprise a synthetic nucleotide analogue described herein. In some embodiments, the oligonucleotide molecule comprises about 23 modifications in which the modifications comprise a synthetic nucleotide analogue described herein. In some embodiments, the oligonucleotide molecule comprises about 24 modifications in which the modifications comprise a synthetic nucleotide analogue described herein. In some embodiments, the oligonucleotide molecule comprises about 25 modifications in which the modifications comprise a synthetic nucleotide analogue described herein. In some embodiments, the oligonucleotide molecule comprises about 26 modifications in which the modifications comprise a synthetic nucleotide analogue described herein. In some embodiments, the oligonucleotide molecule comprises about 27 modifications in which the modifications comprise a synthetic nucleotide analogue described herein. In some embodiments, the oligonucleotide molecule comprises about 28 modifications in which the modifications comprise a synthetic nucleotide analogue described herein. In some embodiments, the oligonucleotide molecule comprises about 29 modifications in which the modifications comprise a synthetic nucleotide analogue described herein. In some embodiments, the oligonucleotide molecule comprises about 30 modifications in which the modifications comprise a synthetic nucleotide analogue described herein. In some embodiments, the oligonucleotide molecule comprises about 31 modifications in which the modifications comprise a synthetic nucleotide analogue described herein. In some embodiments, the oligonucleotide molecule comprises about 32 modifications in which the modifications comprise a synthetic nucleotide analogue described herein. In some embodiments, the oligonucleotide molecule comprises about 33 modifications in which the modifications comprise a synthetic nucleotide analogue described herein. In some embodiments, the oligonucleotide molecule comprises about 34 modifications in which the modifications comprise a synthetic nucleotide analogue described herein. In some embodiments, the oligonucleotide molecule comprises about 35 modifications in which the modifications comprise a synthetic nucleotide analogue described herein. In some embodiments, the oligonucleotide molecule comprises about 36 modifications in which the modifications comprise a synthetic nucleotide analogue described herein. In some embodiments, the oligonucleotide molecule comprises about 37 modifications in which the modifications comprise a synthetic nucleotide analogue described herein. In some embodiments, the oligonucleotide molecule comprises about 38 modifications in which the modifications comprise a synthetic nucleotide analogue described herein. In some embodiments, the oligonucleotide molecule comprises about 39 modifications in which the modifications comprise a synthetic nucleotide analogue described herein. In some embodiments, the oligonucleotide molecule comprises about 40 modifications in which the modifications comprise a synthetic nucleotide analogue described herein.

In some embodiments, an oligonucleotide molecule is assembled from two separate polynucleotides wherein one polynucleotide comprises the sense strand and the second polynucleotide comprises the antisense strand of the oligonucleotide molecule. In other embodiments, the sense strand is connected to the antisense strand via a linker molecule, which in some instances is a polynucleotide linker or a non-nucleotide linker.

In some embodiments, an oligonucleotide molecule comprises a sense strand and antisense strand, wherein pyrimidine nucleotides in the sense strand comprises 2′-O-methylpyrimidine nucleotides and purine nucleotides in the sense strand comprise 2′-deoxy purine nucleotides. In some embodiments, an oligonucleotide molecule comprises a sense strand and antisense strand, wherein pyrimidine nucleotides present in the sense strand comprise 2′-deoxy-2′-fluoro pyrimidine nucleotides and wherein purine nucleotides present in the sense strand comprise 2′-deoxy purine nucleotides.

In some embodiments, an oligonucleotide molecule comprises a sense strand and antisense strand, wherein the pyrimidine nucleotides when present in said antisense strand are 2′-deoxy-2′-fluoro pyrimidine nucleotides and the purine nucleotides when present in said antisense strand are 2′-O-methyl purine nucleotides.

In some embodiments, an oligonucleotide molecule comprises a sense strand and antisense strand, wherein the pyrimidine nucleotides when present in said antisense strand are 2′-deoxy-2′-fluoro pyrimidine nucleotides and wherein the purine nucleotides when present in said antisense strand comprise 2′-deoxy-purine nucleotides.

In some embodiments, an oligonucleotide molecule comprises a sense strand and antisense strand, and at least one of sense strand and antisense strands has a plurality of (e.g., two or more, three or more, four or more, five or more, six or more, seven or more, eight or more, etc) 2′-O-methyl or 2′-deoxy-2′-fluoro modified nucleotides. In some embodiments, at least two, three, four, five, six, or seven out of the a plurality of 2′-O-methyl or 2′-deoxy-2′-fluoro modified nucleotides are consecutive nucleotides. In some embodiments, consecutive 2′-O-methyl or 2′-deoxy-2′-fluoro modified nucleotides are located at the 5′-end of the sense strand and/or the antisense strand. In some embodiments, consecutive 2′-O-methyl or 2′-deoxy-2′-fluoro modified nucleotides are located at the 3′-end of the sense strand and/or the antisense strand. In some embodiments, the sense strand of oligonucleotide molecule includes at least four, at least five, at least six consecutive 2′-O-methyl modified nucleotides at its 5′ end and/or 3′end, or both. Optionally, in such embodiments, the sense strand of oligonucleotide molecule includes at least one, at least two, at least three, at least four 2′-deoxy-2′-fluoro modified nucleotides at the 3′ end of the at least four, at least five, at least six consecutive 2′-O-methyl modified nucleotides at the polynucleotides' 5′ end, or at the 5′ end of the at least four, at least five, at least six consecutive 2′-O-methyl modified nucleotides at polynucleotides' 3′ end. Also optionally, such at least two, at least three, at least four 2′-deoxy-2′-fluoro modified nucleotides are consecutive nucleotides.

In some embodiments, an oligonucleotide molecule comprises a sense strand and antisense strand, and at least one of sense strand and antisense strand has 2′-O-methyl modified nucleotide located at the 5′-end of the sense strand and/or the antisense strand. In some embodiments, at least one of sense strand and antisense strands has 2′-O-methyl modified nucleotide located at the 3′-end of the sense strand and/or the antisense strand. In some embodiments, the 2′-O-methyl modified nucleotide located at the 5′-end of the sense strand and/or the antisense strand is a purine nucleotide. In some embodiments, the 2′-O-methyl modified nucleotide located at the 5′-end of the sense strand and/or the antisense strand is a pyrimidine nucleotide.

In some embodiments, an oligonucleotide molecule comprises a sense strand and antisense strand, and one of sense strand and antisense strand has at least two consecutive 2′-deoxy-2′-fluoro modified nucleotides located at the 5′-end, while another strand has at least two consecutive 2′-O-methyl modified nucleotides located at the 5′-end. In some embodiments, where the strand has at least two consecutive 2′-deoxy-2′-fluoro modified nucleotides located at the 5′-end, the strand also includes at least two, at least three consecutive 2′-O-methyl modified nucleotides at the 3′end of the at least two consecutive 2′-deoxy-2′-fluoro modified nucleotides. In some embodiments, one of sense strand and antisense strand has at least two, at least three, at least four, at least five, at least six, or at least seven consecutive 2′-O-methyl modified nucleotides that are linked to a 2′-deoxy-2′-fluoro modified nucleotide on its 5′-end and/or 3′end. In some embodiments, one of sense strand and antisense strand has at least four, at least five nucleotides that have alternating 2′-O-methyl modified nucleotide and 2′-deoxy-2′-fluoro modified nucleotide.

In some embodiments, the oligonucleotide molecule, such as a siRNA, has the formula as illustrated in Formula III:

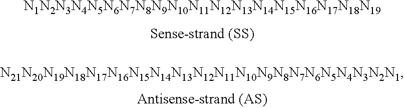

wherein each nucleotide represented by N, is independently, A, U, C, or G or a modified nucleotide base, such as those provided for herein. The N₁ nucleotides of the sense strand and the antisense strand represent the 5′ end of the respective strands. For clarity, although Formula III utilizes N₁, N₂, N₃, etc. in both the sense and the antisense strand, the nucleotide bases do not need to be the same and are not intended to be the same. The siRNA that is illustrated in Formula III would be complementary to a target sequence.

For example, in some embodiments, the sense strand comprises a 2′O-methyl modified nucleotide with a phosphorothioate (PS) modified backbone at N₁ and N₂, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, N₁₈, and N₁₉.

In some embodiments, the antisense strand comprises a vinylphosphonate moiety attached to N₁, a 2′fluoro-modified nucleotide with a phosphorothioate (PS) modified backbone at N₂, a 2′O-methyl modified nucleotide at N₃, N₄, N₅, N₆, N₇, N₈, N₉, N₁₀, N₁₁, N₁₂, N₁₃, N₁₅, N₁₆, N₁₇, N₁₈, and N₁₉, a 2′fluoro- modified nucleotide at N₁₄, and a 2′O-methyl modified nucleotide with a phosphorothioate (PS) modified backbone at N₂₀ and N₂₁.

In some embodiments, an oligonucleotide molecule comprises a sense strand and antisense strand, wherein the sense strand includes a terminal cap moiety at the 5′-end, the 3′-end, or both of the 5′ and 3′ ends of the sense strand. In other embodiments, the terminal cap moiety is an inverted deoxy abasic moiety.

In some embodiments, an oligonucleotide molecule comprises a sense strand and an antisense strand, wherein the antisense strand comprises a glyceryl modification at the 3′ end of the antisense strand.

In some embodiments, an oligonucleotide molecule comprises a sense strand and an antisense strand, in which the sense strand comprises one or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and in which the antisense strand comprises about 1 to about 10 or more, specifically about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate, phosphorodithioate, phosphonate, phosphoramidate, or mesyl phosphoramidate internucleotide linkages and/or a terminal cap molecule at the 3′-end, the 5′-end, or both of the 3′- and 5′-ends, being present in the same or different strand.

In some embodiments, an oligonucleotide molecule comprises a sense strand and an antisense strand, in which the sense strand comprises about 1 to about 25, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10, or more) 2′-deoxy, 2′-O-methyl, 2′-deoxy-2′-fluoro, and/or one or more (e.g., about 1, 2, 3, 4, 5, 6, 7, 8, 9, 10, or more) universal base modified nucleotides, and optionally a terminal cap molecule at the 3-end, the 5′-end, or both of the 3′- and 5′-ends of the sense strand; and in which the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages and/or a terminal cap molecule at the 3′-end, the 5′-end, or both of the 3′- and 5′-ends, being present in the same or different strand.

In some embodiments, an oligonucleotide molecule comprises a sense strand and an antisense strand, in which the antisense strand comprises one or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or about one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides at the 3′-end, the 5′-end, or both of the 3′- and 5′-ends of the sense strand and/or antisense strand, and optionally a terminal cap molecule at the 3′-end, the 5′-end, or both of the 3′- and 5′-ends of the sense strand. In some embodiments, the antisense strand comprises about 1 to about 10 or more, specifically about 1, 2, 3, 4, 5, 6, 7, 8, 9, 10 or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, 11, 12, 13, 14, 15, 16, 17, 18, 19, 20, or more pyrimidine nucleotides of the sense and/or antisense strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate, phosphorodithioate, phosphonate, phosphoramidate, or mesyl phosphoramidate internucleotide linkages and/or a terminal cap molecule at the 3′-end, the 5′-end, or both of the 3′ and 5′-ends, being present in the same or different strand.

In some embodiments, an oligonucleotide molecule comprises a sense strand and an antisense strand, in which the antisense strand comprises about 1 to about 25 or more, for example, about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate, phosphorodithioate, phosphonate, phosphoramidate, or mesyl phosphoramidate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10 or more pyrimidine nucleotides of the sense and/or antisense strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate, phosphorodithioate, phosphonate, phosphoramidate, or mesyl phosphoramidate internucleotide linkages and/or a terminal cap molecule at the 3′-end, the 5′-end, or both of the 3′- and 5′-ends, being present in the same or different strand.

In some embodiments, an oligonucleotide molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate, phosphorodithioate, phosphonate, phosphoramidate, or mesyl phosphoramidate internucleotide linkages in each strand of the oligonucleotide molecule. In some embodiments, an oligonucleotide molecule comprises a sense strand and an antisense strand, and the antisense strand comprises a phosphate backbone modification at the 3′ end of the antisense strand. Alternatively and/or additionally, an oligonucleotide molecule comprises a sense strand and an antisense strand, and the sense strand comprises a phosphate backbone modification at the 5′ end of the antisense strand. In some instances, the phosphate backbone modification is a phosphorothioate. In some instances, the phosphate backbone modification is a phosphorodithioate. In some instances, the phosphate backbone modification is a phosphonate. In some instances, the phosphate backbone modification is a phosphoramidate. In some instances, the phosphate backbone modification is a mesyl phosphoramidate. In some embodiments, the sense or antisense strand has three consecutive nucleosides that are coupled via two phosphorothioate backbone. In some embodiments, the sense or antisense strand has three consecutive nucleosides that are coupled via two phosphorodithioate backbone. In some embodiments, the sense or antisense strand has three consecutive nucleosides that are coupled via two phosphonate backbone. In some embodiments, the sense or antisense strand has three consecutive nucleosides that are coupled via two phosphoramidate backbone. In some embodiments, the sense or antisense strand has three consecutive nucleosides that are coupled via two mesyl phosphoramidate backbone.

In another embodiment, an oligonucleotide molecule described herein comprises 2′-5′ internucleotide linkages. In some instances, the 2′-5′ internucleotide linkage(s) is at the 3′-end, the 5′-end, or both of the 3′- and 5′-ends of one or both sequence strands. In addition instances, the 2′-5′ internucleotide linkage(s) is present at various other positions within one or both sequence strands, for example, about 1, 2, 3, 4, 5, 6, 7, 8, 9, 10, or more including every internucleotide linkage of a pyrimidine nucleotide in one or both strands of the oligonucleotide molecule comprise a 2′-5′ internucleotide linkage, or about 1, 2, 3, 4, 5, 6, 7, 8, 9, 10, or more including every internucleotide linkage of a purine nucleotide in one or both strands of the oligonucleotide molecule comprise a 2′-5′ internucleotide linkage.

In some embodiments, an oligonucleotide molecule is a single stranded molecule that mediates RNAi activity in a cell or reconstituted in vitro system, wherein the oligonucleotide molecule comprises a single stranded polynucleotide having complementarity to a target nucleic acid sequence, and wherein one or more pyrimidine nucleotides present in the oligonucleotide molecule are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the oligonucleotide molecule are 2′-deoxy purine nucleotides (e.g., wherein all purinenucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the oligonucleotide molecule optionally further comprising about 1 to about 4 (e.g., about 1, 2, 3, or 4) terminal 2′-deoxynucleotides at the 3′-end of the oligonucleotide molecule, wherein the terminal nucleotides further comprise one or more (e.g., 1, 2, 3, or 4) phosphorothioate or mesyl phosphoramidate internucleotide linkages, and wherein the oligonucleotide molecule optionally further comprises a terminal phosphate group, such as a 5′-terminal phosphate group.

In some cases, one or more of the synthetic nucleotide analogues described herein are resistant toward nucleases such as for example ribonuclease such as RNase H, deoxyribonuclease such as DNase, or exonuclease such as 5′-3′ exonuclease and 3′-5′ exonuclease when compared to natural polynucleic acid molecules and endonucleases. In some instances, synthetic nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, 2′-O-methyl modified oligonucleotide molecule is nuclease resistance (e.g., RNase H, DNase, 5′-3′ exonuclease or 3′-5′ exonuclease resistance). In some instances, 2′O-methoxyethyl (2′-O-MOE) modified oligonucleotide molecule is nuclease resistance (e.g., RNase H, DNase, 5′-3′ exonuclease or 3′-5′ exonuclease resistance). In some instances, 2′-O-aminopropyl modified oligonucleotide molecule is nuclease resistance (e.g., RNase H, DNase, 5′-3′ exonuclease or 3′-5′ exonuclease resistance). In some instances, 2′-deoxy modified oligonucleotide molecule is nuclease resistance (e.g., RNase H, DNase, 5′-3′ exonuclease or 3′-5′ exonuclease resistance). In some instances, 2′-deoxy-2′-fluoro modified oligonucleotide molecule is nuclease resistance (e.g., RNase H, DNase, 5′-3′ exonuclease or 3′-5′ exonuclease resistance). In some instances, 2′-O-aminopropyl (2′-O-AP) modified oligonucleotide molecule is nuclease resistance (e.g., RNase H, DNase, 5′-3′ exonuclease or 3′-5′ exonuclease resistance). In some instances, 2′-O-dimethylaminoethyl (2′-O-DMAOE) modified oligonucleotide molecule is nuclease resistance (e.g., RNase H, DNase, 5′-3′ exonuclease or 3′-5′ exonuclease resistance). In some instances, 2-O-dimethylaminopropyl (2′-O-DMAP) modified oligonucleotide molecule is nuclease resistance (e.g., RNase H, DNase, 5′-3′ exonuclease or 3′-5′ exonuclease resistance). In some instances, 2′-O-dimethylaminoethyloxyethyl (2′-O-DMAEOE) modified oligonucleotide molecule is nuclease resistance (e.g., RNase H, DNase, 5′-3′ exonuclease or 3′-5′ exonuclease resistance). In some instances, 2′-O—N-methylacetamido (2′-O—NMA) modified oligonucleotide molecule is nuclease resistance (e.g., RNase H, DNase, 5′-3′ exonuclease or 3′-5′ exonuclease resistance). In some instances, LNA modified oligonucleotide molecule is nuclease resistance (e.g., RNase H, DNase, 5′-3′ exonuclease or 3′-5′ exonuclease resistance). In some instances, ENA modified oligonucleotide molecule is nuclease resistance (e.g., RNase H, DNase, 5′-3′ exonuclease or 3′-5′ exonuclease resistance). In some instances, HNA modified oligonucleotide molecule is nuclease resistance (e.g., RNase H, DNase, 5′-3′ exonuclease or 3′-5′ exonuclease resistance). In some instances, morpholinos is nuclease resistance (e.g., RNase H, DNase, 5′-3′ exonuclease or 3′-5′ exonuclease resistance). In some instances, PNA modified oligonucleotide molecule is resistant to nucleases (e.g., RNase H, DNase, 5′-3′ exonuclease or 3′-5′ exonuclease resistance). In some instances, methylphosphonate nucleotides modified oligonucleotide molecule is nuclease resistance (e.g., RNase H, DNase, 5′-3′ exonuclease or 3′-5′ exonuclease resistance). In some instances, thiolphosphonate nucleotides modified oligonucleotide molecule is nuclease resistance (e.g., RNase H, DNase, 5′-3′ exonuclease or 3′-5′ exonuclease resistance). In some instances, oligonucleotide molecule comprising 2′-fluoro N3-P5′-phosphoramidites is nuclease resistance (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

In some embodiments, one or more of the synthetic nucleotide analogues described herein have increased binding affinity toward their mRNA target relative to an equivalent natural polynucleic acid molecule. The one or more of the synthetic nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, 2′-O-methyl modified oligonucleotide molecule has increased binding affinity toward their mRNA target relative to an equivalent natural polynucleic acid molecule. In some instances, 2′-O-methoxyethyl (2′-O-MOE) modified oligonucleotide molecule has increased binding affinity toward their mRNA target relative to an equivalent natural polynucleic acid molecule. In some instances, 2′-O-aminopropyl modified oligonucleotide molecule has increased binding affinity toward their mRNA target relative to an equivalent natural polynucleic acid molecule. In some instances, 2′-deoxy modified oligonucleotide molecule has increased binding affinity toward their mRNA target relative to an equivalent natural polynucleic acid molecule. In some instances, 2′-deoxy-2′-fluoro modified oligonucleotide molecule has increased binding affinity toward their mRNA target relative to an equivalent natural polynucleic acid molecule. In some instances, 2′-O-aminopropyl (2′-O-AP) modified oligonucleotide molecule has increased binding affinity toward their mRNA target relative to an equivalent natural polynucleic acid molecule. In some instances, 2-O-dimethylaminoethyl (2′-O-DMAOE) modified oligonucleotide molecule has increased binding affinity toward their mRNA target relative to an equivalent natural polynucleic acid molecule. In some instances, 2′-O-dimethylaminopropyl (2′-O-DMAP) modified oligonucleotide molecule has increased binding affinity toward their mRNA target relative to an equivalent natural polynucleic acid molecule. In some instances, 2′-O-dimethylaminoethyloxyethyl (2′-O-DMAEOE) modified oligonucleotide molecule has increased binding affinity toward their mRNA target relative to an equivalent natural polynucleic acid molecule. In some instances, 2′-O—N-methylacetamido (2′-O—NMA) modified oligonucleotide molecule has increased binding affinity toward their mRNA target relative to an equivalent natural polynucleic acid molecule. In some instances, LNA modified oligonucleotide molecule has increased binding affinity toward their mRNA target relative to an equivalent natural polynucleic acid molecule. In some instances, ENA modified oligonucleotide molecule has increased binding affinity toward their mRNA target relative to an equivalent natural polynucleic acid molecule. In some instances, PNA modified oligonucleotide molecule has increased binding affinity toward their mRNA target relative to an equivalent natural polynucleic acid molecule. In some instances, HNA modified oligonucleotide molecule has increased binding affinity toward their mRNA target relative to an equivalent natural polynucleic acid molecule. In some instances, morpholino modified oligonucleotide molecule has increased binding affinity toward their mRNA target relative to an equivalent natural polynucleic acid molecule. In some instances, methylphosphonate nucleotides modified oligonucleotide molecule has increased binding affinity toward their mRNA target relative to an equivalent natural polynucleic acid molecule. In some instances, thiolphosphonate nucleotides modified oligonucleotide molecule has increased binding affinity toward their mRNA target relative to an equivalent natural polynucleic acid molecule. In some instances, oligonucleotide molecule comprising 2′-fluoro N3-P5′-phosphoramidites has increased binding affinity toward their mRNA target relative to an equivalent natural polynucleic acid molecule. In some cases, the increased affinity is illustrated with a lower Kd, a higher melt temperature (Tm), or a combination thereof.

In some embodiments, an oligonucleotide molecule described herein is a chirally pure (or stereo pure) polynucleic acid molecule, or a polynucleic acid molecule comprising a single enantiomer. In some instances, the oligonucleotide molecule comprises L-nucleotide. In some instances, the oligonucleotide molecule comprises D-nucleotides. In some instance, an oligonucleotide molecule composition comprises less than 30%, 25%, 20%, 15%, 10%, 5%, 4%, 3%, 2%, 1%, or less of its mirror enantiomer. In some cases, an oligonucleotide molecule composition comprises less than 30%, 25%, 20%, 15%, 10%, 5%, 4%, 3%, 2%, 1T %, or less of a racemic mixture.

In some embodiments, an oligonucleotide molecule described herein is further modified to include an aptamer conjugating moiety. In some instances, the aptamer conjugating moiety is a DNA aptamer conjugating moiety. In some instances, the aptamer conjugating moiety is Alphamer, which comprises an aptamer portion that recognizes a specific cell-surface target and a portion that presents a specific epitope for attaching to circulating antibodies.

In additional embodiments, an oligonucleotide molecule described herein is modified to increase its stability. In some embodiment, the oligonucleotide molecule is RNA (e.g., siRNA). In some instances, the oligonucleotide molecule is modified by one or more of the modifications described above to increase its stability. In some cases, the oligonucleotide molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modification or by a locked or bridged ribose conformation (e.g., LNA or ENA). In some cases, the oligonucleotide molecule is modified by 2′-O-methyl and/or 2′-O-methoxyethyl ribose. In some cases, the oligonucleotide molecule also includes morpholinos, PNAs, HNA, methylphosphonate nucleotides, thiolphosphonate nucleotides, and/or 2′-fluoro N3-P5′-phosphoramidites to increase its stability. In some instances, the oligonucleotide molecule is a chirally pure (or stereo pure) oligonucleotide molecule. In some instances, the chirally pure (orstereo pure) oligonucleotide molecule is modified to increase its stability. Suitable modifications to the RNA to increase stability for delivery will be apparent to the skilled person.

In some embodiments, the oligonucleotide molecule comprises 2′ modifications. In some embodiments, the nucleotides of the oligonucleotide molecule at positions 3, 7, 8, 9, 12, and 17 from the 5′ end of the sense strand are not modified with a 2′O-methyl modification. In some embodiments, the nucleotides of the oligonucleotide molecule at positions 3, 7, 8, 9, 12, and 17 from the 5′ end of the sense strand are modified with a 2′fluoro modification. In some embodiments, the nucleotides of the oligonucleotide molecule at positions 2 and 14 from the 5′ end of the anti-sense strand are not modified with a 2′O-methyl modification. In some embodiments, the nucleotides of the oligonucleotide molecule at positions 2 and 14 from the 5′ end of the anti-sense strand are modified with a 2′fluoro modification. In some embodiments, any of the nucleotides may further comprise a 5′-phosphorothioate group modification. In some embodiments, the nucleotides of the oligonucleotide molecule at positions 1 and 2 from the 5′ end of the sense strand are modified with a 5′-phosphorothioate group modification. In some embodiments, the nucleotides of the oligonucleotide molecule at positions 1, 2, 20, and 21 from the 5′ end of the antisense strand are modified with a 5′-phosphorothioate group modification. In some embodiments, the 5′ end of the sense or antisense strand of the oligonucleotide molecule may further comprise a vinylphosphonate modification. In some embodiments, the nucleotide of the oligonucleotide molecule at position 1 from the 5′ end of the antisense strand is modified with a vinylphosphonate modification.

In some instances, the oligonucleotide molecule is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. In some instances, the oligonucleotide molecule is assembled from two separate polynucleotides, where one strand is the sense strand and the other is the antisense strand, wherein the antisense and sense strands are self-complementary (e.g., each strand comprises nucleotide sequence that is complementary to nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the oligonucleotide molecule is assembled from a single oligonucleotide, where the self-complementary sense and antisense regions of the oligonucleotide molecule are linked by means of a nucleic acid based or non-nucleic acid-based linker(s).

In some cases, the oligonucleotide molecule is a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. In other cases, the oligonucleotide molecule is a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide is processed either in vivo or in vitro to generate an active oligonucleotide molecule capable of mediating RNAi. In additional cases, the oligonucleotide molecule also comprises a single-stranded polynucleotide having nucleotide sequence complementary to nucleotide sequence in a target nucleic acid molecule or a portion thereof (for example, where such oligonucleotide molecule does not require the presence within the oligonucleotide molecule of nucleotide sequence corresponding to the target nucleic acid sequence or a portion thereof), wherein the single stranded polynucleotide further comprises a terminal phosphate group, such as a 5′-phosphate, or 5′, 3′-diphosphate.

In some instances, an asymmetric hairpin is a linear oligonucleotide molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin oligonucleotide molecule comprises an antisense region having length sufficient to mediate RNAi in a cell or in vitro system (e.g., about 19 to about 22 nucleotides) and a loop region comprising about 4 to about 8 nucleotides, and a sense region having about 3 to about 18 nucleotides that are complementary to the antisense region. In some cases, the asymmetric hairpin oligonucleotide molecule also comprises a 5′-terminal phosphate group that is chemically modified. In additional cases, the loop portion of the asymmetric hairpin oligonucleotide molecule comprises nucleotides, non-nucleotides, linker molecules, or conjugate molecules.

In some embodiments, an asymmetric duplex is an oligonucleotide molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex oligonucleotide molecule comprises an antisense region having length sufficient to mediate RNAi in a cell or in vitro system (e.g., about 19 to about 22 nucleotides) and a sense region having about 3 to about 19 nucleotides that are complementary to the antisense region.

In some cases, a universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art.

In some embodiments, the dsRNA agents are 5′ phosphorylated or include a phosphoryl analog at the 5′ prime terminus. 5′-phosphate modifications include those which are compatible with RISC mediated gene silencing. Suitable modifications include: 5′-monophosphate ((HO₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); phosphorodithioate [—O₂PS₂—]; phosphonate [—PO(OH)₂—]; phosphoramidate [—O═P(OH)₂—]; mesyl phosphoramidate (CH₃)(SO₂)(N)P(O)₂—O-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH2-), 5′-alkyletherphosphonates (R=alkylether=methoxymethyl (MeOCH2-), ethoxymethyl, etc., e.g. RP(OH)(O)—O-5′-). In some embodiments, the modification can in placed in the antisense strand of a dsRNA agent.

Other modifications and patterns of modifications can be found in, for example, U.S. Pat. No. 10,233,448, which is hereby incorporated by reference. Other modifications and patterns of modifications can be found in, for example, Anderson et al. Nucleic Acids Research 2021, 49 (16), 9026-9041, which is hereby incorporated by reference. Other modifications and patterns of modifications can be found in, for example, PCT Publication No. WO2021/030778, which is hereby incorporated by reference. Other modifications and patterns of modifications can be found in, for example, PCT Publication No. WO2021/030763, which is hereby incorporated by reference.

In some embodiments, the sequence of the oligonucleotide molecule is at least 40%, 50%, 55%, 60%, 65%, 70%, 75%, 80%, 85%, 90%, 95%, 98%, 99%, or 99.5% complementary to a target sequence of CD40, KRAS, or GYS1. In some embodiments, the target sequence of CD40, KRAS, or GYS1 is a nucleic acid sequence of about 10-50 base pair length, about 15-50 base pair length, 15-40 base pair length, 15-30 base pair length, or 15-25 base pair length sequences in CD40, KRAS, or GYS1, in which the first nucleotide of the target sequence starts at any nucleotide in CD40 mRNA transcript in the coding region, or in the 5′ or 3′-untraslated region (UTR). For example, the first nucleotide of the target sequence can be selected so that it starts at the nucleic acid location (nal, number starting from the 5′-end of the full length of CD40, KRAS, or GYS1 mRNA, e.g., the 5′-end first nucleotide is nal.1) 1, nal 2, nal 3, nal 4, nal 5, nal 6, nal 7, nal 8, nal 9, nal 10, nal 11, nal 12, nal 13, nal 14, nal 15, nal 15, nal 16, nal 17, or any other nucleic acid location in the coding or noncoding regions (5′ or 3′-untraslated region) of CD40, KRAS, or GYS1 mRNA. In some embodiments, the first nucleotide of the target sequence can be selected so that it starts at a location within, or between, nal 10-nal 15, nal 10-nal 20, nal 50-nal 60, nal 55-nal 65, nal 75-nal 85, nal 95-nal 105, nal 135-nal 145, nal 155-nal 165, nal 225-nal 235, nal 265-nal 275, nal 275-nal 245, nal 245-nal 255, nal 285-nal 335, nal 335-nal 345, nal 385-nal 395, nal 515-nal 525, nal 665-nal 675, nal 675-nal 685, nal 695-nal 705, nal 705-nal 715, nal 875-nal 885, nal 885-nal 895, nal 895-nal 905, nal 1035-nal 1045, nal 1045-nal 1055, nal 1125-nal 1135, nal 1135-nal 1145, nal 1145-nal 1155, nal 1155-nal 1165, nal 1125-nal 1135, nal 1155-nal 1165, nal 1225-nal 1235, nal 1235-nal 1245, nal 1275-nal 1245, nal 1245-nal 1255, nal 1265-nal 1275, nal 1125-nal 1135, nal 1155-nal 1165, nal 1225-nal 1235, nal 1235-nal 1245, nal 1275-nal 1245, nal 1245-nal 1255, nal 1265-nal 1275, nal 1275-nal 1285, nal 1335-nal 1345, nal 1345-nal 1355, nal 1525-nal 1535, nal 1535-nal 1545, nal 1605-nal 1615, nal 1615-c.1625, nal 1625-nal 1635, nal 1635-1735, nal 1735-1835, nal 1835-1935, nal. 1836-1856, nal 1935-2000, nal 2000-2100, nal 2100-2200, nal 2200-2260, nal 2260-2400, nal 2400-2500, nal 2500-2600, nal 2600-2700, nal 2700-2800, nal 2800-2500, nal 2500-2600, nal 2600-2700, nal 2700-2800, nal 2800-2860, etc. In some embodiments, the sequence of CD40 mRNA is provided as NCBI Reference Sequence: NM_001250.6.

>NM_001250.6 Homo sapiens CD40 molecule (CD40), transcript variant 1, mRNA
(SEQ ID NO: 977)
AGTGGTCCTGCCGCCTGGTCTCACCTCGCTATGGTTCGTCTGCCTCTGCAG
TGCGTCCTCTGGGGCTGCTTGCTGACCGCTGTCCATCCAGAACCACCCACTGCAT
GCAGAGAAAAACAGTACCTAATAAACAGTCAGTGCTGTTCTTTGTGCCAGCCAG
GACAGAAACTGGTGAGTGACTGCACAGAGTTCACTGAAACGGAATGCCTTCCTT
GCGGTGAAAGCGAATTCCTAGACACCTGGAACAGAGAGACACACTGCCACCAGC
ACAAATACTGCGACCCCAACCTAGGGCTTCGGGTCCAGCAGAAGGGCACCTCAG
AAACAGACACCATCTGCACCTGTGAAGAAGGCTGGCACTGTACGAGTGAGGCCT
GTGAGAGCTGTGTCCTGCACCGCTCATGCTCGCCCGGCTTTGGGGTCAAGCAGAT
TGCTACAGGGGTTTCTGATACCATCTGCGAGCCCTGCCCAGTCGGCTTCTTCTCC
AATGTGTCATCTGCTTTCGAAAAATGTCACCCTTGGACAAGCTGTGAGACCAAAG
ACCTGGTTGTGCAACAGGCAGGCACAAACAAGACTGATGTTGTCTGTGGTCCCC
AGGATCGGCTGAGAGCCCTGGTGGTGATCCCCATCATCTTCGGGATCCTGTTTGC
CATCCTCTTGGTGCTGGTCTTTATCAAAAAGGTGGCCAAGAAGCCAACCAATAAG
GCCCCCCACCCCAAGCAGGAACCCCAGGAGATCAATTTTCCCGACGATCTTCCTG
GCTCCAACACTGCTGCTCCAGTGCAGGAGACTTTACATGGATGCCAACCGGTCAC
CCAGGAGGATGGCAAAGAGAGTCGCATCTCAGTGCAGGAGAGACAGTGAGGCT
GCACCCACCCAGGAGTGTGGCCACGTGGGCAAACAGGCAGTTGGCCAGAGAGCC
TGGTGCTGCTGCTGCTGTGGCGTGAGGGTGAGGGGCTGGCACTGACTGGGCATA
GCTCCCCGCTTCTGCCTGCACCCCTGCAGTTTGAGACAGGAGACCTGGCACTGGA
TGCAGAAACAGTTCACCTTGAAGAACCTCTCACTTCACCCTGGAGCCCATCCAGT
CTCCCAACTTGTATTAAAGACAGAGGCAGAAGTTTGGTGGTGGTGGTGTTGGGGT
ATGGTTTAGTAATATCCACCAGACCTTCCGATCCAGCAGTTTGGTGCCCAGAGAG
GCATCATGGTGGCTTCCCTGCGCCCAGGAAGCCATATACACAGATGCCCATTGCA
GCATTGTTTGTGATAGTGAACAACTGGAAGCTGCTTAACTGTCCATCAGCAGGAG
ACTGGCTAAATAAAATTAGAATATATTTATACAACAGAATCTCAAAAACACTGTT
GAGTAAGGAAAAAAAGGCATGCTGCTGAATGATGGGTATGGAACTTTTTAAAAA
AGTACATGCTTTTATGTATGTATATTGCCTATGGATATATGTATAAATACAATATG
CATCATATATTGATATAACAAGGGTTCTGGAAGGGTACACAGAAAACCCACAGC
TCGAAGAGTGGTGACGTCTGGGGTGGGGAAGAAGGGTCTGGGGGAGGGTTGGTT
AAAGGGAGATTTGGCTTTCCCATAATGCTTCATCATTTTTCCCAAAAGGAGAGTG
AATTCACATAATGCTTATGTAATTAAAAAATCATCAAACATGTAAAAA
In some embodiments, the sequence of GYS1 mRNA is provided as NCBI Reference
Sequence: (NM_001161587)
>NM_001161587.2 Homo sapiens glycogen synthase 1 (GYS1), mRNA
(SE ID NO: 978)
AGTGACGCTGCGGCCTCCTTCTGCCTAGGTCCCAACGCTTCGGGGCAGGG
GTGCGGTCTTGCAATAGGAAGCCGAGCGTCTTGCAAGCTTCCCGTCGGGCACCA
GCTACTCGGCCCCGCACCCTACCTGGTGCATTCCCTAGACACCTCCGGGGTCCCT
ACCTGGAGATCCCCGGAGCCCCCCTTCCTGCGCCAGCCATGCCTTTAAACCGCAC
TTTGTCCATGTCCTCACTGCCAGGACTGGAGGACTGGGAGGATGAATTCGACCTG
GAGAACGCAGTGCTCTTCGAAGTGGCCTGGGAGGTGGCTAACAAGGTGGGTGGC
ATCTACACGGTGCTGCAGACGAAGGCGAAGGTGACAGGGGACGAATGGGGCGA
CAACTACTTCCTGGTGGGGCCGTACACGGAGCAGGGCGTGAGGACCCAGGTGGA
ACTGCTGGAGGCCCCCACCCCGGCCCTGAAGAGGACACTGGATTCCATGAACAG
CAAGGGCTGCAAGTTCCTGGCACAGAGTGAGGAGAAGCCACATGTGGTTGCTCA
CTTCCATGAGTGGTTGGCAGGCGTTGGACTCTGCCTGTGTCGTGCCCGGCGACTG
CCTGTAGCAACCATCTTCACCACCCATGCCACGCTGCTGGGGCGCTACCTGTGTG
CCGGTGCCGTGGACTTCTACAACAACCTGGAGAACTTCAACGTGGACAAGGAAG
CAGGGGAGAGGCAGATCTACCACCGATACTGCATGGAAAGGGCGGCAGCCCACT
GCGCTCACGTCTTCACTACTGTGTCCCAGATCACCGCCATCGAGGCACAGCACTT
GCTCAAGAGGAAACCAGATATTGTGACCCCCAATGGGCTGAATGTGAAGAAGTT
TTCTGCCATGCATGAGTTCCAGAACCTCCATGCTCAGAGCAAGGCTCGAATCCAG
GAGTTTGTGCGGGGCCATTTTTATGGGCATCTGGACTTCAACTTGGACAAGACCT
TATACTTCTTTATCGCCGGCCGCTATGAGTTCTCCAACAAGGGTGCTGACGTCTTC
CTGGAGGCATTGGCTCGGCTCAACTATCTGCTCAGAGTGAACGGCAGCGAGCAG
ACAGTGGTTGCCTTCTTCATCATGCCAGCGCGGACCAACAATTTCAACGTGGAAA
CCCTCAAAGGCCAAGCTGTGCGCAAACAGCTTTGGGACACGGCCAACACGGTGA
AGGAAAAGTTCGGGAGGAAGCTTTATGAATCCTTACTGGTTGGGAGCCTTCCCG
ACATGAACAAGATGCTGGATAAGGAAGACTTCACTATGATGAAGAGAGCCATCT
TTGCAACGCAGCGGCAGTCTTTCCCCCCTGTGTGCACCCACAATATGCTGGATGA
CTCCTCAGACCCCATCCTGACCACCATCCGCCGAATCGGCCTCTTCAATAGCAGT
GCCGACAGGGTGAAGGTGATTTTCCACCCGGAGTTCCTCTCCTCCACAAGCCCCC
TGCTCCCTGTGGACTATGAGGAGTTTGTCCGTGGCTGTCACCTTGGAGTCTTCCCC
TCCTACTATGAGCCTTGGGGCTACACACCGGCTGAGTGCACGGTTATGGGAATCC
CCAGTATCTCCACCAATCTCTCCGGCTTCGGCTGCTTCATGGAGGAACACATCGC
AGACCCCTCAGCTTACGGTATCTACATTCTTGACCGGCGGTTCCGCAGCCTGGAT
GATTCCTGCTCGCAGCTCACCTCCTTCCTCTACAGTTTCTGTCAGCAGAGCCGGC
GGCAGCGTATCATCCAGCGGAACCGCACGGAGCGCCTCTCCGACCTTCTGGACT
GGAAATACCTAGGCCGGTACTATATGTCTGCGCGCCACATGGCGCTGTCCAAGG
CCTTTCCAGAGCACTTCACCTACGAGCCCAACGAGGCGGATGCGGCCCAGGGGT
ACCGCTACCCACGGCCAGCCTCGGTGCCACCGTCGCCCTCGCTGTCACGACACTC
CAGCCCGCACCAGAGTGAGGACGAGGAGGATCCCCGGAACGGGCCGCTGGAGG
AAGACGGCGAGCGCTACGATGAGGACGAGGAGGCCGCCAAGGACCGGCGCAAC
ATCCGTGCACCAGAGTGGCCGCGCCGAGCGTCCTGCACCTCCTCCACCAGCGGC
AGCAAGCGCAACTCTGTGGACACGGCCACCTCCAGCTCACTCAGCACCCCGAGC
GAGCCCCTCAGCCCCACCAGCTCCCTGGGCGAGGAGCGTAACTAAGTCCGCCCC
ACCACACTCCCCGCCTGTCCTGCCTCTCTGCTCCAGAGAGAGGATGCAGAGGGGT
GCTGCTCCTAAACCCCCGCTCCAGATCTGCACTGGGTGTGGCCCCGCAGTGCCCC
CACCCAGTCCGCCAAACACTCCACCCCCTCCAGCTCCAGTTTCCAAGTTCCTGCA
CTCCAGAATCCACAAAGCCGTGCCTTTCTCTGGCTCCAGAATATGCATAATCAGC
GCCCTGGAGTCCCCTGGGCCTGGACCGCTTCCCAGAGGCCAGGAATCTGCCATTA
CTCTGCGGTGGTGCCAGAGGTTTTAGGAAACCTGGCATGGTGCTTTCAGGTCTGG
GGCTTTTAGAGCCCCCCGTGTGGCTTACAAATTCTACAGCATACAGAGCAGGCCA
CGCTCAGGCCCGGCATGCGGGCCACCAAGTTCTGGAAACCACGTGGTGTCCCTG
CGAATGGGGCGATCAAGTCCAGAGCCGGGGCACTTTCAGAGTTTGAAGGTAACT
GAGAGCAGATGGTCCTCCATTTCAACTCCAGAAGTGGGGCTCTGGGAGGGATGT
TCTAGCCCTCCCTGGCATGTCAGAGCCAGGCTCTGCCTGGAGGATCCCTCCATCC
GGCTCCTGTCATCCCCTACACTTTGGCCAAGCAAGAGGTGGTAGAACCACTTGGC
TGCTCATTCCTTCTGGAGGACACACAGTCTCAGTCCAGATGCCTTCCTGTCTTTCT
GGCCCTTTCTGGACCAGATCCTACTCTTCCTTTCTAAATCTGAGATCTCCCTCCAG
GGAATCCGCCTGCAGAGGACAGAGCTGGCTGTCTTCCCCCACCCCTAACCTGGCT
TATTCCCAACTGCTCTGCCCACTGTGAAACCACTAGGTTCTAGGTCCTGGCTTCTA
GATCTGGAACCTTACCACGTTACTGCATACTGATCCCTTTCCCATGATCCAGAAC
TGAGGTCACTGGGTTCTAGAACCCCCACATTTACCTCGAGGCTCTTCCATCCCCA
AACTGTGCCCTGCCTTCAGCTTTGGTGAAAGGGAGGGCCCCTCATGTGTGCTGTG
CTGTGTCTGCACCGCTTGGTTTGCAGTTGAGAGGGGAGGGCAGGAGGGGTGTGA
TTGGAGTGTGTCCGGAGATGAGATGAAAAAAATACATCTATATTTAAGAA

In some embodiments, the antisense strand of the dsRNA agent is 100% complementary to a target RNA to hybridize thereto and inhibits its expression through RNA interference. The target RNA can be any RNA expressed in a cell. In another embodiment, the cell is a tumor cell, a liver cell, a muscle cell, an immune cell, a heart cell, or a cell of the central nervous system. In another embodiment, the antisense strand of the dsRNA agent is at least 99%, at least 98%, at least 97%, at least 96%, 95%, at least 90%, at least 85%, at least 80%, at least 75%, at least 70%, at least 65%, at least 60%, at least 55%, or at least 50% complementary to a target RNA. In some embodiments, the target RNA is CD40, KRAS, or GYS1 RNA. In some embodiments, the siRNA molecule is a siRNA that reduces the expression of CD40, KRAS, or GYS1. In some embodiments, the siRNA molecule is a siRNA that reduces the expression of CD40, KRAS, or GYS1 and does not reduce the expression of other RNAs by more than 50% in an assay described herein at a concentration of no more than 200 nm as described herein.

In some embodiments, the siRNA is linked to a protein, such as a FN3 domain. The siRNA can be linked to multiple FN3 domains that bind to the same target protein or different target proteins. In some embodiments, the linker is attached to the sense strand, which is used to facilitate the linkage of the sense strand to the FN3 domain.

In some embodiments, compositions are provided herein having a formula of (X1)ₙ-(X2)_{q}-(X3)_{y}-L-X4, wherein X₁ is a first FN3 domain, X2 is second FN3 domain, X3 is a third FN3 domain or half-life extender molecule, L is a linker, and X₄ is a nucleic acid molecule, such as, but not limited to a siRNA molecule, wherein n, q, and y are each independently 0 or 1. In some embodiments, X1, X2, and X3 bind to different target proteins. In some embodiments, y is 0. In some embodiments, n is 1, q is 0, and y is 0. In some embodiments, n is 1, q is 1, and y is 0. In some embodiments, n is 1, q is 1, and y is 1. In some embodiments, the third FN3 domain increases the half-life of the molecule as a whole as compared to a molecule without X3. In some embodiments, the half-life extending moiety is a FN3 domain that binds to albumin. Examples of such FN3 domains include, but are not limited to, those described in U.S. Patent Application Publication No. 20170348397 and U.S. Pat. No. 9,156,887, which is hereby incorporated by reference in its entirety. The FN3 domains may incorporate other subunits for example via covalent interaction. In some embodiments, the FN3 domains further comprise a half-life extending moiety. Exemplary half-life extending moieties are albumin, albumin variants, albumin-binding proteins and/or domains, an aliphatic chain that binds to serum proteins, transferrin and fragments and analogues thereof, and Fc regions. Amino acid sequences of the human Fc regions are well known, and include IgG1, IgG2, IgG3, IgG4, IgM, IgA and IgE Fc regions. In some embodiments, the FN3 domains may incorporate a second FN3 domain that binds to a molecule that extends the half-life of the entire molecule, such as, but not limited to, any of the half-life extending moieties described herein. In some embodiments, the second FN3 domain binds to albumin, albumin variants, albumin-binding proteins and/or domains, and fragments and analogues thereof.

In some embodiments, compositions are provided herein having a formula of (X1)-(X2)-L-(X4), wherein X₁ is a first FN3 domain, X₂ is second FN3 domain, L is a linker, and X4 is a nucleic acid molecule. In some embodiments, X4 is a siRNA molecule. In some embodiments, X1 is a FN3 domain that binds to one of CD71. In some embodiments, X2 is a FN3 domain that binds to one of CD71. In some embodiments X1 and X2 do not bind to the same target protein. In some embodiments, X1 and X2 bind to the same target protein, but at different binding sites on the protein. In some embodiments, X1 and X2 bind to the same target protein. In some embodiments, X1 and X2 are FN3 domains that bind to CD71. In some embodiments, the composition does not comprise (e.g. is free of) a compound or protein that binds to ASGPR.

In some embodiments, compositions are provided herein having a formula of C—(X1)ₙ-(X2)_{q}[L-X4]-(X3)_{y}, wherein X1 is a first FN3 domain; X2 is second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions are provided herein having a formula of (X1)ₙ-(X2)_{q}[L-X4]-(X3)_{y}-C, wherein X1 is a first FN3 domain; X2 is second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions are provided herein having a formula of C—(X1)ₙ-(X2)_{q}[L-X4]L-(X3)_{y}, wherein X1 is a first FN3 domain; X2 is second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions are provided herein having a formula of (X1)ₙ-(X2)_{q}[L-X4]L-(X3)_{y}-C, wherein X1 is a first FN3 domain; X2 is second FN3 domain; X3 is a third FN3 domain or half-life extender molecule; L is a linker; X4 is an oligonucleotide molecule; and C is a polymer, wherein n, q, and y are each independently 0 or 1, are provided.

In some embodiments, compositions or complexes are provided having a formula of A₁-B₁, wherein A₁ has a formula of C-L₁-Xₛ and B₁ has a formula of X_{AS}-L₂-F₁, wherein:
C is a polymer, such as PEG;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule;
F₁ is a polypeptide comprising at least one FN3 domain;
wherein X_{S} and X_{AS} form a double stranded oligonucleotide molecule to form the composition/complex.

In some embodiments, compositions or complexes are provided having a formula of A₁-B₁, wherein A₁ has a formula of Xₛ and B₁ has a formula of X_{AS}-L₂-F₁.

In some embodiments, compositions or complexes are provided having a formula of A₁-B₁, wherein A₁ has a formula of C-L₁-Xₛ and B₁ has a formula of X_{AS}.

In some embodiments, the sense strand is a sense strand as provided for herein. In some embodiments, the antisense strand is an antisense strand as provided for herein. In some embodiments, the sense and antisense strand form a double stranded siRNA molecule that targets CD40, KRAS, or GYS1. In some embodiments, the double stranded oligonucleotide is about 21-23 nucleotides base pairs in length. In certain embodiments, C is optional.

In some embodiments, compositions or complexes are provided having a formula of A₁-B₁, wherein A₁ has a formula of F₁-L₁-Xₛ and B₁ has a formula of X_{AS}-L₂-C, wherein:
F₁ is a polypeptide comprising at least one FN3 domain;
L₁ and L₂ are each, independently, a linker;
C is a polymer, such as PEG;
X_{S} is a 5′ to 3′ oligonucleotide sense strand of a double stranded siRNA molecule;
X_{AS} is a 3′ to 5′ oligonucleotide antisense strand of a double stranded siRNA molecule;
wherein X_{S} and X_{AS} form a double stranded oligonucleotide molecule to form the composition/complex. In certain embodiments, C is optional.

In some embodiments, compositions or complexes are provided having a formula of A₁-B₁, wherein A₁ has a formula of Xₛ and B₁ has a formula of X_{AS}-L₂-C.

In some embodiments, compositions or complexes are provided having a formula of A₁-B₁, wherein A₁ has a formula of F₁-L₁-X₈ and B₁ has a formula of X_{AS}.

In some embodiments, A₁ and B₁ interact with each other through hydrogen bonding. In some embodiments, A₁ and B₁ interact with each other through Watson-Crick base pairing.

In some embodiments, compositions described a polymer (polymer moiety C, or just C). In some embodiments, C can be a molecule that extends the half-life of the molecule. In some embodiments, the polymer is a natural or synthetic polymer, consisting of long chains of branched or unbranched monomers, and/or cross-linked network of monomers in two or three dimensions In some instances, the polymer includes a polysaccharide, lignin, rubber, or polyalkylen oxide (e.g., polyethylene glycol). In some instances, the at least one polymer includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer comprises polyalkylene oxide. In some instances, the polymer comprises PEG. In some instances, the polymer comprises polyethylene imide (PEI) or hydroxy ethyl starch (HES).

In some embodiments, C is a PEG moiety. In some embodiments, the PEG moiety is conjugated at the 5′ terminus of the oligonucleotide molecule while the binding moiety is conjugated at the 3′ terminus of the oligonucleotide molecule. In some embodiments, the PEG moiety is conjugated at the 3′ terminus of the oligonucleotide molecule while the binding moiety is conjugated at the 5′ terminus of the oligonucleotide molecule. In some embodiments, the PEG moiety is conjugated to an internal site of the oligonucleotide molecule. In some embodiments, the PEG moiety, the binding moiety, or a combination thereof, are conjugated to an internal site of the oligonucleotide molecule. In some embodiments, the conjugation is a direct conjugation. In some embodiments, the conjugation is via native ligation.

In some embodiments, the polyalkylene oxide (e.g., PEG) is a polydisperse or monodisperse compound. In some embodiments, polydisperse material comprises disperse distribution of different molecular weight of the material, characterized by mean weight (weight average) size and dispersity. In some embodiments, the monodisperse PEG comprises one size of molecules. In some embodiments, C is poly- or monodispersed polyalkylene oxide (e.g., PEG) and the indicated molecular weight represents an average of the molecular weight of the polyalkylene oxide, e.g., PEG, molecules.

In some embodiments, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, C is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, the molecular weight of C is about 200 Da. In some embodiments, the molecular weight of C is about 300 Da. In some embodiments, the molecular weight of C is about 400 Da. In some embodiments, the molecular weight of C is about 500 Da. In some embodiments, the molecular weight of C is about 600 Da. In some embodiments, the molecular weight of C is about 700 Da. In some embodiments, the molecular weight of C is about 800 Da. In some embodiments, the molecular weight of C is about 900 Da. In some embodiments, the molecular weight of C is about 1000 Da. In some embodiments, the molecular weight of C is about 1100 Da. In some embodiments, the molecular weight of C is about 1200 Da. In some embodiments, the molecular weight of C is about 1300 Da. In some embodiments, the molecular weight of C is about 1400 Da. In some embodiments, the molecular weight of C is about 1450 Da. In some embodiments, the molecular weight of C is about 1500 Da. In some embodiments, the molecular weight of C is about 1600 Da. In some embodiments, the molecular weight of C is about 1700 Da. In some embodiments, the molecular weight of C is about 1800 Da. In some embodiments, the molecular weight of C is about 1900 Da. In some embodiments, the molecular weight of C is about 2000 Da. In some embodiments, the molecular weight of C is about 2100 Da. In some embodiments, the molecular weight of C is about 2200 Da. In some embodiments, the molecular weight of C is about 2300 Da. In some embodiments, the molecular weight of C is about 2400 Da. In some embodiments, the molecular weight of C is about 2500 Da. In some embodiments, the molecular weight of C is about 2600 Da. In some embodiments, the molecular weight of C is about 2700 Da. In some embodiments, the molecular weight of C is about 2800 Da. In some embodiments, the molecular weight of C is about 2900 Da. In some embodiments, the molecular weight of C is about 3000 Da. In some embodiments, the molecular weight of C is about 3250 Da. In some embodiments, the molecular weight of C is about 3350 Da. In some embodiments, the molecular weight of C is about 3500 Da. In some embodiments, the molecular weight of C is about 3750 Da. In some embodiments, the molecular weight of C is about 4000 Da. In some embodiments, the molecular weight of C is about 4250 Da. In some embodiments, the molecular weight of C is about 4500 Da. In some embodiments, the molecular weight of C is about 4600 Da. In some embodiments, the molecular weight of C is about 4750 Da. In some embodiments, the molecular weight of C is about 5000 Da. In some embodiments, the molecular weight of C is about 5500 Da. In some embodiments, the molecular weight of C is about 6000 Da. In some embodiments, the molecular weight of C is about 6500 Da. In some embodiments, the molecular weight of C is about 7000 Da. In some embodiments, the molecular weight of C is about 7500 Da. In some embodiments, the molecular weight of C is about 8000 Da. In some embodiments, the molecular weight of C is about 10,000 Da. In some embodiments, the molecular weight of C is about 12,000 Da. In some embodiments, the molecular weight of C is about 20,000 Da. In some embodiments, the molecular weight of C is about 35,000 Da. In some embodiments, the molecular weight of C is about 40,000 Da. In some embodiments, the molecular weight of C is about 50,000 Da. In some embodiments, the molecular weight of C is about 60,000 Da. In some embodiments, the molecular weight of C is about 100,000 Da.

In some embodiments, the polyalkylene oxide (e.g., PEG) is a discrete PEG, in which the discrete PEG is a polymeric PEG comprising more than one repeating ethylene oxide units. In some embodiments, a discrete PEG (dPEG) comprises from 2 to 60, from 2 to 50, or from 2 to 48 repeating ethylene oxide units. In some embodiments, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some embodiments, a dPEG comprises about 2 or more repeating ethylene oxide units. In some embodiments, a dPEG comprises about 3 or more repeating ethylene oxide units. In some embodiments, a dPEG comprises about 4 or more repeating ethylene oxide units. In some embodiments, a dPEG comprises about 5 or more repeating ethylene oxide units. In some embodiments, a dPEG comprises about 6 or more repeating ethylene oxide units. In some embodiments, a dPEG comprises about 7 or more repeating ethylene oxide units. In some embodiments, a dPEG comprises about 8 or more repeating ethylene oxide units. In some embodiments, a dPEG comprises about 9 or more repeating ethylene oxide units. In some embodiments, a dPEG comprises about 10 or more repeating ethylene oxide units. In some embodiments, a dPEG comprises about 11 or more repeating ethylene oxide units. In some embodiments, a dPEG comprises about 12 or more repeating ethylene oxide units. In some embodiments, a dPEG comprises about 13 or more repeating ethylene oxide units. In some embodiments, a dPEG comprises about 14 or more repeating ethylene oxide units. In some embodiments, a dPEG comprises about 15 or more repeating ethylene oxide units. In some embodiments, a dPEG comprises about 16 or more repeating ethylene oxide units. In some embodiments, a dPEG comprises about 17 or more repeating ethylene oxide units. In some embodiments, a dPEG comprises about 18 or more repeating ethylene oxide units. In some embodiments, a dPEG comprises about 19 or more repeating ethylene oxide units. In some embodiments, a dPEG comprises about 20 or more repeating ethylene oxide units. In some embodiments, a dPEG comprises about 22 or more repeating ethylene oxide units. In some embodiments, a dPEG comprises about 24 or more repeating ethylene oxide units. In some embodiments, a dPEG comprises about 26 or more repeating ethylene oxide units. In some embodiments, a dPEG comprises about 28 or more repeating ethylene oxide units. In some embodiments, a dPEG comprises about 30 or more repeating ethylene oxide units. In some embodiments, a dPEG comprises about 35 or more repeating ethylene oxide units. In some embodiments, a dPEG comprises about 40 or more repeating ethylene oxide units. In some embodiments, a dPEG comprises about 42 or more repeating ethylene oxide units. In some embodiments, a dPEG comprises about 48 or more repeating ethylene oxide units. In some embodiments, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, C is an albumin binding domain. In some embodiments, the albumin binding domain specifically binds to serum albumin, e.g., human serum albumin (HSA) to prolong the half-life of the domain or of another therapeutic to which the albumin-binding domain is associated or linked with. In some embodiments, the human serum albumin-binding domain comprises an initiator methionine (Met) linked to the N-terminus of the molecule. In some embodiments, the human serum albumin-binding domain comprise a cysteine (Cys) linked to a C-terminus or the N-terminus of the domain. The addition of the N-terminal Met and/or the C-terminal Cys may facilitate expression and/or conjugation to another molecule, which can be another half-life extending molecules, such as PEG, a Fc region, and the like.

In some embodiments, the albumin binding domain comprises the amino acid sequence of SEQ ID NOs: 5, 6, 7, 8, 9, 10, 11, 12, 13, 14, 15, 16, 17, 18, 19, 20, 21, 22, or 23, provided in Table 5 above. In some embodiments, the albumin binding domain (protein) is isolated. In some embodiments, the albumin binding domain comprises an amino acid sequence that is at least, or is, 85%, 86%, 87%, 88%, 89%, 90%, 91%, 92%, 93%, 94%, 95%, 96%, 97%, 98%, or 99% identical to the amino acid sequence of SEQ ID NO: 5, 6, 7, 8, 9, 10, 11, 12, 13, 14, 15, 16, 17, 18, 19, 20, 21, 22, or 23. In some embodiments, the albumin binding domain comprises an amino acid sequence that is at least, or is, 85%, 86%, 87%, 88%, 89%, 90%, 901%, 92%, 93%, 94%, 95%, 96%, 97%, 98%, or 99% identical to the amino acid sequence of SEQ ID NO: 5, 6, 7, 8, 9, 10, 11, 12, 13, 14, 15, 16, 17, 18, 19, 20, 21, 22, or 23 provided that the protein has a substitution that corresponds to position 10 of SEQ ID NO: 5, 6, 7, 8, 9, 10, 11, 12, 13, 14, 15, 16, 17, 18, 19, 20, 21, 22, or 23. In some embodiments, the substitution is A1OV. In some embodiments, the substitution is A10G, A10L, A10I, A10T, or A10S. In some embodiments, the substitution at position 10 is any naturally occurring amino acid. In some embodiments, the isolated albumin binding domain comprises an amino acid sequence that has 1, 2, 3, 4, 5, 6, 7, 8, 9, 10, 11, 12, 13, or 14 substitutions when compared to the amino acid sequence of SEQ ID NOs: 5, 6, 7, 8, 9, 10, 11, 12, 13, 14, 15, 16, 17, 18, 19, 20, 21, 22, or 23. In some embodiments, the substitution is at a position that corresponds to position 10 of SEQ ID NOs: 5, 6, 7, 8, 9, 10, 11, 12, 13, 14, 15, 16, 17, 18, 19, 20, 21, 22, or 23. In some embodiments, FN3 domains provided comprises a cysteine residue in at least one residue position corresponding to residue positions 6, 11, 22, 25, 26, 52, 53, 61, 88 or positions 6, 8, 10, 11, 14, 15, 16, 20, 30, 34, 38, 40, 41, 45, 47, 48, 53, 54, 59, 60, 62, 64, 70, 88, 89, 90, 91, or 93 of SEQ ID NO: 5, 6, 7, 8, 9, 10, 11, 12, 13, 14, 15, 16, 17, 18, 19, 20, 21, 22, or 23, or at a C-terminus. Although the positions are listed in a series, each position can also be chosen individually. In some embodiments, the cysteine is at a position that corresponds to position 6, 53, or 88. In some embodiments, additional examples of albumin binding domains can be found in U.S. Pat. No. 10,925,932, which hereby incorporated by reference. In some embodiments, additional examples of albumin binding domains can be found in U.S. Pat. Nos. 8,969,289, 9,540,424, 10,221,438, 10,934,572, 10,442,851, 11,203,630, 10,766,946, 11,434,275; and in U.S. Publication Nos. 2022/0204589 and 2023/0145413; each of which is hereby incorporated by reference in its entirety.

As provided for herein, the FN3 domains can be linked to a siRNA molecule. Although certain FN3 domains are illustrated with the methionine, it should be understood that the FN3 domain can be linked to the siRNA without the N-terminal methionine. Additionally, one of skill in the art would appreciate that the numbering for the cysteine residue location, which is provided for herein would be shifted to one residue lower without the N-terminal methionine being present.

In some embodiments, C can also be Endoporter, INF-7, TAT, polyarginine, polylysine, or an amphipathic peptide. These moieties can be used in place of or in addition to other half-life extending moieties provided for herein. In some embodiments, C can be a molecule that delivers the complex into the cell, the endosome, or the ER; said molecules are selected from those peptides listed in Table 6 above.

In some embodiments, L₁ is any linker that can be used to link the polymer C to the sense strand X_{S} or to link the polypeptide of F₁ to the sense strand X_{S}. In some embodiments, L₁ has a formula of.

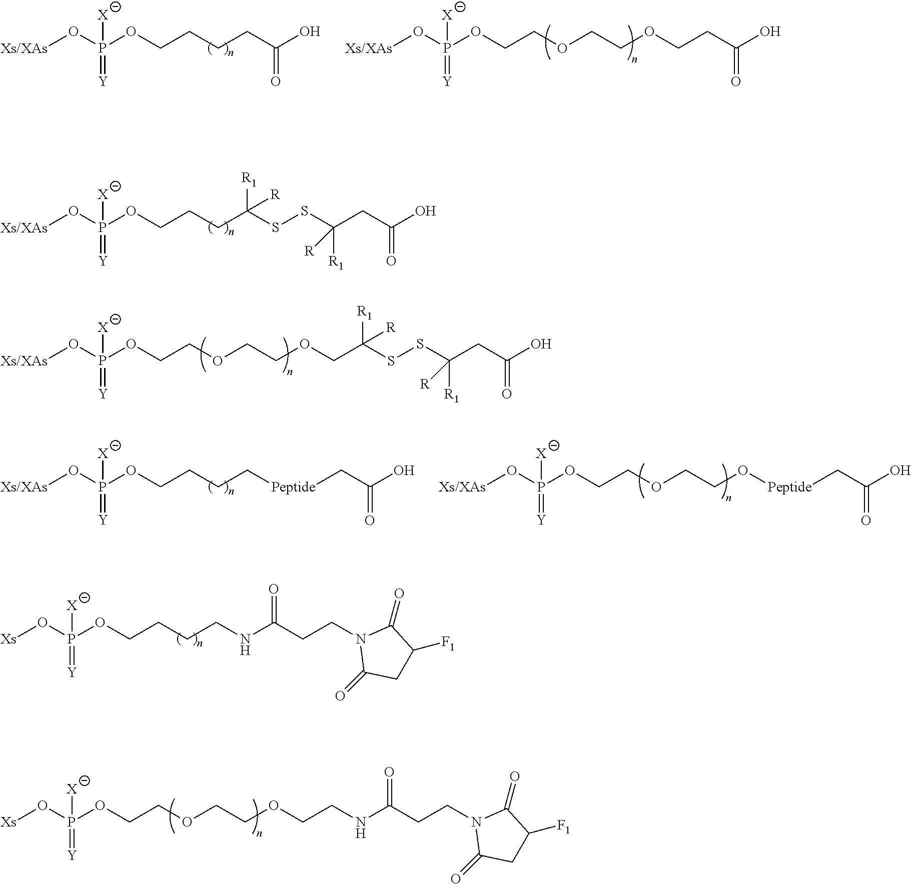

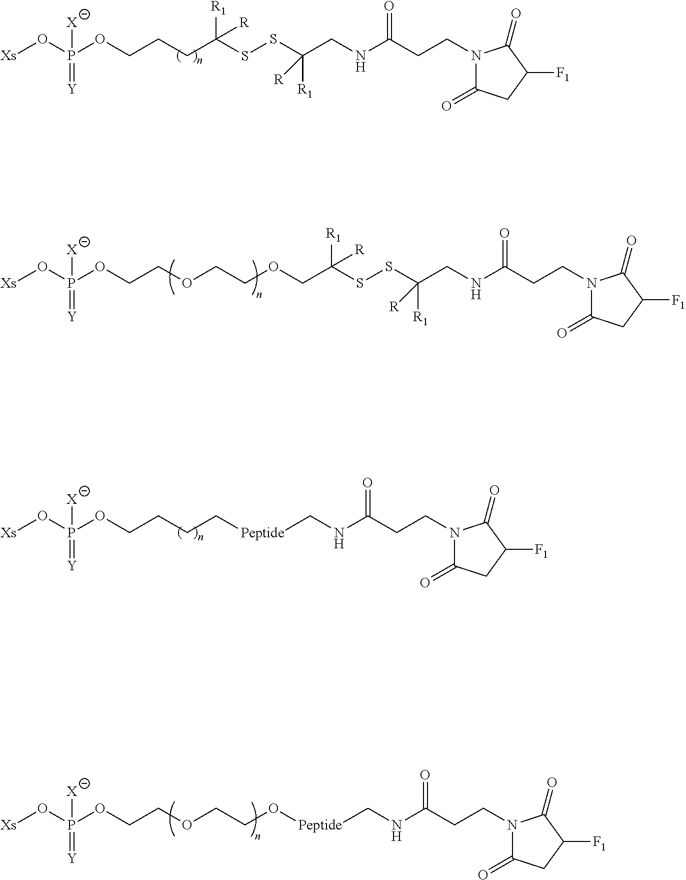

wherein X_{S}, X_{AS}, and F₁ are as defined above.

In some embodiments, n=0-20. In some embodiments, R and R1 are independently methyl. In some embodiments, R and RI are independently present or both are absent. In some embodiments, X and Y are independently S. In some embodiments, X and Y are independently present or absent. In some embodiments, Peptide is an enzymatically cleavable peptide, such as, but not limited to, Val-Cit, Val-Ala etc.

In some embodiments, L₂ is any linker that can be used to link the polypeptide of F₁ to the antisense strand X_{AS} or to link the polymer C to the antisense strand X_{AS}.

In some embodiments, L₂ has a formula of in the complex of

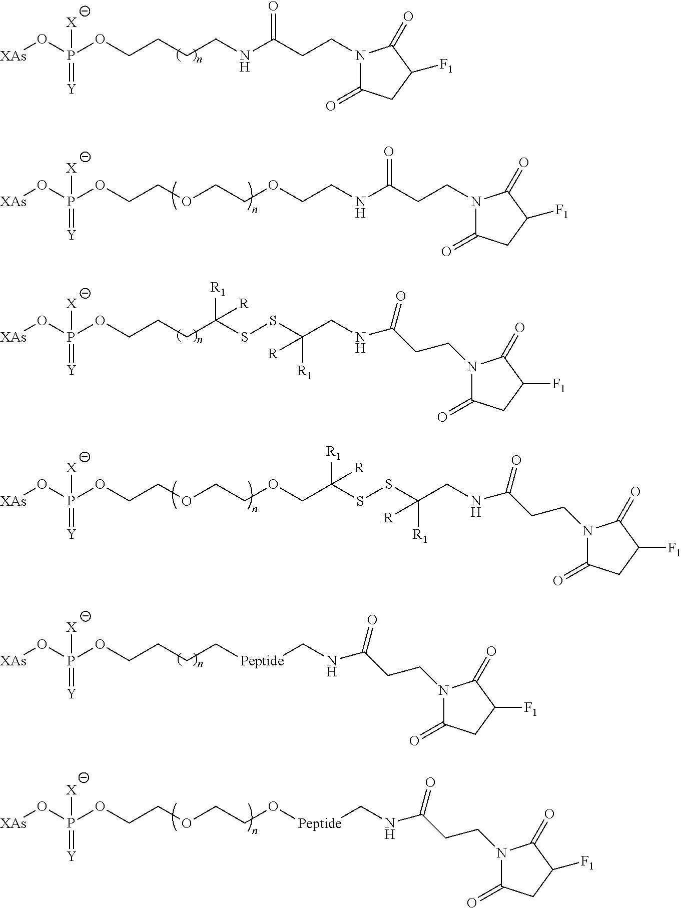

wherein X_{AS} and F₁ are as defined above.

In some embodiments, n=0-20. In some embodiments, R and RI are independently methyl. In some embodiments, R and R1 are independently present or both are absent. In some embodiments, X and Y are independently S. In some embodiments, X and Y are independently present or absent. In some embodiments, Peptide is an enzymatically cleavable peptide, such as, but not limited to, Val-Cit, Val-Ala etc.

In some embodiments, the linker is covalently attached to F1 through a cysteine residue present on F1, which can be illustrated as follows:

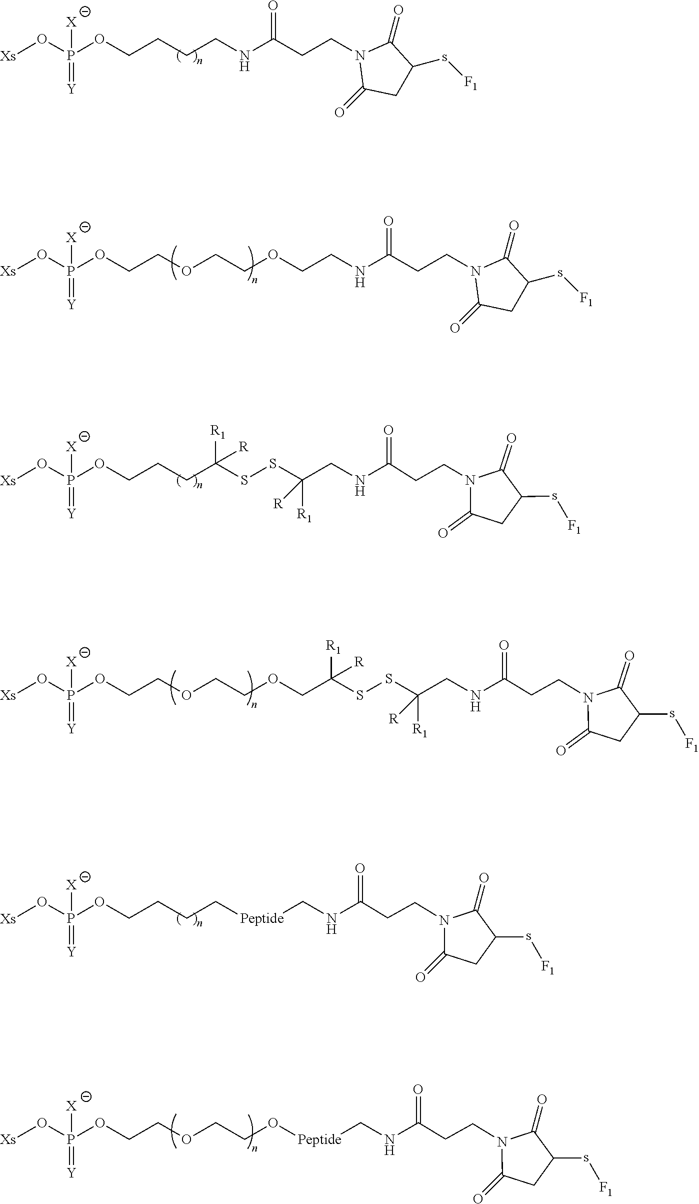

wherein X_{S} is a 5′ to 3′ oligonucleotide sense strand of a double stranded siRNA molecule; X_{AS} is a 3′ to 5′ oligonucleotide antisense strand of a double stranded siRNA molecule; and F₁ is a polypeptide comprising at least one FN3 domain, wherein X_{S} and X_{AS} form a double stranded siRNA molecule.

In some embodiments, A₁-B₁ has a formula of

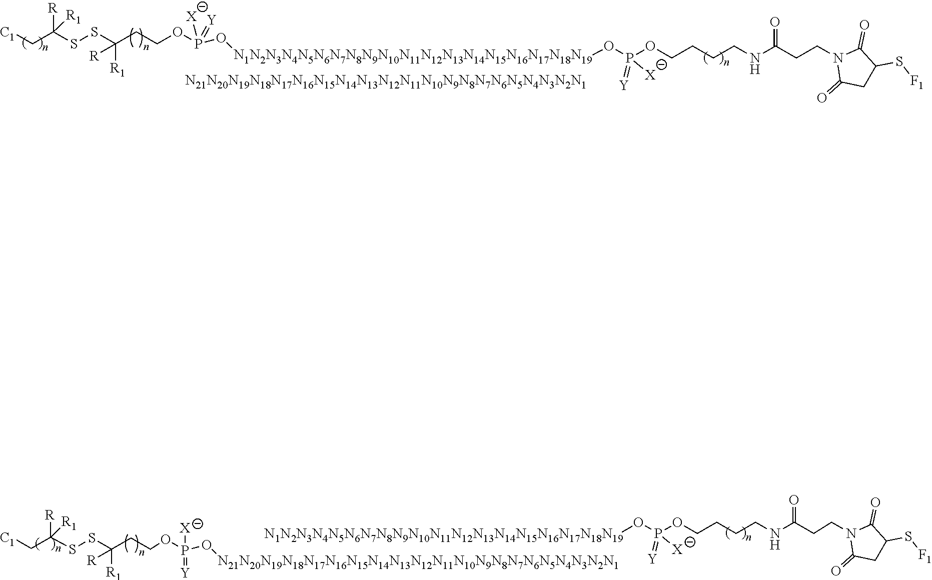

wherein C is the polymer, such as PEG, Endoporter, INF-7, TAT, polyarginine, polylysine, an amphipathic peptide, or as provided for herein; and F₁ is a polypeptide comprising at least one FN3 domain. The sense and antisense strands are represented by the “N” notations, wherein each nucleotide represented by N, is independently, A, U, C, or G or a modified nucleobase, such as those provided for herein. The N₁ nucleotides of the sense strand and the antisense strand represent the 5′ end of the respective strands. For clarity, although Formula III utilizes N₁, N₂, N₃, etc. in both the sense and the antisense strand, the nucleotide bases do not need to be the same and are not intended to be the same. The siRNA that is illustrated in Formula III would be complementary to a target sequence. For example, in some embodiments, the sense strand comprises a 2′O-methyl modified nucleotide with a phosphorothioate (PS) modified backbone at N₁ and N₂, a 2′-fluoro modified nucleotide at N₃, N₇, N₈, N₉, N₁₂, and N₁₇, and a 2′O-methyl modified nucleotide at N₄, N₅, N₆, N₁₀, N₁₁, N₁₃, N₁₄, N₁₅, N₁₆, N₁₈, and N₁₉.

In some embodiments, the antisense strand comprises a vinylphosphonate moiety attached to N₁, a 2′fluoro-modified nucleotide with a phosphorothioate (PS) modified backbone at N₂, a 2′O-methyl modified nucleotide at N₃, N₄, N₅, N₆, N₇, N₈, N₉, N₁₀, N₁₁, N₁₂, N₁₃, N₁₅, N₁₆, N₁₇, N₁₈, and N₁₉, a 2′fluoro-modified nucleotide at N₁₄, and a 2′O-methyl modified nucleotide with a phosphorothioate (PS) modified backbone at N₂₀ and N₂₁.

In some embodiments, a compound having a formula of:

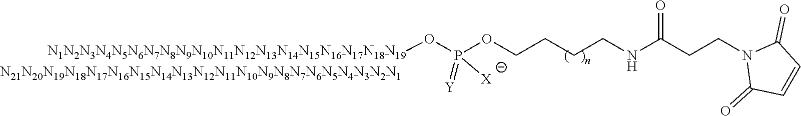

is provided.

In some embodiments, a compound having a formula of:

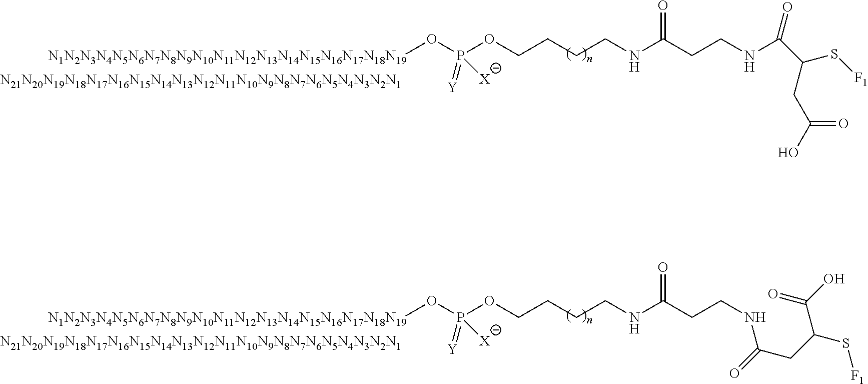

is provided, wherein F₁ is a polypeptide comprising at least one FN3 domain and is conjugated to a linker. The linker illustrated above, is a non-limiting example, and other types of linkers can be used.

In some embodiments, F₁ comprises polypeptide having a formula of (X₁)ₙ—(X₂)_{q}—(X₃)_{y}, wherein X₁ is a first FN3 domain; X₂ is second FN3 domain; X₃ is a third FN3 domain or half-life extender molecule; wherein n, q, and y are each independently 0 or 1, provided that at least one of n, q, and y is 1. In some embodiments, n, q, and y are each 1. In some embodiments, n and q are 1 and y is 0. In some embodiments n and y are 1 and q is 0.

In some embodiment X₁ is a CD71 FN3 binding domain, such as one provided herein. In some embodiments, X₂ is a CD71 FN3 binding domain. In some embodiments, X₁ and X₂ are different CD71 FN3 binding domains. In some embodiments, the binding domains are the same. In some embodiments, X₃ is a FN3 domain that binds to human serum albumin. In some embodiments, X₃ is a Fc domain without effector function that extends the half-life of a protein. In some embodiments, X₁ is a first CD71 binding domain, X₂ is a second CD71 binding domain, and X₃ is a FN3 albumin binding domain. Examples of such polypeptides are provided herein and below. In some embodiments, compositions are provided herein having a formula of C—(X₁)ₙ—(X₂)_{q}-(X₃)_{y}-L-X₄, wherein C is a polymer, such as PEG, Endoporter, INF-7, TAT, polyarginine, polylysine, an amphipathic peptide, or peptides provided in Table 2; X₁ is a first FN3 domain; X₂ is second FN3 domain; X₃ is a third FN3 domain or half-life extender molecule; L is a linker; and X₄ is a nucleic acid molecule, wherein n, q, and y are each independently 0 or 1.

In some embodiments, compositions are provided herein having a formula of (X₁)ₙ—(X₂)_{q}—(X₃)_{y}-L-X₄-C, wherein X₁ is a first FN3 domain; X₂ is second FN3 domain; X₃ is a third FN3 domain or half-life extender molecule; L is a linker; X₄ is a nucleic acid molecule; and C is a polymer, wherein n, q, and y are each independently 0 or 1.

In some embodiments, compositions are provided herein having a formula of X₄-L-(X₁)ₙ—(X₂)_{q}—(X₃)_{y}, wherein X₁ is a first FN3 domain; X₂ is second FN3 domain; X₃ is a third FN3 domain or half-life extender molecule; L is a linker; and X₄ is a nucleic acid molecule, wherein n, q, and y are each independently 0 or 1.

In some embodiments, compositions are provided herein having a formula of C—X₄-L-(X₁)ₙ—(X₂)_{q}—(X₃)_{y}, wherein C is a polymer; X₁ is a first FN3 domain; X₂ is second FN3 domain; X₃ is a third FN3 domain or half-life extender molecule; L is a linker; and X₄ is a nucleic acid molecule, wherein n, q, and y are each independently 0 or 1.

In some embodiments, compositions are provided herein having a formula of X₄-L-(X₁)ₙ—(X₂)_{q}—(X₃)_{y}-C, wherein X₁ is a first FN3 domain; X₂ is second FN3 domain; X₃ is a third FN3 domain or half-life extender molecule; L is a linker; X₄ is a nucleic acid molecule; and C is a polymer, wherein n, q, and y are each independently 0 or 1.

In some embodiments, the siRNA pair may follow the sequence: sense strand (5′-3′) nsnsnnnnNfNfNfnnnnnnnnsnsa or (5′-3′) nsnsnnnnNfNfNfnnnnnnnnna; and antisense strand (5′-3′) UfsNfsnnnNfnnnnnnnNfnNfnnnsusu, wherein (n) is 2′-O-Me (methyl), (Nf) is 2′-F (fluoro), (s) is phosphorothioate backbone modification. Each nucleotide in both sense and antisense strands are modified independently or in combination at ribosugar and nucleobase positions.

In some embodiments, the siRNA molecule comprises a sequence pair from Table 7a or Table 7b that interacts with GYS1. In some embodiments, the FN3 domain is conjugated to an siRNA molecule that inhibits the expression of GYS1. In some embodiments, examples of siRNA molecules that inhibit the expression of GYS1, and how they can be linked to FN3 domains, are provided in PCT International Publication No. WO2022/221550, which is hereby incorporated by reference in its entirety.

In some embodiments, any siRNA molecule provided for herein may comprise a linker molecule as disclosed herein. In some embodiments, the FN3 domain is conjugated to an oligonucleotide that interacts with KRAS. In some embodiments, the FN3 domain is conjugated to an siRNA molecule that inhibits the expression of KRAS. In some embodiments, examples of siRNA molecules that inhibit the expression of KRAS, and how they can be linked to FN3 domains, are provided in PCT International Publication No. WO2021/076574, which is hereby incorporated by reference in its entirety.

In some embodiments, any siRNA molecule provided for herein may comprise a linker molecule as disclosed herein. In some embodiments, the FN3 domain is conjugated to an oligonucleotide that interacts with CD40. In some embodiments, the FN3 domain is conjugated to an siRNA molecule that inhibits the expression of CD40. In some embodiments, examples of siRNA molecules that inhibit the expression of CD40, and how they can be linked to FN3 domains, are provided in U.S. Provisional Application No. 63/380,112, U.S. Provisional Application No. 63/505,898, U.S. patent application Ser. No. 18/490,450, and International Patent Application No. PCT/US23/77298, each of which is hereby incorporated by reference in its entirety.

TABLE 7a
siRNA Sense and Anti-sense Sequences

| siRNA | SEQ | | SEQ | |
|---|---|---|---|---|
| Pair | ID NO: | Sense Strand 5′-3′ | ID NO: | Antisense Strand 5′-3′ |
| A1 | 600 | [fC] [*mU] [*fG] [mG] | 660 | [mU] [*fU] [*mC] [fG] |
| | | [fG] [mA] [fG] [fG] [fA] | | [mA] [fA] [fU] [fU] [mC] |
| | | [mU] [fG] [mA] [mA] [mU] | | [fA] [mU] [mC] [mC] [fU] |
| | | [fU] [mC] [fG] [mA] | | [mC] [fC] [mC] [fA] |
| | | [mA] [T] | | [mG] [*fU] [*mU] |
| B1 | 601 | [fC] [*mA] [*fA] [mG] | 661 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fU] [mC] [fU] [mA] | | [mC] [fC] [mU] [fU] |
| | | [mA] [T] | | [mG] [*fU] [*mU] |
| C1 | 602 | [fU] [*mC] [*fC] [mG] | 662 | [mU] [*fA] [*mA] [fU] |
| | | [fC] [mA] [fG] [fC] [fC] | | [mC] [fA] [fU] [fC] [mC] |
| | | [mU] [fG] [mG] [mA] [mU] | | [fA] [mG] [mG] [mC] [fU] |
| | | [fG] [mA] [fU] [mU] | | [mG] [fC] [mG] [fG] |
| | | [mA] [T] | | [mA] [*fU] [*mU] |
| D1 | 603 | [mG] [*mC] [*fG] [mA] | 663 | [fU] [*fG] [*mU] [mU] |
| | | [mC] [mU] [fG] [fC] [fC] | | [mG] [mC] [mU] [mA] [mC] |
| | | [mU] [mG] [fU] [mA] [mG] | | [mA] [mG] [mG] [mC] [fA] |
| | | [mC] [mA] [fA] [mC] | | [mG] [mU] [mC] [mG] |
| | | [mA] [idT] | | [mA] [*mU] [*mU] |
| E1 | 604 | [mU] [*mU] [*fU] [mA] | 664 | [fU] [*fA] [*mG] [mU] |
| | | [mU] [mG] [fG] [fG] [fC] | | [mC] [mC] [mA] [mG] [mA] |
| | | [mA] [mU] [fC] [mU] [mG] | | [mU] [mG] [mC] [mC] [fC] |
| | | [mG] [mA] [fC] [mU] | | [mA] [mU] [mA] [mA] |
| | | [mA] [idT] | | [mA] [*mU] [*mU] |
| F1 | 605 | [mG] [*mC] [*fG] [mC] | 665 | [fU] [*fG] [*mA] [mA] |
| | | [mG] [mG] [fA] [fC] [fC] | | [mA] [mU] [mU] [mG] [mU] |
| | | [mA] [mA] [fC] [mA] [mA] | | [mU] [mG] [mG] [mU] [fC] |
| | | [mU] [mU] [fU] [mC] | | [mC] [mG] [mC] [mG] |
| | | [mA] [idT] | | [mC] [*mU] [*mU] |
| G1 | 606 | [mG] [*mG] [*fA] [mC] | 666 | [fU] [*fC] [*mG] [mU] |
| | | [mC] [mA] [fA] [fC] [fA] | | [mU] [mG] [mA] [mA] [mA] |
| | | [mA] [mU] [fU] [mU] [mC] | | [mU] [mU] [mG] [mU] [fU] |
| | | [mA] [mA] [fC] [mG] | | [mG] [mG] [mU] [mC] |
| | | [mA] [idT] | | [mC] [*mU] [*mU] |
| H1 | 607 | [mA] [*mC] [*fC] [mA] | 667 | [fU] [*fC] [*mA] [mC] |
| | | [mA] [mC] [fA] [fA] [fU] | | [mG] [mU] [mU] [mG] [mA] |
| | | [mU] [mU] [fC] [mA] [mA] | | [mA] [mA] [mU] [mU] [fG] |
| | | [mC] [mG] [fU] [mG] | | [mU] [mU] [mG] [mG] |
| | | [mA] [idT] | | [mU] [*mU] [*mU] |
| I1 | 608 | [mG] [*mC] [*fG] [mC] | 668 | [fU] [*fU] [*mC] [mC] |
| | | [mA] [mA] [fA] [fC] [fA] | | [mC] [mA] [mA] [mA] [mG] |
| | | [mG] [mC] [fU] [mU] [mU] | | [mC] [mU] [mG] [mU] [fU] |
| | | [mG] [mG] [fG] [mA] | | [mU] [mG] [mC] [mG] |
| | | [mA] [idT] | | [mC] [*mU] [*mU] |
| J1 | 609 | [mA] [*mG] [*fA] [mG] | 669 | [fU] [*fC] [*mG] [mU] |
| | | [mC] [mC] [fA] [fU] [fC] | | [mU] [mG] [mC] [mA] [mA] |
| | | [mU] [mU] [fU] [mG] [mC] | | [mA] [mG] [mA] [mU] [fG] |
| | | [mA] [mA] [fC] [mG] | | [mG] [mC] [mU] [mC] |
| | | [mA] [idT] | | [mU] [*mU] [*mU] |
| K1 | 610 | [mG] [*mA] [*fG] [mC] | 670 | [fU] [*fG] [*mC] [mG] |
| | | [mC] [mA] [fU] [fC] [fU] | | [mU] [mU] [mG] [mC] [mA] |
| | | [mU] [mU] [fG] [mC] [mA] | | [mA] [mA] [mG] [mA] [fU] |
| | | [mA] [mC] [fG] [mC] | | [mG] [mG] [mC] [mU] |
| | | [mA] [idT] | | [mC] [*mU] [*mU] |
| L1 | 611 | [mA] [*mG] [*fC] [mC] | 671 | [fU] [*fU] [*mG] [mC] |
| | | [mA] [mU] [fC] [fU] [fU] | | [mG] [mU] [mU] [mG] [mC] |
| | | [mU] [mG] [fC] [mA] [mA] | | [mA] [mA] [mA] [mG] [fA] |
| | | [mC] [mG] [fC] [mA] | | [mU] [mG] [mG] [mC] |
| | | [mA] [idT] | | [mU] [*mU] [*mU] |
| M1 | 612 | [mC] [*mC] [*fA] [mU] | 672 | [fU] [*fG] [*mC] [mU] |
| | | [mC] [mU] [fU] [fU] [fG] | | [mG] [mC] [mG] [mU] [mU] |
| | | [mC] [mA] [fA] [mC] [mG] | | [mG] [mC] [mA] [mA] [fA] |
| | | [mC] [mA] [fG] [mC] | | [mG] [mA] [mU] [mG] |
| | | [mA] [idT] | | [mG] [*mU] [*mU] |
| N1 | 613 | [mA] [*mC] [*fG] [mC] | 673 | [fU] [*fG] [*mA] [mA] |
| | | [mA] [mG] [fC] [fG] [fG] | | [mA] [mG] [mA] [mC] [mU] |
| | | [mC] [mA] [fG] [mU] [mC] | | [mG] [mC] [mC] [mG] [fC] |
| | | [mU] [mU] [fU] [mC] | | [mU] [mG] [mC] [mG] |
| | | [mA] [idT] | | [mU] [*mU] [*mU] |
| O1 | 614 | [mU] [*mG] [*fA] [mC] | 674 | [fU] [*fU] [*mU] [mC] |
| | | [mC] [mA] [fC] [fC] [fA] | | [mG] [mG] [mC] [mG] [mG] |
| | | [mU] [mC] [fC] [mG] [mC] | | [mA] [mU] [mG] [mG] [fU] |
| | | [mC] [mG] [fA] [mA] | | [mG] [mG] [mU] [mC] |
| | | [mA] [idT] | | [mA] [*mU] [*mU] |
| P1 | 615 | [mC] [*mG] [*fA] [mA] | 675 | [fU] [*fA] [*mU] [mU] |
| | | [mU] [mC] [fG] [fG] [fC] | | [mG] [mA] [mA] [mG] [mA] |
| | | [mC] [mU] [fC] [mU] [mU] | | [mG] [mG] [mC] [mC] [fG] |
| | | [mC] [mA] [fA] [mU] | | [mA] [mU] [mU] [mC] |
| | | [mA] [idT] | | [mG] [*mU] [*mU] |
| Q1 | 616 | [mC] [*mU] [*fU] [mC] | 676 | [fU] [*fU] [*mC] [mG] |
| | | [mA] [mA] [fU] [fA] [fG] | | [mG] [mC] [mA] [mC] [mU] |
| | | [mC] [mA] [fG] [mU] [mG] | | [mG] [mC] [mU] [mA] [fU] |
| | | [mC] [mC] [fG] [mA] | | [mU] [mG] [mA] [mA] |
| | | [mA] [idT] | | [mG] [*mU] [*mU] |
| R1 | 617 | [mC] [*mA] [*fG] [mU] | 677 | [fU] [*fA] [*mG] [mA] |
| | | [mA] [mU] [fC] [fU] [fC] | | [mU] [mU] [mG] [mG] [mU] |
| | | [mC] [mA] [fC] [mC] [mA] | | [mG] [mG] [mA] [mG] [fA] |
| | | [mA] [mU] [fC] [mU] | | [mU] [mA] [mC] [mU] |
| | | [mA] [idT] | | [mG] [*mU] [*mU] |
| S1 | 618 | [mC] [*mC] [*fA] [mC] | 678 | [fU] [*fA] [*mG] [mC] |
| | | [mC] [mA] [fA] [fU] [fC] | | [mC] [mG] [mG] [mA] [mG] |
| | | [mU] [mC] [fU] [mC] [mC] | | [mA] [mG] [mA] [mU] [fU] |
| | | [mG] [mG] [fC] [mU] | | [mG] [mG] [mU] [mG] |
| | | [mA] [idT] | | [mG] [*mU] [*mU] |
| T1 | 619 | [mC] [*mA] [*fC] [mC] | 679 | [fU] [*fA] [*mA] [mG] |
| | | [mA] [mA] [fU] [fC] [fU] | | [mC] [mC] [mG] [mG] [mA] |
| | | [mC] [mU] [fC] [mC] [mG] | | [mG] [mA] [mG] [mA] [fU] |
| | | [mG] [mC] [fU] [mU] | | [mU] [mG] [mG] [mU] |
| | | [mA] [idT] | | [mG] [*mU] [*mU] |
| U1 | 620 | [mC] [*mC] [*fC] [mC] | 680 | [fU] [*fA] [*mU] [mA] |
| | | [mU] [mC] [fA] [fG] [fC] | | [mC] [mC] [mG] [mU] [mA] |
| | | [mU] [mU] [fA] [mC] [mG] | | [mA] [mG] [mC] [mU] [fG] |
| | | [mG] [mU] [fA] [mU] | | [mA] [mG] [mG] [mG] |
| | | [mA] [idT] | | [mG] [*mU] [*mU] |
| V1 | 62 | [mC] [*mC] [*fC] [mU] | 681 | [fU] [*fG] [*mA] [mU] |
| | | [mC] [mA] [fG] [fC] [fU] | | [mA] [mC] [mC] [mG] [mU] |
| | | [mU] [mA] [fC] [mG] [mG] | | [mA] [mA] [mG] [mC] [fU] |
| | | [mU] [mA] [fU] [mC] | | [mG] [mA] [mG] [mG] |
| | | [mA] [idT] | | [mG] [*mU] [*mU] |
| W1 | 622 | [mU] [*mU] [*fA] [mC] | 682 | [fU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mU] [fC] [mU] | | [mC] [mG] [mU] [mA] |
| | | [mA] [idT] | | [mA] [*mU] [*mU] |
| X1 | 623 | [mG] [*mG] [*fA] [mA] | 683 | [fU] [*fG] [*mG] [mC] |
| | | [mC] [mC] [fG] [fC] [fA] | | [mG] [mC] [mU] [mC] [mC] |
| | | [mC] [mG] [fG] [mA] [mG] | | [mG] [mU] [mG] [mC] [fG] |
| | | [mC] [mG] [fC] [mC] | | [mG] [mU] [mU] [mC] |
| | | [mA] [idT] | | [mC] [*mU] [*mU] |
| Y1 | 624 | [mA] [*mC] [*fG] [mG] | 684 | [fU] [*fG] [*mU] [mC] |
| | | [mA] [mG] [fC] [fG] [fC] | | [mG] [mG] [mA] [mG] [mA] |
| | | [mC] [mU] [fC] [mU] [mC] | | [mG] [mG] [mC] [mG] [fC] |
| | | [mC] [mG] [fA] [mC] | | [mU] [mC] [mC] [mG] |
| | | [mA] [idT] | | [mU] [*mU] [*mU] |
| Z1 | 625 | [mA] [*mG] [*fC] [mA] | 685 | [fU] [*fC] [*mA] [mC] |
| | | [mA] [mG] [fC] [fG] [fC] | | [mA] [mG] [mA] [mG] [mU] |
| | | [mA] [mA] [fC] [mU] [mC] | | [mU] [mG] [mC] [mG] [fC] |
| | | [mU] [mG] [fU] [mG] | | [mU] [mU] [mG] [mC] |
| | | [mA] [idT] | | [mU] [*mU] [*mU] |
| A2 | 626 | [mC] [*mA] [*fG] [mA] | 686 | [fU] [*fG] [*mA] [mA] |
| | | [mU] [mG] [fG] [fU] [fC] | | [mA] [mU] [mG] [mG] [mA] |
| | | [mC] [mU] [fC] [mC] [mA] | | [mG] [mG] [mA] [mC] [fC] |
| | | [mU] [mU] [fU] [mC] | | [mA] [mU] [mC] [mU] |
| | | [mA] [idT] | | [mG] [*mU] [*mU] |
| B2 | 627 | [mU] [*mC] [*fU] [mA] | 687 | [fU] [*fU] [*mA] [mA] |
| | | [mG] [mU] [fU] [fC] [fU] | | [mG] [mG] [mU] [mU] [mC] |
| | | [mG] [mG] [fA] [mA] [mC] | | [mC] [mA] [mG] [mA] [fU] |
| | | [mC] [mU] [fU] [mA] | | [mC] [mU] [mA] [mG] |
| | | [mA] [idT] | | [mA] [*mU] [*mU] |
| C2 | 628 | [mC] [*mC] [*fC] [mA] | 688 | [fU] [*fC] [*mC] [mU] |
| | | [mC] [mA] [fU] [fU] [fU] | | [mC] [mG] [mA] [mG] [mG] |
| | | [mA] [mC] [fC] [mU] [mC] | | [mU] [mA] [mA] [mA] [fU] |
| | | [mG] [mA] [fG] [mG] | | [mG] [mU] [mG] [mG] |
| | | [mA] [idT] | | [mG] [*mU] [*mU] |
| D2 | 629 | [mA] [*mC] [*fC] [mG] | 689 | [fU] [*fA] [*mA] [mC] |
| | | [mC] [mU] [fU] [fG] [fG] | | [mU] [mG] [mC] [mA] [mA] |
| | | [mU] [mU] [fU] [mG] [mC] | | [mA] [mC] [mC] [mA] [fA] |
| | | [mA] [mG] [fU] [mU] | | [mG] [mC] [mG] [mG] |
| | | [mA] [idT] | | [mU] [*mU] [*mU] |
| E2 | 630 | [fC] [*mU] [*fG] [mG] | 690 | [mU] [*fU] [*mC] [fG] |
| | | [fG] [mA] [fG] [fG] [fA] | | [mA] [fA] [fU] [fU] [mC] |
| | | [mU] [fG] [mA] [mA] [mU] | | [fA] [mU] [mC] [mC] [fU] |
| | | [fU] [mC] [fG] [mG] | | [mC] [fC] [mC] [fA] |
| | | [mA] [T] | | [mG] [*fU] [*mU] |
| F2 | 631 | [fC] [*mU] [*fG] [mG] | 691 | [mU] [*fU] [*mC] [fG] |
| | | [fG] [mA] [fG] [fG] [fA] | | [mA] [fA] [fU] [fU] [mC] |
| | | [mU] [fG] [mA] [mA] [mU] | | [fA] [mU] [mC] [mC] [fU] |
| | | [fU] [mA] [fG] [mG] | | [mC] [fC] [mC] [fA] |
| | | [mA] [T] | | [mG] [*fU] [*mU] |
| G2 | 632 | [fC] [*mU] [*fG] [mG] | 692 | [mU] [*fU] [*mC] [fG] |
| | | [fG] [mA] [fG] [fG] [fA] | | [mA] [fA] [fU] [fU] [mC] |
| | | [mU] [fG] [mA] [mA] [mU] | | [fA] [mU] [mC] [mC] [fU] |
| | | [fG] [mA] [fG] [mG] | | [mC] [fC] [mC] [fA] |
| | | [mA] [T] | | [mG] [*fU] [*mU] |
| H2 | 633 | [fC] [*mU] [*fG] [mG] | 693 | [fU] [*fU] [*fC] [fG] |
| | | [fG] [mA] [fG] [fG] [fA] | | [fA] [fA] [fU] [fU] [fC] |
| | | [mU] [fG] [mA] [mA] [mU] | | [fA] [fU] [fC] [fC] [fU] |
| | | [fU] [mC] [fG] [mA] | | [fC] [fC] [fC] [fA] |
| | | [mA] [T] | | [fG] [*fU] [*fU] |
| I2 | 634 | [fC] [*mU] [*fG] [mG] | 694 | [mU] [*fU] [*fC] [fG] |
| | | [fG] [mA] [fG] [fG] [fA] | | [fA] [fA] [fU] [fU] [mC] |
| | | [mU] [fG] [mA] [mA] [mU] | | [fA] [mU] [fC] [fC] [fU] |
| | | [fU] [mC] [fG] [mA] | | [fC] [fC] [fC] [fA] |
| | | [mA] [T] | | [fG] [*fU] [*mU] |
| J2 | 635 | [fC] [*mU] [*fG] [mG] | 695 | [mU] [*fU] [*mC] [fG] |
| | | [fG] [mA] [fG] [fG] [fA] | | [mA] [fA] [fU] [fU] [mC] |
| | | [mU] [fG] [mA] [mA] [*fU] | | [fA] [mU] [mC] [mC] [fU] |
| | | [idT] | | [mC] [fC] [mC] [fA] |
| | | | | [*mG] [*fU] [*mU] |
| K2 | 636 | [fC] [*mA] [*fA] [mG] | 696 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fU] [mC] [fU] [mA] | | [mC] [fC] [mU] [fU] |
| | | [mA] [idT] | | [mG] [*fU] [*mU] |
| L2 | 637 | [fC] [*mA] [*fA] [mG] | 697 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fU] [mC] [fU] [mA] | | [mC] [fC] [mU] [fU] |
| | | [mG] [idT] | | [mG] [*fU] [*mU] |
| M2 | 638 | [fC] [*mA] [*fA] [mG] | 698 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fU] [mC] [fU] [mG] | | [mC] [fC] [mU] [fU] |
| | | [mA] [idT] | | [mG] [*fU] [*mU] |
| N2 | 639 | [fC] [*mA] [*fA] [mG] | 699 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fU] [mC] [fG] [mA] | | [mC] [fC] [mU] [fU] |
| | | [mA] [idT] | | [mG] [*fU] [*mU] |
| O2 | 640 | [fC] [*mA] [*fA] [mG] | 700 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fU] [mA] [fU] [mA] | | [mC] [fC] [mU] [fU] |
| | | [mA] [idT] | | [mG] [*fU] [*mU] |
| P2 | 641 | [fC] [*mA] [*fA] [mG] | 701 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fG] [mC] [fU] [mA] | | [mC] [fC] [mU] [fU] |
| | | [mA] [idT] | | [mG] [*fU] [*mU] |
| Q2 | 642 | [fC] [*mA] [*fA] [mG] | 702 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fG] [mC] [fU] [mA] | | [mC] [fC] [mU] [fU] |
| | | [mG] [idT] | | [mG] [*fU] [*mU] |
| R2 | 643 | [mG] [*mA] [*fG] [mC] | 703 | [mU] [*fG] [*mC] [mG] |
| | | [mC] [mA] [fU] [fC] [fU] | | [mU] [mU] [mG] [mC] [mA] |
| | | [mU] [mU] [fG] [mC] [mA] | | [mA] [mA] [mG] [mA] [fU] |
| | | [mA] [mC] [fG] [mC] | | [mG] [mG] [mC] [mU] |
| | | [mA] [idT] | | [mC] [*mU] [*mU] |
| S2 | 644 | [mG] [*mA] [*fG] [mC] | 704 | [mU] [*fG] [*mC] [mG] |
| | | [mC] [mA] [fU] [fC] [fU] | | [mU] [mU] [mG] [mC] [mA] |
| | | [mU] [mU] [fG] [mC] [mA] | | [mA] [mA] [mG] [mA] [fU] |
| | | [mA] [mC] [fG] [mC] | | [mG] [mG] [mC] [mU] |
| | | [mG] [idT] | | [mC] [*mU] [*mU] |
| T2 | 645 | [mG] [*mA] [*fG] [mC] | 705 | [mU] [*fG] [*mC] [mG] |
| | | [mC] [mA] [fU] [fC] [fU] | | [mU] [mU] [mG] [mC] [mA] |
| | | [mU] [mU] [fG] [mC] [mA] | | [mA] [mA] [mG] [mA] [fU] |
| | | [mA] [mC] [fG] [mA] | | [mG] [mG] [mC] [mU] |
| | | [mA] [idT] | | [mC] [*mU] [*mU] |
| U2 | 646 | [mG] [*mA] [*fG] [mC] | 706 | [mU] [*fG] [*mC] [mG] |
| | | [mC] [mA] [fU] [fC] [fU] | | [mU] [mU] [mG] [mC] [mA] |
| | | [mU] [mU] [fG] [mC] [mA] | | [mA] [mA] [mG] [mA] [fU] |
| | | [mA] [mC] [fA] [mC] | | [mG] [mG] [mC] [mU] |
| | | [mA] [idT] | | [mC] [*mU] [*mU] |
| V2 | 647 | [mG] [*mA] [*fG] [mC] | 707 | [mU] [*fG] [*mC] [mG] |
| | | [mC] [mA] [fU] [fC] [fU] | | [mU] [mU] [mG] [mC] [mA] |
| | | [mU] [mU] [fG] [mU] [mA] | | [mA] [mA] [mG] [mA] [fU] |
| | | [mA] [mA] [fG] [mC] | | [mG] [mG] [mC] [mU] |
| | | [mA] [idT] | | [mC] [*mU] [*mU] |
| W2 | 648 | [mG] [*mA] [*fG] [mC] | 708 | [mU] [*fG] [*mC] [mG] |
| | | [mC] [mA] [fU] [fC] [fU] | | [mU] [mU] [mG] [mC] [mA] |
| | | [mU] [mU] [fG] [mC] [mA] | | [mA] [mA] [mG] [mA] [fU] |
| | | [mG] [mC] [fG] [mC] | | [mG] [mG] [mC] [mU] |
| | | [mA] [idT] | | [mC] [*mU] [*mU] |
| X2 | 649 | [mG] [*mA] [*fG] [mC] | 709 | [mU] [*fG] [*mC] [mG] |
| | | [mC] [mA] [fU] [fC] [fU] | | [mU] [mU] [mG] [mC] [mA] |
| | | [mU] [mU] [fG] [mC] [mA] | | [mA] [mA] [mG] [mA] [fU] |
| | | [mG] [mC] [fG] [mC] | | [mG] [mG] [mC] [mU] |
| | | [mG] [idT] | | [mC] [*mU] [*mU] |
| Y2 | 650 | [mU] [*mU] [*fA] [mC] | 710 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mU] [fC] [mU] | | [mC] [mG] [mU] [mA] |
| | | [mA] [idT] | | [mA] [*mU] [*mU] |
| Z2 | 651 | [mU] [*mU] [*fA] [mC] | 711 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mU] [fC] [mU] | | [mC] [mG] [mU] [mA] |
| | | [mG] [idT] | | [mA] [*mU] [*mU] |
| A3 | 652 | [mU] [*mU] [*fA] [mC] | 712 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mU] [fC] [mG] | | [mC] [mG] [mU] [mA] |
| | | [mA] [idT] | | [mA] [*mU] [*mU] |
| B3 | 653 | [mU] [*mU] [*fA] [mC] | 713 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mU] [fG] [mU] | | [mC] [mG] [mU] [mA] |
| | | [mA] [idT] | | [mA] [*mU] [*mU] |
| C3 | 654 | [mU] [*mU] [*fA] [mC] | 714 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mG] [fC] [mU] | | [mC] [mG] [mU] [mA] |
| | | [mA] [idT] | | [mA] [*mU] [*mU] |
| D3 | 655 | [mU] [*mU] [*fA] [mC] | 715 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mG] [fC] [mU] | | [mC] [mG] [mU] [mA] |
| | | [mA] [idT] | | [mA] [*mU] [*mU] |
| E3 | 656 | [mU] [*mU] [*fA] [mC] | 716 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mG] [mU] [fC] [mU] | | [mC] [mG] [mU] [mA] |
| | | [mA] [idT] | | [mA] [*mU] [*mU] |
| F3 | 657 | [mU] [*mU] [*fA] [mC] | 717 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mG] [mU] [fC] [mU] | | [mC] [mG] [mU] [mA] |
| | | [mG] [idT] | | [mA] [*mU] [*mU] |
| G3 | 658 | [fC] [*mA] [*fA] [mG] | 718 | [mU] [*fU] [*mA] [fG] |
| | | [fG] [mU] [fG] [fG] [fG] | | [mA] [fU] [fG] [fC] [mC] |
| | | [mU] [fG] [mG] [mC] [mA] | | [fA] [mC] [mC] [mC] [fA] |
| | | [fU] [mC] [fU] [mA] | | [mC] [fC] [mU] [fU] |
| | | [mG] [idT] | | [mG] [*fU] [*mU] |
| H3 | 659 | [mU] [*mU] [*fA] [mC] | 719 | [mU] [*fA] [*mG] [mA] |
| | | [mG] [mG] [fU] [fA] [fU] | | [mA] [mU] [mG] [mU] [mA] |
| | | [mC] [mU] [fA] [mC] [mA] | | [mG] [mA] [mU] [mA] [fC] |
| | | [mU] [mU] [fC] [mU] | | [mC] [mG] [mU] [mA] |
| | | [mA] | | [mA] [*mU] [*mU] |

Abbreviations Key:
n/N = any nucleotide
mN = 2′-O-methyl residues
fN = 2′-F residues
idT = inverted Dt
vinmN = 2′-O methyl vinyl phosphonate uridine
*= phosphorothioate
The brackets indicate the individual bases.

TABLE 7b
siRNA Sense and Anti-sense Sequences

| SEQ | | SEQ | |
|---|---|---|---|
| ID NO: | Sense Strand 5′-3′ | ID NO: | Antisense Strand 5′-3′ |
| 720 | caggggugcg gucuugcaa | 846 | caggggugcg gucuugcaa |
| 721 | ggugcggucu ugcaauagg | 847 | ggugcggucu ugcaauagg |
| 722 | gugcggucuu gcaauagga | 848 | gugcggucuu gcaauagga |
| 723 | agccaugccu uuaaaccgc | 849 | agccaugccu uuaaaccgc |
| 724 | augccuuuaa accgcacuu | 850 | augccuuuaa accgcacuu |
| 725 | ugggaggaug aauucgacc | 851 | ugggaggaug aauucgacc |
| 726 | aacgcagugc ucuucgaag | 852 | aacgcagugc ucuucgaag |
| 727 | ggacgaaugg ggcgacaac | 853 | ggacgaaugg ggcgacaac |
| 728 | ggcgacugcc uguagcaac | 854 | ggcgacugcc uguagcaac |
| 729 | cacgcugcug gggcgcuac | 855 | cacgcugcug gggcgcuac |
| 730 | ugcgcucacg ucuucacua | 856 | ugcgcucacg ucuucacua |
| 731 | ccuuauacuu cuuuaucgc | 857 | ccuuauacuu cuuuaucgc |
| 732 | cuuuaucgcc ggccgcuau | 858 | cuuuaucgcc ggccgcuau |
| 733 | ucgccggccg cuaugaguu | 859 | ucgccggccg cuaugaguu |
| 734 | gccgcuauga guucuccaa | 860 | gccgcuauga guucuccaa |
| 735 | ccgcuaugag uucuccaac | 861 | ccgcuaugag uucuccaac |
| 736 | ggcucggcuc aacuaucug | 862 | ggcucggcuc aacuaucug |
| 737 | gcucggcuca acuaucugc | 863 | gcucggcuca acuaucugc |
| 738 | ucggcucaac uaucugcuc | 864 | ucggcucaac uaucugcuc |
| 739 | ccagcgcgga ccaacaauu | 865 | ccagcgcgga ccaacaauu |
| 740 | agcgcggacc aacaauuuc | 866 | agcgcggacc aacaauuuc |
| 741 | ucgggaggaa gcuuuauga | 867 | ucgggaggaa gcuuuauga |
| 742 | acgcagcggc agucuuucc | 868 | acgcagcggc agucuuucc |
| 743 | gaccaccauc cgccgaauc | 869 | gaccaccauc cgccgaauc |
| 744 | ccgaaucggc cucuucaau | 870 | ccgaaucggc cucuucaau |
| 745 | aaucggccuc uucaauagc | 871 | aaucggccuc uucaauagc |
| 746 | caauagcagu gccgacagg | 872 | caauagcagu gccgacagg |
| 747 | aggaguuugu ccguggcug | 873 | aggaguuugu ccguggcug |
| 748 | cggcugagug cacgguuau | 874 | cggcugagug cacgguuau |
| 749 | ugggaauccc caguaucuc | 875 | ugggaauccc caguaucuc |
| 750 | cagaccccuc agcuuacgg | 876 | cagaccccuc agcuuacgg |
| 751 | ccccucagcu uacgguauc | 877 | ccccucagcu uacgguauc |
| 752 | cucagcuuac gguaucuac | 878 | cucagcuuac gguaucuac |
| 753 | gcuuacggua ucuacauuc | 879 | gcuuacggua ucuacauuc |
| 754 | ccggcggcag cguaucauc | 880 | ccggcggcag cguaucauc |
| 755 | agcguaucau ccagcggaa | 881 | agcguaucau ccagcggaa |
| 756 | gcguaucauc cagcggaac | 882 | gcguaucauc cagcggaac |
| 757 | uaccuaggcc gguacuaua | 883 | uaccuaggcc gguacuaua |
| 758 | cuaggccggu acuauaugu | 884 | cuaggccggu acuauaugu |
| 759 | aggccgguac uauaugucu | 885 | aggccgguac uauaugucu |
| 760 | ggccgguacu auaugucug | 886 | ggccgguacu auaugucug |
| 761 | gccgguacua uaugucugc | 887 | gccgguacua uaugucugc |
| 762 | cgguacuaua ugucugcgc | 888 | cgguacuaua ugucugcgc |
| 763 | gguacuauau gucugcgcg | 889 | gguacuauau gucugcgcg |
| 764 | cuauaugucu gcgcgccac | 890 | cuauaugucu gcgcgccac |
| 765 | uaugucugcg cgccacaug | 891 | uaugucugcg cgccacaug |
| 766 | augucugcgc gccacaugg | 892 | augucugcgc gccacaugg |
| 767 | ucgcccucgc ugucacgac | 893 | ucgcccucgc ugucacgac |
| 768 | cggcgagcgc uacgaugag | 894 | cggcgagcgc uacgaugag |
| 769 | caaggaccgg cgcaacauc | 895 | caaggaccgg cgcaacauc |
| 770 | acauccgugc accagagug | 896 | acauccgugc accagagug |
| 771 | cugggcgagg agcguaacu | 897 | cugggcgagg agcguaacu |
| 772 | gggcgaggag cguaacuaa | 898 | gggcgaggag cguaacuaa |
| 773 | gcgaggagcg uaacuaagu | 899 | gcgaggagcg uaacuaagu |
| 774 | gaggagcgua acuaagucc | 900 | gaggagcgua acuaagucc |
| 775 | aguccgccaa acacuccac | 901 | aguccgccaa acacuccac |
| 776 | ggcgaucaag uccagagcc | 902 | ggcgaucaag uccagagcc |
| 777 | cccuaaccug guuauucc | 903 | cccuaaccug gcuuauucc |
| 778 | ugugaaacca cuagguucu | 904 | ugugaaacca cuagguucu |
| 779 | accacuaggu ucuaggucc | 905 | accacuaggu ucuaggucc |
| 780 | caggggugcg gucuugcaa | 906 | caggggugcg gucuugcaa |
| 781 | ggugcggucu ugcaauaga | 907 | ggugcggucu ugcaauaga |
| 782 | gugcggucuu gcaauagga | 908 | gugcggucuu gcaauagga |
| 783 | agccaugccu uuaaaccga | 909 | agccaugccu uuaaaccga |
| 784 | augccuuuaa accgcacua | 910 | augccuuuaa accgcacua |
| 785 | ugggaggaug aauucgaca | 911 | ugggaggaug aauucgaca |
| 786 | aacgcagugc ucuucgaaa | 912 | aacgcagugc ucuucgaaa |
| 787 | ggacgaaugg ggcgacaaa | 913 | ggacgaaugg ggcgacaaa |
| 788 | ggcgacugcc uguagcaaa | 914 | ggcgacugcc uguagcaaa |
| 789 | cacgcugcug gggcgcuaa | 915 | cacgcugcug gggcgcuaa |
| 790 | ugcgcucacg ucuucacua | 916 | ugcgcucacg ucuucacua |
| 791 | ccuuauacuu cuuuaucga | 917 | ccuuauacuu cuuuaucga |
| 792 | cuuuaucgcc ggccgcuaa | 918 | cuuuaucgcc ggccgcuaa |
| 793 | ucgccggccg cuaugagua | 919 | ucgccggccg cuaugagua |
| 794 | gccgcuauga guucuccaa | 920 | gccgcuauga guucuccaa |
| 795 | ccgcuaugag uucuccaaa | 921 | ccgcuaugag uucuccaaa |
| 796 | ggcucggcuc aacuaucua | 922 | ggcucggcuc aacuaucua |
| 797 | gcucggcuca acuaucuga | 923 | gcucggcuca acuaucuga |
| 798 | ucggcucaac uaucugcua | 924 | ucggcucaac uaucugcua |
| 799 | ccagcgcgga ccaacaaua | 925 | ccagcgcgga ccaacaaua |
| 800 | agcgcggacc aacaauuua | 926 | agcgcggacc aacaauuua |
| 801 | ucgggaggaa gcuuuauga | 927 | ucgggaggaa gcuuuauga |
| 802 | acgcagcggc agucuuuca | 928 | acgcagcggc agucuuuca |
| 803 | gaccaccauc cgccgaaua | 929 | gaccaccauc cgccgaaua |
| 804 | ccgaaucggc cucuucaaa | 930 | ccgaaucggc cucuucaaa |
| 805 | aaucggccuc uucaauaga | 931 | aaucggccuc uucaauaga |
| 806 | caauagcagu gccgacaga | 932 | caauagcagu gccgacaga |
| 807 | aggaguuugu ccguggcua | 933 | aggaguuugu ccguggcua |
| 808 | cggcugagug cacgguuaa | 934 | cggcugagug cacgguuaa |
| 809 | ugggaauccc caguaucua | 935 | ugggaauccc caguaucua |
| 810 | cagaccccuc agcuuacga | 936 | cagaccccuc agcuuacga |
| 811 | ccccucagcu uacgguaua | 937 | ccccucagcu uacgguaua |
| 812 | cucagcuuac gguaucuaa | 938 | cucagcuuac gguaucuaa |
| 813 | gcuuacggua ucuacauua | 939 | gcuuacggua ucuacauua |
| 814 | ccggcggcag cguaucaua | 940 | ccggcggcag cguaucaua |
| 815 | agcguaucau ccagcggaa | 941 | agcguaucau ccagcggaa |
| 816 | gcguaucauc cagcggaaa | 942 | gcguaucauc cagcggaaa |
| 817 | uaccuaggcc gguacuaua | 943 | uaccuaggcc gguacuaua |
| 818 | cuaggccggu acuauauga | 944 | cuaggccggu acuauauga |
| 819 | aggccgguac uauauguca | 945 | aggccgguac uauauguca |
| 820 | ggccgguacu auaugucua | 946 | ggccgguacu auaugucua |
| 821 | gccgguacua uaugucuga | 947 | gccgguacua uaugucuga |
| 822 | cgguacuaua ugucugcga | 948 | cgguacuaua ugucugcga |
| 823 | gguacuauau gucugcgca | 949 | gguacuauau gucugcgca |
| 824 | cuauaugucu gcgcgccaa | 950 | cuauaugucu gcgcgccaa |
| 825 | uaugucugcg cgccacaua | 951 | uaugucugcg cgccacaua |
| 826 | augucugcgc gccacauga | 952 | augucugcgc gccacauga |
| 827 | ucgcccucgc ugucacgaa | 953 | ucgcccucgc ugucacgaa |
| 828 | cggcgagcgc uacgaugaa | 954 | cggcgagcgc uacgaugaa |
| 829 | caaggaccgg cgcaacaua | 955 | caaggaccgg cgcaacaua |
| 830 | acauccgugc accagagua | 956 | acauccgugc accagagua |
| 831 | cugggcgagg agcguaaca | 957 | cugggcgagg agcguaaca |
| 832 | gggcgaggag cguaacuaa | 958 | gggcgaggag cguaacuaa |
| 833 | gcgaggagcg uaacuaaga | 959 | gcgaggagcg uaacuaaga |
| 834 | gaggagcgua acuaaguca | 960 | gaggagcgua acuaaguca |
| 835 | aguccgccaa acacuccaa | 961 | aguccgccaa acacuccaa |
| 836 | ggcgaucaag uccagagca | 962 | ggcgaucaag uccagagca |
| 837 | cccuaaccug gcuuauuca | 963 | cccuaaccug gcuuauuca |
| 838 | ugugaaacca cuagguuca | 964 | ugugaaacca cuagguuca |
| 839 | accacuaggu ucuagguca | 965 | accacuaggu ucuagguca |
| 840 | uggacuucaaccuagacaa | 966 | uggacuucaaccuagacaa |
| 841 | cugggaggaugaauucgaa | 967 | cugggaggaugaauucgaa |
| 842 | gggugacaacuacuaucua | 968 | gggugacaacuacuaucua |
| 843 | cugggaggaugaauucgaa | 969 | cugggaggaugaauucgaa |
| 844 | caagguggguggcaucuaa | 970 | caagguggguggcaucuaa |
| 845 | uccgcagccuggaugauua | 971 | uccgcagccuggaugauua |

In some embodiments, the polynucleotides illustrated above include those that do not include a 2′-O methyl vinyl phosphonate uridine as the 5′ nucleotide on the antisense strand of the siRNA.

In some embodiments, a polynucleotide is as provided for herein. In some embodiments, the polynucleotide comprises a first strand and a second strand to for a portion that comprises a duplex. In some embodiments, the polynucleotide comprises a sense strand and an antisense strand. In some embodiments, comprises the sequences as illustrated in Table 7a or Table 7b. In some embodiments, comprises the sequences as illustrated in Table 3a or Table 4a but without the base modifications. In some embodiments, a pharmaceutical composition comprises a siRNA pair as provided herein. In some embodiments, the siRNA pair is not conjugated to a FN3 domain.

In some embodiments, an oligonucleotide molecule described herein is constructed using chemical synthesis and/or enzymatic ligation reactions using procedures known in the art. For example, an oligonucleotide molecule is chemically synthesized using naturally occurring nucleotides or variously modified nucleotides designed to increase the biological stability of the molecules or to increase the physical stability of the duplex formed between the oligonucleotide molecule and target nucleic acids. Alternatively, the oligonucleotide molecule is produced biologically using an expression vector into which a oligonucleotide molecule has been subcloned in an antisense orientation (i.e., RNA transcribed from the inserted oligonucleotide molecule will be of an antisense orientation to a target polynucleic acid molecule of interest).

In some embodiments, an oligonucleotide molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, an oligonucleotide molecule is also assembled from two distinct nucleic acid strands or fragments wherein one fragment includes the sense region and the second fragment includes the antisense region of the molecule.

In some instances, while chemical modification of the oligonucleotide molecule internucleotide linkages with phosphorothioate, phosphorodithioate, phosphonate, phosphoramidate, or mesyl phosphoramidate, linkages improves stability. Excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

As described herein, in some embodiments, any nucleic acid molecules disclosed herein can be modified to include a linker at the 5′ end of the of the sense strand of the dsRNA. In some embodiments, any nucleic acid molecules disclosed herein can be modified to include a vinyl phosphonate or modified vinyl phosphonate at the 5′ end of the of the anti-sense strand of the dsRNA. In some embodiments, any nucleic acid molecules disclosed herein can be modified to include a linker at the 3′ end of the of the sense strand of the dsRNA. In some embodiments, any nucleic acid molecules disclosed herein can be modified to include a vinyl phosphonate at the 3′ end of the of the anti-sense strand of the dsRNA. The linker can be used to link the dsRNA to the FN3 domain. The linker can covalently attach, for example, to a cysteine residue on the FN3 domain that is there naturally or that has been substituted as described herein, and for example, in U.S. Pat. No. 10,196,446, which is hereby incorporated by reference in its entirety.

In some embodiments, the siRNA pairs of A1-A6 as shown in Table 3a provided for above comprise a linker at the 3′ end of the sense strand. In some embodiments, the siRNA pairs of AT-A6 as shown in Table 3a provided for above comprise a vinyl phosphonate at the 5′ end of the sense strand.

Structure of exemplary linkers (L) are provided in Table 8.

TABLE 8
Representative Examples of Linkers (L)

| Linker Structure | Linker Name |
|---|---|
| 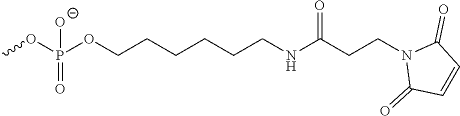 | Mal-C₂H₄C(O) (NH)—(CH₂)₆ |
| 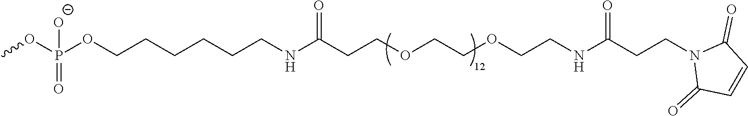 | Mal- (PEG)₁₂(NH)(CH₂)₆ |
| 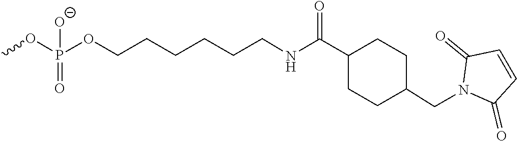 | Mal-NH—(CH₂)₆ or Aminohexyl linker- (CH₂)₆— |
| 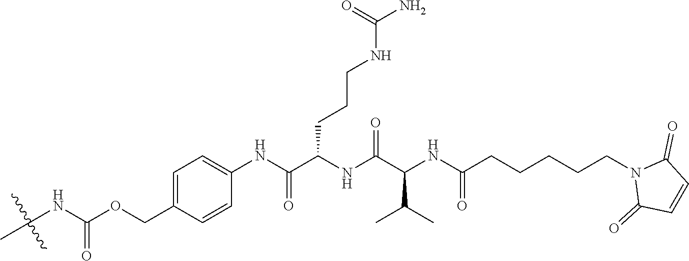 [SMILES: *NC(=O)OCc1ccc(NC(=O)[C@H](CCCNC(N)=O)NC(=O)[C@@H](NC(=O)CCCCCN2C(=O)C=CC2=O)C(C)C)cc1] | Val-Cit-PABA |

Other linkers can also be used, such as, linkers formed with click chemistry, amide coupling, reductive amination, oxime, enzymatic couplings such as transglutaminase and shortage conjugations. The linkers provided here are exemplary in nature and other linkers made with other such methods can also be used. For example, linkers connected through phosphate groups can be phosphorothioates or phosphorodithioates.

When connected to the siRNA, the structures, L-(X4) can be represented by the following formulas:

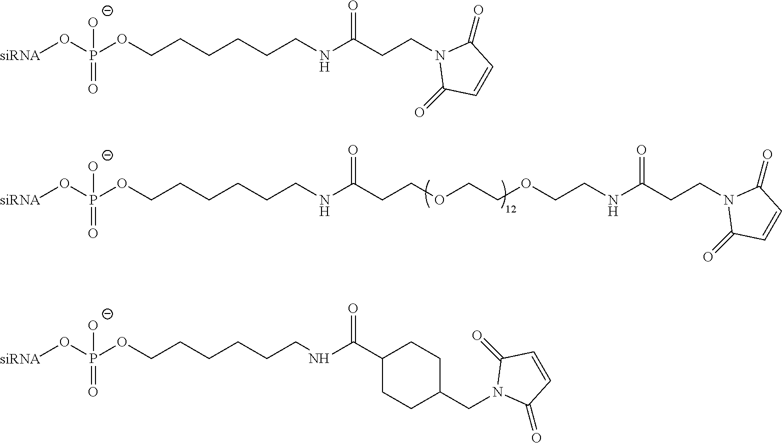

Although certain siRNA sequences are illustrated herein with certain modified nucleobases, the sequences without such modifications are also provided herein. That is, the sequence can comprise the sequences illustrated in the tables provided herein without any modifications. The unmodified siRNA sequences can still comprise, in some embodiments, a linker at the 5′ end of the of the sense strand of the dsRNA. In some embodiments, the nucleic acid molecules can be modified to include a vinyl phosphonate at the 5′ end of the of the anti-sense strand of the dsRNA. In some embodiments, the nucleic acid molecules can be modified to include a linker at the 3′ end of the of the sense strand of the dsRNA. In some embodiments, the nucleic acid molecules can be modified to include a vinyl phosphonate at the 3′ end of the of the anti-sense strand of the dsRNA. The linker can be as provided herein.

Isolation of CD71 Binding FN3 Domains from a Library Based on Tencon Sequence

Tencon (SEQ ID NO: 1) is a non-naturally occurring fibronectin type III (FN3) domain designed from a consensus sequence of fifteen FN3 domains from human tenascin-C (Jacobs et al., Protein Engineering, Design, and Selection, 25:107-117, 2012; U.S. Pat. Publ. No. 2010/0216708). The crystal structure of Tencon shows six surface-exposed loops that connect seven beta-strands as is characteristic to the FN3 domains, the beta-strands referred to as A, B, C, D, E, F, and G, and the loops referred to as AB, BC, CD, DE, EF, and FG loops (Bork and Doolittle, Proc Natl Acad Sci USA 89:8990-8992, 1992; U.S. Pat. No. 6,673,901). These loops, or selected residues within each loop, may be randomized in order to construct libraries of fibronectin type III (FN3) domains that may be used to select novel molecules that bind CD71. Table 9 shows positions and sequences of each loop and beta-strand in Tencon (SEQ ID NO: 1).

TABLE 9
Tencon topology

| FN3 domain | Tencon (SEQ ID NO: 1) |
|---|---|
| A strand | 1-12 |
| AB loop | 13-16 |
| B strand | 17-21 |
| BC loop | 22-28 |
| C strand | 29-37 |
| CD loop | 38-43 |
| D strand | 44-50 |
| DE loop | 51-54 |
| E strand | 55-59 |
| EF loop | 60-64 |
| F strand | 65-74 |
| FG loop | 75-81 |
| G strand | 82-89 |

Library designed based on Tencon sequence may thus have randomized FG loop, or randomized BC and FG loops, such as libraries TCL1 or TCL2 as described below. The Tencon BC loop is 7 amino acids long, thus 1, 2, 3, 4, 5, 6 or 7 amino acids may be randomized in the library diversified at the BC loop and designed based on Tencon sequence. The Tencon FG loop is 7 amino acids long, thus 1, 2, 3, 4, 5, 6 or 7 amino acids may be randomized in the library diversified at the FG loop and designed based on Tencon sequence. Further diversity at loops in the Tencon libraries may be achieved by insertion and/or deletions of residues at loops. For example, the FG and/or BC loops may be extended by 1-22 amino acids, or decreased by 1-3 amino acids. The FG loop in Tencon is 7 amino acids long, whereas the corresponding loop in antibody heavy chains ranges from 4-28 residues. To provide maximum diversity, the FG loop may be diversified in sequence as well as in length to correspond to the antibody CDR3 length range of 4-28 residues. For example, the FG loop can further be diversified in length by extending the loop by additional 1, 2, 3, 4 or 5 amino acids.

Library designed based on Tencon sequence may also have randomized alternative surfaces that form on a side of the FN3 domain and comprise two or more beta strands, and at least one loop. One such alternative surface is formed by amino acids in the C and the F beta-strands and the CD and the FG loops (a C-CD-F-FG surface). A library design based on Tencon alternative C-CD-F-FG surface is described in U.S. Pat. Publ. No. 2013/0226834. Library designed based on Tencon sequence also includes libraries designed based on Tencon variants, such as Tencon variants having substitutions at residues positions 11, 14, 17, 37, 46, 73, or 86 (residue numbering corresponding to SEQ ID NO: 1), and which variants display improve thermal stability. Exemplary Tencon variants are described in US Pat. Publ. No. 2011/0274623, and include Tencon27 (SEQ ID NO: 2) having substitutions E1IR, L17A, N46V and E86I when compared to Tencon of SEQ ID NO: 1.

Tencon and other FN3 sequence based libraries may be randomized at chosen residue positions using a random or defined set of amino acids. For example, variants in the library having random substitutions may be generated using NNK codons, which encode all 20 naturally occurring amino acids. In other diversification schemes, DVK codons may be used to encode amino acids Ala, Trp, Tyr, Lys, Thr, Asn, Lys, Ser, Arg, Asp, Glu, Gly, and Cys. Alternatively, NNS codons may be used to give rise to all 20 amino acid residues and simultaneously reducing the frequency of stop codons. Libraries of FN3 domains with biased amino acid distribution at positions to be diversified may be synthesized for example using Slonomics® technology (http:_//www_sloning_com). This technology uses a library of pre-made double stranded triplets that act as universal building blocks sufficient for thousands of gene synthesis processes. The triplet library represents all possible sequence combinations necessary to build any desired DNA molecule. The codon designations are according to the well-known IUB code.

The FN3 domains that specifically bind CD71 may be isolated by producing the FN3 library such as the Tencon library using cis display to ligate DNA fragments encoding the scaffold proteins to a DNA fragment encoding RepA to generate a pool of protein-DNA complexes formed after in vitro translation wherein each protein is stably associated with the DNA that encodes it (U.S. Pat. No. 7,842,476; Odegrip et al., Proc Natl Acad Sci USA 101, 2806-2810, 2004), and assaying the library for specific binding to PSMA by any method known in the art and described in the Example. Exemplary well known methods which can be used are ELISA, sandwich immunoassays, and competitive and non-competitive assays (see, e.g., Ausubel et al., eds, 1994, Current Protocols in Molecular Biology, Vol. 1, John Wiley & Sons, Inc., New York). The identified FN3 domains that specifically bind CD71 are further characterized for their binding to CD71, modulation of CD71 activity, internalization, stability, and other desired characteristics.

The FN3 domains that specifically bind CD71 may be generated using any FN3 domain as a template to generate a library and screening the library for molecules specifically binding CD71 using methods provided within. Exemplar FN3 domains that may be used are the 3rd FN3 domain of tenascin C (TN3), Fibcon, and the 10^{th} FN3 domain of fibronectin (FN10). Accordingly, PCT applications WO 2010/051274, WO 2011/137319, and WO 2013/049275 are incorporated herein in their entirety. Standard cloning and expression techniques are used to clone the libraries into a vector or synthesize double stranded cDNA cassettes of the library, to express, or to translate the libraries in vitro. For example ribosome display (Hanes and Pluckthun, Proc Natl Acad Sci USA, 94, 4937-4942, 1997), mRNA display (Roberts and Szostak, Proc Natl Acad Sci USA, 94, 12297-12302, 1997), or other cell-free systems (U.S. Pat. No. 5,643,768) can be used. The libraries of the FN3 domain variants may be expressed as fusion proteins displayed on the surface for example of any suitable bacteriophage. Methods for displaying fusion polypeptides on the surface of a bacteriophage are well known (U.S. Pat. Publ. No. 2011/0118144; Int. Pat. Publ. No. WO2009/085462; U.S. Pat. Nos. 6,969,108; 6,172,197; 5,223,409; 6,582,915; 6,472,147).

In some embodiments, the FN3 domain that binds CD71 is based on Tencon sequence of SEQ ID NO: 1 or Tencon27 sequence of SEQ ID NO: 2, optionally having substitutions at residues positions 11, 14, 17, 37, 46, 73, and/or 86.

In some embodiments, the FN3 protein or polypeptide is one that binds to human CD71 at a site on CD71 that does not compete with transferrin binding to CD71. As used herein, a site on CD71 that does not compete with transferrin binding to CD71 refers to an epitope or part of CD71 where the binding of the FN3 protein does not compete or inhibit the binding of transferrin to CD71. The competition, or lack thereof, can be complete or partial. In some embodiments, the binding also does not inhibit the internalization of transferrin into the cell through its interaction with CD71.

In some embodiments, methods for identifying a FN3 protein that binds to CD71 at a site that does not compete or inhibit transferrin binding to CD71 are provided. In some embodiments, the methods comprise contacting CD71 in the presence of transferrin or an agent that binds to the CD71 transferrin binding site with a test FN3 protein; and identifying a test FN3 protein that binds to CD71 in the presence of transferrin or an agent that binds to the CD71 transferrin binding site. In some embodiments, the method comprises isolating the test FN3 protein that binds to CD71 in the presence of transferrin or an agent that binds to the CD71 transferrin binding site. In some embodiments, the methods comprise sequencing the test FN3 protein that binds to CD71 in the presence of transferrin or an agent that binds to the CD71 transferrin binding site. In some embodiments, the methods comprise preparing or obtaining a nucleic acid sequence encoding the test FN3 protein that binds to CD71 in the presence of transferrin or an agent that binds to the CD71 transferrin binding site. In some embodiments, the methods comprise expressing the test FN3 protein that binds to CD71 in the presence of transferrin or an agent that binds to the CD71 transferrin binding site from a nucleic acid sequence encoding the test FN3 protein that binds to CD71 in the presence of transferrin or an agent that binds to the CD71 transferrin binding site. In some embodiments, the test FN3 protein is expressed in a cell. In some embodiments, the methods comprise isolating and/or purifying the expressed test FN3 protein.

In some embodiments a FN3 protein is provided, wherein the FN3 protein is identified according to any method provided herein.

The FN3 domains that specifically bind CD71 may be modified to improve their properties such as improve thermal stability and reversibility of thermal folding and unfolding. Several methods have been applied to increase the apparent thermal stability of proteins and enzymes, including rational design based on comparison to highly similar thermostable sequences, design of stabilizing disulfide bridges, mutations to increase alpha-helix propensity, engineering of salt bridges, alteration of the surface charge of the protein, directed evolution, and composition of consensus sequences (Lehmann and Wyss, Curr. Opin. Biotechnol., 12, 371-375, 2001). High thermal stability may increase the yield of the expressed protein, improve solubility or activity, decrease immunogenicity, and minimize the need of a cold chain in manufacturing. Residues that may be substituted to improve thermal stability of Tencon (SEQ ID NO: 1) are residue positions 11, 14, 17, 37, 46, 73, or 86, and are described in US Pat. Publ. No. 2011/0274623. Substitutions corresponding to these residues may be incorporated to the FN3 domain containing molecules disclosed herein.

Measurement of protein stability and protein lability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“Tₘ”) temperature, the temperature in ° Celsius (° C.) at which half of the molecules become unfolded, using standard methods. Typically, the higher the Tₘ, the more stable the molecule. In addition to heat, the chemical environment also changes the ability of the protein to maintain a particular three dimensional structure.

In some embodiments, the FN3 domain that binds CD71 may exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same domain prior to engineering measured by the increase in the Tₘ.

Chemical denaturation can likewise be measured by a variety of methods. Chemical denaturants include guanidinium hydrochloride, guanidinium thiocyanate, urea, acetone, organic solvents (DMF, benzene, acetonitrile), salts (ammonium sulfate, lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HCl), acetic acid (CH₃COOH), halogenated acetic acids), hydrophobic molecules (e.g. phospholipids), and targeted denaturants. Quantitation of the extent of denaturation can rely on loss of a functional property, such as ability to bind a target molecule, or by physiochemical properties, such as tendency to aggregation, exposure of formerly solvent inaccessible residues, or disruption or formation of disulfide bonds.

### Polynucleotides, Vectors, and Host Cells

In some embodiments, provided herein are nucleic acids encoding the FN3 domains specifically binding CD71 as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof. Certain exemplary polynucleotides are disclosed herein, however, other polynucleotides which, given the degeneracy of the genetic code or codon preferences in a given expression system, encode the FN3 domains disclosed herein are also within the scope of the disclosure.

In some embodiments, an isolated polynucleotide encodes the FN3 domain specifically binding CD71 comprising the amino acid sequence of SEQ ID NOs: 100-209, 211-301, 303-317, 319-552, and 972-976.

The polynucleotides disclosed herein may be produced by chemical synthesis such as solid phase polynucleotide synthesis on an automated polynucleotide synthesizer and assembled into complete single or double stranded molecules. Alternatively, the polynucleotides disclosed herein may be produced by other techniques such as PCR followed by routine cloning. Techniques for producing or obtaining polynucleotides of a given known sequence are well known in the art.

The polynucleotides disclosed herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

In some embodiments, a vector comprising at least one polynucleotide disclosed herein is provided. Such vectors may be plasmid vectors, viral vectors, vectors for baculovirus expression, transposon based vectors or any other vector suitable for introduction of the polynucleotides disclosed herein into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

In some embodiments, a host cell comprising the vector is provided. The FN3 domain that specifically bind CD71 may be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, NY (1989); Harlow and Lane, Antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

The host cell chosen for expression may be of mammalian origin or may be selected from COS-1, COS-7, HEK293, BHK21, CHO, BSC-1, He G2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21(DE3), BL21-GOLD(DE3), XL1-Blue, JM109, HMS174, HMS174(DE3), and any of the natural or engineered E. coli spp. Klebsiella spp., or Pseudomonas spp strains.

In some embodiments, a method of producing the isolated FN3 domain that binds CD71, comprising culturing the isolated host cell under conditions such that the isolated FN3 domain that binds CD71 is expressed, and purifying the FN3 domain.

The FN3 domains that bind CD71 may be purified from recombinant cell cultures by well-known methods, for example by protein A purification, ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography, or high performance liquid chromatography (HPLC).

### Kits

In some embodiments, provided herein is a kit comprising the FN3 domain that binds CD71 as described herein.

The kit may be used for therapeutic uses and as a diagnostic kit.

In some embodiments, the kit comprises the FN3 domain that binds CD71 and reagents for detecting the FN3 domain. In some embodiments, the kit comprises a bivalent FN3 domain. The kit can include one or more other elements including: instructions for use; other reagents, e.g., a label, an agent useful for chelating, or otherwise coupling, a radioprotective composition; devices or other materials for preparing the FN3 domain that binds CD71 for administration for imaging, diagnostic or therapeutic purpose; pharmaceutically acceptable carriers; and devices or other materials for administration to a subject.

In some embodiments, the kit comprises the FN3 domain that binds CD71 comprising the amino acid sequences of one of SEQ ID NOs: 100-209, 211-301, 303-317, 319-552, and 972-976.

### Uses of CD71-Binding FN3 Domains and Conjugates Thereof

The FN3 domains that specifically bind CD71 or conjugates thereof may be used to diagnose, monitor, modulate, treat, alleviate, help prevent the incidence of, or reduce the symptoms of human disease or specific pathologies in cells, tissues, organs, fluid, or, generally, a host.

In some embodiments, the FN3 domain can facilitate delivery into CD71 positive tissues (e.g., skeletal muscle, smooth muscle) for treatment of muscle diseases.

In some embodiments, the FN3 domain can facilitate delivery to activated lymphocytes, dendritic cells, or other immune cells for treatment of immunological diseases. Thus, in some embodiments, the polypeptide that binds to CD71 is directed to immune cells. In some embodiments, the polypeptide that binds to CD71 is directed to B cells. In some embodiments, the polypeptide that binds to CD71 is directed to T cells. In some embodiments, the polypeptide that binds to CD71 is directed to dendritic cells. In some embodiments, the polypeptide that binds to CD71 is directed to monocytes.

In some embodiments, the polypeptide that binds to CD71 does not have an anti-proliferative effect on immune cells. For example, in some embodiments, the polypeptide that binds to CD71 does not have an anti-proliferative effect on B cells, T cells, dendritic cells, monocytes, or any combination thereof.

In some embodiments, methods of treating an autoimmune disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID NOs: 100-209, 211-301, 303-317, 319-552, and 972-976, or a polypeptide as provided herein that is linked to or conjugated to a therapeutic agent. In some embodiments, a method of treating an autoimmune disease in a subject, the method comprising administering to the subject a FN3 domain that binds CD71 and the FN3 domain is conjugated to a therapeutic agent (e.g. cytotoxic agent, an oligonucleotide, such as a siRNA, ASO, and the like, a FN3 domain that binds to another target, and the like).

In some embodiments, the autoimmune disease is selected from the group consisting of rheumatoid arthritis, Hashimoto's autoimmune thyroiditis, celiac disease, diabetes mellitus type 1, vitiligo, rheumatic fever, pernicious anemia/atrophic gastritis, alopecia areata, immune thrombocytopenic purpura, psoriasis, inflammatory bowel disease, systemic lupus erythematosus, pemphigus, Siogren's syndrome, myositis, lupus nephritis, neuroinflammatory diseases such as multiple sclerosis, or prevention of solid organ transplant rejection.

In some embodiments, methods of reducing the expression of a target gene in a cell are provided. In some embodiments, the methods comprise delivering to the cell with a composition or a pharmaceutical composition as provided herein. In some embodiments, the cell is ex-vivo. In some embodiments, the cell is in-vivo. In some embodiments, the target gene is CD40, KRAS, or GYS1. The target gene, however, can be any target gene as the evidence provided herein demonstrates that siRNA molecules can be delivered efficiently when conjugated to a FN3 domain. In some embodiments, the siRNA targeting CD40, KRAS, or GYS1 is linked to a FN3 domain. In some embodiments, the FN3 polypeptide (domain) is one that binds to CD71. In some embodiments, the FN3 polypeptide is as provided for herein or as provided for in PCT Application No. PCT/US20/55509, U.S. application Ser. No. 17/070,337, now U.S. Pat. No. 11,781,138, PCT Application No. PCT/US20/55470, or U.S. Pat. No. 11,628,222, each of which is hereby incorporated by reference in its entirety. In some embodiments, the siRNA is not conjugated to a FN3 domain. In some embodiments, a method of reducing the expression of a target gene results in a reduction of about 99%, 90-99%, 50-90%, or 10-50% in the expression of the target gene.

In some embodiments, methods of delivering a siRNA molecule to a cell in a subject are provided. In some embodiments, the methods comprise administering to the subject a pharmaceutical composition comprising a composition as provided for herein. In some embodiments, the cell is a CD71 positive cell. The term “positive cell” in reference to a protein refers to a cell that expresses the protein. In some embodiments, the protein is expressed on the cell surface. In some embodiments, the cell is a tumor cell, a liver cell, an immune cell, a heart cell, a muscle cell, a cell of the CNS, or a cell inside the blood brain barrier. In some embodiments, the siRNA downregulates the expression of a target gene in the cell. In some embodiments, the target gene is CD40, KRAS, or GYS1.

In some embodiments, a method of reducing the expression of CD40, KRAS, or GYS1 is provided. In some embodiments, the reduced expression is the expression (amount) of CD40 mRNA. In some embodiments, a method of reducing the expression of CD40, KRAS, or GYS1 results in a reduction of about 99%, 90-99%, 50-90%, or 10-50% in the expression of CD40, KRAS, or GYS1. In some embodiments, the reduced expression is the expression (amount) of CD40, KRAS, or GYS1 protein. In some embodiments, the reduced protein is glycogen. In some embodiments, reduction of glycogen occurs in muscle cells. In some embodiments, reduction of glycogen occurs in heart cells. In some embodiments, the method comprises delivering to a cell with a siRNA molecule as provided herein that targets CD40, KRAS, or GYS1. In some embodiments, the siRNA is conjugated to a FN3 domain. In some embodiments, the FN3 domain is a FN3 domain that binds to CD71. In some embodiments, the FN3 domain is as provided for herein. In some embodiments, the FN3 domain is a dimer of two FN3 domains that bind to CD71. In some embodiments, the FN3 domains are the same. In some embodiments, the two FN3 domains are different, i.e., bind to different regions or amino acid residues of CD71, i.e. a different epitope. In some embodiments, the method comprises administering to a subject (patient) a CD40, KRAS, or GYS1 siRNA molecule, such as those provided herein. In some embodiments, the CD40, KRAS, or GYS1 siRNA administered to the subject is conjugated or linked to a FN3 domain. In some embodiments, the FN3 domain is a FN3 domain that binds to CD71. In some embodiments, the FN3 domain is as provided for herein. In some embodiments, the FN3 domain is a dimer of two FN3 domains that bind to CD71. In some embodiments, the FN3 domains are the same. In some embodiments, the two FN3 domains are different, i.e., bind to different regions or amino acid residues of CD71, i.e. a different epitope. In some embodiments, the CD71 binding domain is a polypeptide as provided for herein.

In some embodiments, methods of selectively reducing GYS1 mRNA and protein in skeletal muscle. In certain embodiments, GYS1 mRNA and protein is not reduced in the liver and/or the kidney. In some embodiments, the reduction in the GYS1 mRNA and protein is sustained for about 1 week, about 2 weeks, about 3 weeks, about 4 weeks, about 5 weeks, or greater than 5 weeks after administration of the conjugate described herein.

In some embodiments, the FN3 domain can facilitate delivery into CD71 positive tissues (e.g., skeletal muscle, smooth muscle) for treatment of muscle diseases.

In some embodiments, a method of treating a subject having Pompe Disease (GSD2, acid alpha-glucosidase (GAA) deficiency) is provided, the method comprising administering to the subject a composition provided for herein. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID NOs: 100-209, 211-301, 303-317, 319-552, and 972-976, or a polypeptide as provided herein that is linked to or conjugated to a therapeutic agent.

In some embodiments, methods of treating glycogen storage disease in a subject in need thereof, the method comprising administering a composition provided herein are provided. In some embodiments, the glycogen storage disease is selected from the group consisting of Cori's disease or Forbes' disease (GSD3, Glycogen debranching enzyme (AGL) deficiency), McArdle disease (GSD5, Muscle glycogen phosphorylase (PYGM) deficiency), type II Diabetes/diabetic nephropathy, Aldolase A Deficiency GSD12, Lafora Disease, hypoxia, Andersen disease (GSD4, Glycogen debranching enzyme (GBE1) deficiency), Tarui's Disease (GSD7, Muscle phosphofructokinase (PFKM) deficiency), and adult polyglucosan body disease. In some embodiments, the glycogen storage disease is selected from the group consisting of Glycogen synthase (GYS2) deficiency (GSDO), Glucose-6-phosphatase (G6PC/SLC37A4) deficiency (GSD1, von Gierke's disease), Hers' disease (GSD6, Liver glycogen phosphorylase (PYGL) or Muscle phosphoglycerate mutase (PGAM2) deficiency), Phosphorylase kinase (PHKA2/PHKB/PHKG2/PHKA1) deficiency (GSD9), Phosphoglycerate mutase (PGAM2) deficiency (GSD10), Muscle lactate dehydrogenase (LDHA) deficiency (GSD11), Fanconi-Bickel syndrome (GSD 11, Glucose transporter (GLUT2) deficiency, Aldolase A deficiency (GSD 12), P-enolase (ENO3) deficiency (GSD13), and Glycogenin-1 (GYGI) deficiency (GSD15).

In some embodiments, the CD71 cell is a cell involved in a CNS diseases, inflammatory/immune diseases, such as MS & infectious diseases of the brain. In some embodiments, the polypeptide that binds to CD71 is directed to the central nervous system. In some embodiments, methods of treating a neurological condition and/or a brain tumor in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID NOs: 100-209, 211-301, 303-317, 319-552, and 972-976, or a polypeptide as provided herein that is linked to or conjugated to a therapeutic agent. In some embodiments, the brain tumor is selected from the group consisting of nonmalignant, benign, and malignant brain tumors. In some embodiments, the neurological condition is selected from the group consisting of Alzheimer's Disease, Amyotrophic Lateral Sclerosis, Parkinson's Disease, Lafora Disease, Pompe Disease, adult polyglucosan body disease, stroke, spinal cord injury, ataxia, Bell's Palsy, cerebral aneurysm, epilepsy, seizures, Guillain-Barre Syndrome, multiple sclerosis, muscular dystrophy, neurocutaneous syndromes, migraine, encephalitis, septicemia, and myasthenia gravis.

In some embodiments, the FN3 domains that specifically bind CD71 or conjugates thereof may also be used in imaging CD71 positive tumor tissue in a subject. The methods disclosed herein may be used with an animal patient belonging to any classification. Examples of such animals include mammals such as humans, rodents, dogs, cats and farm animals.

In some embodiments, a method of diagnosing a subject having, or who is likely to develop cancer of a tissue based on the expression of CD71 by cells of the cancer tissue, methods of predicting success of immunotherapy, methods of prognosis, and methods of treatment are provided.

In some embodiments, a method of detecting CD71-expressing cancer cells in a tumor tissue is provided, the method comprising: obtaining a sample of the tumor tissue from a subject; detecting whether CD71 is expressed in the tumor tissue by contacting toe sample of the tumor tissues with the FN3 domain that binds CD71 comprising the amino acid sequence of one of SEQ ID NOs: 100-209, 211-301, 303-317, 319-552, and 972-976, and detecting the binding between CD71 and the FN3 domain.

In some embodiments, the CD71 cell is a cell involved in a CNS diseases, inflammatory/immune diseases, such as MS & infectious diseases of the brain.

In some embodiments, the tissue can be tissue of any organ or anatomical system, that expresses CD71.

In some embodiments, CD71 expression may be evaluated using known methods, such as immunohistochemistry or ELISA.

In some embodiments, a method of isolating CD71 expressing cells is provided, the method comprising: obtaining a sample from a subject; contacting the sample with the FN3 domain that binds CD71 comprising the amino acid sequence of one of SEQ ID NOs: 100-209, 211-301, 303-317, 319-552, and 972-976, and isolating the cells bound to the FN3 domains.

In some embodiments, a method of detecting CD71-expressing cancer cells in a tumor tissue is provided, the method comprising: conjugating the FN3 domain that binds CD71 comprising the amino acid sequence of one of SEQ ID NOs: 100-209, 211-301, 303-317, 319-552, and 972-976 to a detectable label to form a conjugate; administering the conjugate to a subject; and visualizing the CD71 expressing cancer cells to which the conjugate is bound.

In some embodiments, a method of treating a subject having cancer is provided, the method comprising administering to the subject a FN3 domain that binds CD71. In some embodiments, the FN3 domain is conjugated to a therapeutic agent (e.g. cytotoxic agent, an oligonucleotide, such as a siRNA, antisense, and the like, a FN3 domain that binds to another target, and the like).

In some embodiments, the subject has a solid tumor.

In some embodiments, the solid tumor is a melanoma.

In some embodiments, the solid tumor is a lung cancer. In some embodiments, the solid tumor is a non-small cell lung cancer (NSCLC). In some embodiments, the solid tumor is a squamous non-small cell lung cancer (NSCLC). In some embodiments, the solid tumor is a non-squamous NSCLC. In some embodiments, the solid tumor is a lung adenocarcinoma.

In some embodiments, the solid tumor is a renal cell carcinoma (RCC).

In some embodiments, the solid tumor is a mesothelioma.

In some embodiments, the solid tumor is a nasopharyngeal carcinoma (NPC).

In some embodiments, the solid tumor is a colorectal cancer.

In some embodiments, the solid tumor is a prostate cancer. In some embodiments, the solid tumor is castration-resistant prostate cancer.

In some embodiments, the solid tumor is a stomach cancer.

In some embodiments, the solid tumor is an ovarian cancer.

In some embodiments, the solid tumor is a gastric cancer.

In some embodiments, the solid tumor is a liver cancer.

In some embodiments, the solid tumor is pancreatic cancer.

In some embodiments, the solid tumor is a thyroid cancer.

In some embodiments, the solid tumor is a squamous cell carcinoma of the head and neck.

In some embodiments, the solid tumor is a carcinomas of the esophagus or gastrointestinal tract.

In some embodiments, the solid tumor is a breast cancer.

In some embodiments, the solid tumor is a fallopian tube cancer.

In some embodiments, the solid tumor is a brain cancer.

In some embodiments, the solid tumor is an urethral cancer.

In some embodiments, the solid tumor is a genitourinary cancer.

In some embodiments, the solid tumor is an endometriosis.

In some embodiments, the solid tumor is a cervical cancer.

In some embodiments, the solid tumor is a metastatic lesion of the cancer.

In some embodiments, the subject has a hematological malignancy.

In some embodiments, the hematological malignancy is a lymphoma, a myeloma or a leukemia. In some embodiments, the hematological malignancy is a B cell lymphoma. In some embodiments, the hematological malignancy is Burkitt's lymphoma. In some embodiments, the hematological malignancy is Hodgkin's lymphoma. In some embodiments, the hematological malignancy is a non-Hodgkin's lymphoma.

In some embodiments, the hematological malignancy is a myelodysplastic syndrome.

In some embodiments, the hematological malignancy is an acute myeloid leukemia (AML). In some embodiments, the hematological malignancy is a chronic myeloid leukemia (CML). In some embodiments, the hematological malignancy is a chronic myelomoncytic leukemia (CMML).

In some embodiments, the hematological malignancy is a multiple myeloma (MM).

In some embodiments, the hematological malignancy is a plasmacytoma.

In some embodiments, the compositions or pharmaceutical compositions provided herein may be administered alone or in combination with other therapeutics, that is, simultaneously or sequentially. In some embodiments, the other or additional therapeutics are other anti-tumor agent or therapeutics. Different tumor types and stages of tumors can require the use of various auxiliary compounds useful for treatment of cancer. For example, the compositions provided herein can be used in combination with various chemotherapeutics such as taxol, tyrosine kinase inhibitors, leucovorin, fluorouracil, irinotecan, phosphatase inhibitors, MEK inhibitors, among others. The composition may also be used in combination with drugs which modulate the immune response to the tumor such as anti-PD-1 or anti-CTLA-4, among others. Additional treatments can be agents that modulate the immune system, such antibodies that target PD-1 or PD-L₁.

In some embodiments, the FN3 domains that specifically bind CD71 or conjugates thereof that may be used to diagnose, monitor, modulate, treat, alleviate, help prevent the incidence of, or reduce the symptoms of human disease or specific pathologies in cells, tissues, organs, fluid, or, generally, a host, also exhibit the property of being able to cross the blood brain barrier. The blood-brain barrier (BBB) prevents most macromolecules (e.g., DNA, RNA, and polypeptides) and many small molecules from entering the brain. The BBB is principally composed of specialized endothelial cells with highly restrictive tight junctions, consequently, passage of substances, small and large, from the blood into the central nervous system is controlled by the BBB. This structure makes treatment and management of patients with neurological diseases and disorders (e.g., brain cancer) difficult as many therapeutic agents cannot be delivered across the BBB with desirable efficiency. Additional conditions that involve disruptions of the BBB include: stroke, diabetes, seizures, hypertensive encephalopathy, acquired immunodeficiency syndrome, traumatic brain injuries, multiple sclerosis, Parkinson's disease (PD) and Alzheimer disease. This ability is especially useful for treating brain cancers including for example: astrocytoma, medulloblastoma, glioma, ependymoma, germinoma (pinealoma), glioblastoma multiform, oligodendroglioma, schwannoma, retinoblastoma, and congenital tumors; or a cancer of the spinal cord, e.g., neurofibroma, meningioma, glioma, and sarcoma. In certain embodiments, the FN3 domains that specifically bind CD71 comprising the amino acid sequence of one of SEQ ID NOs: 100-209, 211-301, 303-317, 319-552, and 972-976 or conjugates thereof, are useful to deliver a therapeutic or cytotoxic agent, for example, across the blood brain barrier.

In some embodiments, the polypeptide that can facilitates the transport of a therapeutic across the BBB is a protein comprising a sequence of SEQ ID NO: 100-209, 211-301, 303-317, 319-552, and 972-976.

In some embodiments, the compositions or pharmaceutical compositions provided herein may be administered alone or in combination with other therapeutics, that is, simultaneously or sequentially.

“Treat” or “treatment” refers to the therapeutic treatment and prophylactic measures, wherein the object is to prevent or slow down (lessen) an undesired physiological change or disorder, such as the development or spread of cancer. In some embodiments, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the FN3 domains that specifically bind CD71 may vary according to factors such as the disease state, age, sex, and weight of the individual. Exemplary indicators of an effective FN3 domain that binds CD71 is improved well-being of the patient, decrease or shrinkage of the size of a tumor, arrested or slowed growth of a tumor, and/or absence of metastasis of cancer cells to other locations in the body.

### Administration/Pharmaceutical Compositions

In some embodiments, pharmaceutical compositions of the FN3 domains that specifically bind CD71, optionally conjugated to a detectable label, therapeutic, or a cytotoxic agent disclosed herein and a pharmaceutically acceptable carrier, are provided. For therapeutic use, the FN3 domains that specifically bind CD71 may be prepared as pharmaceutical compositions containing an effective amount of the domain or molecule as an active ingredient in a pharmaceutically acceptable carrier. “Carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules disclosed herein in such pharmaceutical formulation can vary widely, i.e., from less than about 0.5%, usually at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, ₂₁ₛₜEdition, Troy, D. B. ed., Lipincott Williams and Wilkins, Philadelphia, PA 2006, Part 5, Pharmaceutical Manufacturing pp 691-1092, See especially pp. 958-989.

The mode of administration for therapeutic use of the FN3 domains disclosed herein may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intra-articular, intrabronchial, intra-abdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardial, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

### Enumerated Embodiments

Embodiments provided herein also include, but are not limited to, the following:
1. A CD71-binding FN3 domain polypeptide comprising an amino acid sequence that is at least 70%, 75%, 80%, 85%, 90%, or 95% identical to an amino acid sequence selected from the group consisting of SEQ ID NOs: 100-209, 211-301, 303-317, 319-552, and 972-976.
2. The polypeptide of embodiment 1, wherein the polypeptide comprises an amino acid sequence selected from the group consisting of SEQ ID NOs: 100-209, 211-301, 303-317, 319-552, and 972-976.
3. The polypeptide of embodiment 1, wherein the polypeptide comprises two of SEQ ID NOs: 100-209, 211-301, 303-317, 319-552, and 972-976.
4. A polypeptide that binds to human CD71 at a site on CD71 that does not compete with transferrin binding to CD71.
5. The polypeptide of embodiment 4, wherein the polypeptide comprises an amino acid sequence that is at least 70%, 75%, 80%, 85%, 90%, or 95% identical to an amino acid sequence selected from the group consisting of SEQ ID NOs: 100-209, 211-301, 303-317, 319-552, and 972-976.
6. The polypeptide of embodiment 4, wherein the polypeptide comprises an amino acid sequence that is selected from the group consisting of SEQ ID NOs: 100-209, 211-301, 303-317, 319-552, and 972-976.
7. The polypeptide of any one of embodiments 1-6, wherein the polypeptide is conjugated to a detectable label, an oligonucleotide, a therapeutic agent, or any combination thereof.
8. The polypeptide of embodiment 7, wherein the detectable label is a radioactive isotope, magnetic beads, metallic beads, colloidal particles, a fluorescent dye, an electron-dense reagent, an enzyme, biotin, digoxigenin, or hapten.
9. The polypeptide of embodiments 7 or 8, wherein the detectable label is auristatin, monomethyl auristatin phenylalanine, dolastatin, a chemotherapeutic agent, a drug, a growth inhibitory agent, a toxin, or a radioactive isotope.
10. The polypeptide of embodiment 7, wherein the therapeutic agent is a chemotherapeutic agent, a drug, an antibody, a growth inhibitory agent, a toxin, a radioactive isotope, an anti-tubulin agent, a polynucleotide, a siRNA molecule or a sense or an antisense strand thereof, an antisense molecule or a strand thereof, a RNA molecule, a DNA molecule, a DNA minor groove binder, a DNA replication inhibitor, an alkylating agent, an antibiotic, an antifolate, an antimetabolite, a chemotherapy sensitizer, a topoisomerase inhibitor, or a vinca alkaloid.
11. The polypeptide of embodiment 7, wherein the therapeutic agent can elicit one or more cytotoxic effects by modulating gene expression, RNA expression or levels, tubulin binding, DNA binding, topoisomerase inhibition, DNA cross linking, chelation, spliceosome inhibition, NAMPT inhibition, or HDAC inhibition.
12. The polypeptide of any one of embodiments 1-11, further comprising a methionine at the N-terminus of the polypeptide.
13. The polypeptide of any one of embodiments 1-12, wherein the polypeptide is coupled to a half-life extending moiety.
14. The polypeptide of embodiment 13, wherein the half-life extending moiety is an albumin binding molecule, a polyethylene glycol (PEG), albumin, an albumin variant, or at least a portion of an Fc region of an immunoglobulin.
15. The polypeptide of embodiment 14, wherein the albumin binding molecule is a second polypeptide that binds albumin or an albumin variant.
16. An isolated polynucleotide encoding the polypeptide of any one of embodiments 1-15.
17. A vector comprising the polynucleotide of embodiment 16.
18. A host cell comprising the vector of embodiment 17.
19. A method of producing an polypeptide that binds CD71, comprising culturing the isolated host cell of embodiment 18 under conditions that the polypeptide is expressed, and purifying the polypeptide.
20. A pharmaceutical composition comprising the polypeptide of any one of embodiments 1-15 and a pharmaceutically acceptable carrier.
21. The pharmaceutical composition of embodiment 20, wherein the pharmaceutically acceptable carrier is water.
22. A kit comprising the polypeptide of any one of embodiments 1-15.
23. A method of treating cancer in a subject in need thereof, the method comprising administering to the subject a polypeptide provided for herein, such as SEQ ID NOs: 100-209, 211-301, 303-317, 319-552, and 972-976, with a therapeutic agent.
24. A method of treating cancer in a subject in need thereof, the method comprising administering to the subject a polypeptide described herein, such as SEQ ID NOs: 100-209, 211-301, 303-317, 319-552, and 972-976, conjugated with an antiviral agent, an immune system modulating agent, or a nucleic acid molecule.
25. A method of detecting CD71-expressing cancer cells in a tumor tissue, comprising
   a) obtaining a sample of the tumor tissue from a subject; and
   b) detecting whether CD71 is expressed in the tumor tissue by contacting the sample of the tumor tissue with a polypeptide comprising the amino acid sequence of one of SEQ ID NOs: 100-209, 211-301, 303-317, 319-552, and 972-976, and detecting the binding between CD71 and the polypeptide.
26. A method of isolating CD71 expressing cells, comprising
   a) obtaining a sample from a subject;
   b) contacting the sample with the polypeptide comprising the amino acid sequence of one of SEQ ID NOs: 100-209, 211-301, 303-317, 319-552, and 972-976, and
   c) isolating the cells bound to the polypeptide.
27. A method of detecting CD71-expressing cancer cells in a tumor tissue, comprising
   a) conjugating a polypeptide comprising the amino acid sequence of one of SEQ ID NOs: 100-209, 211-301, 303-317, 319-552, and 972-976 to a detectable label to form a conjugate;
   b) administering the conjugate to a subject; and
   c) visualizing the CD71 expressing cancer cells to which the conjugate is bound.
28. A method of treating a neurological condition and/or a brain tumor, comprising administering to the subject the pharmaceutical composition of embodiment 20.
29. The method of embodiment 28, wherein the brain tumor is selected from the group consisting of nonmalignant brain tumors, benign brain tumors, and malignant brain tumors.
30. The method of embodiment 29, wherein the malignant brain tumor is selected from the group consisting of astrocytoma, medulloblastoma, glioma, ependymoma, germinoma (pinealoma), glioblastoma multiform, oligodendroglioma, schwannoma, retinoblastoma, or a cancer of the spinal cord, e.g., neurofibroma, meningioma, glioma, and sarcoma.
31. The method of embodiment 28, wherein the neurological condition is selected from the group consisting of stroke, diabetes, seizures, hypertensive encephalopathy, acquired immunodeficiency syndrome, traumatic brain injury, multiple sclerosis, Parkinson's disease (PD), and Alzheimer's disease.
32. A method of delivering an agent of interest to a CD71 positive cell, the method comprising contacting a cell with the agent of interest coupled to a FN3 domain that binds to CD71, such as a polypeptide of any one of embodiments 1-15.
33. The method of embodiment 32, wherein the agent of interest is internalized into the cell through the CD71 mediated interaction.
34. The method of embodiment 32, wherein the FN3 domain does not compete with transferrin binding to CD71.
35. The method of any one of embodiments 32-34, wherein the agent of interest is a chemotherapeutic agent, a drug, a growth inhibitory agent, a toxin, a radioactive isotope, an anti-tubulin agent, a polynucleotide, a siRNA molecule, an antisense molecule, a RNA molecule, a DNA molecule, a DNA minor groove binder, a DNA replication inhibitor, an alkylating agent, an antibiotic, an antifolate, an antimetabolite, a chemotherapy sensitizer, a topoisomerase inhibitor, or a vinca alkaloid.
36. The method of any one of embodiments 32-35, wherein the FN3 domain comprises the amino acid sequence of any one of SEQ ID NOs: 100-209, 211-301, 303-317, 319-552, and 972-976.
37. The method of any one of embodiments 32-36, wherein the cell is a muscle cell.
38. The method of any one of embodiments 32-36, wherein the cell is a brain cell or a cell inside of the blood brain barrier.
39. A method of identifying a FN3 protein that binds to CD71 at a site that does not compete or inhibit transferrin binding to CD71, the method comprising:
   a) contacting CD71 in the presence of transferrin or an agent that binds to the CD71 transferrin binding site with a test FN3 protein; and
   b) identifying a test FN3 protein that binds to CD71 in the presence of transferrin or an agent that binds to the CD71 transferrin binding site.
40. The method of embodiment 39, further comprising isolating the test FN3 protein that binds to CD71 in the presence of transferrin or an agent that binds to the CD71 transferrin binding site.
41. The method of embodiments 39 or 40, further comprising sequencing the test FN3 protein that binds to CD71 in the presence of transferrin or an agent that binds to the CD71 transferrin binding site.
42. The method of any one of embodiments 39-41, further comprising preparing or obtaining a nucleic acid sequence encoding the test FN3 protein that binds to CD71 in the presence of transferrin or an agent that binds to the CD71 transferrin binding site.
43. The method of any one of embodiments 39-42, further comprising expressing the test FN3 protein that binds to CD71 in the presence of transferrin or an agent that binds to the CD71 transferrin binding site from a nucleic acid sequence encoding the test FN3 protein that binds to CD71 in the presence of transferrin or an agent that binds to the CD71 transferrin binding site.
44. The method of embodiment 43, wherein the test FN3 protein is expressed in a cell.
45. The method of embodiment 43, further comprising isolating and/or purifying the expressed test FN3 protein.
46. A FN3 protein identified according to any one of embodiments 39-45.
47. A pharmaceutical composition comprising the FN3 protein of embodiment 46.
48. An isolated polynucleotide encoding the polypeptide of embodiment 46.
49. A vector comprising the polynucleotide of embodiment 48.
50. A host cell comprising the vector of embodiment 49.
51. A composition having a formula of

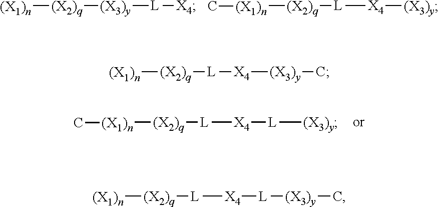

wherein:
X₁ is a first FN3 domain;
X₂ is a second FN3 domain;
X₃ is a third FN3 domain or half-life extender molecule;
L is a linker; and
X₄ is a nucleic acid molecule, such as a siRNA molecule provided herein,
C is a polymer, such as PEG, albumin binding protein, or an aliphatic chain that binds to serum proteins;
wherein n, q, and y are each independently 0 or 1.
52. The composition of embodiment 51, wherein X₁, X₂, and X₃ bind to the same or different target proteins.
53. The composition of embodiments 51 or 52, wherein y is 0.
54. The composition of embodiments 51 or 52, wherein n is 1, q is 0, and y is 0.
55. The composition of embodiments 51 or 52, wherein n is 1, q is 1, and y is 0.
56. The composition of embodiments 51 or 52, wherein n is 1, q is 1, and y is 1.
57. The composition of any one of embodiments 51-56, wherein the third FN3 domain increases the half-life of the molecule as a whole as compared to a molecule without X₃.
58. The composition of embodiment 51, wherein the third FN3 domain is a FN3 domain that binds to albumin.
59. The composition of any one of embodiments 51-58, wherein the linker is a linker as provided herein.
60. The composition of any one of embodiments 51-59, wherein the FN3 domains are connected by a peptide linker.
61. The composition of embodiment 60, wherein the peptide linker is selected from the group consisting of SEQ ID NOs: 46-62 and any combination thereof.
62. The composition of any one of embodiments 51-61, wherein the first, second, or third FN3 domain has an amino acid sequence as provided herein.
63. The composition of any one of embodiments 51-62, wherein X₄ is a siRNA molecule that targets CD40, KRAS, or GYS1.
64. The composition of embodiment 63, wherein the siRNA molecule is a siRNA molecule provided herein.
65. The composition of embodiment 63, wherein the siRNA molecule is a siRNA that reduces the expression of CD40, KRAS, or GYS1.
66. The composition of embodiment 63, wherein the siRNA molecule is a siRNA that specifically reduces the expression of CD40, KRAS, or GYS1.
67. The composition of embodiment 63, wherein the siRNA molecule is a siRNA that reduces the expression of CD40, KRAS, or GYS1 and does not significantly reduce the expression of other RNAs.
68. The composition of embodiment 63, wherein the siRNA molecule is a siRNA that reduces the expression of CD40, KRAS, or GYS1 and does not reduce the expression of other RNAs by more than 50% in an assay described herein at a concentration of no more than 200 nm as described herein.
69. The composition of embodiment 63, wherein the siRNA molecule is a siRNA that reduces the expression of CD40, KRAS, or GYS1 and reduces the concentration of CD40 protein, KRAS protein, or GYS1 protein respectively.
70. The composition of embodiment 63, wherein the siRNA molecule is a siRNA that reduces the expression of GYS1 and reduces the concentration of glycogen in a cell.
71. The composition of embodiment 70, wherein the cell is a muscle cell or a heart cell.
72. The composition of any one of embodiments 63-71, wherein the siRNA is a siRNA pair comprising a sense strand and an antisense strand as provided in the following formula:

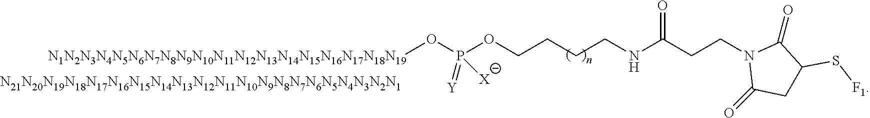

73. The composition of embodiment 72, wherein N₁ of the antisense strand comprises a vinyl phosphonate modification.
74. The composition of embodiment 72, wherein maleimide is hydrolyzed to form the following mixture of compounds, or one or both of each compound, or exclusively one of the compounds:

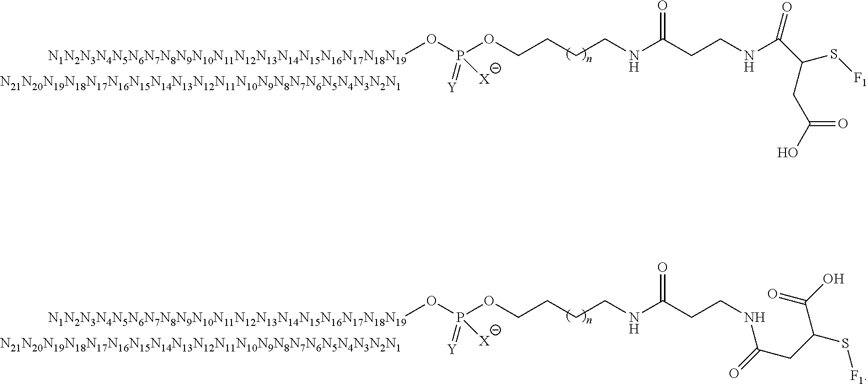

75. The composition of any one of embodiments 51-74, wherein the siRNA is a siRNA Pair as provided herein or a siRNA pair selected from the group consisting of siRNA pairs as provided for in Table 7a or Table 7b, or as provided for herein.
76. A composition having a formula A₁-B₁, wherein A₁ has a formula of (C)ₙ-(L₁)ₜ-Xₛ and B₁ has a formula of X_{AS}-(L₂)_{q}-(F₁)_{y},
   wherein:
   C is a polymer, such as PEG, albumin binding protein, or an aliphatic chain that binds to serum proteins;
   L₁ and L₂ are each, independently, a linker;
   X_{S} is a 5′ to 3′ oligonucleotide sense strand of a double stranded siRNA molecule;
   X_{AS} is a 3′ to 5′ oligonucleotide antisense strand of a double stranded siRNA molecule;
   F₁ is a polypeptide comprising at least one FN3 domain;
   wherein n, t, q, and y are each independently 0 or 1;
   wherein X_{S} and X_{AS} form a double stranded oligonucleotide molecule.
77. A composition having a formula A₁-B₁, wherein A₁ has a formula of (F₁)ₙ-(L₁)ₜ-Xₛ and B₁ has a formula of X_{AS}-(L₂)_{q}-(C)_{y},
   wherein:
   C is a polymer, such as PEG, albumin binding protein, or an aliphatic chain that binds to serum proteins;
   L₁ and L₂ are each, independently, a linker;
   X_{S} is a 5′ to 3′ oligonucleotide sense strand of a double stranded siRNA molecule;
   X_{AS} is a 3′ to 5′ oligonucleotide antisense strand of a double stranded siRNA molecule;
   F₁ is a polypeptide comprising at least one FN3 domain;
   wherein n, t, q, and y are each independently 0 or 1;
   wherein X_{S} and X_{AS} form a double stranded oligonucleotide molecule.
78. The composition of embodiment 76 or 77, wherein L₁ has the formula:

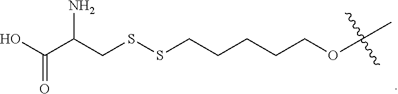 [SMILES: *OCCCCCSSCC(N)C(=O)O]

79. The composition of embodiment 76 or 77, wherein L₂ has the formula:

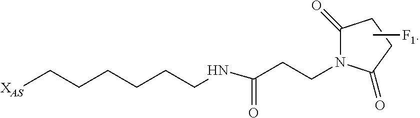

80. The composition of embodiment 76 or 77, wherein A₁-B₁ has a formula of:

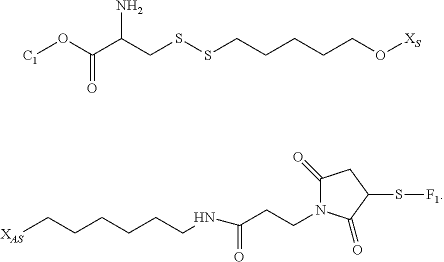

81. The composition of embodiment 76 or 77, where in A₁-B₁ has a formula of:

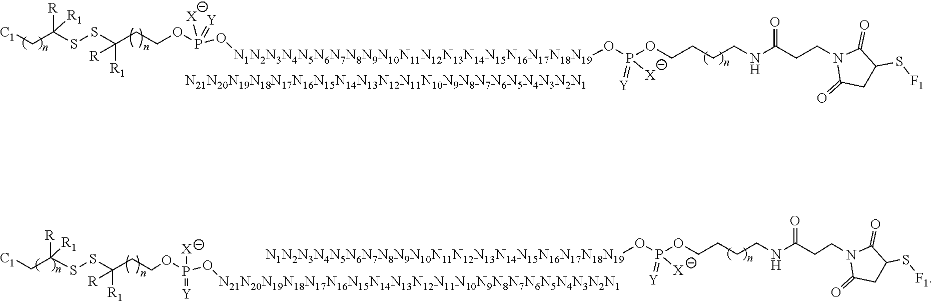

82. The composition of embodiment 76 or 77, wherein F₁ comprises polypeptide having a formula of (X₁)ₙ—(X₂)_{q}—(X₃)_{y}, wherein X₁ is a first FN3 domain; X₂ is a second FN3 domain; X₃ is a third FN3 domain or half-life extender molecule; wherein n, q, and y are each independently 0 or 1, provided that at least one of n, q, and y is 1.
83. The composition of embodiment 76 or 77, wherein X₁ is a CD71 binding FN3 domain.
84. The composition of embodiment 76 or 77, wherein X₂ is a CD71 binding FN3 domain.
85. The composition of embodiment 76 or 77, wherein X₃ is a FN3 domain that binds to human serum albumin.
86. The composition of embodiment 76 or 77, wherein X₃ is a Fc domain without effector function that extends the half-life of a protein.
87. The composition of any one of embodiments 76-86, wherein X_{S} comprises a nucleic acid sequence selected from the group consisting of SEQ ID NOs: 600-659 and SEQ ID NOs: 46-178, 312-331, 352-356, 673-805, and 939-958 of U.S. Provisional Application No. 63/380,112, or as otherwise provided for herein.
88. The composition of any one of embodiments 76-86, wherein X_{AS} comprises a nucleic acid sequence selected from the group consisting of SEQ ID NOs: 660-719 and SEQ ID NOs: 179-311, 332-351, 356-359, 806-938, and 959-978 of U.S. Provisional Application No. 63/380,112, or as otherwise provided for herein.
89. The composition of any one of embodiments 76-86, wherein X_{S} and X_{AS} form a siRNA pair selected from the group consisting of A1, B1, C1, D1, E1, F1, G1, H1, I1, J1, K1, L1, M1, N1, O1, P1, Q1, R1, S1, T1, U1, V1, W1, X1, Y1, Z1, A2, B2, C2, D2, E2, F2, G2, H2, I2, J2, K2, L2, M2, N2, O2, P2, Q2, R2, S2, T2, U2, V2, W2, X2, Y2, Z2, A3, B3, C3, D3, E3, F₃, G3, H3, or as set forth in Table 7a, Table 7b, or any siRNA pair provided in U.S. Provisional Application No. 63/380,112, or as otherwise provided for herein.
90. The composition of any one of embodiments 76-86, wherein F₁ comprises an amino acid sequence that is at least 87% identical to or is identical to a sequence selected from the group consisting of SEQ ID NO: 100-209, 211-301, 303-317, 319-552, and 972-976.
91. The composition of any one of embodiments 76-86, wherein F₁ comprises a polypeptide that binds to albumin.
92. A pharmaceutical composition comprising a composition of any one of embodiments 51-91.
93. A kit comprising a composition of any one of embodiments 51-91.
94. A method of treating immunological diseases in a subject in need thereof, the method comprising administering to the subject a composition of any one of embodiments 51-91 or any composition provided herein.
95. A use of a composition of any one of embodiments 51-91 or any composition provided herein in the preparation of a pharmaceutical composition or medicament for treating immunological diseases, such as autoimmune diseases provided herein.
96. Use of a composition of any one of embodiments 51-91 or any composition provided herein for treating immunological diseases.
97. A method of reducing the expression of a target gene in a cell, such as an immune cell, the method comprising contacting the immune cell with a composition of any one of embodiments 51-91 or any composition as provided herein.
98. The method of embodiment 97, wherein the target gene is CD40, KRAS, or GYS1.
99. A method of delivering a siRNA molecule to a cell in a subject, the method comprising administering to the subject a pharmaceutical composition comprising a composition of any one of embodiments 51-91.
100. The method of embodiment 99, wherein the cell is a CD71 positive cell.
101. The method of embodiments 99 or 100, wherein the cell is an immune cell or a muscle cell.
102. The method of embodiment 101, wherein the immune cell is a B cell, a T cell, or a dendritic cell.
103. The method of any one of embodiments 99-102, wherein the siRNA downregulates the expression of a target gene in the cell.
104. The method of embodiment 103, wherein the downregulation of the expression of the target gene results in a reduction of about 99%, 90-99%, 50-90%, or 10-50%.
105. The method of embodiment 103, wherein the target gene is CD40, KRAS, or GYS1.
106. A method of delivering a siRNA molecule that targets CD40, KRAS, or GYS1 to a CD71 expressing cell in a subject, the method comprising administering to the subject a pharmaceutical composition comprising a composition of any one of embodiments 51-91, wherein the siRNA molecule downregulates the expression of CD40, KRAS, or GYS1 in the CD71 expressing cell.

### EXAMPLES

The following examples are illustrative of the embodiments disclosed herein. These examples are provided for the purpose of illustration only and the embodiments should in no way be construed as being limited to these examples, but rather should be construed to encompass any and all variations which become evidence as a result of the teaching provided herein. Those of skill in the art will readily recognize a variety of non-critical parameters that could be changed or modified to yield essentially similar results.

### Example 1: Construction of Tencon Libraries with Randomized Loops

As described herein, Tencon (SEQ ID NO: 1) is an immunoglobulin-like scaffold, fibronectin type III (FN3) domain, designed from a consensus sequence of fifteen FN3 domains from human tenascin-C(Jacobs et al., Protein Engineering, Design, and Selection, 25:107-117, 2012; U.S. Pat. No. 8,278,419). The crystal structure of Tencon shows six surface-exposed loops that connect seven beta-strands. These loops, or selected residues within each loop, can be randomized in order to construct libraries of fibronectin type III (FN3) domains that can be used to select novel molecules that bind to specific targets.

Various libraries were generated using the Tencon scaffold and various design strategies. In general, libraries TCL1 and TCL2 produced good binders. Generation of TCL1 and TCL2 libraries are described in detail in Int. Pat. Publ. No. WO/2014081944A2, which is hereby incorporated by reference in its entirety.

### Example 2: Generation of Tencon Libraries Having Alternative Binding Surfaces

The choice of residues to be randomized in a particular library design governs the overall shape of the interaction surface created. X-ray crystallographic analysis of an FN3 domain containing scaffold protein selected to bind maltose binding protein (MBP) from a library in which the BC, DE, and FG loops were randomized was shown to have a largely curved interface that fits into the active site of MBP (Koide et al., Proc. Natl. Acad. Sci. USA 104: 6632-6637, 2007). In contrast, an ankyrin repeat scaffold protein that was selected to bind to MBP was found to have a much more planar interaction surface and to bind to the outer surface of MBP distant from the active (Binz et al., Nat. Biotechnol. 22: 575-582, 2004). These results suggest that the shape of the binding surface of a scaffold molecule (curved vs. flat) may dictate what target proteins or specific epitopes on those target proteins are able to be bound effectively by the scaffold. Published efforts around engineering protein scaffolds containing FN3 domains for protein binding has relied on engineering adjacent loops for target binding, thus producing curved binding surfaces. This approach may limit the number of targets and epitopes accessible by such scaffolds.

Tencon and other FN3 domains contain two sets of CDR-like loops lying on the opposite faces of the molecule, the first set formed by the BC, DE, and FG loops, and the second set formed by the AB, CD, and EF loops. The two sets of loops are separated by the beta-strands that form the center of the FN3 structure. If the image of the Tencon is rotated by 90 degrees, an alternative surface can be visualized. This slightly concave surface is formed by the CD and FG loops and two antiparallel beta-strands, the C and the F beta-strands, and is herein called the C-CD-F-FG surface. The C-CD-F-FG surface can be used as a template to design libraries of protein scaffold interaction surfaces by randomizing a subset of residues that form the surface. Beta-strands have a repeating structure with the side chain of every other residue exposed to the surface of the protein. Thus, a library can be made by randomizing some or all surface exposed residues in the beta strands. By choosing the appropriate residues in the beta-strands, the inherent stability of the Tencon scaffold should be minimally compromised while providing a unique scaffold surface for interaction with other proteins.

A full description of the methods used to construct this library is described in US. Pat. Publ. No. 2013/0226834, which is hereby incorporated by reference in its entirety.

The two beta-strands forming the C-CD-F-FG surface in Tencon27 (SEQ ID NO: 2) have a total of 9 surface exposed residues that could be randomized: C-strand S30, L32, Q34, Q36; F-strand E66, T68, S70, Y72, and V74. The CD loop has 6 potential residues: S38, E39, K40, V41, G42, and E43, and the FG loop has 7 potential residues: K75, G76, G77, H78, R79, S80, and N81. Select residues were chosen for inclusion in the TCL14 design due to the larger theoretical size of the library if all 22 residues were randomized.

Thirteen positions in Tencon were chosen for randomizing: L32, Q34 and Q36 in C-strand, S38, E39, K40 and V41 in CD-loop, T68, S70 and Y72 in F-strand, H78, R79, and N81 in FG-loop. In the C and F strands S30 and E66 were not randomized as they lie just beyond the CD and FG loops and do not appear to be a part of the C-CD-F-FG surface. For the CD loop, G42 and E43 were not randomized as glycine, which provides flexibility, can be valuable in loop regions, and E43 lies at the junction of the surface. The FG loop had K75, G76, G77, and S80 excluded. The glycine residues were excluded for the reasons above, while careful inspection of the crystal structures revealed that S80 makes key contacts with the core to help form the stable FG loop. K75 faces away from the surface of the C-CD-F-FG surface and was a less appealing candidate for randomization. Although the above-mentioned residues were not randomized in the original TCL14 design, they could be included in subsequent library designs to provide additional diversity for de novo selection or, for example, for an affinity maturation library on a select TCL14 target-specific hit.

Subsequent to the production of TCL14, three additional Tencon libraries of similar design were produced, as described below. These libraries, TCL19, TCL21 and TCL23, were randomized at the same positions as TCL14 as described above except that the distribution of amino acids occurring at these positions was altered. TCL19 and TCL21 were designed to include an equal distribution of 18 natural amino acids at every position (5.55% of each), excluding only cysteine and methionine. TCL23 was designed such that each randomized position approximates the amino acid distribution found in the HCDR3 loops of functional antibodies (Birtalan et al., J. Mol. Biol. 377: 1518-1528, 2008). As with the TCL21 library, cysteine and methionine were excluded.

A fourth additional library, TCL24, was built to expand potential target binding surface of the other libraries. In TCL24, four additional Tencon positions were randomized as compared to libraries TCL14, TCL19, TCL21, and TCL23. These positions include N46 and T48 from the D strand, and S84 and 186 from the G strand. Positions 46, 48, 84, and 86 were chosen because the side chains of these residues are surface-exposed from beta-strands D and G, and lie structurally adjacent to the randomized portions of the C and F strand, thus increasing the surface area accessible for binding to target proteins. The amino acid distribution used at each position for TCL24 is identical to that described for TCL19 and TCL21.

### Generation of TCL19, TCL21, TCL23, and TCL24 Libraries

The TCL21 library was generated using Colibra® library technology (Isogenica®) in order to control amino acid distributions. TCL19, TCL23, and TCL24 gene fragments were generated using Slonomics® gene synthesis technology (Morphosys®) to control amino acid distributions. PCR was used to amplify each library following initial synthesis followed by ligation to the gene for RepA in order to be used in selections using the CIS display system (Isogenica®) as described above for the loop libraries (Odegrip et al., Proc. Natl. Acad. Sci. USA 101: 2806-2810, 2004).

### Example 3: Selection of Fibronectin Type III (FN3) Domains that Bind CD71

### Panning and Biochemical Screening

FN3 domains specific for human CD71 were selected via CIS display (Isogenica®) using recombinant biotinylated CD71 extracellular domain (Sino Biological®) with an N-terminal 6His tag (SEQ ID NO: 63). For in vitro transcription and translation (ITT), 3 μg of DNA from FN3 domain libraries TCL18, TCL19, TCL21, TCL23, and/or TCL24 were used, with unbound library members removed by washing. DNA was eluted from the target protein by heating and amplified by PCR using DNA polymerase for further rounds of panning. High affinity binders were isolated by successively lowering the concentration of target CD71 during each round from 400 nM to 100 nM and increasing the washing stringency. Outputs from the fifth round panning were subjected to four additional rounds of off-rate selection. The biotinylated target antigen concentration was reduced from 25 nM in rounds 6 and 7 to 2.5 nM in rounds 8 and 9.

Following panning, genes encoding the selected FN3 domains were amplified by PCR, subcloned into a pET bacterial recombinant protein vector modified to include a ligase independent cloning site, and transformed into BL21 (DE3) (Stratagene™) cells for soluble expression in E. coli using standard molecular biology techniques. A gene sequence encoding a C-terminal poly-histidine tag was added to each FN3 domain to enable purification and detection.

To screen for FN3 domains that specifically bind CD71, streptavidin-coated MaxiSorp™ 96-well plates (Nunc™) were blocked for 1 hour in StartingBlock™ T20 blocking buffer (Pierce™) and then coated with either biotinylated CD71 (using same antigen as in panning) or negative controls (an unrelated Fc-fused recombinant protein and human serum albumin) for 1 hour. Plates were rinsed with tris-buffered saline with 0.1% Tween® 20 detergent (TBST) buffer, and diluted lysate was applied to the plates for 1 hour. Following additional rinses, wells were treated with horseradish peroxidase (HRP)-conjugated anti-V5 tag antibody (Abcam®), for 1 hour and then assayed via ELISA with peroxidase. The DNA from FN3 domain lysates with signals at least 10-fold ELISA signal above that of streptavidin controls were sequenced, resulting in 23 unique, readable FN3 domain sequences isolated from Round 9 screening.

### Size Exclusion Chromatography Analysis

Size exclusion chromatography was used to determine the aggregation state of anti-CD71 FN3 domains. Aliquots (10 μL) of each purified FN3 domain were injected onto a Superdex® 75 5/150 column (GE Healthcare®) at a flow rate of 0.3 mL/min in a mobile phase of phosphate-buffered saline (PBS) at pH 7.4. Elution from the column was monitored by absorbance at 280 nm. Tencon protein was included in each run as a control. ChemStation® software (Agilent®) was used to analyze the elution profiles.

### High-throughput Expression and Conjugation

Identified clones were grown in duplicate 5 mL cultures in 24-well deep block plates. Briefly, 5 mL/well of Terrific Broth nutritionally rich media supplemented with 50 μg/mL Kanamycin was seeded with 150 μL of overnight culture and grown for about 3 hours at 370 C with shaking at 220 rpm (OD600˜1). Cultures were induced with IPTG to a final concentration of 1 mM for an additional 4 hours at 37° C., 220 rpm. Bacterial pellets were recovered by centrifugation at 2250×g for 15 minutes. 600 μL/well BugBuster® HT (Novagen®) supplemented with lysozyme at 0.2 mg/mL was added to each well; pellets were dissociated by pipette and then shaken vigorously on a platform shake for about 30 minutes until pellets were lysed. Plates were spun at 2250×g for 15 minutes to clarify lysates and the two 600 μL aliquots for each sample were combined. His-tagged FN3 domains were purified on HisTrap® 96-well filter plates (GE Healthcare®) according to the manufacturer's instructions followed by buffer exchange into tris-buffered saline (TBS) using Zeba™ Spin 7K desalting plates (Thermo Scientific®). Protein concentrations were assessed by UV visible spectrophotometry.

For conjugation to GlyGly-VC-MMAF, FN3 domain (30 μM) was mixed with 150 μM GlyGlyVC-MMAF (Concortis) and 1 μM Sortase A in a total volume of 200 μL. Conjugations were allowed to proceed for 1.5 hours at room temperature and purified again using a His MultiTrap™ HP 96-well filter plate (GE Healthcare®) according to the manufacturer's instructions. Buffer exchange into PBS was achieved using Zeba™ desalting plates followed by sterile filtering using MultiScreenHTS GV filter plates (Durapore®) with centrifugation at 3000×g for 2 mins. Protein concentrations were assessed by UV visible spectrophotometry.

### Identification of SK-BR3 Binding FN3 Domains

SK-BR-3 cells are cultured in McCoy's 5A Medium+10% Fetal Bovine Serum. FN3 dilutions are prepared in FACS buffer. 50,000 SK-BR-3 cells are added to each well; media was aspirated after centrifugation and cells are resuspended in 100 μL of FACS buffer containing fluorescently-labeled FN3 domains. Cells are incubated for 2 hours at 37° C., 5% CO2. Cells are rinsed 3× with FACS buffer and finally resuspended in 100 μL of FACS buffer. Fluorescence was detected by flow cytometry. Cell populations were identified by the forward scatter (FSC)-side scatter (SSC) dot plot followed by recording of the median fluorescence intensity (MFI) of cells on the FL4 Proliferation dye channel. Data are normalized to the average of 8 unstained cells and dose response curves are fit using GraphPad®.

### Binding of Selected Clones by Dose-Response ELISA

Selected clones were analyzed by ELISA to determine EC50 values for binding. Briefly, MaxiSorp™ 96-well plates (Nunc™) were coated with streptavidin at 5 μg/ml overnight at 4° C. Plates were then blocked with StartingBlock™ blocking buffer (Pierce™) at room temperature for 1 hour and then washed with TBST. Biotinylated CD71 (2 μg/ml) was captured onto the streptavidin plates and serially diluted FN3 proteins were added to appropriate wells for 1 hour at room temperature. After washing, bound FN3 proteins was detected with anti-V5 tag antibody, which is conjugated to HRP and POD substrate and a luminescence plate reader. Luminescence values are plotted as a function of concentration and fit to a dose response using GraphPad PRISM® to determine EC50 values for binding.

Identification of internalizing FN3 domains via toxin conjugates. The FN3 domains were conjugated to the cytotoxic tubulin inhibitor monomethyl auristatin F (MMAF) via an enzyme-cleavable Val-Cit linker or a non-cleavable PEG4 linker (VC-MMAF) using the methodology described for the NEM conjugation. Cell killing was assessed by measuring viability of the SKBR-3 cells following exposure to the cysteine variant-cytotoxin conjugates. Cells are plated in white-well, opaque bottomed, tissue culture-treated plates (Thermo Scientific™ Pierce™) at 3000/well in 50 μL/well of phenol red RPMI media (Gibco™) with 10% fetal bovine serum. Cells are allowed to attach overnight at 370 C in a humidified 5% CO2 atmosphere. Cells are treated with 25 μL of fresh media and 25 μL of 4× inhibitor made up in fresh media. Cell viability is determined by an endpoint assay with CellTiter-Glo® luminescent cell viability assay (Promega®) at 72 hours. IC50 values are determined by fitting data to the equation for a sigmoidal dose response with variable slope using GraphPad Prism®.

### Bivalent FN3 Protein

A bivalent FN3 protein is produced using two FN3 domains connected by a 4 repeat G/S linker or other appropriate polypeptide linker. The bivalent FN3 protein is conjugated to VC-MMAF as described and assessed for cytotoxicity in SK-BR3 cells. The IC50 value for bivalent molecule is often found to be better than the monovalent version.

### Competition for Transferrin Binding and Internalization

FN3 domain vcMMAF conjugates were screened for competition with human transferrin using the cytotoxicity assay described above. FN3 domains were screened in the absence or presence of 0.6 uM holo-human transferrin (T0665-100MG).

pHrodo™-Transferrin Assay

CD71-targeting Centyrins were evaluated for their ability to compete with transferrin for binding to the transferrin receptor. Cells are treated with transferrin that is directly conjugated to pHrodo™ Red, a dye that fluoresces in acidic compartments and is therefore visible upon cellular uptake into endosomal and lysosomal compartments. Imaging of pHrodo™-transferrin (pHrodo™-Tf) is performed on an Incucyte® lice-cell analysis system, allowing real-time measurement of transferrin uptake. When cells are incubated with pHrodo™-Tf and a molecule that competes with transferrin for CD71 binding, the pHrodo™ signal is reduced or eliminated. Centyrins that do not compete with transferrin for CD71 binding have no impact on the pHrodo™ signal.

### Example 4. Affinity Maturation Panning

Four sequences that demonstrated selective CD71 apical domain binding were the basis of affinity maturation library: SEQ ID NOs: 91, 92, 93 and 94. In each sequence, 4 amino acids (double underlined) part of extended sheet library, were randomized to 18 amino acids (alanine, arginine, asparagine, aspartic acid, cysteine, glutamine, glutamic acid, glycine, histidine, isoleucine, leucine, lysine, phenylalanine, serine, threonine, tryptophan, tyrosine, or valine, not including proline, methionine). Four different screens were performed as described below.

| SEQ ID | |
|---|---|
| NO: | SEQUENCE |
| 91 | MLPAPKNLVVSRVTEDSARLSWTAPDAAFDSFYIAYAEPRPDGEAIVLT |
| | VPGSERSYDLTGLKPGTEYSVLIHGVKGGLLSSPLSAIFTT |
| 92 | MLPAPKNLVVSRVTEDSARLSWTAPDAAFDSFFIGYLEPQPPGEAIVLT |
| | VPGSERSYDLTGLKPGTEYNVTIQGVKGGFPSMPLSAIFTT |
| 93 | MLPAPKNLVVSRVTEDSARLSWTAPDAAFDSFHIVYHEPRPSGEAIVLT |
| | VPGSERSYDLTGLKPGTEYEVGIVSVKGGDLSVPLSAIFTT |
| 94 | MLPAPKNLVVSRVTEDSARLSWTAPDAAFDSFDIGYTEYGGYGEAIVLT |
| | VPGSERSYDLTGLKPGTEYWVLIQGVKGGGSSVPLSAIFTT |

### Screen 1 (ISOP873, 875, 806)

The libraries underwent selection against 5 rounds of panning against decreasing concentrations of human apical domain of CD71 (which was 30% biotinylated) or intermittent against human apical domain or human CD71 extracellular domain (CD71-ECD). No follow-up off-rate selection was performed. Colonies were screened in primary ELISA against human CD71-ECD and apical domain of human CD71 (and compared to the negative control of human serum albumin (HSA)) and only selected hits that exhibited binding to both antigens. ELISA data are shown in Table 10. Purification data shown in Table 11.

TABLE 10
Summary of Screening Hits: Primary ELISA
Screen Against CD71 and Apical Domain

| | Apical | | | | |
|---|---|---|---|---|---|
| hCD71, | domain, | HSA, | | Apical | SEQ |
| RLU | RLU | RLU | hCD71:HSA | domain:HSA | ID NO |
| 6949000 | 6103000 | 7750 | 897 | 787 | 100 |
| 340450 | 541950 | 4350 | 78 | 125 | 101 |
| 307700 | 5868100 | 4550 | 68 | 1290 | 102 |
| 6369550 | 5303250 | 18200 | 350 | 291 | 103 |
| 6359650 | 4428250 | 29450 | 216 | 150 | 104 |
| 1509050 | 3838000 | 3000 | 503 | 1279 | 105 |
| 3128900 | 4811350 | 2550 | 1227 | 1887 | 106 |
| 6029900 | 4230650 | 3250 | 1855 | 1302 | 107 |
| 3809850 | 5767700 | 3350 | 1137 | 1722 | 108 |
| 3936850 | 3348850 | 3250 | 1211 | 1030 | 109 |
| 5174600 | 5995250 | 2500 | 2070 | 2398 | 110 |
| 3704650 | 5037000 | 2600 | 1425 | 1937 | 111 |
| 5407100 | 4781300 | 2650 | 2040 | 1804 | 112 |
| 4601450 | 5942050.0 | 2800 | 1643 | 2122 | 113 |
| 1501800 | 3587700.0 | 11550 | 130 | 311 | 114 |
| 5192800 | 4580250.0 | 5200 | 999 | 881 | 115 |
| 4051650 | 6057550.0 | 2750 | 1473 | 2203 | 116 |
| 4213400 | 5326700.0 | 3750 | 1124 | 1420 | 117 |
| 641000 | 3747350.0 | 3200 | 200 | 1171 | 118 |

TABLE 11
Summary of Size Exclusion Chromatography Analysis
of Hits from the Apical Domain Panning

| RT (min) | Height (mAU) | Y/N | SEQ ID NO |
|---|---|---|---|
| 5.12 | 75917 | Y | 100 |
| 5.27 | 109113 | Y | 101 |
| 5.21 | 112787 | Y | 102 |
| 5.10 | 27418 | N | 103 |
| 4.99 | 23104 | N | 104 |
| 5.29 | 91590 | Y | 105 |
| 5.18 | 78482 | Y | 106 |
| 5.08 | 70783 | Y | 107 |
| 5.11 | 88256 | Y | 108 |
| 5.07 | 63823 | Y | 109 |
| 5.08 | 64261 | Y | 110 |
| 5.18 | 83785 | Y | 111 |
| 5.13 | 72015 | Y | 112 |
| 5.09 | 73671 | Y | 113 |
| 5.25 | 95320 | Y | 114 |
| 5.08 | 65420 | Y | 115 |
| 5.16 | 80017 | Y | 116 |
| 5.11 | 80891 | Y | 117 |
| 5.21 | 75645 | Y | 118 |

### Screen 2 (ISOP878-879)

The libraries underwent selection against 9 rounds of panning against decreasing concentrations of human apical domain of CD71 (100% biotinylated), including 4 rounds of off-rate selection. Libraries were panned separately (to isolate extended sheet library). Colonies were screened in primary ELISA against human CD71-ECD and apical domain of cynomolgus CD71 (and compared to the negative control of HSA) and only selected hits that exhibited binding to both antigens. ELISA data are shown in Table 12. Purification data shown in Table 13.

TABLE 12
Summary of Screening Hits: Primary ELISA
Screen Against CD71 and Apical Domain

| | Cyno apical | | | | |
|---|---|---|---|---|---|
| hCD71, | domain, | HSA, | | Cyno apical | SEQ |
| RLU | RLU | RLU | hCD71:HSA | domain:HSA | ID NO |
| 7035150 | 6290050 | 15950 | 441 | 394 | 119 |
| 4256800 | 5126100 | 9600 | 443 | 534 | 120 |
| 5418450 | 6218400 | 49650 | 109 | 125 | 121 |
| 5502800 | 4197800 | 52650 | 105 | 80 | 122 |
| 4506050 | 5198250 | 41650 | 108 | 125 | 123 |
| 5011100 | 4888000 | 115500 | 43 | 42 | 124 |
| 3635350 | 5088350 | 33350 | 109 | 153 | 125 |
| 3131100 | 5269850 | 13800 | 227 | 382 | 126 |
| 4730050 | 6515200 | 25150 | 188 | 259 | 127 |
| 4616800 | 5557750 | 21450 | 215 | 259 | 128 |
| 2485300 | 4890700 | 15650 | 159 | 313 | 129 |
| 3756400 | 5001250 | 25500 | 147 | 196 | 130 |
| 3347100 | 4808150 | 33150 | 101 | 145 | 131 |
| 3634950 | 5148650 | 25650 | 142 | 201 | 132 |
| 1827650 | 5108950 | 13200 | 138 | 387 | 133 |
| 4516050 | 5765350 | 23500 | 192 | 245 | 134 |
| 5863150 | 6071800 | 26200 | 224 | 232 | 135 |
| 4080300 | 4455000 | 14950 | 273 | 298 | 136 |
| 7033900 | 5962650 | 27400 | 257 | 218 | 137 |
| 5908400 | 6592600 | 24000 | 246 | 275 | 138 |
| 3699700 | 4492250 | 9050 | 409 | 496 | 139 |
| 5723000 | 7009650 | 35150 | 163 | 199 | 140 |
| 4219900 | 5398750 | 22000 | 192 | 245 | 141 |
| 3573650 | 5272150 | 55300 | 65 | 95 | 142 |
| 2912450 | 4412500 | 31050 | 94 | 142 | 143 |
| 4737800 | 5300500 | 35450 | 134 | 150 | 144 |
| 5142300 | 6364300 | 32500 | 158 | 196 | 145 |
| 4339750 | 4958650 | 38600 | 112 | 128 | 146 |
| 5555500 | 6671250 | 32050 | 173 | 208 | 147 |
| 2453200 | 4716950 | 11550 | 212 | 408 | 148 |
| 3765100 | 5004800 | 11000 | 342 | 455 | 149 |
| 3493700 | 4185000 | 35500 | 98 | 118 | 150 |
| 4036200 | 5096250 | 183050 | 22 | 28 | 151 |
| 2362600 | 4207050 | 14450 | 164 | 291 | 152 |
| 4571150 | 4113450 | 32350 | 141 | 127 | 153 |
| 2475050 | 4430150 | 27500 | 90 | 161 | 154 |
| 3545350 | 5186400 | 23550 | 151 | 220 | 155 |
| 3679700 | 4425900 | 53550 | 69 | 83 | 156 |
| 2891400 | 4322050 | 8150 | 355 | 530 | 157 |
| 4480150 | 4629250 | 46700 | 96 | 99 | 158 |
| 2637250 | 4768800 | 66600 | 40 | 72 | 159 |
| 4376100 | 5023200 | 50000 | 88 | 100 | 160 |
| 5727250 | 6089800 | 447200 | 13 | 14 | 161 |
| 4456300 | 5493900 | 18550 | 240 | 296 | 162 |
| 1495500 | 5142500 | 22900 | 65 | 225 | 163 |
| 5548400 | 3780500 | 19400 | 286 | 195 | 164 |
| 4252950 | 3617800 | 13950 | 305 | 259 | 165 |
| 4436000 | 5019700 | 29050 | 153 | 173 | 166 |
| 4722400 | 5932150 | 71650 | 66 | 83 | 167 |
| 3941950 | 4842800 | 124750 | 32 | 39 | 168 |
| 2631250 | 4346600 | 9100 | 289 | 478 | 169 |
| 3808500 | 5001200 | 30100 | 127 | 166 | 170 |
| 4402850 | 4927500 | 40500 | 109 | 122 | 171 |
| 4340300 | 5234050 | 139950 | 31 | 37 | 172 |
| 4412900 | 5653500 | 18850 | 234 | 300 | 173 |
| 3613050 | 4707650 | 28500 | 127 | 165 | 174 |
| 1010100 | 4292250 | 47200 | 21 | 91 | 175 |
| 7011000 | 6062200 | 40750 | 172 | 149 | 176 |
| 3219300 | 4541300 | 29200 | 110 | 156 | 177 |
| 7009900 | 5989250 | 22000 | 319 | 272 | 178 |
| 4111050 | 4709300 | 104650 | 39 | 45 | 179 |
| 6676800 | 6094350 | 24300 | 275 | 251 | 180 |
| 2838000 | 4605700 | 21300 | 133 | 216 | 181 |
| 3811550 | 4961900 | 37900 | 101 | 131 | 182 |
| 3194150 | 4471300 | 29650 | 108 | 15 | 183 |
| 3739000 | 5081300 | 50350 | 74 | 101 | 184 |
| 2264500 | 4083850 | 15300 | 148 | 267 | 185 |
| 3630250 | 4754900 | 31050 | 117 | 153 | 186 |
| 3061800 | 4222200 | 45600 | 67 | 93 | 187 |
| 1110850 | 3367500 | 33650 | 33 | 100 | 188 |
| 3447650 | 4872900 | 2500 | 1379 | 1949 | 189 |
| 3663500 | 5203450 | 76350 | 48 | 68 | 190 |
| 4199000 | 4992300 | 45800 | 92 | 109 | 191 |
| 2666750 | 3910750 | 43500 | 61 | 90 | 192 |
| 2325350 | 3946850 | 34050 | 68 | 116 | 193 |
| 2563000 | 3784150 | 48900 | 52 | 77 | 194 |
| 2879850 | 4200350 | 27050 | 106 | 155 | 195 |
| 3447100 | 4609600 | 10950 | 315 | 42 | 196 |
| 3400950 | 6234350 | 94750 | 36 | 66 | 197 |
| 3616500 | 4947700 | 42800 | 84 | 116 | 198 |
| 4453900 | 4896800 | 26150 | 170 | 187 | 199 |
| 3493350 | 4715100 | 27450 | 127 | 172 | 200 |
| 1591050 | 3008600 | 39500 | 40 | 76 | 201 |
| 1114350 | 3505550 | 19100 | 58 | 184 | 202 |
| 2253250 | 4847800 | 13400 | 168 | 362 | 203 |
| 4085600 | 5456950 | 16800 | 243 | 325 | 204 |
| 1019500 | 4100650 | 19250 | 53 | 213 | 205 |
| 3896850 | 5327500 | 79400 | 49 | 67 | 206 |
| 3582100 | 4936850 | 161700 | 22 | 31 | 207 |
| 4060300 | 5452750 | 28500 | 142 | 19 | 208 |
| 4271000 | 5071150 | 12000 | 356 | 423 | 209 |
| 1241650 | 5401500 | 8450 | 147 | 639 | 210 |
| 2021150 | 4671150 | 26450 | 76 | 177 | 211 |
| 2816850 | 5129300 | 29100 | 97 | 176 | 212 |
| 2459150 | 4743550 | 18900 | 130 | 251 | 213 |
| 1014950 | 4200250 | 28450 | 36 | 148 | 214 |
| 3344250 | 4954950 | 70150 | 48 | 71 | 215 |
| 3371050 | 5036150 | 42300 | 80 | 119 | 216 |
| 2329500 | 3982500 | 54050 | 43 | 74 | 217 |
| 2423150 | 4719700 | 31650 | 77 | 149 | 218 |
| 3006750 | 4578300 | 45300 | 66 | 101 | 219 |
| 2425600 | 4690800 | 6250 | 388 | 751 | 220 |
| 5753150 | 6077750 | 32600 | 176 | 186 | 221 |
| 5784450 | 6007850 | 26950 | 215 | 223 | 222 |
| 2248850 | 4112650 | 88150 | 26 | 47 | 223 |
| 3095650 | 4793500 | 41650 | 74 | 115 | 224 |
| 2434600 | 4218000 | 12750 | 191 | 331 | 225 |
| 3096500 | 4334050 | 153550 | 20 | 28 | 226 |
| 2624550 | 4228500 | 86200 | 30 | 49 | 227 |
| 2664000 | 4859050 | 20100 | 133 | 242 | 228 |
| 3415100 | 4521850 | 37850 | 90 | 119 | 229 |
| 2537200 | 4726450 | 16750 | 151 | 282 | 230 |
| 4934550 | 5518900 | 87650 | 56 | 63 | 231 |
| 3576600 | 5407200 | 18350 | 195 | 295 | 232 |
| 2686850 | 4776150 | 13300 | 202 | 359 | 233 |

TABLE 13
Summary of Size Exclusion Chromatography Analysis
of Hits from the Apical Domain Panning

| RT (min) | Height (mAU) | Y/N | SEQ ID NO |
|---|---|---|---|
| 5.13 | 97341 | Y | 119 |
| 5.30 | 202525 | Y | 120 |
| 5.11 | 212892 | Y | 121 |
| 5.02 | 17319 | N | 122 |
| 5.16 | 163808 | Y | 123 |
| 5.11 | 180381 | Y | 124 |
| 5.06 | 168533 | Y | 125 |
| 5.29 | 279608 | Y | 126 |
| 5.15 | 180002 | Y | 127 |
| 5.13 | 374608 | Y | 128 |
| 5.09 | 349857 | Y | 129 |
| 5.22 | 316888 | Y | 130 |
| 5.17 | 227278 | Y | 131 |
| 5.15 | 249072 | Y | 132 |
| 5.10 | 149059 | Y | 133 |
| 5.20 | 293513 | Y | 134 |
| 5.25 | 239709 | Y | 135 |
| 5.22 | 204492 | Y | 136 |
| 5.19 | 206270 | Y | 137 |
| 5.14 | 187433 | Y | 138 |
| 5.09 | 261820 | Y | 139 |
| 5.06 | 185184 | Y | 140 |
| 5.14 | 234171 | Y | 141 |
| 5.21 | 20593 | Y | 142 |
| 5.07 | 161200 | Y | 143 |
| 5.10 | 225971 | Y | 144 |
| 5.19 | 237103 | Y | 145 |
| 5.16 | 170620 | Y | 146 |
| 5.04 | 169116 | Y | 147 |
| 5.06 | 324630 | Y | 148 |
| 5.19 | 218030 | Y | 149 |
| 5.20 | 112050 | Y | 150 |
| 5.30 | 205083 | Y | 151 |
| 5.18 | 221387 | Y | 152 |
| 5.05 | 208057 | Y | 153 |
| 5.13 | 250210 | Y | 154 |
| 5.11 | 199077 | Y | 155 |
| 5.13 | 275311 | Y | 156 |
| 5.27 | 264590 | Y | 157 |
| 5.31 | 294216 | Y | 158 |
| 5.18 | 308429 | Y | 159 |
| 5.11 | 310553 | Y | 160 |
| 5.07 | 205574 | Y | 161 |
| 5.15 | 330298 | Y | 162 |
| 4.81 | 195288 | Y | 163 |
| 4.45 | 217863 | Y | 164 |
| 5.00 | 257404 | Y | 165 |
| 5.20 | 363173 | Y | 166 |
| 5.06 | 181900 | Y | 167 |
| 5.03 | 157693 | Y | 168 |
| 5.09 | 262864 | Y | 169 |
| 5.19 | 284546 | Y | 170 |
| 5.12 | 235334 | Y | 171 |
| 5.24 | 187931 | Y | 172 |
| 5.12 | 276494 | Y | 173 |
| 5.22 | 403872 | Y | 174 |
| 5.29 | 196410 | Y | 175 |
| 5.16 | 253161 | Y | 176 |
| 5.09 | 355881 | Y | 177 |
| 5.13 | 169590 | Y | 178 |
| 5.08 | 74343 | N | 179 |
| 5.07 | 173625 | Y | 180 |
| 5.14 | 341298 | Y | 181 |
| 5.15 | 205277 | Y | 182 |
| 5.09 | 181448 | Y | 183 |
| 5.14 | 30409 | N | 184 |
| 5.09 | 275336 | Y | 185 |
| 5.12 | 309922 | Y | 186 |
| 5.16 | 257924 | Y | 187 |
| 5.06 | 234843 | Y | 188 |
| 5.17 | 233037 | Y | 189 |
| 5.09 | 27253 | N | 190 |
| 5.08 | 332748 | Y | 191 |
| 5.08 | 380761 | Y | 192 |
| 5.07 | 258871 | Y | 193 |
| 5.14 | 325495 | Y | 194 |
| 5.11 | 217781 | Y | 195 |
| 5.09 | 276446 | Y | 196 |
| 5.01 | 173766 | Y | 197 |
| 5.10 | 145430 | Y | 198 |
| 5.13 | 291954 | Y | 199 |
| 5.10 | 291400 | Y | 200 |
| 5.13 | 240219 | Y | 201 |
| 5.16 | 214165 | Y | 202 |
| 5.11 | 258922 | Y | 203 |
| 5.04 | 246923 | Y | 204 |
| 5.06 | 155497 | Y | 205 |
| 5.11 | 179013 | Y | 206 |
| 5.10 | 141660 | N | 207 |
| 5.10 | 366068 | Y | 208 |
| 5.09 | 229919 | Y | 209 |
| 5.07 | 13935 | N | 210 |
| 5.85 | 7632 | N | 211 |
| 5.17 | 234946 | Y | 212 |
| 5.33 | 88393 | Y | 213 |
| 5.22 | 168588 | Y | 214 |
| 5.33 | 119897 | Y | 215 |
| 5.21 | 127724 | Y | 216 |
| 5.16 | 233135 | Y | 217 |
| 5.14 | 233897 | Y | 218 |
| 5.15 | 150139 | Y | 219 |
| 5.23 | 205723 | Y | 220 |
| 5.11 | 234561 | Y | 221 |
| 5.11 | 214579 | Y | 222 |
| 5.13 | 285516 | Y | 223 |
| 5.09 | 89108 | Y | 224 |
| 5.15 | 221043 | Y | 225 |
| 5.33 | 157 | N | 226 |
| 5.18 | 35551 | Y | 227 |
| 5.14 | 41850 | Y | 228 |
| 5.14 | 47916 | Y | 229 |
| 4.55 | 7476 | N | 230 |
| 4.53 | 8493 | Y | 231 |
| 5.17 | 38545 | Y | 232 |
| 5.02 | 30318 | N | 233 |

### Screen 3 (ISOP880-881)

The libraries underwent selection against 5 rounds of Panning against decreasing concentrations of human apical domain of CD71 (100% biotinylated) intermittent against cynomolgus apical domain (100% biotinylated), followed by 4 rounds of off-rate selection against with cold antigen human CD71 or cynomolgus CD71 (non-biotinylated). Colonies were screened in primary ELISA against human CD71-ECD and cynomolgus apical domain of human CD71 (and compared to the negative control of HSA) and only selected hits that exhibited binding to both antigens. ELISA data are shown in Table 14. Purification data shown in Table 15.

TABLE 14
Summary of Screening Hits: Primary Elisa
Screen Against CD71 and Apical Domain

| | Apical | | | | |
|---|---|---|---|---|---|
| hCD71, | domain, | HSA, | | Apical | SEQ |
| RLU | RLU | RLU | hCD71:HSA | domain:HSA | ID NO |
| 6416250 | 7505150 | 9400 | 683 | 798 | 234 |
| 3798050 | 5634950 | 10100 | 376 | 558 | 235 |
| 3293550 | 5316700 | 32450 | 101 | 164 | 236 |
| 3984600 | 5008700 | 7450 | 535 | 672 | 237 |
| 3861850 | 6393900 | 6700 | 576 | 954 | 238 |
| 1876750 | 5396200 | 12200 | 154 | 442 | 239 |
| 3715650 | 6830850 | 95650 | 39 | 71 | 240 |
| 3404000 | 6212300 | 19150 | 178 | 324 | 241 |
| 1612200 | 4836250 | 9800 | 165 | 493 | 242 |
| 2784250 | 4568200 | 7850 | 355 | 582 | 243 |
| 3253950 | 5225400 | 8300 | 392 | 630 | 244 |
| 3750100 | 6069400 | 9950 | 377 | 610 | 245 |
| 3677050 | 6180350 | 10150 | 362 | 609 | 246 |
| 2892400 | 4447600 | 6550 | 442 | 679 | 247 |
| 3487200 | 5118350 | 9300 | 375 | 550 | 248 |
| 3023600 | 4808050 | 8650 | 350 | 556 | 249 |
| 3406100 | 5110450 | 24250 | 140 | 211 | 250 |
| 4090400 | 5101750 | 5650 | 724 | 903 | 251 |
| 2857700 | 5163350 | 9100 | 314 | 567 | 252 |
| 3529650 | 5779000 | 10900 | 324 | 530 | 253 |
| 3292100 | 5107200 | 11400 | 289 | 448 | 254 |
| 3725700 | 5410250 | 41200 | 90 | 131 | 255 |
| 2388100 | 4921750 | 12350 | 193 | 399 | 256 |
| 2831550 | 5149700 | 7500 | 378 | 687 | 257 |
| 4766200 | 6791100 | 22500 | 212 | 302 | 258 |
| 3769450 | 5157850 | 12550 | 300 | 411 | 259 |
| 3481700 | 5953250 | 20700 | 168 | 288 | 260 |
| 3426900 | 4566500 | 6400 | 535 | 714 | 261 |
| 6776100 | 7198850 | 12800 | 529 | 562 | 262 |
| 3320300 | 5510850 | 16000 | 208 | 344 | 263 |
| 5770150 | 6230000 | 11350 | 508 | 549 | 264 |
| 5660100 | 6758000 | 13850 | 409 | 488 | 265 |
| 5405050 | 7414350 | 36050 | 150 | 206 | 266 |
| 3118750 | 6049000 | 34000 | 92 | 178 | 267 |
| 4101200 | 5800650 | 30900 | 133 | 188 | 268 |
| 4353600 | 6010900 | 12500 | 348 | 481 | 269 |
| 5497050 | 6297800 | 85150 | 65 | 74 | 270 |
| 2570950 | 4940950 | 14250 | 180 | 347 | 271 |
| 2834550 | 4663800 | 9800 | 289 | 476 | 272 |
| 3783000 | 6045150 | 5700 | 664 | 1061 | 273 |
| 2371400 | 4645000 | 8000 | 296 | 581 | 274 |
| 3400500 | 5438450 | 10550 | 322 | 515 | 275 |
| 3131900 | 4538400 | 50700 | 62 | 90 | 276 |
| 4134850 | 5752400 | 15000 | 276 | 383 | 277 |
| 3456800 | 5096550 | 7950 | 435 | 641 | 278 |
| 3656100 | 5542200 | 8150 | 449 | 680 | 279 |
| 3212750 | 4974050 | 31100 | 103 | 160 | 280 |
| 6372750 | 6926000 | 16750 | 380 | 413 | 281 |
| 3198500 | 4854650 | 13300 | 240 | 365 | 282 |
| 2065100 | 6135650 | 26750 | 77 | 229 | 283 |
| 2368200 | 4405200 | 12150 | 195 | 363 | 284 |
| 2809600 | 5128800 | 13950 | 201 | 368 | 285 |
| 2661800 | 5307700 | 20250 | 131 | 262 | 286 |
| 3211300 | 4894400 | 9850 | 326 | 497 | 287 |
| 3869050 | 5420700 | 7400 | 523 | 733 | 288 |
| 3570350 | 5200450 | 46500 | 77 | 112 | 289 |
| 5982850 | 6869250 | 43350 | 138 | 158 | 290 |
| 3555650 | 5567050 | 14850 | 239 | 375 | 291 |
| 4330300 | 5290250 | 14900 | 291 | 355 | 292 |
| 6226900 | 6715100 | 11100 | 561 | 605 | 293 |
| 6484200 | 7402150 | 30200 | 215 | 245 | 294 |
| 6201800 | 6710200 | 14150 | 438 | 474 | 295 |
| 3388400 | 4818900 | 34100 | 99 | 141 | 296 |
| 2638400 | 5294050 | 5150 | 512 | 1028 | 297 |
| 3667400 | 5299400 | 12800 | 287 | 414 | 298 |
| 3263200 | 5345700 | 8700 | 375 | 614 | 299 |
| 3883950 | 5568500 | 13700 | 284 | 406 | 300 |
| 3188450 | 4530750 | 20950 | 152 | 216 | 301 |
| 2089850 | 5555300 | 2500 | 836 | 2222 | 302 |
| 1862350 | 5600200 | 8800 | 212 | 636 | 303 |
| 3386250 | 5503100 | 12350 | 274 | 446 | 304 |
| 3618600 | 5691550 | 11500 | 315 | 495 | 305 |
| 1170750 | 4737000 | 14800 | 79 | 320 | 306 |
| 3747850 | 5362500 | 74650 | 50 | 72 | 307 |
| 3552300 | 5260300 | 27950 | 127 | 188 | 308 |
| 3574200 | 5624200 | 12450 | 287 | 452 | 309 |
| 2217100 | 4830100 | 28750 | 77 | 168 | 310 |
| 2378200 | 4655900 | 17900 | 133 | 260 | 311 |
| 3096050 | 5305300 | 9550 | 324 | 556 | 312 |
| 2974700 | 5174450 | 6200 | 480 | 835 | 313 |
| 3150000 | 5253150 | 12300 | 256 | 427 | 314 |
| 2485750 | 4399000 | 60500 | 41 | 73 | 315 |
| 3028550 | 4440500 | 10050 | 301 | 442 | 316 |
| 2552650 | 4090450 | 4800 | 532 | 852 | 317 |
| 3439000 | 4634950 | 4750 | 724 | 976 | 318 |
| 3352750 | 4571850 | 24500 | 137 | 187 | 319 |
| 2360000 | 3988600 | 13750 | 172 | 290 | 320 |
| 3181000 | 4326350 | 11050 | 288 | 392 | 321 |
| 3375150 | 4986200 | 19750 | 171 | 252 | 322 |
| 2745650 | 4215700 | 44200 | 62 | 95 | 323 |
| 3132150 | 4917350 | 10650 | 294 | 462 | 324 |
| 2434000 | 4372750 | 21300 | 114 | 205 | 325 |
| 1503000 | 4071950 | 18950 | 79 | 215 | 326 |
| 2660400 | 4242850 | 18650 | 143 | 227 | 327 |
| 2178800 | 3698600 | 87550 | 25 | 42 | 328 |
| 3284600 | 4637000 | 36300 | 90 | 128 | 329 |
| 2536300 | 4309250 | 46750 | 54 | 92 | 330 |
| 4660050 | 6242300 | 8900 | 524 | 701 | 331 |
| 1703150 | 4938450 | 13000 | 131 | 380 | 332 |
| 5321700 | 6197900 | 36350 | 146 | 171 | 333 |
| 2702800 | 5449550 | 14400 | 188 | 378 | 334 |
| 5341150 | 5898350 | 57300 | 93 | 103 | 335 |
| 1147100 | 4308150 | 16800 | 68 | 256 | 336 |
| 5276700 | 6618850 | 17200 | 307 | 385 | 337 |
| 2292600 | 5017450 | 40150 | 57 | 125 | 338 |
| 2137050 | 4861600 | 7050 | 303 | 690 | 339 |
| 2995700 | 5095350 | 28300 | 106 | 180 | 340 |
| 5350500 | 5310950 | 58000 | 92 | 92 | 341 |
| 4444550 | 5410400 | 425300 | 10 | 13 | 342 |
| 5246200 | 5424050 | 30450 | 172 | 178 | 343 |
| 1332300 | 3401950 | 36900 | 36 | 92 | 344 |
| 2549800 | 4586800 | 23550 | 108 | 195 | 345 |

TABLE 15
Summary of Size Exclusion Chromatography Analysis
of Hits from the Apical Domain Panning

| RT (min) | Height (mAU) | Y/N | SEQ ID NO |
|---|---|---|---|
| 5.23 | 159604 | Y | 234 |
| 5.18 | 112730 | Y | 235 |
| 5.16 | 120428 | Y | 236 |
| 5.27 | 200587 | Y | 237 |
| 5.26 | 122045 | Y | 238 |
| 5.16 | 135855 | Y | 239 |
| 5.13 | 116489 | Y | 240 |
| 5.25 | 259319 | Y | 241 |
| 5.17 | 194940 | Y | 242 |
| 5.16 | 137690 | Y | 243 |
| 5.18 | 157121 | Y | 244 |
| 5.25 | 167336 | Y | 245 |
| 5.25 | 198565 | Y | 246 |
| 5.16 | 177970 | Y | 247 |
| 5.19 | 120246 | Y | 248 |
| 5.22 | 124461 | Y | 249 |
| 5.15 | 162158 | Y | 250 |
| 5.17 | 201934 | Y | 251 |
| 5.18 | 190590 | Y | 252 |
| 5.20 | 205248 | Y | 253 |
| 5.25 | 372644 | Y | 254 |
| 5.25 | 240322 | Y | 255 |
| 5.18 | 193652 | Y | 256 |
| 5.18 | 93582 | Y | 257 |
| 5.11 | 95468 | Y | 258 |
| 5.09 | 129414 | Y | 259 |
| 5.17 | 197883 | Y | 260 |
| 5.26 | 186519 | Y | 261 |
| 5.20 | 108088 | Y | 262 |
| 5.19 | 199653 | Y | 263 |
| 5.27 | 91719 | Y | 264 |
| 5.20 | 97269 | Y | 265 |
| 5.13 | 80658 | Y | 266 |
| 5.21 | 190594 | Y | 267 |
| 5.21 | 15540 | N | 268 |
| 5.27 | 109973 | Y | 269 |
| 5.06 | 25269 | N | 270 |
| 5.18 | 183538 | Y | 271 |
| 5.13 | 153793 | Y | 272 |
| 5.27 | 98513 | Y | 273 |
| 5.12 | 118915 | Y | 274 |
| 5.16 | 166550 | Y | 275 |
| 5.19 | 69950 | Y | 276 |
| 5.26 | 153288 | Y | 277 |
| 5.19 | 162365 | Y | 278 |
| 5.21 | 158144 | Y | 279 |
| 5.16 | 184773 | Y | 280 |
| 5.20 | 86678 | Y | 281 |
| 5.44 | 157323 | Y | 282 |
| 5.24 | 10062 | N | 283 |
| 5.19 | 166917 | Y | 284 |
| 5.19 | 141207 | Y | 285 |
| 5.18 | 153272 | Y | 286 |
| 5.20 | 171129 | Y | 287 |
| 5.18 | 180935 | Y | 288 |
| 5.24 | 186261 | Y | 289 |
| 5.17 | 122862 | Y | 290 |
| 5.22 | 164619 | Y | 291 |
| 5.17 | 184513 | Y | 292 |
| 5.12 | 112152 | Y | 293 |
| 5.25 | 97549 | Y | 294 |
| 5.07 | 109730 | Y | 295 |
| 5.32 | 183183 | Y | 296 |
| 5.26 | 145537 | Y | 297 |
| 5.15 | 178190 | Y | 298 |
| 5.17 | 254468 | Y | 299 |
| 5.16 | 129224 | Y | 300 |
| 5.17 | 222741 | Y | 301 |
| 3.86 | 10594 | N | 302 |
| 5.29 | 176944 | Y | 303 |
| 5.23 | 156643 | Y | 304 |
| 5.18 | 150873 | Y | 305 |
| 3.76 | 14913 | N | 306 |
| 5.18 | 170593 | Y | 307 |
| 5.19 | 197150 | Y | 308 |
| 5.17 | 227036 | Y | 309 |
| 5.20 | 103336 | Y | 310 |
| 5.22 | 165569 | Y | 311 |
| 5.21 | 221802 | Y | 312 |
| 5.22 | 236955 | Y | 313 |
| 5.20 | 169207 | Y | 314 |
| 5.15 | 27041 | N | 315 |
| 5.20 | 200806 | Y | 316 |
| 5.20 | 233347 | Y | 317 |
| 5.20 | 62240 | Y | 318 |
| 5.22 | 220344 | Y | 319 |
| 5.15 | 268025 | Y | 320 |
| 5.21 | 241412 | Y | 321 |
| 5.31 | 198147 | Y | 322 |
| 5.22 | 209517 | Y | 323 |
| 5.18 | 12221 | N | 324 |
| 5.13 | 163609 | Y | 325 |
| 5.11 | 230271 | Y | 326 |
| 5.12 | 192181 | Y | 327 |
| 5.19 | 150792 | Y | 328 |
| | | N | 329 |
| 5.12 | 189574 | N | 330 |
| 5.12 | 102760 | Y | 331 |
| 5.13 | 158316 | Y | 332 |
| 5.12 | 118085 | Y | 333 |
| 5.20 | 164575 | Y | 334 |
| 5.09 | 120605 | Y | 335 |
| 5.12 | 120392 | Y | 336 |
| 5.16 | 191574 | Y | 337 |
| 5.15 | 197840 | Y | 338 |
| 5.17 | 162216 | Y | 339 |
| 5.15 | 134469 | Y | 340 |
| 5.14 | 106164 | Y | 341 |
| 5.11 | 110880 | Y | 342 |
| 5.12 | 131970 | Y | 343 |
| 5.15 | 167831 | Y | 344 |
| 5.28 | 154623 | Y | 345 |

### Screen 4 (ISOP876-877)

The libraries underwent selection against 5 rounds of panning against decreasing concentrations of human apical domains (which was 10000 biotinylated) followed by 3 rounds of off-rate selection against “cold” non-biotinylated apical domain. Colonies were screened in primary ELISA against CD71-ECD and apical domain (compared to the negative control of HSA) and only selected hits that exhibited binding to both antigens. ELISA data are shown in Table 16. Purification data shown in Table 17.

TABLE 16
Summary of Screening Hits: Primary Elisa
Screen Against CD71 and Apical Domain

| | Apical | | | | SEQ |
|---|---|---|---|---|---|
| hCD71, | domain, | HSA, | | Apical | ID |
| RLU | RLU | RLU | hCD71:HSA | domain:HSA | NO |
| 5004250 | 6819200 | 82300 | 61 | 83 | 346 |
| 4102600 | 5950000 | 35500 | 116 | 168 | 347 |
| 1827400 | 4797150 | 15900 | 115 | 302 | 348 |
| 6371800 | 6870350 | 295450 | 22 | 23 | 349 |
| 5957550 | 6701850 | 47000 | 127 | 143 | 350 |
| 2668900 | 2865800 | 8300 | 322 | 345 | 351 |
| 2381350 | 5424850 | 37650 | 63 | 144 | 352 |
| 4541400 | 6869200 | 85450 | 53 | 80 | 353 |
| 5747900 | 5249400 | 37050 | 155 | 142 | 354 |
| 3584550 | 5914650 | 13950 | 257 | 424 | 355 |
| 3921700 | 6368950 | 18900 | 207 | 337 | 356 |
| 5623550 | 6340400 | 343250 | 16 | 18 | 357 |
| 2344500 | 5019350 | 60650 | 39 | 83 | 358 |
| 3569550 | 6968750 | 29850 | 120 | 233 | 359 |
| 5784400 | 5365550 | 37200 | 155 | 144 | 360 |
| 3077900 | 5005250 | 14650 | 210 | 342 | 361 |
| 2236550 | 4864600 | 47550 | 47 | 102 | 362 |
| 4183450 | 4909000 | 15350 | 273 | 320 | 363 |
| 5833250 | 6503950 | 97200 | 60 | 67 | 364 |
| 6849650 | 6695550 | 102850 | 67 | 65 | 365 |
| 2294650 | 3368650 | 10750 | 213 | 313 | 366 |
| 4618800 | 6510400 | 99000 | 47 | 66 | 367 |
| 5530000 | 6232300 | 25350 | 218 | 246 | 368 |
| 3817900 | 3662100 | 22250 | 172 | 165 | 369 |
| 2131500 | 4827000 | 10050 | 212 | 480 | 370 |
| 2084600 | 4403000 | 46400 | 45 | 95 | 371 |
| 2801450 | 4631400 | 22600 | 124 | 205 | 372 |
| 4860150 | 5730000 | 49700 | 98 | 115 | 373 |
| 1004100 | 3637750 | 13150 | 76 | 277 | 374 |
| 2818900 | 2093350 | 15100 | 187 | 139 | 375 |
| 5955400 | 6470750 | 38800 | 153 | 167 | 376 |
| 1572750 | 3434700 | 12550 | 125 | 274 | 377 |
| 6117750 | 6119200 | 80750 | 76 | 76 | 378 |
| 1395250 | 5134200 | 21450 | 65 | 239 | 379 |
| 5318400 | 6496600 | 257300 | 21 | 25 | 380 |
| 2477650 | 5139100 | 22600 | 110 | 227 | 381 |
| 2013650 | 2552300 | 12550 | 160 | 203 | 382 |
| 1849700 | 4325500 | 19650 | 94 | 220 | 383 |
| 2035200 | 5554250 | 96600 | 21 | 57 | 384 |
| 5744150 | 5912400 | 40450 | 142 | 146 | 385 |
| 1283450 | 3955800 | 9900 | 130 | 400 | 386 |
| 1786250 | 3878100 | 8800 | 203 | 441 | 387 |
| 1091100 | 4293900 | 31700 | 34 | 135 | 388 |
| 5821400 | 6193000 | 48150 | 121 | 129 | 389 |
| 1929750 | 4775750 | 36550 | 53 | 131 | 390 |
| 2778350 | 4192850 | 25750 | 108 | 163 | 391 |
| 4766650 | 3899600 | 20100 | 237 | 194 | 392 |
| 4387800 | 6085300 | 105500 | 42 | 58 | 393 |
| 2862800 | 5657250 | 13150 | 218 | 430 | 394 |
| 5114250 | 6587550 | 71250 | 72 | 92 | 395 |
| 2226050 | 2473050 | 13500 | 165 | 183 | 396 |
| 1652800 | 5570450 | 66100 | 25 | 84 | 397 |
| 2120550 | 2069050 | 16100 | 132 | 129 | 398 |
| 5882550 | 5040200 | 19800 | 297 | 255 | 399 |
| 5667150 | 6538700 | 40150 | 141 | 163 | 400 |
| 5643950 | 5822150 | 138400 | 41 | 42 | 401 |
| 2171000 | 4774700 | 10400 | 209 | 459 | 402 |
| 3637750 | 5340950 | 15600 | 233 | 342 | 403 |
| 4717400 | 5655300 | 38650 | 122 | 146 | 404 |
| 1947700 | 3467050 | 18000 | 108 | 193 | 405 |
| 1421750 | 3683750 | 7700 | 185 | 478 | 406 |
| 1726650 | 4635450 | 75650 | 23 | 61 | 407 |
| 2262650 | 3227050 | 19550 | 116 | 165 | 408 |
| 4914500 | 6203750 | 21850 | 225 | 284 | 409 |
| 3895900 | 3204700 | 8450 | 46 | 379 | 410 |
| 5229050 | 6359400 | 20000 | 261 | 318 | 411 |
| 4369000 | 5997650 | 39150 | 112 | 153 | 412 |
| 3862850 | 4980700 | 13450 | 287 | 370 | 413 |
| 6714950 | 6175350 | 78500 | 86 | 79 | 414 |
| 3507100 | 3614150 | 7000 | 501 | 516 | 415 |
| 6599800 | 6322050 | 121450 | 54 | 52 | 416 |
| 4506600 | 6173800 | 14800 | 305 | 417 | 417 |
| 1953100 | 5053000 | 65450 | 30 | 77 | 418 |
| 3599900 | 4590450 | 17050 | 211 | 269 | 419 |
| 3198550 | 3934600 | 7950 | 402 | 495 | 420 |
| 1763000 | 4197200 | 10550 | 167 | 398 | 42 |
| 2965150 | 5879300 | 17300 | 171 | 340 | 422 |
| 6626050 | 6555600 | 114300 | 58 | 57 | 423 |
| 3901400 | 5158950 | 33350 | 117 | 155 | 424 |
| 4439100 | 6232500 | 17950 | 247 | 347 | 425 |
| 4428600 | 5949700 | 33800 | 13 | 176 | 426 |
| 2252100 | 3520550 | 5750 | 392 | 612 | 427 |
| 6188900 | 5895050 | 31700 | 195 | 186 | 428 |
| 1954600 | 4390450 | 15700 | 124 | 280 | 429 |
| 3635200 | 3580050 | 28900 | 126 | 124 | 430 |
| 4403450 | 4347000 | 49400 | 89 | 88 | 431 |
| 3734050 | 3109850 | 39600 | 94 | 79 | 432 |
| 3260500 | 3565200 | 19350 | 169 | 184 | 433 |
| 3133150 | 4033450 | 10000 | 313 | 403 | 434 |
| 3621350 | 5499100 | 17250 | 210 | 319 | 435 |
| 4459600 | 5658350 | 19650 | 227 | 288 | 436 |
| 3224550 | 2824600 | 19200 | 168 | 147 | 437 |
| 4962250 | 5590750 | 34750 | 143 | 161 | 438 |
| 5470400 | 6560700 | 25450 | 215 | 258 | 439 |
| 2255000 | 4877400 | 25350 | 89 | 192 | 440 |
| 5923500 | 5735500 | 186600 | 32 | 31 | 441 |
| 4923000 | 6227400 | 28700 | 172 | 217 | 442 |
| 5383550 | 4400500 | 38000 | 142 | 116 | 443 |
| 2799300 | 5124950 | 57850 | 48 | 89 | 444 |
| 6649900 | 6734300 | 87850 | 76 | 77 | 445 |
| 4746250 | 5506550 | 129950 | 37 | 42 | 446 |
| 5856050 | 5252550 | 38550 | 152 | 136 | 447 |
| 4727700 | 4760900 | 63950 | 74 | 74 | 448 |
| 6747250 | 6506750 | 98000 | 69 | 66 | 449 |
| 4097550 | 6262600 | 24750 | 166 | 253 | 450 |
| 5226150 | 6084450 | 40400 | 129 | 151 | 451 |
| 5241100 | 4230600 | 59700 | 88 | 71 | 452 |
| 4947200 | 5120050 | 34750 | 142 | 147 | 453 |
| 4184500 | 4201700 | 18550 | 226 | 227 | 454 |
| 1561500 | 4830350 | 28300 | 55 | 171 | 455 |
| 4504650 | 4308400 | 14900 | 302 | 289 | 456 |
| 5539400 | 5784300 | 109150 | 51 | 53 | 457 |
| 3443650 | 4640900 | 67500 | 51 | 69 | 458 |
| 2502200 | 2607750 | 13100 | 191 | 199 | 459 |
| 2686650 | 2797850 | 18850 | 143 | 148 | 460 |
| 3766600 | 5351050 | 32200 | 117 | 166 | 461 |
| 2827700 | 3278300 | 37400 | 76 | 88 | 462 |
| 5619750 | 5512100 | 61700 | 91 | 89 | 463 |
| 4943900 | 5678950 | 35400 | 140 | 160 | 464 |
| 3858800 | 4152400 | 30000 | 129 | 138 | 465 |
| 1082500 | 4303950 | 15400 | 70 | 279 | 466 |
| 1178600 | 2003000 | 44350 | 27 | 45 | 467 |
| 1052750 | 3583350 | 14850 | 71 | 24 | 468 |
| 5395400 | 5563700 | 74000 | 73 | 75 | 469 |
| 2469650 | 4742400 | 31100 | 79 | 152 | 470 |
| 1648500 | 3885000 | 39500 | 42 | 98 | 47 |
| 4516950 | 4966350 | 27950 | 162 | 178 | 472 |
| 2909200 | 3025100 | 33100 | 88 | 91 | 473 |
| 3041450 | 2715850 | 9750 | 312 | 279 | 474 |
| 3463850 | 3208200 | 34300 | 101 | 94 | 475 |
| 4146900 | 3397750 | 15800 | 262 | 215 | 476 |
| 1879300 | 2405000 | 6500 | 289 | 370 | 477 |
| 5389450 | 5897000 | 96900 | 56 | 61 | 478 |
| 2661550 | 4825300 | 12950 | 206 | 373 | 479 |
| 1658050 | 4271500 | 23600 | 70 | 181 | 480 |
| 3711450 | 3653600 | 4550 | 816 | 803 | 481 |
| 2226750 | 2657150 | 9000 | 247 | 295 | 482 |
| 3047450 | 3468200 | 22450 | 136 | 154 | 483 |
| 1467450 | 4691300 | 41400 | 35 | 113 | 484 |
| 2473900 | 3229250 | 37650 | 66 | 86 | 485 |
| 1749700 | 1978400 | 5900 | 297 | 335 | 486 |
| 2274250 | 3425700 | 15600 | 146 | 220 | 487 |
| 3177550 | 6136200 | 59650 | 53 | 103 | 488 |
| 2807050 | 4063100 | 11950 | 235 | 340 | 489 |
| 4150050 | 2896850 | 10300 | 403 | 281 | 490 |
| 4663900 | 5067900 | 17600 | 265 | 288 | 49 |
| 4846500 | 5103800 | 31600 | 153 | 162 | 492 |
| 2750900 | 2500900 | 14500 | 190 | 172 | 493 |
| 3202500 | 3322400 | 9000 | 356 | 369 | 494 |
| 1139500 | 4029450 | 8000 | 142 | 504 | 495 |
| 5271250 | 5662150 | 121450 | 43 | 47 | 496 |
| 2931300 | 4584650 | 12800 | 229 | 358 | 497 |
| 5697200 | 5673000 | 50550 | 113 | 112 | 498 |
| 2777050 | 3127300 | 21100 | 132 | 148 | 499 |
| 2951000 | 3390300 | 20050 | 147 | 169 | 500 |
| 3214050 | 4880350 | 8050 | 399 | 606 | 501 |
| 1487350 | 2341100 | 10550 | 141 | 222 | 502 |
| 2352400 | 2263300 | 15650 | 150 | 145 | 503 |
| 1220600 | 2678250 | 15550 | 78 | 172 | 504 |
| 5383750 | 5644050 | 103800 | 52 | 54 | 505 |
| 1640300 | 4735650 | 42150 | 39 | 112 | 506 |
| 3516600 | 4834200 | 19750 | 178 | 245 | 507 |
| 4218800 | 3553800 | 16000 | 264 | 222 | 508 |
| 1742650 | 4600200 | 13050 | 134 | 353 | 509 |
| 3389500 | 5212700 | 12900 | 263 | 404 | 510 |
| 5027150 | 5475550 | 102600 | 49 | 53 | 511 |
| 2060350 | 3866750 | 16600 | 124 | 233 | 512 |
| 2516300 | 2716650 | 13500 | 186 | 201 | 513 |
| 5919150 | 5942000 | 49850 | 119 | 119 | 514 |
| 5141400 | 4672900 | 53150 | 97 | 88 | 515 |
| 4732700 | 4671350 | 17700 | 267 | 264 | 516 |
| 3115050 | 3641650 | 21900 | 142 | 166 | 517 |
| 1849900 | 3336350 | 12300 | 150 | 271 | 518 |
| 3245000 | 5241250 | 20750 | 156 | 253 | 519 |
| 5339400 | 5259900 | 124150 | 43 | 42 | 520 |
| 5622600 | 5380000 | 30300 | 186 | 178 | 521 |
| 3913150 | 3483450 | 12300 | 318 | 283 | 522 |
| 4186250 | 4308000 | 14350 | 292 | 300 | 523 |
| 3224600 | 3950200 | 17000 | 190 | 232 | 524 |
| 6024450 | 5926200 | 123100 | 49 | 48 | 525 |
| 3700800 | 5367950 | 7450 | 497 | 721 | 526 |
| 6590800 | 6400300 | 436050 | 15 | 15 | 527 |
| 5178400 | 5578200 | 30650 | 169 | 182 | 528 |
| 4128100 | 3243300 | 8850 | 466 | 366 | 529 |
| 4610450 | 4477100 | 15950 | 289 | 281 | 530 |
| 1604800 | 4158200 | 7100 | 226 | 586 | 531 |
| 1446900 | 4586850 | 23300 | 62 | 197 | 532 |
| 3181650 | 5252150 | 21800 | 146 | 241 | 533 |
| 5196200 | 5756200 | 27750 | 187 | 207 | 534 |
| 4291650 | 3376650 | 11850 | 362 | 285 | 535 |
| 2988550 | 3264700 | 21250 | 141 | 154 | 536 |
| 2420650 | 5138800 | 103450 | 23 | 50 | 537 |
| 5568900 | 4932850 | 30050 | 185 | 164 | 538 |
| 4939100 | 6082350 | 29650 | 167 | 205 | 539 |
| 3786700 | 5848350 | 19100 | 198 | 306 | 540 |
| 1790050 | 3424850 | 24500 | 73 | 140 | 541 |
| 2133500 | 3002750 | 12650 | 169 | 237 | 542 |
| 5290750 | 4648050 | 25600 | 207 | 182 | 543 |
| 4040250 | 4067200 | 16750 | 241 | 243 | 544 |
| 4215900 | 4219400 | 12900 | 327 | 327 | 545 |
| 3533250 | 4404100 | 9450 | 374 | 466 | 546 |
| 4594300 | 5347650 | 16600 | 277 | 322 | 547 |
| 4734600 | 5287950 | 20550 | 230 | 257 | 548 |
| 2855850 | 3490350 | 14450 | 198 | 242 | 549 |
| 4793550 | 5803750 | 34100 | 141 | 170 | 550 |
| 1166050 | 4067700 | 25700 | 45 | 158 | 551 |
| 4951150 | 5697200 | 41700 | 119 | 137 | 552 |

TABLE 17
Summary of Size Exclusion Chromatography Analysis
of Hits from the Apical Domain Panning

| RT (min) | Height (mAU) | Y/N | SEQ ID NO |
|---|---|---|---|
| 5.08 | 20782 | Y | 346 |
| 5.10 | 214536 | Y | 347 |
| 5.32 | 311623 | Y | 348 |
| 5.09 | 174959 | Y | 349 |
| 5.12 | 235609 | Y | 350 |
| 5.09 | 213666 | Y | 351 |
| 5.23 | 325250 | Y | 352 |
| 5.13 | 195580 | Y | 353 |
| 5.09 | 224675 | Y | 354 |
| 5.13 | 178885 | Y | 355 |
| 5.10 | 194286 | Y | 356 |
| 5.14 | 269862 | Y | 357 |
| 5.15 | 324794 | Y | 358 |
| 5.07 | 226548 | Y | 359 |
| 5.12 | 219501 | Y | 360 |
| 5.09 | 187372 | Y | 361 |
| 5.23 | 268526 | Y | 362 |
| 5.14 | 203392 | Y | 363 |
| 5.04 | 182426 | Y | 364 |
| 5.17 | 185848 | Y | 365 |
| 5.13 | 299132 | Y | 366 |
| 5.09 | 289015 | Y | 367 |
| 5.15 | 212034 | Y | 368 |
| 5.30 | 160950 | N | 369 |
| 5.11 | 213295 | Y | 370 |
| 5.21 | 236161 | N | 371 |
| 5.06 | 186116 | Y | 372 |
| 5.11 | 248229 | Y | 373 |
| 5.10 | 248499 | Y | 374 |
| 5.05 | 198152 | Y | 375 |
| 5.09 | 211623 | Y | 376 |
| 5.06 | 198167 | Y | 377 |
| 5.14 | 207850 | Y | 378 |
| 5.25 | 315070 | Y | 379 |
| 5.12 | 222227 | Y | 380 |
| 5.24 | 386569 | Y | 381 |
| 5.03 | 171410 | Y | 382 |
| 5.08 | 236517 | Y | 383 |
| 5.21 | 382595 | Y | 384 |
| 5.12 | 225346 | Y | 385 |
| 5.18 | 187737 | Y | 386 |
| 5.14 | 327986 | Y | 387 |
| 5.22 | 373098 | Y | 388 |
| 5.08 | 184050 | Y | 389 |
| 5.21 | 333818 | Y | 390 |
| 5.19 | 122289 | Y | 391 |
| 5.08 | 205702 | Y | 392 |
| 5.17 | 225672 | Y | 393 |
| 5.10 | 215908 | Y | 394 |
| 5.15 | 227519 | Y | 395 |
| 5.08 | 213911 | Y | 396 |
| 5.15 | 193936 | N | 397 |
| 5.09 | 214587 | Y | 398 |
| 5.13 | 211676 | Y | 399 |
| 5.03 | 221861 | Y | 400 |
| 5.15 | 233719 | Y | 401 |
| 5.26 | 175921 | Y | 402 |
| 5.04 | 249944 | Y | 403 |
| 5.06 | 186208 | Y | 404 |
| 5.06 | 250946 | Y | 405 |
| 5.08 | 218970 | Y | 406 |
| 5.22 | 370359 | Y | 407 |
| 5.20 | 283952 | Y | 408 |
| 5.10 | 205863 | Y | 409 |
| 5.24 | 201927 | Y | 410 |
| 5.09 | 207997 | Y | 411 |
| 5.10 | 238297 | Y | 412 |
| 5.17 | 245260 | Y | 413 |
| 5.16 | 245827 | Y | 414 |
| 5.07 | 245255 | Y | 415 |
| 5.15 | 311750 | Y | 416 |
| 5.08 | 191229 | Y | 417 |
| 5.20 | 28297 | N | 418 |
| 5.11 | 199365 | Y | 419 |
| 5.10 | 246124 | Y | 420 |
| 5.10 | 200926 | Y | 421 |
| 5.03 | 240173 | Y | 422 |
| 5.12 | 240604 | Y | 423 |
| 5.08 | 221842 | Y | 424 |
| 5.11 | 232202 | Y | 425 |
| 5.14 | 201571 | Y | 426 |
| 5.18 | 226458 | Y | 427 |
| 5.19 | 233268 | Y | 428 |
| 5.28 | 228232 | Y | 429 |
| 5.19 | 331225 | Y | 430 |
| 5.29 | 153716 | N | 431 |
| 5.09 | 240745 | Y | 432 |
| 5.08 | 209161 | Y | 433 |
| 5.08 | 184413 | Y | 434 |
| 5.03 | 258759 | Y | 435 |
| 5.11 | 234176 | Y | 436 |
| 5.22 | 292682 | Y | 437 |
| 5.24 | 125117 | Y | 438 |
| 5.17 | 170548 | Y | 439 |
| 5.37 | 105993 | Y | 440 |
| 5.36 | 201456 | Y | 441 |
| 5.20 | 76477 | N | 442 |
| 5.15 | 142752 | Y | 443 |
| 5.26 | 187989 | Y | 444 |
| 5.17 | 159068 | Y | 445 |
| 5.23 | 110048 | Y | 446 |
| 5.25 | 72838 | Y | 447 |
| 5.26 | 149198 | Y | 448 |
| 5.19 | 210081 | Y | 449 |
| 5.16 | 155090 | Y | 450 |
| 5.16 | 127494 | Y | 451 |
| 5.18 | 120868 | Y | 452 |
| 5.27 | 161437 | Y | 453 |
| 5.17 | 158883 | Y | 454 |
| 5.27 | 47905 | Y | 455 |
| 5.24 | 143380 | Y | 456 |
| 5.21 | 152378 | Y | 457 |
| 5.17 | 167637 | Y | 458 |
| 5.20 | 135082 | Y | 459 |
| 5.21 | 96649 | Y | 460 |
| 5.22 | 40718 | N | 461 |
| 5.23 | 181620 | Y | 462 |
| 4.82 | 160505 | Y | 463 |
| 5.19 | 166460 | Y | 464 |
| 5.17 | 189283 | Y | 465 |
| 5.26 | 176942 | Y | 466 |
| 4.61 | 45187 | N | 467 |
| 5.22 | 215380 | Y | 468 |
| 5.22 | 101529 | Y | 469 |
| 5.20 | 108402 | Y | 470 |
| 5.26 | 60971 | Y | 471 |
| 5.16 | 146811 | Y | 472 |
| 5.27 | 171478 | Y | 473 |
| 5.15 | 110923 | Y | 474 |
| 5.19 | 123571 | Y | 475 |
| 5.18 | 145925 | Y | 476 |
| 5.21 | 124544 | Y | 477 |
| 5.26 | 184123 | Y | 478 |
| 5.23 | 151123 | Y | 479 |
| 5.22 | 204830 | Y | 480 |
| 5.26 | 61290 | Y | 481 |
| 5.17 | 95471 | Y | 482 |
| 5.17 | 192045 | Y | 483 |
| 4.54 | 120472 | N | 484 |
| 4.99 | 153668 | Y | 485 |
| 5.15 | 144204 | Y | 486 |
| 5.18 | 120403 | Y | 487 |
| 5.24 | 251317 | Y | 488 |
| 5.22 | 109481 | Y | 489 |
| 5.20 | 91950 | Y | 490 |
| 5.21 | 153373 | Y | 49 |
| 5.40 | 193558 | Y | 492 |
| 5.24 | 113050 | Y | 493 |
| 5.23 | 143252 | Y | 494 |
| 5.18 | 40953 | Y | 495 |
| 5.38 | 176055 | Y | 496 |
| 5.29 | 150560 | Y | 497 |
| 5.36 | 155352 | Y | 498 |
| 5.25 | 104525 | Y | 499 |
| 5.17 | 125973 | Y | 500 |
| 5.18 | 93761 | Y | 50 |
| 5.17 | 136313 | Y | 502 |
| 5.27 | 192389 | Y | 503 |
| 5.19 | 137135 | Y | 504 |
| 5.13 | 190127 | Y | 505 |
| 5.29 | 66575 | N | 506 |
| 5.18 | 131428 | Y | 507 |
| 5.17 | 117733 | Y | 508 |
| 5.27 | 131576 | Y | 509 |
| 5.23 | 267973 | Y | 510 |
| 5.19 | 121126 | Y | 511 |
| 5.18 | 172260 | Y | 512 |
| 5.21 | 122955 | Y | 513 |
| 5.14 | 140797 | Y | 514 |
| 5.28 | 150991 | Y | 515 |
| 5.28 | 161288 | Y | 516 |
| 5.30 | 104897 | Y | 517 |
| 5.25 | 117192 | Y | 518 |
| 5.18 | 25319 | N | 519 |
| 5.41 | 172925 | Y | 520 |
| 5.42 | 174298 | Y | 521 |
| 5.28 | 44791 | Y | 522 |
| 5.14 | 60451 | Y | 523 |
| 5.12 | 138040 | Y | 524 |
| 5.23 | 90777 | Y | 525 |
| 5.20 | 149998 | Y | 526 |
| 5.23 | 127718 | Y | 527 |
| 5.22 | 172047 | Y | 528 |
| 5.22 | 120203 | Y | 529 |
| 5.14 | 244655 | Y | 530 |
| 5.08 | 255853 | Y | 531 |
| 5.28 | 145771 | Y | 532 |
| 5.28 | 288899 | Y | 533 |
| 5.11 | 134382 | N | 534 |
| 5.07 | 202318 | Y | 535 |
| 5.18 | 352815 | Y | 536 |
| 5.06 | 257381 | Y | 537 |
| 5.13 | 214393 | Y | 538 |
| 5.14 | 178860 | Y | 539 |
| 5.16 | 259554 | Y | 540 |
| 5.12 | 247679 | Y | 541 |
| 5.07 | 230388 | Y | 542 |
| 5.08 | 203297 | Y | 543 |
| 5.09 | 227841 | Y | 544 |
| 5.19 | 266682 | Y | 545 |
| 5.07 | 204094 | Y | 546 |
| 5.22 | 33838 | Y | 547 |
| 5.16 | 181831 | Y | 548 |
| 5.15 | 240700 | Y | 549 |
| 5.09 | 254408 | Y | 550 |
| 5.11 | 245458 | Y | 551 |
| 5.13 | 145390 | Y | 552 |

### Example 5. Binding to Human and Cynomolgus CD71

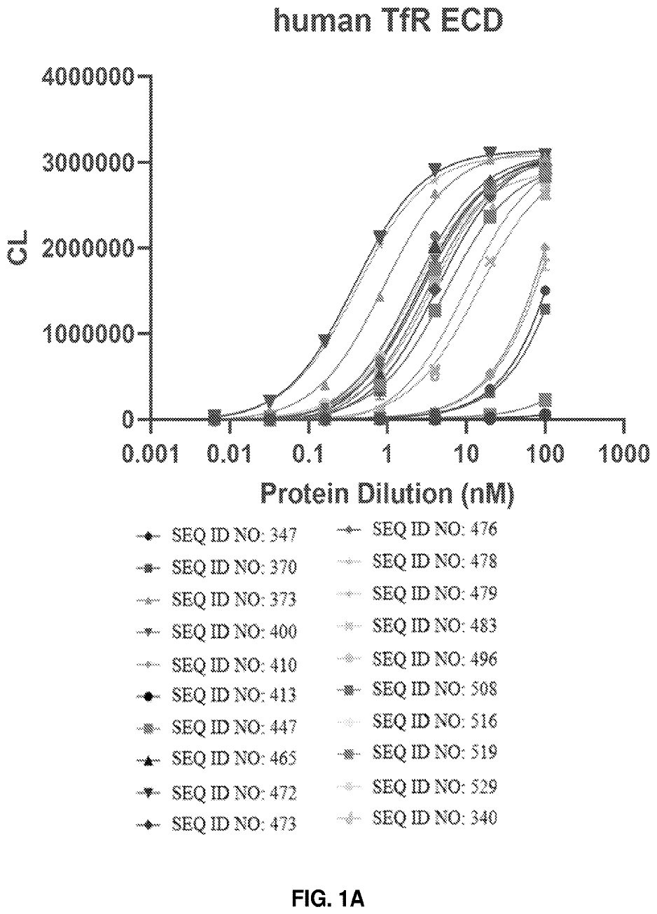

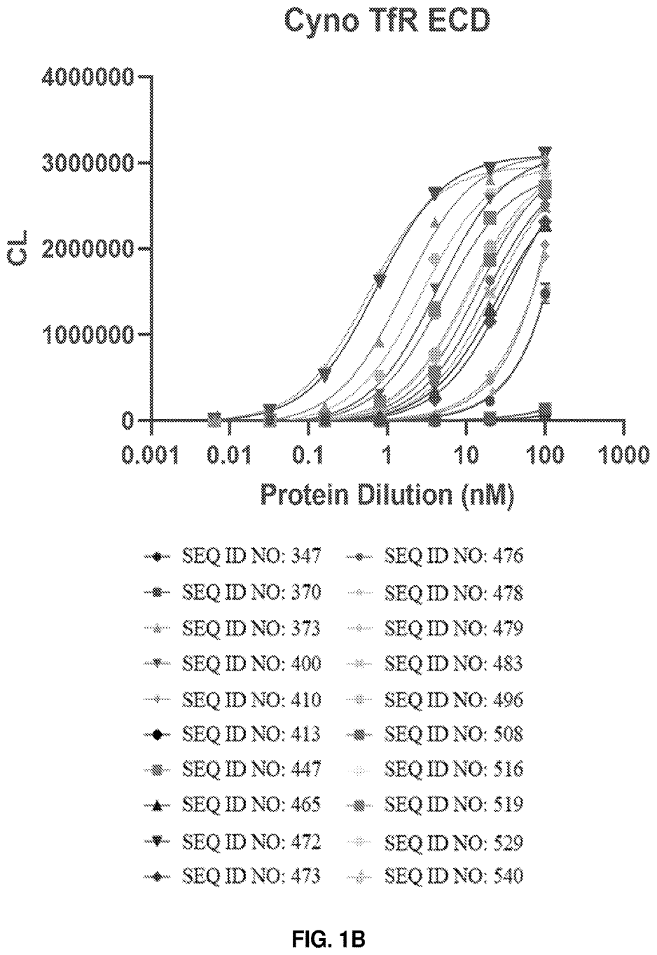

Select CD71-binding FN3 domains identified in Example 4 above were chosen to determine their binding to both human and cynomolgus monkey CD71 extracellular domain (CD71-ECD). Capture ELISA using CD71 binding to FN3 domains was performed, with the FN3 domains being titrated from 250 nM to 0.3 nM. 20 nM of antigen was incubated on a Neutravidin® plate for 1 hour at room temperature. The FN3 domains were incubated for 16 hours at room temperature. The results for human CD71-ECD and cynomolgus CD71-ECD are shown in FIGS. 1A and 1B, respectively. The EC50 results are shown below in Table 18.

TABLE 18
Human and Cynomolgus EC50 Binding Results

| SEQ ID NO | Human EC50 nM | Cyno EC50 nM |
|---|---|---|
| 347 | ND | ND |
| 370 | ND | ND |
| 373 | 0.91 | 1.6 |
| 400 | 2.8 | 4.3 |
| 410 | ND | ND |
| 413 | ND | ND |
| 447 | 3.2 | 5.0 |
| 465 | 2.5 | 24.9 |
| 472 | 0.4 | 0.7 |
| 473 | 4.3 | 34.9 |
| 476 | 2.1 | 17.7 |
| 478 | 0.4 | 0.6 |
| 479 | ND | ND |
| 483 | 14.1 | 23.0 |
| 496 | 3.9 | 10.6 |
| 508 | 5.5 | 14.2 |
| 516 | 2.2 | 10.6 |
| 519 | ND | ND |
| 529 | 2.3 | 2.7 |
| 540 | 10.0 | ND |

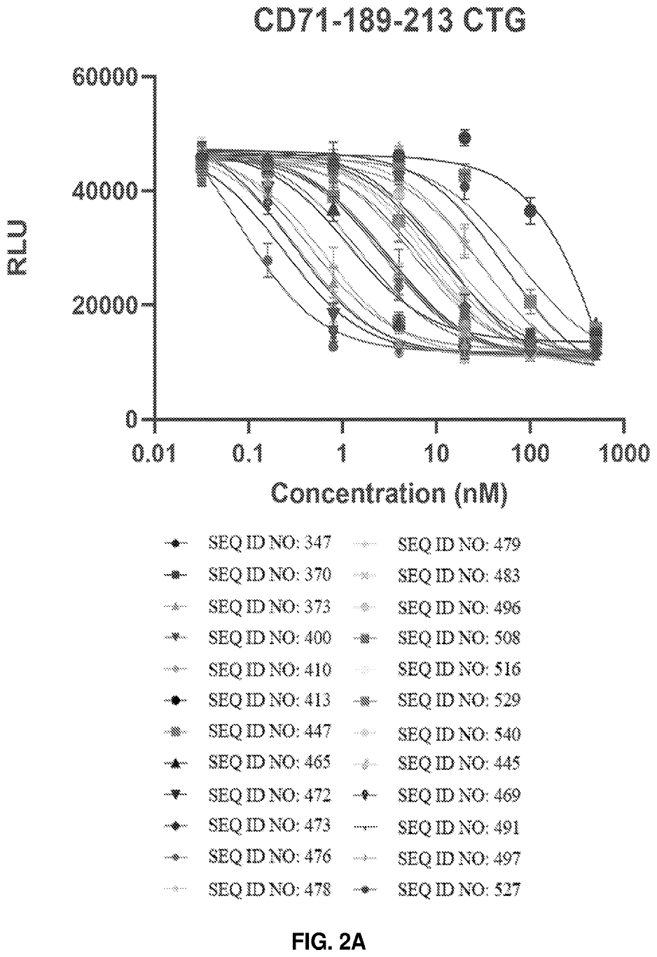

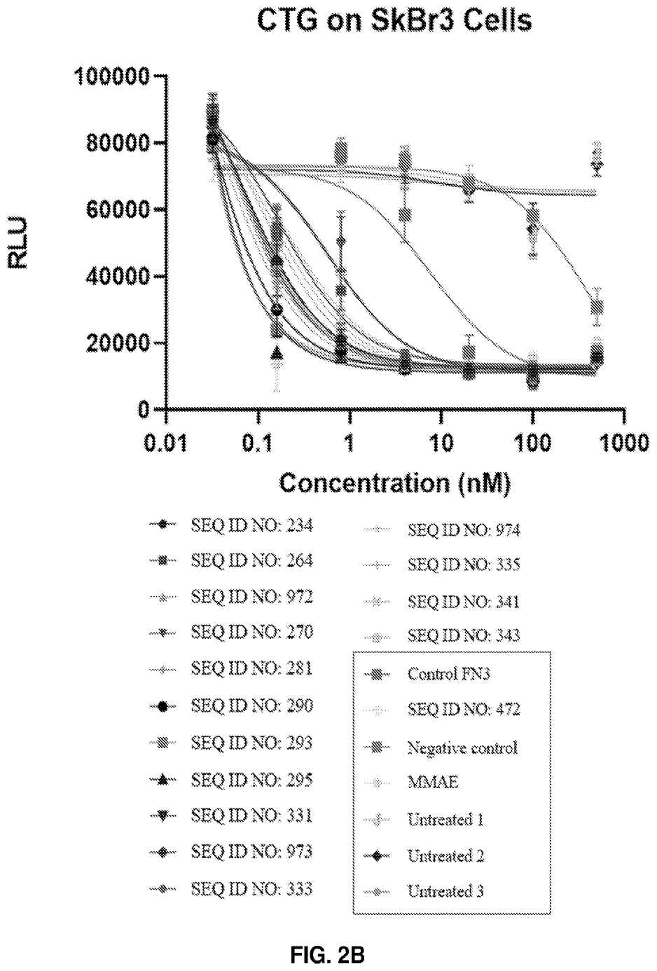

To determine cell internalization, a CellTiter-Glo® luminescent assay was performed with two different groups of selected CD71-binding FN3 domains identified in Example 4 above. SKBR3 human breast cancer cells were used, with 5,000 cells plated per well. The assay was performed with 7-point, 5-fold titration from 500 nM, and luminescence was run 72 hours after treatment. Results from group 1 and group 2 are shown in FIGS. 2A and 2B, respectively, with EC50 results shown in Tables 19 and 20, respectively.

TABLE 19
Group 1 CTG EC50 Binding Results

| SEQ ID NO | EC50 nM |
|---|---|
| 347 | 53.4 |
| 370 | 10.9 |
| 373 | 0.4 |
| 400 | 2.9 |
| 410 | 5.8 |
| 413 | ND |
| 447 | 71.5 |
| 465 | 1.2 |
| 472 | 0.3 |
| 473 | 12.6 |
| 476 | 2.7 |
| 478 | 0.6 |
| 479 | 30.6 |
| 483 | 9.8 |
| 496 | 3.0 |
| 508 | 1.5 |
| 516 | 8.4 |
| 529 | 7.0 |
| 540 | 14.4 |
| 445 | 0.4 |
| 469 | 0.2 |
| 491 | 3.2 |
| 497 | 31.3 |
| 527 | 0.1 |

TABLE 20
Group 2 CTG EC50 Binding Results

| SEQ ID NO | EC50 nM |
|---|---|
| 234 | 0.07 |
| 264 | 0.16 |
| 972 | 0.04 |
| 270 | 0.57 |
| 281 | 0.06 |
| 290 | 0.01 |
| 293 | 0 |
| 295 | 0 |
| 331 | 0.05 |
| 973 | 0.07 |
| 333 | 0.57 |
| 974 | 0.15 |
| 335 | 0.04 |
| 341 | 0.11 |
| 343 | 0.21 |

### Example 6. Luciferase Reporter Assay for CD71-Binding FN3 Domains Conjugated to KRAS-Targeting siRNA

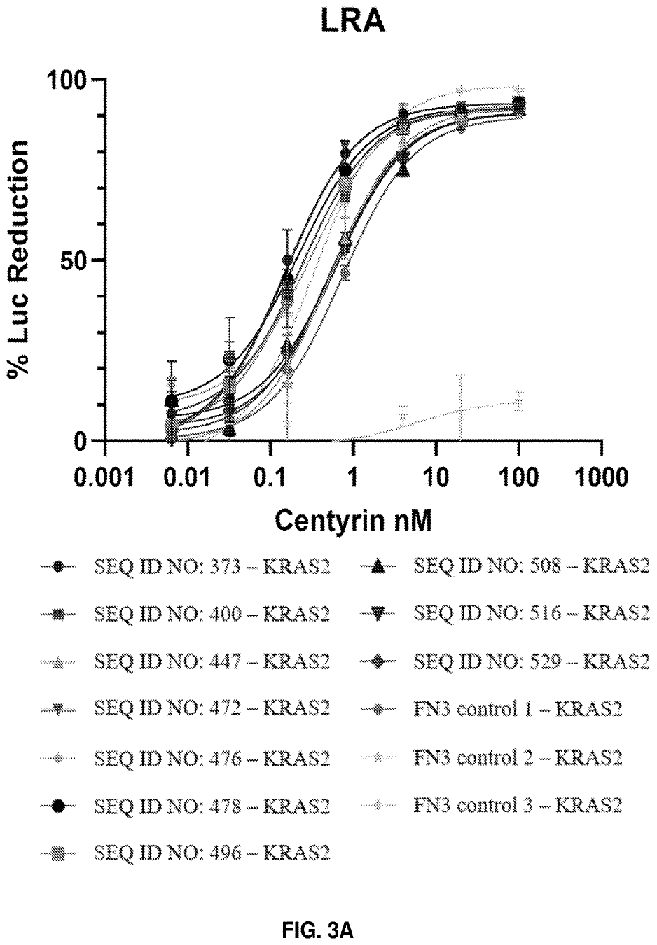

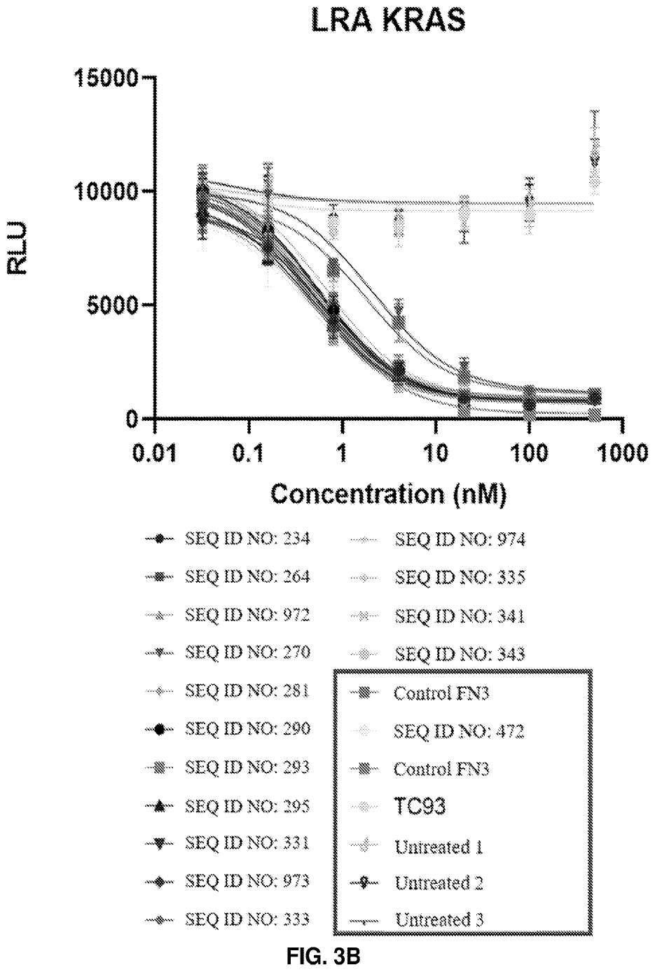

In order to determine if CD71-binding FN3 domains can deliver a gene cargo, a luciferase reporter assay (LSA) was performed with the same two groups of CD71-binding FN3 domains as in Example 5. SW620-rLuc-KRAS cells were plated in 96-well tissue culture treated plates, with 3000 cells per well. The FN3 domains were conjugated with KRAS2 tool siRNA and then were diluted in media from 100 mM to 0.006 mM. The cells were treated with the FN3-KRAS2 siRNA conjugates for 72 hours. Finally, EnduRen™ live cell substrate (Promega®) was added to the cells for 4 hours before analysis. Results from group 1 and group 2 are shown in FIGS. 3A and 3B, respectively, with EC50 results shown in Tables 21 and 22, respectively.

TABLE 21
Group 1 LSA EC50 Binding Results

| SEQ ID NO of FN3 Domain | |
|---|---|
| (Conjugated to KRAS2 siRNA) | EC50 nM |
| 373 | 0.15 |
| 400 | 0.24 |
| 447 | 0.53 |
| 472 | 0.15 |
| 476 | 0.31 |
| 478 | 0.21 |
| 496 | 0.22 |
| 508 | 0.6 |
| 516 | 0.6 |
| 529 | 0.52 |
| FN3 control 1 | 0.72 |
| FN3 control 2 | 0.33 |
| Negative control | ND |

TABLE 22
Group 2 LSA EC50 Binding Results

| SEQ ID NO of FN3 Domain | |
|---|---|
| (Conjugated to KRAS2 siRNA) | EC50 nM |
| 234 | 0.63 |
| 264 | 0.45 |
| 972 | 0.49 |
| 270 | 2.1 |
| 281 | 0.39 |
| 290 | 0.57 |
| 293 | 0.54 |
| 295 | 0.47 |
| 331 | 0.75 |
| 973 | 0.48 |
| 333 | 0.58 |
| 974 | 0.53 |
| 335 | 0.47 |
| 341 | 0.67 |
| 343 | 0.64 |
| FN3 control 1 | 1.79 |
| FN3 control 2 | 0.59 |
| Negative control | ND |

### Example 7. CD71-Binding FN3 Domains Conjugated to AHSA1-Targeting siRNA

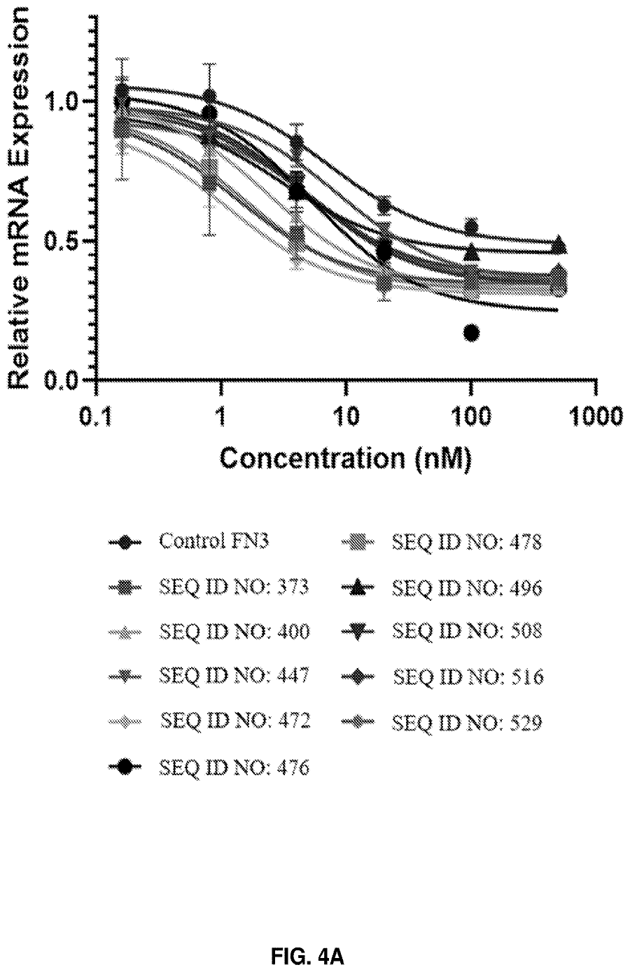

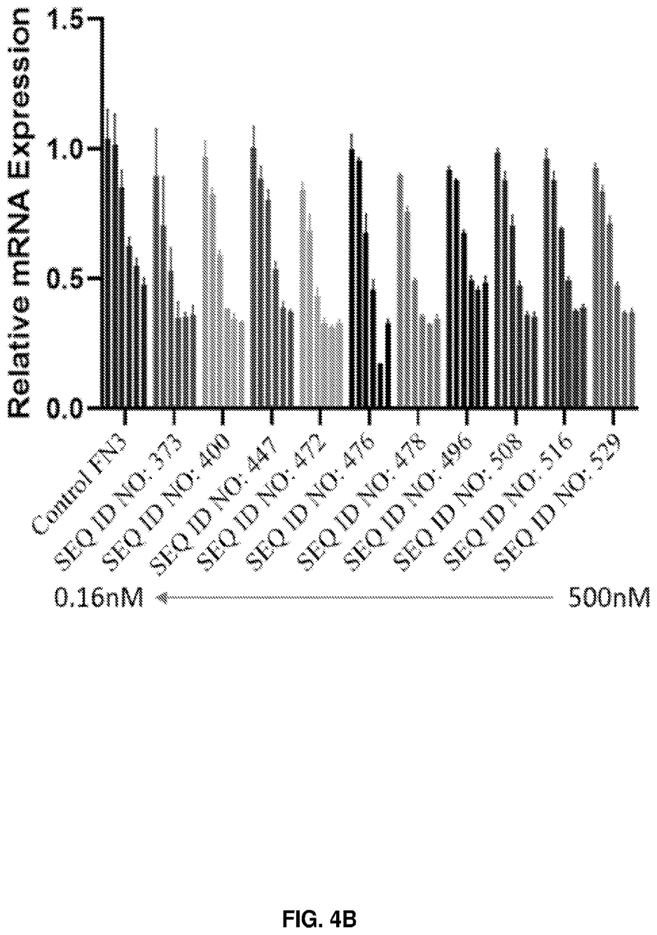

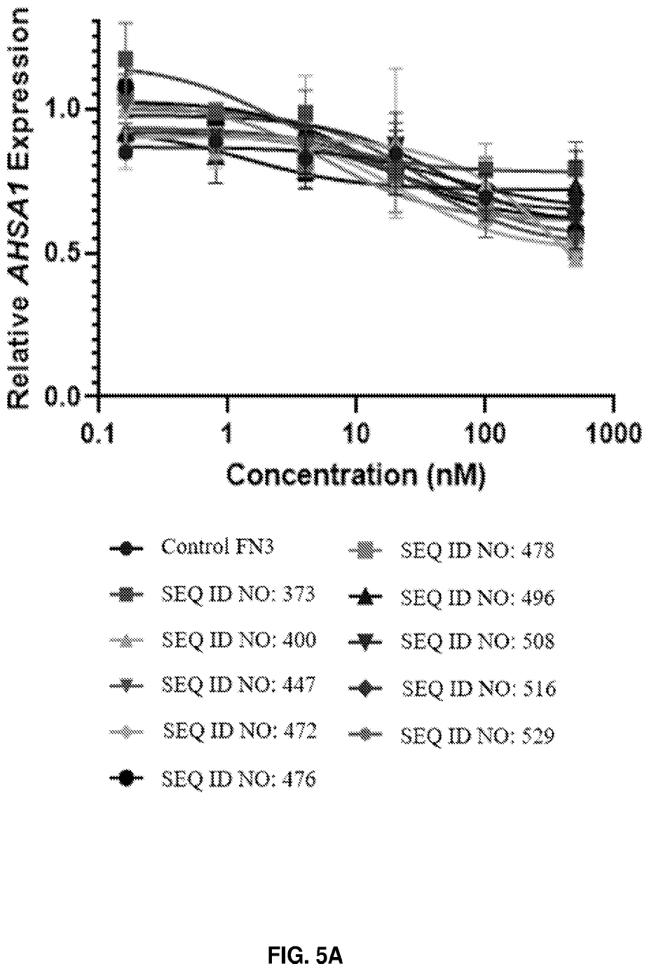

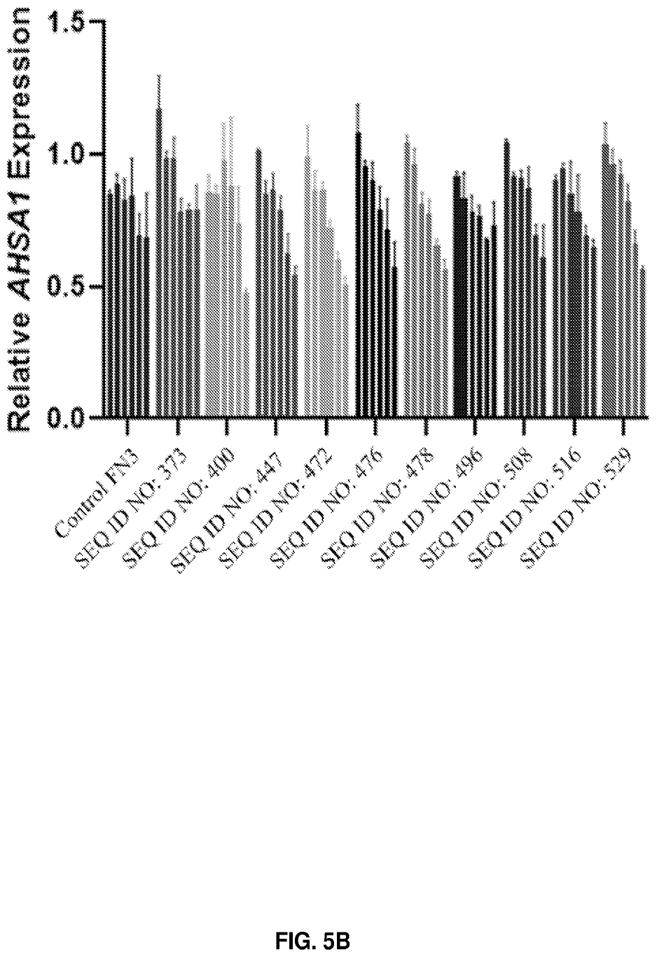

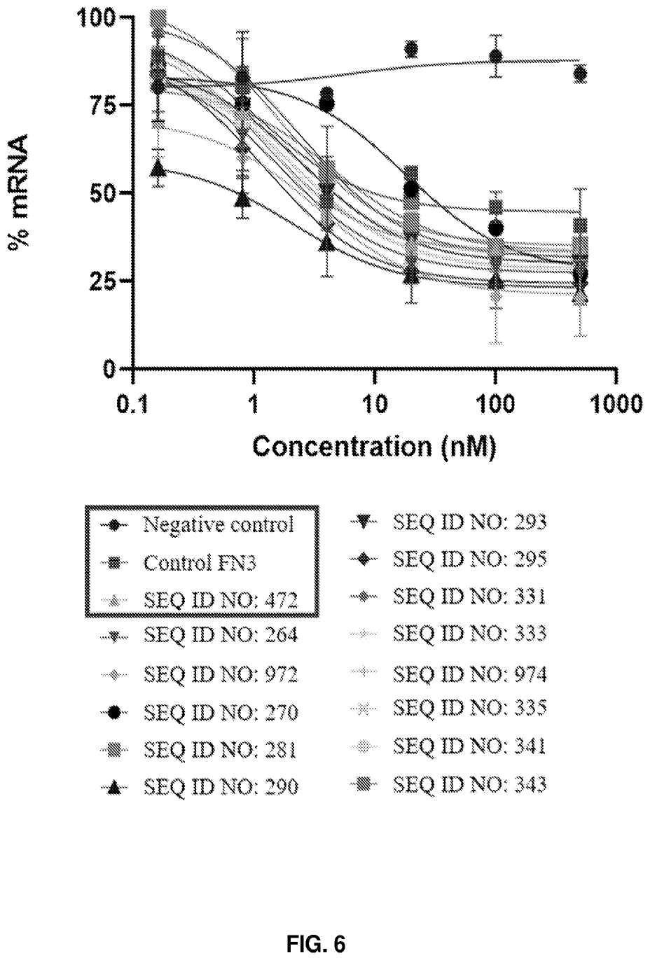

CD71-binding FN3 domains were conjugated to an AHSA1 (e.g., AHA1)-targeting siRNA and administered to SKBR3 cells and primary cynomolgus dermal fibroblast cells in a 6-point dose curve of 0.16, 0.8, 4, 20, 100, and 500 nM. The target mRNA expression was quantified at 96 hours post-treatment by RT-qPCR. The results from SKBR3 cells are shown in FIGS. 4A and 4B. The results from the cynomolgus fibroblasts are shown in FIGS. 5A and 5B. The results from a second group of CD71-binding FN3 domains administered to SKBR3 cells are shown in FIG. 6, with max KD % and EC50 values shown in Table 23 below.

TABLE 23
Max KD % and EC50 results

| SEQ ID NO of FN3 Domain | | |
|---|---|---|
| (Conjugated to AHA1 siRNA) | Max KD % | EC50 nM |
| 472 | 66.74% | 1.21 |
| 264 | 72.66% | 1.66 |
| 972 | 78.93% | 3.26 |
| 270 | 72.30% | 17.91 |
| 281 | 64.92% | 1.68 |
| 290 | 76.79% | 2.1 |
| 293 | 69.93% | 2.51 |
| 295 | 75.60% | 1.2 |
| 331 | 68.35% | 2.47 |
| 333 | 71.85% | 2.29 |
| 974 | 66.25% | 1.03 |
| 335 | 68.25% | 2.34 |
| 341 | 71.30% | 2.26 |
| 343 | 66.61% | 4.39 |
| FN3 control | 55.31% | 1.11 |
| Negative control | ND | ND |

### Example 8. CD71-Binding FN3 Domains do not have an Anti-Proliferative Effect on Immune Cells

CD71-binding FN3 domains were tested to determine if they had an anti-proliferative effect on immune cells. The FN3 domains do not inhibit proliferation of immune cells, such as B cells, T cells, dendritic cells, and peripheral blood mononuclear cells, up to a tested concentration of 1,000 mM (data not shown).

### General Methods

Standard methods in molecular biology are described Sambrook, Fritsch and Maniatis (1982 & 1989 2^{nd} Edition, 2001 3^{rd} Edition)Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols.1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications of the embodiments in addition to those described herein will become apparent to those skilled in the art from the foregoing description. Such modifications are intended to fall within the scope of the appended claims.

The foregoing written specification is considered to be sufficient to enable one skilled in the art to practice the embodiments. Various modifications of the embodiments in addition to those shown and described herein will become apparent to those skilled in the art from the foregoing description and fall within the scope of the appended claims.

What is claimed:

1. A CD71 binding FN3 domain polypeptide comprising an amino acid sequence that is at least 70%, 75%, 80%, 85%, 90%, or 95% identical to, or is identical to, an amino acid sequence selected from the group consisting of SEQ ID NOs: 100-209, 211-301, 303-317, 319-552, and 972-976.

2. The polypeptide of claim 1, wherein the polypeptide is conjugated to a detectable label, a therapeutic agent, or any combination thereof.

3. The polypeptide of claim 2, wherein the detectable label is a radioactive isotope, magnetic beads, metallic beads, colloidal particles, a fluorescent dye, an electron-dense reagent, an enzyme, biotin, digoxigenin, or hapten.

4. The polypeptide of claim 2, wherein the therapeutic agent is a polynucleotide, a chemotherapeutic agent, a drug, an antibody, a growth inhibitory agent, a toxin, an anti-tubulin agent, a siRNA molecule or a sense or an antisense strand thereof, an antisense molecule or a strand thereof, a RNA molecule, a DNA molecule, a DNA minor groove binder, a DNA replication inhibitor, an alkylating agent, an antibiotic, an antifolate, an antimetabolite, a chemotherapy sensitizer, a topoisomerase inhibitor, or a vinca alkaloid.

5. The polypeptide of claim 1, further comprising a methionine at the N-terminus of the polypeptide.

6. The polypeptide of claim 1, wherein the polypeptide is coupled to a half-life extending moiety.

7. The polypeptide of claim 6, wherein the half-life extending moiety is an albumin binding molecule, a polyethylene glycol (PEG), an albumin, an albumin variant, or an Fc region of an immunoglobulin or a fragment thereof.

8. An isolated polynucleotide encoding the polypeptide of claim 1.

9. A vector comprising the polynucleotide of claim 8.

10. A host cell comprising the vector of claim 9.

11. A method of producing a polypeptide that binds CD71, comprising culturing the isolated host cell of claim 10 under conditions that the polypeptide is expressed, and purifying the polypeptide.

12. A pharmaceutical composition comprising the polypeptide of claim 1 and a pharmaceutically acceptable carrier.

13. A kit comprising the polypeptide of claim 1.

14. A method of treating cancer in a subject in need thereof, the method comprising administering to the subject the polypeptide of claim 2 conjugated to a therapeutic agent.

15. A method of detecting CD71 expressing cancer cells in a tumor tissue, comprising:
a) obtaining a sample of the tumor tissue from a subject; and
b) detecting whether CD71 is expressed in the tumor tissue by contacting the sample of the tumor tissue with a polypeptide comprising the amino acid sequence of any one of SEQ ID NOs: 100-209, 211-301, 303-317, 319-552, and 972-976, and detecting the binding between CD71 and the polypeptide.

16. A method of isolating CD71 expressing cells, comprising
a) obtaining a sample from a subject;
b) contacting the sample with a polypeptide comprising the amino acid sequence of any one of SEQ ID NOs: 100-209, 211-301, 303-317, 319-552, and 972-976; and
c) isolating the cells bound to the polypeptide.

17. A method of delivering an agent of interest to a CD71 positive cell, the method comprising contacting the cell with the agent of interest coupled to the polypeptide of claim 1.

18. The method of claim 17, wherein the cell is a muscle cell, a brain cell, or a cell inside of the blood brain barrier.

19. A method of identifying a FN3 protein that binds to CD71 at a site that does not compete or inhibit transferrin binding to CD71, the method comprising:
a) contacting CD71 in the presence of transferrin or an agent that binds to the CD71 transferrin binding site with a test FN3 protein; and
b) identifying a test FN3 protein that binds to CD71 in the presence of transferrin or an agent that binds to the CD71 transferrin binding site.

20. The method of claim 19, further comprising isolating the test FN3 protein that binds to CD71 in the presence of transferrin or an agent that binds to the CD71 transferrin binding site.

21. The method of claim 19, further comprising sequencing the test FN3 protein that binds to CD71 in the presence of transferrin or an agent that binds to the CD71 transferrin binding site.

22. The method of claim 19, further comprising preparing or obtaining a nucleic acid sequence encoding the test FN3 protein that binds to CD71 in the presence of transferrin or an agent that binds to the CD71 transferrin binding site.

23. The method of claim 22, further comprising expressing the test FN3 protein that binds to CD71 in the presence of transferrin or an agent that binds to the CD71 transferrin binding site from a nucleic acid sequence encoding the test FN3 protein that binds to CD71 in the presence of transferrin or an agent that binds to the CD71 transferrin binding site.

24. The method of claim 23, wherein the test FN3 protein is expressed in a cell.

25. The method of claim 23, further comprising isolating and/or purifying the expressed test FN3 protein.

26. A FN3 protein identified according to claim 19.

27. A pharmaceutical composition comprising the FN3 protein of claim 26.

28. An isolated polynucleotide encoding the polypeptide of claim 26.

29. A vector comprising the polynucleotide of claim 28.

30. A host cell comprising the vector of claim 29.

31. A composition having a formula A₁-B₁, wherein A₁ has a formula of (C)ₙ-(L₁)ₜ-Xₛ and B₁ has a formula of X_{AS}-(L₂)_{q}-(F₁)_{y},
wherein:
C is a polymer, such as PEG, albumin binding protein, or an aliphatic chain that binds to serum proteins;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule; and
F₁ is a polypeptide comprising at least one FN3 domain,
wherein n, t, q, and y are each independently 0 or 1, and
wherein X_{S} and X_{AS} form a double stranded siRNA molecule.

32. A composition having a formula A₁-B₁, wherein A₁ has a formula of (F₁)ₙ-(L₁)ₜ-Xₛ and B₁ has a formula of X_{AS}-(L₂)_{q}-(C)_{y},
wherein:
C is a polymer, such as PEG, albumin binding protein, or an aliphatic chain that binds to serum proteins;
L₁ and L₂ are each, independently, a linker;
X_{S} is a 5′ to 3′ oligonucleotide sense strand of a double stranded siRNA molecule;
X_{AS} is a 3′ to 5′ oligonucleotide antisense strand of a double stranded siRNA molecule; and
F₁ is a polypeptide comprising at least one FN3 domain,
wherein n, t, q, and y are each independently 0 or 1, and
wherein X_{S} and X_{AS} form a double stranded siRNA molecule.

33. The composition of claim 31 or 32, wherein L₁ has the formula:

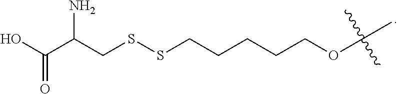 [SMILES: *OCCCCCSSCC(N)C(=O)O]

34. The composition of claim 31 or 32, wherein L₂ has the formula:

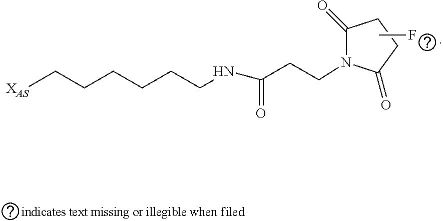

35. The composition of claim 31 or 32, wherein A₁-B₁ has a formula of:

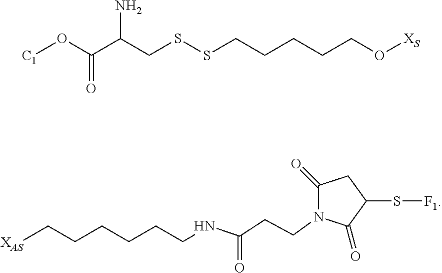

36. The composition of claim 31 or 32, where in A₁-B₁ has a formula of:

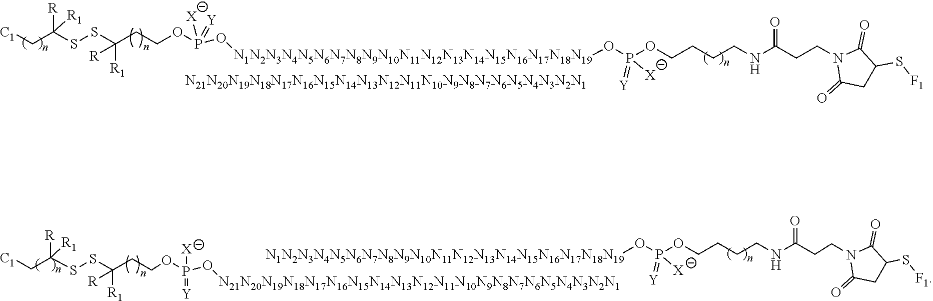

37. The composition of claim 31 or 32, wherein F₁ comprises polypeptide having a formula of (X₁)ₙ—(X₂)_{q}—(X₃)_{y}, wherein X₁ is a first FN3 domain; X₂ is a second FN3 domain; and X₃ is a third FN3 domain or half-life extender molecule, and wherein n, q, and y are each independently 0 or 1, provided that at least one of n, q, and y is 1.

38. The composition of claim 31 or 32, wherein X₁ is a CD71 binding FN3 domain.

39. The composition of claim 31 or 32, wherein X₂ is a CD71 binding FN3 domain.

40. The composition of claim 31 or 32, wherein X₃ is a FN3 domain that binds to human serum albumin.

41. The composition of claim 31 or 32, wherein X_{S} comprises a nucleic acid sequence selected from the group consisting of SEQ ID NOs: 600-659 and SEQ ID NOs: 46-178, 312-331, 352-356, 673-805, and 939-958 of U.S. Provisional Application No. 63/380,112, or as otherwise provided for herein.

42. The composition of claim 31 or 32, wherein X_{AS} comprises a nucleic acid sequence selected from the group consisting of SEQ ID NOs: 660-719 and SEQ ID NOs: 179-311, 332-351, 356-359, 806-938, and 959-978 of U.S. Provisional Application No. 63/380,112, or as otherwise provided for herein.

43. The composition of claim 31 or 32, wherein X_{S} and X_{AS} form a siRNA pair selected from the group consisting of A1, B1, C1, D1, E1, F1, G1, H1, I1, J1, K1, L1, M1, N1, O1, P1, Q1, R1, S1, T1, U1, V1, W1, X1, Y1, Z1, A2, B2, C2, D2, E2, F₂, G2, H2, I2, J2, K2, L2, M2, N2, O2, P2, Q2, R2, S2, T2, U2, V2, W2, X2, Y2, Z2, A3, B3, C3, D3, E3, F3, G3, H3, and any siRNA pair as set forth in Table 7a, Table 7b, and any siRNA pair provided in U.S. Provisional Application No. 63/380,112, and any siRNA pair as otherwise provided for herein.

44. The composition of claim 31 or 32, wherein F₁ comprises an amino acid sequence that is at least 87% identical to or is identical to a sequence selected from the group consisting of SEQ ID NO: 100-209, 211-301, 303-317, 319-552, and 972-976.

45. A method of treating an immunological disease in a subject in need thereof, the method comprising administering to the subject the composition of claim 31 or 32.

46. A use of the composition of claim 31 or 32 in the preparation of a pharmaceutical composition or medicament for treating immunological diseases.

47. Use of the composition of claim 31 or 32 for treating immunological diseases.

48. A method of reducing the expression of a target gene in a cell, the method comprising contacting the cell with the composition of claim 31 or 32.

49. The method of claim 48, wherein the target gene is CD40, KRAS, or GYS1.


SEQUENCE LISTING (978 sequences)

SEQ ID NO: 1 - protein, 89 aa, synthetic construct
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 2 - protein, 89 aa, synthetic construct
LPAPKNLVVSRVTEDSARLSWTAPDAAFDSFLIQYQESEKVGEAIVLTVPGSERSYDLTGLKPGTEYTVSIYGVKGGHRSNPLSAIFTT

SEQ ID NO: 3 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 4 - protein, 672 aa, Homo sapiens
CKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 5 - protein, 90 aa, synthetic construct
MLPAPKNLVASRVTEDSARLSWTAPDAAFDSFNIAYWEPGIGGEAIWLRVPGSERSYDLTGLKPGTEYKVWIHGVKGGASSPPLIARFTT

SEQ ID NO: 6 - protein, 90 aa, synthetic construct
MLPAPKNLVVSRVTEDSARLSWTAPDAAFDSFNIAYWEPGIGGEAIWLRVPGSERSYDLTGLKPGTEYKVWIHGVKGGASSPPLIARFTT

SEQ ID NO: 7 - protein, 90 aa, synthetic construct
MLPAPKNLVVSRVTEDSARLSWTAPDAAFDSFAIAYWEPGIGGEAIWLRVPGSERSYDLTGLKPGTEYKVWIHGVKGGASSPPLIARFTT

SEQ ID NO: 8 - protein, 90 aa, synthetic construct
MLPAPKNLVVSRVTEDSARLSWTAPDAAFDSFNISYWEPGIGGEAIWLRVPGSERSYDLTGLKPGTEYKVWIHGVKGGASSPPLIARFTT

SEQ ID NO: 9 - protein, 89 aa, synthetic construct
MLPAPKNLVVSRVTEDSARLSWTAPDAAFDSFNIAYAEPGIGGEAIWLRVPGSRSYDLTGLKPGTEYKVWIHGVKGGASSPPLIARFTT

SEQ ID NO: 10 - protein, 90 aa, synthetic construct
MLPAPKNLVVSRVTEDSARLSWTAPDAAFDSFNIAYWEAGIGGEAIWLRVPGSERSYDLTGLKPGTEYKVWIHGVKGGASSPPLIARFTT

SEQ ID NO: 11 - protein, 90 aa, synthetic construct
MLPAPKNLVVSRVTEDSARLSWTAPDAAFDSFNIAYWEPAIGGEAIWLRVPGSERSYDLTGLKPGTEYKVWIHGVKGGASSPPLIARFTT

SEQ ID NO: 12 - protein, 90 aa, synthetic construct
MLPAPKNLVVSRVTEDSARLSWTAPDAAFDSFNIAYWEPGAGGEAIWLRVPGSERSYDLTGLKPGTEYKVWIHGVKGGASSPPLIARFTT

SEQ ID NO: 13 - protein, 90 aa, synthetic construct
MLPAPKNLVVSRVTEDSARLSWTAPDAAFDSFNIAYWEPGIAGEAIWLRVPGSERSYDLTGLKPGTEYKVWIHGVKGGASSPPLIARFTT

SEQ ID NO: 14 - protein, 90 aa, synthetic construct
MLPAPKNLVVSRVTEDSARLSWTAPDAAFDSFNIAYWEPGIGGEAIALRVPGSERSYDLTGLKPGTEYKVWIHGVKGGASSPPLIARFTT

SEQ ID NO: 15 - protein, 90 aa, synthetic construct
MLPAPKNLVVSRVTEDSARLSWTAPDAAFDSFNIAYWEPGIGGEAIWLAVPGSERSYDLTGLKPGTEYKVWIHGVKGGASSPPLIARFTT

SEQ ID NO: 16 - protein, 90 aa, synthetic construct
MLPAPKNLVVSRVTEDSARLSWTAPDAAFDSFNIAYWEPGIGGEAIWLRVPGSERSYDLTGLKPGTEYAVWIHGVKGGASSPPLIARFTT

SEQ ID NO: 17 - protein, 90 aa, synthetic construct
MLPAPKNLVVSRVTEDSARLSWTAPDAAFDSFNIAYWEPGIGGEAIWLRVPGSERSYDLTGLKPGTEYKVAIHGVKGGASSPPLIARFTT

SEQ ID NO: 18 - protein, 90 aa, synthetic construct
MLPAPKNLVVSRVTEDSARLSWTAPDAAFDSFNIAYWEPGIGGEAIWLRVPGSERSYDLTGLKPGTEYKVWIAGVKGGASSPPLIARFTT

SEQ ID NO: 19 - protein, 90 aa, synthetic construct
MLPAPKNLVVSRVTEDSARLSWTAPDAAFDSFNIAYWEPGIGGEAIWLRVPGSERSYDLTGLKPGTEYKVWIHGVKGGSSSPPLIARFTT

SEQ ID NO: 20 - protein, 90 aa, synthetic construct
MLPAPKNLVVSRVTEDSARLSWTAPDAAFDSFNIAYWEPGIGGEAIWLRVPGSERSYDLTGLKPGTEYKVWIHGVKGGAASPPLIARFTT

SEQ ID NO: 21 - protein, 90 aa, synthetic construct
MLPAPKNLVVSRVTEDSARLSWTAPDAAFDSFNIAYWEPGIGGEAIWLRVPGSERSYDLTGLKPGTEYKVWIHGVKGGASSAPLIARFTT

SEQ ID NO: 22 - protein, 90 aa, synthetic construct
MLPAPKNLVVSRVTEDSARLSWTAPDAAFDSFNIAYWEPGIGGEAIWLRVPGSERSYDLTGLKPGTEYKVWIHGVKGGASSPPLAARFTT

SEQ ID NO: 23 - protein, 90 aa, synthetic construct
MLPAPKNLVVSRVTEDSARLSWTAPDAAFDSFNIAYWEPGIGGEAIWLRVPGSERSYDLTGLKPGTEYKVWIHGVKGGASSPPLIAAFTT

SEQ ID NO: 24 - protein, 9 aa, synthetic construct
RKKRRQRRR

SEQ ID NO: 25 - protein, 16 aa, synthetic construct
RQIKIWFQNRRMKWKK

SEQ ID NO: 26 - protein, 26 aa, synthetic construct
GWTLNSAGYLLGKINKALAALAKKIL

SEQ ID NO: 27 - protein, 18 aa, synthetic construct
KLALKLALKALKAALKLA

SEQ ID NO: 28 - protein, 21 aa, synthetic construct
KETWWETWWTEWSQPKKKRKV

SEQ ID NO: 29 - protein, 4 aa, synthetic construct
KDEL

SEQ ID NO: 30 - protein, 29 aa, synthetic construct
WEAALAEALAELAEHLAEALAEALEALAA

SEQ ID NO: 31 - protein, 21 aa, synthetic construct
GDIMGEWGNEIFGAIAGFLGC

SEQ ID NO: 32 - protein, 13 aa, synthetic construct
GLFDIIKKIAESF

SEQ ID NO: 33 - protein, 24 aa, synthetic construct
GALFLGWLGAAGSTMGAPKSKRKV

SEQ ID NO: 34 - protein, 21 aa, synthetic construct
AGYLLGKINLKALAALAKKIL

SEQ ID NO: 35 - protein, 23 aa, synthetic construct
LIRLWSHLIHIWFQNRRLKWKKK

SEQ ID NO: 36 - protein, 36 aa, synthetic construct
GLFGAIAGFIENGWEGMIDGRQIKIWFQNRRMKWKK

SEQ ID NO: 37 - protein, 26 aa, synthetic construct
FFKKLAHALHLLALLALHLAHALKKA

SEQ ID NO: 38 - protein, 21 aa, synthetic construct
LFEAIEGFIENGWGMIDGWYG

SEQ ID NO: 39 - protein, 31 aa, synthetic construct
LFEAIEGFIENGWEGMIDGWYGRKKRRQRRR

SEQ ID NO: 40 - protein, 25 aa, synthetic construct
IGAVLKVLTTGLPALISWIKRKRQQ

SEQ ID NO: 41 - protein, 21 aa, synthetic construct
MKLAVTLTLVTLALSSSSASA

SEQ ID NO: 42 - protein, 22 aa, synthetic construct
RLIEDICLPRWGCLWEDDKDEL

SEQ ID NO: 43 - protein, 17 aa, synthetic construct
MIRTLLLSTLVAGALSK

SEQ ID NO: 44 - protein, 19 aa, synthetic construct
ILSSLTVTQLLRRLHQWIK

SEQ ID NO: 45 - protein, 20 aa, synthetic construct
MIRTLLLSTLVAGALSKDEL

SEQ ID NO: 46 - protein, 4 aa, synthetic construct
GSGS

SEQ ID NO: 47 - protein, 8 aa, synthetic construct
GGGSGGGS

SEQ ID NO: 48 - protein, 25 aa, synthetic construct
  VARIANT 6..25: note = GGGGS repeats may be deleted
GGGGSGGGGSGGGGSGGGGSGGGGS

SEQ ID NO: 49 - protein, 25 aa, synthetic construct
GGGGSGGGGSGGGGSGGGGSGGGGS

SEQ ID NO: 50 - protein, 25 aa, synthetic construct
  VARIANT 6..25: note = GGGGA repeats may be deleted
GGGGAGGGGAGGGGAGGGGAGGGGA

SEQ ID NO: 51 - protein, 40 aa, synthetic construct
  VARIANT 3..40: note = AP repeats may be deleted
APAPAPAPAPAPAPAPAPAPAPAPAPAPAPAPAPAPAPAP

SEQ ID NO: 52 - protein, 40 aa, synthetic construct; identical to SEQ ID NO: 51
  VARIANT 5..40: note = AP repeats may be deleted

SEQ ID NO: 53 - protein, 4 aa, synthetic construct
APAP

SEQ ID NO: 54 - protein, 10 aa, synthetic construct
APAPAPAPAP

SEQ ID NO: 55 - protein, 20 aa, synthetic construct
APAPAPAPAPAPAPAPAPAP

SEQ ID NO: 56 - protein, 40 aa, synthetic construct; identical to SEQ ID NO: 51

SEQ ID NO: 57 - protein, 29 aa, synthetic construct
AEAAAKEAAAKEAAAKEAAAKEAAAKAAA

SEQ ID NO: 58 - protein, 25 aa, synthetic construct
  VARIANT 6..25: note = EAAAK repeats may be deleted
EAAAKEAAAKEAAAKEAAAKEAAAK

SEQ ID NO: 59 - protein, 20 aa, synthetic construct
EAAAKEAAAKEAAAKEAAAK

SEQ ID NO: 60 - protein, 20 aa, synthetic construct
GGGGSGGGGSGGGGSGGGGS

SEQ ID NO: 61 - protein, 10 aa, synthetic construct
APAPAPAPAP

SEQ ID NO: 62 - protein, 5 aa, synthetic construct
EAAAK

SEQ ID NO: 63 - protein, 6 aa, synthetic construct
HHHHHH

SEQ ID NO: 64-89 - skipped (26 entries)

SEQ ID NO: 90 - protein, 90 aa, synthetic construct
  VARIANT 33: note = D, F, Y or H
  VARIANT 35: note = Y, G, A or V
  VARIANT 37: note = I, T, L, A or H
  VARIANT 39: note = S, Y or P
  VARIANT 40: note = Y, G, Q or R
  VARIANT 41: note = G or P
  VARIANT 42: note = A, Y, P, D or S
  VARIANT 47: note = any amino acid other than methionine or proline
  VARIANT 49: note = any amino acid other than methionine or proline
  VARIANT 69: note = W, N, S or E
  VARIANT 71: note = L, Y or G
  VARIANT 73: note = D, Q, H or V
  VARIANT 74: note = G or S
  VARIANT 79: note = R, G, F, L or D
  VARIANT 80: note = W, S, P or L
  VARIANT 82: note = T, V, M or S
  VARIANT 85: note = any amino acid other than methionine or proline
  VARIANT 87: note = any amino acid other than methionine or proline
MLPAPKNLVVSRVTEDSARLSWTAPDAAFDSFXIXYXEXXXXGEAIXLXVPGSERSYDLTGLKPGTEYXVXIXXVKGGXXSXPLXAXFTT

SEQ ID NO: 91 - protein, 90 aa, synthetic construct
MLPAPKNLVVSRVTEDSARLSWTAPDAAFDSFYIAYAEPRPDGEAIVLTVPGSERSYDLTGLKPGTEYSVLIHGVKGGLLSSPLSAIFTT

SEQ ID NO: 92 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLSAIFTT

SEQ ID NO: 93 - protein, 90 aa, synthetic construct
MLPAPKNLVVSRVTEDSARLSWTAPDAAFDSFHIVYHEPRPSGEAIVLTVPGSERSYDLTGLKPGTEYEVGIVSVKGGDLSVPLSAIFTT

SEQ ID NO: 94 - protein, 90 aa, synthetic construct
MLPAPKNLVVSRVTEDSARLSWTAPDAAFDSFDIGYTEYGGYGEAIVLTVPGSERSYDLTGLKPGTEYWVLIQGVKGGGSSVPLSAIFTT

SEQ ID NO: 95-99 - skipped (5 entries)

SEQ ID NO: 100 - protein, 90 aa, synthetic construct
MLPAPKNLVVSRVTEDSARLSWTAPDAAFDSFYIAYAEPRPDGEAILLLVPGSERSYDLTGLKPGTEYSVLIHGVKGGLLSSPLVASFTT

SEQ ID NO: 101 - protein, 90 aa, synthetic construct
MLPAPKNLVVSRVTEDSARLSWTAPDAAFDSFGIGYWERRWYGEAIVLTVPGSERSYDLTGLKPGTEYEVTIRGVKGGGYSGPLSAIFTT

SEQ ID NO: 102 - protein, 90 aa, synthetic construct
MLPAPKNLVVSRVTEDSARLSWTAPDAAFDSFDIGYTEYGGYGEAIILQVPGSERSYDLTGLKPGTEYWVLIQGVKGGGSSVPLVAYFTT

SEQ ID NO: 103 - protein, 90 aa, synthetic construct
MLPAPKNLVVSRVTEDSARLSWTAPDAAFDSFYIAYAEPRPDGEAIILGVPGSERSYDLTGLKPGTEYSVLIHGVKGGLLSSPLSAYSTT

SEQ ID NO: 104 - protein, 90 aa, synthetic construct
MLPAPKNLVVSRVTEDSARLSWTAPDAAFDSFFIGYLEPQPPGEAIHLIVPGSERSYDLTGQKPGTEYNVTIQGVKGGFPSDPLVASFTT

SEQ ID NO: 105 - protein, 90 aa, synthetic construct
MLPAPKNLVVSRVTEDSARLSWTAPDAAFDSFHIVYHEPRPSGEAIWLWVPGSERSYDLTGLKPGTEYEVGIVSVKGGDLSVPLSAIFTT

SEQ ID NO: 106 - protein, 90 aa, synthetic construct
MLPAPKNLVVSRVTEDSARLSWTAPDAVFDSFYIAYAEPRPDGEAIGLYVPGSERSYDLTGLKPGTEYSVLIHGVKGGLLSSPLTASFTT

SEQ ID NO: 107 - protein, 90 aa, synthetic construct
MLPAPKNLVVSRVTEDSARLSWTAPDAAFDSFFIGYLEPQPPGEAIGLQVPGSERSYDLTGLKPGTEYNVTIQGVKGGFPSMPLSAIFTT

SEQ ID NO: 108 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLVAKFTT

SEQ ID NO: 109 - protein, 90 aa, synthetic construct
MLPAPKNLVVSRVTEDSARLSWTAPDAAFDSFFIGYLEPQPPGEAIILHVPGSERSYDLTGLKPGTEYNVTIQGVKGGFPSLPLNANFTT

SEQ ID NO: 110 - protein, 90 aa, synthetic construct
MLPAPKNLVVSRVTEDSARLSWTAPDAAFDSFHIVYHEPRPSGEAIYLEVPGSERSYDLTGLKPGTEYEVGIVSVKGGDLSVPLRAHFTT

SEQ ID NO: 111 - protein, 90 aa, synthetic construct
MLPAPKNLVVSRVTEDSARLSWTAPDAAFDSFFIGYLEPQPPGEAISLWVPGSERSYDLTGLKPGTEYNVTIQGVKGGFPSIPLWAIFTT

SEQ ID NO: 112 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSMPLSAIFTT

SEQ ID NO: 113 - protein, 90 aa, synthetic construct
MLPAPKNLVVSRVTEDSARLSWTAPDAAFDSFFIGYLEPQPPGEAIYLLVPGSERSYDLTGLKPGTEYNVTIQGVKGGFPSDPLSAIFTT

SEQ ID NO: 114 - protein, 90 aa, synthetic construct
MLPAPKNLVVSRVTEDSARLSWTAPDAAFDSFHIVYHEPRPSGEAIWLFVPGSERSYDLTGLKPGTEYEVGIVSVKGGDLSVPLSAIFTT

SEQ ID NO: 115 - protein, 90 aa, synthetic construct
MLPAPKNLVVSRVTEDSARLSWTAPDAAFDSFFIGYLEPQPPGEAIDLYVPGSERSYDLTGLKPGIEYNVTIQGVKGGFPSLPLQAHFTT

SEQ ID NO: 116 - protein, 90 aa, synthetic construct
MLPAPKNLVVSRVTEDSARLSWTAPDAAFDSFYIAYAEPRPDGEAILLLVPGSERSYDLTGLKPGTEYSVLIHGVKGGLLSSPLIAGFTT

SEQ ID NO: 117 - protein, 90 aa, synthetic construct
MLPAPKNLVVSRVTEDSARLSWTAPDAAFDSFFIGYLEPQPPGEAIWLLVPGSERSYDLTGLKPGTEYNVTIQGVKGGFPSTPLSAIFTT

SEQ ID NO: 118 - protein, 90 aa, synthetic construct
MLPAPKNLVVSRVTEDSARLSWTAPDAAFDSFYIAYAEPRPDGEAITLWVPGSERSYDLTGLKPGTEYSVTIHGVKGGLLSSPLSAIFTT

SEQ ID NO: 119 - protein, 91 aa, synthetic construct
MSLPAPKNLVVSRVTEDSARLSWTAPDAAFDSFFIGYLEPQPPGEAIELYVPGSERSYDLTGLKPGTEYNVTIQGVKGGFPSDPLVAFFTT

SEQ ID NO: 120 - protein, 91 aa, synthetic construct
MSLPAPKNLVVSRVTEDSARLSWTAPDAAFDSFEIAYQELSLWGEAIVLTVPGSERSYDLTGLKPGTEYWVQIGGVKGGEWSAPLSAIFTT

SEQ ID NO: 121 - protein, 91 aa, synthetic construct
MSLPAPKNLVVSRVTEDSARLSWTAPDAAFDSFFIGYLEPQPPGEAITLYVPGSERSYDLTGLKPGTEYNVTIQGVKGGFPSAPLSAIFTT

SEQ ID NO: 122 - protein, 91 aa, synthetic construct
MSLPAPKNLVVSRVTEDSARPSWTAPDAAFDSFEIGYFENLYLGEAIVLTVPGSERSYDLTGLKPGTEYYVHIGGVKGGIWSSPLSAYFTT

SEQ ID NO: 123 - protein, 91 aa, synthetic construct
MSLPAPKNLVVSRVTEDSARLSWTAPDAAFDSFAIRYWELRATGEAIPLSVPGSERSYDLTGLKPGTEYHVAISGVKGGKSSYPLRASFTT

SEQ ID NO: 124 - protein, 91 aa, synthetic construct
MSLPAPKNLVVSRVIEDSARLSWTAPDAAFDSFEIGYFENLYLGEAIVLTVPGSERSYDLTGLKPGTEYWVQIGGVKGGNWSAPLSAIFTT

SEQ ID NO: 125 - protein, 91 aa, synthetic construct
MSLPAPKNLVVSRVTEDSARLSWTAPDAAFDSFYIAYAEPRPDGEAIILLVPGSERSYDLTGLKPGTEYSVLIHGVKGGLLSSPLHAHFTT

SEQ ID NO: 126 - protein, 91 aa, synthetic construct
MSLPAPKNLVVSRVTEDSARLSWTAPDAVFDSFEIAYQELSLWGEAIVLTVPGSERSYDLTGLKPGTEYYVHIGGVKGGIWSSPLSAYFTT

SEQ ID NO: 127 - protein, 91 aa, synthetic construct
MSLPAPKNLVVSRVTEDSARLSWTAPDAAFDSFFIGYLEPQPPGEAITLLVPGSERSYDLTGLKPGTEYNVTIQGVKGGFPSGPLQASFTT

SEQ ID NO: 128 - protein, 92 aa, synthetic construct
MSSLPAPKNLVVSRVTEDSARLSWTAPDAAFDSFEIGYFENLYLGEAIVLTVPGSERSYDLTGLKPGTEYWVQIGGVKGGNWSAPLSAIFTT

SEQ ID NO: 129 - protein, 93 aa, synthetic construct
MGGSLPAPKNLVVSRVTEDSARLSWTAPDAAFDSFEIGYFENLYLGEAIVLTVPGSERSYDLTGLKPGTEYWVQIGGVKGGNWSAPLSAIFTT

SEQ ID NO: 130 - protein, 91 aa, synthetic construct
MSLPAPKNLVVSRVTEDSARLSWTAPDAAFDSFEIAYFERTWFGEAIVLTVPGSERSYDLTGLKPGTEYWVQIGGVKGGDWSAPLSAIFTT

SEQ ID NO: 131 - protein, 91 aa, synthetic construct
MSLPAPKNLVVSRVTEDSARLSWTAPDAAFDSFEIAYFENDWAGEAIVLTVPGSERSYDLTGLKPGTEYVVFIGGVKGGDFSPPLSAIFTT

SEQ ID NO: 132 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGVNWSAPLSAIFTT

SEQ ID NO: 133 - protein, 91 aa, synthetic construct
MSLPAPKNLVVSRVTEDSARLSWTAPDAAFDSFKIKYAEYDRYGEAIALFVPGSERSYDLTGLKPGTEYFVHIDGVKGGTDSQPLVASFTT

SEQ ID NO: 134 - protein, 91 aa, synthetic construct
MSLPAPKNLVVSRVTEDSARLSWTAPDAAFDSFDINYWENESGGEAIALFVPGSERSYDLTGLKPGTEYLVTIAGVKGGWWSKPLYATFTT

SEQ ID NO: 135 - protein, 91 aa, synthetic construct
MSLPAPKNLVVSRVTEDSARLSWTAPDAAFDSFFIGYLEPQSPGEAIALYVPGSERSYDLTGLKPGTEYNVTIQGVKGGFPSEPLIANFTT

SEQ ID NO: 136 - protein, 91 aa, synthetic construct
MSLPAPKNLVVSRVTEDSARLSWTAPDAAFDSFEIDYVEFIFTGEAIGLIVPGSERSYDLTGLKPGTEYWVTIAGVKGGEWSTPLQAFLTT

SEQ ID NO: 137 - protein, 91 aa, synthetic construct
MSLPAPKNLVVSRVTEDSARLSWTAPDAAFDSFFIGYLEPQPPGEAITLYVPGSERSYDLTGLKPGTEYNVTIQGVKGGFPSDPLVAHFTT

SEQ ID NO: 138 - protein, 91 aa, synthetic construct
MSLPAPKNLVVSRVTEDSARLSWTAPDAAFDSFFIGYLEPQPPGEVIHLFVPGSERSYDLTGLKPGTEYNVTIQGVKGGFPSMPLSAIFTT

SEQ ID NO: 139 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGAYSTPLSAIFTT

SEQ ID NO: 140 - protein, 91 aa, synthetic construct
MSLPAPKNLVVSRVTEDSARLSWTAPDAAFDSFFIGYLEPQPPGEAIHLYVPGSERSYDLTGLKPGTEYNVTIQGVKGGFPSMPLSAIFTT

SEQ ID NO: 141 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLSAYFTT

SEQ ID NO: 142 - protein, 91 aa, synthetic construct
MSLPAPKNLVVSRVTEDSARLSWTASDAAFDSFHIWYFEKANEGEAIPLVVPGSERSYDLTGLKPGTEYDVDIGGVKGGAWSIPLGARFTT

SEQ ID NO: 143 - protein, 91 aa, synthetic construct
MSLPAPKNLVVSRVTEDSARLSWTAPDAAFDSFAIQYVEVIGSGEAIELIVPGSERSYDLTGLKPGTEYHVYIDGVKGGKDSKPLYAGFTT

SEQ ID NO: 144 - protein, 91 aa, synthetic construct
MSLPAPKNLVVSRVTEDSARLSWTAPDAAFDSFEIGYFENLYLGEAIVLTVPGSERSYDLTSLKPGTEYWVQIGGVKGGNWSAPLSATFTT

SEQ ID NO: 145 - protein, 91 aa, synthetic construct
MSLPAPKNLVVSRVTEDSARLSWTAPDAAFDSFFIGYLEPQPPGEAIALLVPGSERSYDLTGLKPGTEYNVTIQGVKGGFPSHPLSAKFTT

SEQ ID NO: 146 - protein, 91 aa, synthetic construct
MSLPAPKNLVVSRVTEDSARLSWTAPDAAFDSFDIGYFENTYEGEAIVLTVPGSERSYDLTGLKPGTEYVVWIGGVKGGSYSSPLSAIFTT

SEQ ID NO: 147 - protein, 91 aa, synthetic construct
MSLPAPKNLVVSRVTEDSARLSWTAPDAAFDSFFIGYLEPQPPGEAIELFVPGSERSYDLTGLKPGTEYNVTIQGVKGGFPSGPLIASFTT

SEQ ID NO: 148 - protein, 91 aa, synthetic construct
MSLPAPKNLVVSRVTEDSARLSWTAPDAAFDSFEIVYQENSAYGEAIVLTVPGSERSYDLTGLKPGTEYWVQIGGVKGGEWSAPLSAIFTT

SEQ ID NO: 149 - protein, 91 aa, synthetic construct
MSLPAPKNLVVSRVTEDSARLSWTAPDAAFDSFDIGYFELRYYGEAIVLTVPGSERSYDLTGLKPGTEYWVSIGGVKGGTYSAPLSAIFTT

SEQ ID NO: 150 - protein, 91 aa, synthetic construct
MSLPAPKNLVVSRVTEDSARLSWTAPDAAFDSFDIGYFENSYAGEAIVLTVPGSERSYDLTGLKPGTEYYVSIAGVKGGRFSPPLSAIFTT

SEQ ID NO: 151 - protein, 91 aa, synthetic construct
MSLPAPKNLVVSRITEDSARLSWTAPDAAFDSFEIAYFETCRTGEAIVLTVPGSERSYDLTGLKPGTEYRVWIGGVKGGVWSRPLSAIVTT

SEQ ID NO: 152 - protein, 91 aa, synthetic construct
MSLPAPKNLVVSRVTEDSARLSWTAPDAAFDSFDIGYFEATYYGEAIVLTVPGSERSYDLTGLKPGTEYWVQIGGVKGGNWSAPLSAIFTT

SEQ ID NO: 153 - protein, 91 aa, synthetic construct
MSLPAPKNLVVSRVTEDSARLSWTAPDAAFDSFYIAYAEPRPDGEAIVLAVPGSERSYDLTGLKPGTEYSVLIHGVKGGLLSSPLGADFTT

SEQ ID NO: 154 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLSAIFTT

SEQ ID NO: 155 - protein, 91 aa, synthetic construct
MSLPAPENLVVSRVTEDSARLSWTAPDAAFDSFEIGYFENLYLGEAIVLTVPGSERSYDLTGLKPGTEYWVQIGGVKGGVWSRPLSAIFTT

SEQ ID NO: 156 - protein, 91 aa, synthetic construct
MSLPAPKNLVVSRVTEDSARLSWTAPDAAFDSFEIGYFEMSWTGEAIVLTVPGSERSYDLTGLKPGTEYVVFIGGVKGGNWSAPLSAIFTT

SEQ ID NO: 157 - protein, 91 aa, synthetic construct
MSLPAPKNLVVSRVTEDSARLSWTAPDAAFDSFEIGYGEFTYYGEAIVLTVPGSERSYDLTGLKPGTEYWVQIGGVKGGNWSNPLSAIFTT

SEQ ID NO: 158 - protein, 91 aa, synthetic construct
MSLPAPKNLVVSRVTEDSARLSWTAPDAAFDSFEIAYFERTWFGEAIVLTVPGSERSYDLTGLKPGTEYWVQIGGVKGGDWSKPLSAIFTT

SEQ ID NO: 159 - protein, 91 aa, synthetic construct
MSLPAPKNLVVSRVTEDSARLSWTAPDAAFDSFEIVYQENSAYGEAIVLTVPGSERSYDLTGLKPGTEYWVQIGGVKGGNWSAPLSAISTT

SEQ ID NO: 160 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSVPLSAIFTT

SEQ ID NO: 161 - protein, 91 aa, synthetic construct
MSLPAPKNLVVSRVTEDSARLSWTAPDAAFDSFFIGYLEPQPPGEAIVLTVPGSERSYDLTGLKPGTEYNVTIQGVKGGFPSEPLVAHFTT

SEQ ID NO: 162 - protein, 91 aa, synthetic construct
MSLPAPKNLVVSRVTEDSARLSWTALDAAFDSFEISYWEHTFNGEAIILFVPGSERSYDLTGLKPGTEYYVTIGGVKGGAWSPPLWAEFTT

SEQ ID NO: 163 - protein, 91 aa, synthetic construct
MSLPAPKNLVVSRVTEDSARLSWTAPDAAFDSFFIGYLEPQPPGEVIHLYVPGSERSYDLTGLKPGTEYVVFIGGVKGGVWSVPLSAIFTT

SEQ ID NO: 164 - protein, 91 aa, synthetic construct
MSLPAPKNLVVSRVTEDSARLSWTAPDAAFDSFFIGYLEPQPPGEAIHLYVPGSERSYDLTGLKPGTEYWVQIGGVKGGNWSAPLSAIFTT

SEQ ID NO: 165 - protein, 91 aa, synthetic construct
MSLPAPKNLVVSRVTEDSARLSWTAPDAAFDSFYIAYAEPRPDGEAILLEVPGSERSYDLAGLKPGTEYSVLIHGVKGGLLSSPLGAEFTT

SEQ ID NO: 166 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGIKGGNWSAPLSAIFTT

SEQ ID NO: 167 - protein, 91 aa, synthetic construct
MSLPAPKNLVVSRVTEDSARLSWTAPDAAFDSFFIGYLEPQPPGEAIVLTVPGSERSYDLTGLKPGTEYNVTIQGVKGGFPSVPLSAAFTT

SEQ ID NO: 168 - protein, 91 aa, synthetic construct
MSLPAPKNLVVSRVTEDSARLSWTAPDAAFDSFIIGYLEPQPPDEAIALYVPGSERSYDLTGLKPGTEYNVTIQGVKGGFPSLPLVATFTT

SEQ ID NO: 169 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLSATFTT

SEQ ID NO: 170 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSTPLSAIFTT

SEQ ID NO: 171 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGEWSSPLSAIFTT

SEQ ID NO: 172 - protein, 91 aa, synthetic construct
MSRPAPKNLVVSRVTEDSARLSWTAPGAAFDSFEIAYFERTWFGEAIVLTVPSSERSYDLTGLKPGTEYWVQIGGVKGGDWSKPLSAIFTT

SEQ ID NO: 173 - protein, 91 aa, synthetic construct
MSLPAPKNLVVSRVTEDSARLSWTAPDAAFDSFEIAYQELSLWGEAIVLTVPGSERSYDLTGLKPGTEYYVHIGGVKGGIWSSPLVAHFTT

SEQ ID NO: 174 - protein, 91 aa, synthetic construct
MSLPAPKNLVVSRVTEDSARLSWTAPNAAFDSFEIGYFENLYLGEAIVLTVPGSERSYDLTGLKPGTEYWVQIGGVKGGNWSAPLSAIFTT

SEQ ID NO: 175 - protein, 91 aa, synthetic construct
MSLPAPKNLVVSRVTEDSARLSWTAPDAAFDSFEIAYQELSLWGEAIVLTVPGSERSYDLTGLKPGTEYVVFIGGVKGGVWSVPLSAIFTT

SEQ ID NO: 176 - protein, 91 aa, synthetic construct
MSLPAPKNLVVSRVTEDSARLSWTAPDAAFDSFFIGYLEPQPPGEAIHLFVPGSERSYDLTGLKPGTEYWVQIGGVKGGNWSAPLSAIFTT

SEQ ID NO: 177 - protein, 91 aa, synthetic construct
MSLPAPKNLVVSRVTEDSARLSWTAPDAAFDSFEIGYFENLYLGEAIVLTVPGSERSYDLTSLKPGTEYWVQIGGVKGGNWSAPLSAIFTT

SEQ ID NO: 178 - protein, 92 aa, synthetic construct
MGSLPAPKNLVVSRVTEDSARLSWTAPDAAFDSFFIGYLEPQPPGEAISLYVPGSERSYDLTGLKPGTEYNVTIQGVKGGFPSDPLVAHFTT

SEQ ID NO: 179 - protein, 91 aa, synthetic construct
MSLPAPKNLVVSRVTEDSARLSWTAPDAAFDSFFIGYLEPQPPGEAITLLVPGSERSYDLTGLKPGTEYNVTIQGVKGGFPSHPLVATTTT

SEQ ID NO: 180 - protein, 91 aa, synthetic construct
MSLPAPKNLVVSRVTEDSARLSWTAPDAAFDSFFIGYLEPQPPGEAISLYVPGSERSYDLTGLKPGTEYNVTIQGVKGGFPSEPLIANFTT

SEQ ID NO: 181 - protein, 91 aa, synthetic construct
MSLPAPKNLVVSRVTEDSARLSWTAPDAAFDSFEIGYFENDYFGEAIVLTVPGSERSYDLTGLKPGTEYWVQIGGVKGGNWSAPLSAIFTT

SEQ ID NO: 182 - protein, 91 aa, synthetic construct
MSLPAPENLVVSRVTEDSARLSWTAPDAAFDSFEIGYWENAYKGEAIVLTVPGSERSYDLTGLKPGTEYVVFIGGVKGGVWSVPLSVIFTT

SEQ ID NO: 183 - protein, 91 aa, synthetic construct
MSLPAPKNLVISRVTEDSARLSWTAPDAAFDSFEIVYAEPVRFGEAIVLTVPGSERSYDLTGLKPGTEYWVQIGGVKGGEWSSPLSAIFTT

SEQ ID NO: 184 - protein, 91 aa, synthetic construct
MSLPAPKNLVVSRVTEDSARPSWTAPDAAFDSFEIAYFERTWFGEAIVLTVPGSERSYDLTGLKPGTEYWVQIGGVKGGDWSKPLSAIFTT

SEQ ID NO: 185 - protein, 92 aa, synthetic construct
MGSLPAPKNLVVSRVTEDSARLSWTAPDAAFDSFEISYWEHTFNGEAIILFVPGSERSYDLTGLKPGTEYWVQIGGVKGGNWSAPLSAIFTT

SEQ ID NO: 186 - protein, 91 aa, synthetic construct
MSLPAPKNLVVSRVTEDSARLSWTAPDAAFDSFEIGYFENPYLGEAIVLTVPGSERSYDLTGLKPGTEYWVQIGGVKGGNWSAPLSAIFTT

SEQ ID NO: 187 - protein, 91 aa, synthetic construct
MSLPAPKNLVVSRVTEDSARLSWTAPDAAFDSFEIGYWENAYKGEAIVLTVPGSERSYDLTGLKPGTEYRVWIGGVKGGVWSRPLSAIFTT

SEQ ID NO: 188 - protein, 91 aa, synthetic construct
MSLPAPKNLVVSRVTEDSARLSWTAPDAAFDSFEIGYWENAYKGEAIVLTVPGSERSYDLTGLKPGTEYVVFIGGVKGGHGSQPLSAIFTT

SEQ ID NO: 189 - protein, 91 aa, synthetic construct
MSLPAPKNLVVSRVTEDSARLSWTAPDAAFDSFEIGYFEHTYYGEAIVLTVPGSERSYDLTGLKPGTEYWVQIGGVKGGNWSAPLSAIFTT

SEQ ID NO: 190 - protein, 91 aa, synthetic construct
MSLPAPKNLVISRVTEDSARLSWTAPDAAFDSFEIGYFENLYLGEAIVLTVPGSERSYDLTGLKPGTEYWVQIGGVKGGNWSAPLSAISTT

SEQ ID NO: 191 - protein, 91 aa, synthetic construct
MSLPAPKNLVVSRVTEDSARLSWTAPDAAFDSFEIGYFENLYLGEAIVLTVPGSERSYDLAGLKPGTEYWVQIGGVKGGNWSAPLSAIFTT

SEQ ID NO: 192 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGWGSNPLSAIFTT

SEQ ID NO: 193 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSEPLSAIFTT

SEQ ID NO: 194 - protein, 91 aa, synthetic construct
MSLPAPKNLVVSRVTEDSARLSWTAPDAAFDSFEIGYFENLYLGEAIVLTVPGSERSYDLTGLKPGTEYPVWIGGVKGGTWSVPLSTIFTT

SEQ ID NO: 195 - protein, 91 aa, synthetic construct
MSLPAPKNLIVSRVTEDSARLSWTAPDAAFDSFEIGYFENLYLGEAIVLTVPGSERSYDLTGLKPGTEYWVQIGGVKGSNWSAPLSAIFTT

SEQ ID NO: 196 - protein, 91 aa, synthetic construct; identical to SEQ ID NO: 154

SEQ ID NO: 197 - protein, 91 aa, synthetic construct
MSLPAPKNLVVSRVTEDSARLSWTAPDAAFDSFFIGYLEPQPPGEAISLLVPGSERSYDLTGLKPGTEYNVTIQGVKGGFPSEPLHAIFTT

SEQ ID NO: 198 - protein, 91 aa, synthetic construct
MSLPAPKNLVVSRVTEDSARLSWTAPDAAFDSFLIGYTEYSVYGEAIVLTVPGSERSYDLTGLKPGTEYTVWIMGVKGGIKSTPLSAISTT

SEQ ID NO: 199 - protein, 91 aa, synthetic construct
MSLPAPKNLVVSRVTEDSARLSWTAPDAAFDSFEIGYFENLYLGEAIVLTVPGYERSYDLTGLKPGTEYWVQIGGVKGGNWSAPLSAIFTT

SEQ ID NO: 200 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGSNWSAPLSAIFTT

SEQ ID NO: 201 - protein, 91 aa, synthetic construct
MSLPAPKNLVVSRVTEDSARLSWTAPDAAFDSFEIAYFEGTFHGEAIVLFVPGSERSYDLTGLKPGTEYAVWIGGVKGGDYSRPLSAAFTT

SEQ ID NO: 202 - protein, 91 aa, synthetic construct
MSLPAPKNLVVSRVTEDSARLSWTAPDAAFDSFEIGYFENLYLGEAIVLTVPGSERSYDLTGLKPGTEYYVHIGGVKGGIWSSPLSAIFTT

SEQ ID NO: 203 - protein, 91 aa, synthetic construct
MSLPAPKNLVVSRVTEDFARLSWTAPDAAFDSFEIAYWEQSYTGEAIVLTVPGSERSYDLTGLKPGTEYFVHIGGVKGGVWSTPLSAIFTT

SEQ ID NO: 204 - protein, 91 aa, synthetic construct
MSLPAPKNLVVSRVTEDSARLSWTAPDAAFDSFLIQYDEWPTYGEAIVLTVPGSERSYDLTGLKPGTEYLVEIVGVKGGNLSGPLSAIFTT

SEQ ID NO: 205 - protein, 91 aa, synthetic construct
MSLPAPKNLVVSRVTEDSARLSWTAPDAAFDSFEIGYFENLYLGEAIHLFVPGSERSYDLTGLKPGTEYNVTIQGVKGGFPSEPLIANFTT

SEQ ID NO: 206 - protein, 91 aa, synthetic construct
MSLPAPKNLVASRVTEDSARLSWTAPDAAFDSFEIGYFENLYLGEAIVLTVPGSERSYDLTGLKPGTEYWVQIGGVKGGNWSAPLSAIFTT

SEQ ID NO: 207 - protein, 91 aa, synthetic construct
MSLPAPKNLVVSRVTEDSARLSWTAPDAAFDSFEIGYWENAYKGEAIVLTVPGSERSYDLTGLKPGTEYVVFIGGVKGGVWSVPLSAISTT

SEQ ID NO: 208 - protein, 91 aa, synthetic construct
MSLPAPKNLVVSRVTEDSARLSWTAPDAAFDSFFIGYFENLYLGEAIVLTVPGSERSYDLTGLKPGTEYWVQIGGVKGGNWSAPLSAIFTT

SEQ ID NO: 209 - protein, 91 aa, synthetic construct
MSLPAPKNLVVSRVTEDSARLSWTAPDAAFDSFEIAYFERTWFGEAIVLTVPGSERSYDLTGLKPGTEYWVQIGGVKGGDWSRPLSAIFTT

SEQ ID NO: 210 - skipped

SEQ ID NO: 211 - protein, 91 aa, synthetic construct
MSLPAPKNLVVSRVTEDSARLSWTAPDAAFDSFEIVYAEPVRFGEAIVLTVPGSERSYDLTGLKPGTEYWVQIGGVKGGEWSSLLSAIFTT

SEQ ID NO: 212 - protein, 91 aa, synthetic construct
MSLPAPKNLVVSRVTEDSARLSWTAPDAAFDSFEIGYFENLYLGEAIVLTVPGSERYYDLTGLKPGTEYWVQIGGVKGGNWSAPLSAIFTT

SEQ ID NO: 213 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGAKGGNWSAPLSAIFTT

SEQ ID NO: 214 - protein, 91 aa, synthetic construct
MSLPAPKNLVVSRVTEDSARLSWTAPDAAFDSFEIDYGEYSQAGEAIGLLVPGSERSYDLTGLKPGTEYWVQIGGVKGGNWSAPLSAIFTT

SEQ ID NO: 215 - protein, 91 aa, synthetic construct
MSLPAPKNLVVSRVTEDSARLSWTAPDAAFDSLEIGYFENLYLGEAIVLTVPGSERSYDLTGLKPGTEYWVQIGGVKGGNWSAPLSAIFTT

SEQ ID NO: 216 - protein, 91 aa, synthetic construct
MSLPAPKNLVVSRVTEDSARLSWTAPDAAFDSFEIAYFEQTYTGEAIVLTVPGSERSYDLTGLKPGTEYWVQIGGVKGGTWSAPLSAISTT

SEQ ID NO: 217 - protein, 91 aa, synthetic construct
MSLPAPKNLVVSRVTEDSARLSWTAPDAAFDSFEIAYFEQTYTGEAIVLTVPGSERSYDLTGLKPGTEYWVAIGGVKGGLWSLPLSAIFTT

SEQ ID NO: 218 - protein, 91 aa, synthetic construct
MSLPAPKNLVVSRVTEDSARLSWTAPDAAFDSFEIAYHEDDFYGEAIALLVPGSERSYDLTGLKPGTEYIVHIGGVKGGFFSSPLYAWFTT

SEQ ID NO: 219 - protein, 91 aa, synthetic construct
MSLPAPKNLVVSRVTEDSARLSWTAPDAAFDSFAIMYGERGNPGEAIVLTVPGSERSYDLTGLKPGTEYAVWIYGVKGGNYSYPLSAIFTT

SEQ ID NO: 220 - protein, 91 aa, synthetic construct
MSLPAPKNLVVSRVTEDSARLSWTAPDAAFDSFEIAYWEQSYTGEAIVLTVPGSERSYDLTGLKPGTEYWVQIGGVKGGNWSAPLSAIFTT

SEQ ID NO: 221 - protein, 91 aa, synthetic construct
MSLPAPKNLVVSRVTEDSARLSWTAPDAAFDSFFIGYLEPRPPGEAIHLYVPGSERSYDLTGLKPGTEYNITIQGVKGGFPSIPLIASFTT

SEQ ID NO: 222 - protein, 91 aa, synthetic construct
MSLPAPKNLVVSRVTEDSARLSWTAPDAAFDSFFIGYLEPQPPGEAITLYVPGSERSYDLTGLKPGTEYNVTIQGVKGGFPSGPLIASFTT

SEQ ID NO: 223 - protein, 91 aa, synthetic construct
MSLPAPKNLVVSRVTEDSARLSWTAPDAAFDSFEIAYFERTWFGEAIVLTVPGSERSYDLTGLKPGTEYWVAIGGVKGGLWSLPLSVIFTT

SEQ ID NO: 224 - protein, 91 aa, synthetic construct
MSLPAPKNLVVSRVTEDSARLSWTAPDAAFDSFEIGYWENAYKGEAIVLTVPGSERSYDLTGLKPGTEYVVFIGGVKGGGWSGPLSAIFTT

SEQ ID NO: 225 - protein, 91 aa, synthetic construct; identical to SEQ ID NO: 154

SEQ ID NO: 226 - protein, 91 aa, synthetic construct
MSLPAPKNLVISRVTEDSARLSWTAPDAAFDSFEIGYFENLYLGEAIVLTVPGSERSYDLTGLKPGTEYWVAIGGVKGGLWSLPLSAIFTT

SEQ ID NO: 227 - protein, 90 aa, synthetic construct
MSLPAPKNLVVSRVTEDSARLSWTAPDAAFDSFEIAYAELSTGEAIVLTVPGSERSYDLTGLKPGTEYYVHIGGVKGGSWSIPLSAIFTT

SEQ ID NO: 228 - protein, 91 aa, synthetic construct
MSLPAPKNLVVSRVTEDSARLSWTAPDAAFDSFEIGYWENAYKGEAIVLTVPGSERSYDLTGLKPGTEYVVFIGGVKGGVWSKPLSVIFTT

SEQ ID NO: 229 - protein, 91 aa, synthetic construct
MSLPAPKNLVVSRVTEDSARLSWTAPDAAFDSFSIMYAEQKVNGEAIVLTVPGSERSYDLTGLKPGTEYLVLIWGVKGGGRSLPLSAIFTT

SEQ ID NO: 230 - protein, 91 aa, synthetic construct
MSLPAPKNLIVSRVTEDSARLSWTAPDAAFDSLEIGYFENLYLGEAIVLTVPGSERSYDLTGLKPGTEYWVQIGGVKGGNWSAPLSAIFTT

SEQ ID NO: 231 - protein, 91 aa, synthetic construct
MSLPAPKNLVVSRVTEDSARLSWTAPDAAFDSFFIGYLEPQPPGEAIHLFVPGSERSYDLTGLKPGTEYNVTIQGVKGGFPSEPLIANSTT

SEQ ID NO: 232 - protein, 91 aa, synthetic construct
MSLPAPKNLVVSRVTEDSARLSWTAPDAAFDSFEISYWEHTFNGEAIILFVPGSERSYDLTGLKPGTEYVVFIGGVKGGVWSVPLSAIFTT

SEQ ID NO: 233 - protein, 91 aa, synthetic construct
MSLPAPKNLVVSRVTEDSARLSWTAPDAAFDSFFIGYLEPQPPGEAIHLSVPGSERSYDLTGLKPGTEYWVQIGGVKGGNWSAPLSAIFTT

SEQ ID NO: 234 - protein, 91 aa, synthetic construct
MSLPAPKNLVVSRVTEDSARLSWTAPDAAFDSFFIGYLEPQPPGEAIALYVPGSERSYDLTGLKPGTEYNVTIQGVKGGFPSEPLVAHFTT

SEQ ID NO: 235 - protein, 91 aa, synthetic construct
MSLPAPKNLVVSRVTEDSARLSWTAPDAAFDSFEIVYAEPVRFGEAIVLTVPGSERSYDLTGLKPGTEYWVQIGGVKGGNWSAPLSAIFTT

SEQ ID NO: 236 - protein, 91 aa, synthetic construct
MSLPAPKNLVVSRVTEDSARLSWTEPDAAFDSFEIGYFENLYLGEAIVLTVPGSERSYDLTGLKPGTEYWVQIGGVKGGNWSAPLSAIFTT

SEQ ID NO: 237 - protein, 91 aa, synthetic construct; identical to SEQ ID NO: 158

SEQ ID NO: 238 - protein, 91 aa, synthetic construct
MSLPAPKNLVVSRVTEDSARLSWTAPDAAFDSFEIGYFENDYFGEAIVLTVPGSERSYDLTGLKPGTEYVVFIGGVKGGKWSAPLSAIFTT

SEQ ID NO: 239 - protein, 91 aa, synthetic construct
MSLPAPKNLVVSRVTEDSARLSWTAPDAAFDSFEIVYQENSAYGEAIVLTVPGSERSYDLTGLKPGTEYWVQIGGVKGGNWSAPLSAIFTT

SEQ ID NO: 240 - protein, 91 aa, synthetic construct
MSLPAPKNLVVSRVTEDSARLSWTAPDAAFDSFHIWYFEKANEGEAIPLVVPGSERSYDLTGLKPGTEYDVDIGGVKGGAWSIPLGARFTT

SEQ ID NO: 241 - protein, 91 aa, synthetic construct
MSLPAPKNLVVSRVTEDSARLSWTAPDAAFDSFMIAYDEFIVWGEAVVLTVPGSERSYDLTGLKPGTEYLVEILGVKGGTISGPLSAIFTT

SEQ ID NO: 242 - protein, 91 aa, synthetic construct
MSLPAPKNLVVSRVTEDSARLSWTAPDAAFDSFEIGYFENLYLGEAIVLTVPGSERSYDLTGLKPGTKYWVQIGGVKGGNWSAPLSAIFTT

SEQ ID NO: 243 - protein, 91 aa, synthetic construct
MSLPAPKNLVVSRVTEDSARLSWTAPDAAFDSFFIGYLEPQPPGEAIHLYVPGSERSYDLTGLKPGTEYNVTIQGVKGGEWSAPLSAIFTT

SEQ ID NO: 244 - protein, 91 aa, synthetic construct
MSLPAPKNLVVSRVTEDSARLSWTAPDAAFDSFEIGYFENLYLGEAIVLTVPGSERSYDLTGLKPGTEYVVFIGGVKGGEYSIPLSAIFTT

SEQ ID NO: 245 - protein, 91 aa, synthetic construct
MSLPAPKNLVVSRVTEDSARLSWTAPDAAFDSFEISYWEHTFNGEAIILFVPGSERSYDLTGLKPGTEYYVTIGGVKGGAWSPPLWAHFTT

SEQ ID NO: 246 - protein, 91 aa, synthetic construct
MSLPAPKNLVVSRVTEDSARLSWTAPDAAFDSFEIAYQELSLWGEAIVLTVPGSERSYDLTGLKPGTEYYVHIGGVKGGIWSSPLSAYFTT

SEQ ID NO: 247 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGEWSAPLSAIFTT

SEQ ID NO: 248 - protein, 91 aa, synthetic construct
MSLPAPKNLVVSRVTEDSARLSWTAPDAAFDSFEIGYFENLYLGEAIVLTVPGSERSYDLTGLKPGTEYVVFIGGVKGGDFSPPLSAIFTT

SEQ ID NO: 249 - protein, 91 aa, synthetic construct
MSLPAPKNLVVSRVTEDSARLSWTAPDAAFDSFEIGYWENAYRGEAIVLTVPGSERSYDLTGLKPGTEYVVFIGGVKGGVWSVPLSAIFTT

SEQ ID NO: 250 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVNGGNWSAPLSAIFTT

SEQ ID NO: 251 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLVATFTT

SEQ ID NO: 252 - protein, 91 aa, synthetic construct; identical to SEQ ID NO: 154

SEQ ID NO: 253 - protein, 91 aa, synthetic construct
MSLPAPKNLVVSRVTEDSARLSWTAPDAAFDSFDINYWENESGGEAIALFVPGSERSYDLTGLKPGTEYWVSIGGVKGGRFSEPLYARFTT

SEQ ID NO: 254 - protein, 91 aa, synthetic construct
MSLPAPKNLVVSRVTEDSARLSWTAPDAAFDSFEIGYFENLYLGEAIVLWVPGSERSYDLTGLKPGTEYWVQIGGVKGGNWSAPLSAIFTT

SEQ ID NO: 255 - protein, 91 aa, synthetic construct
MSLPAPKNLVVSRVTEDSARLSWTTPDAAFDSFEIAYFEIAWLGEAIVLTVPGSERSYDLTGLKPGTEYWVQIGGVKGGAYSTPLSAIFTT

SEQ ID NO: 256 - protein, 91 aa, synthetic construct
MSLPVPKNLVVSRVTEDSARLSWTAPDAAFDSFEIGYFENLYLGEAIVLTVPGSERSYDLTGLKPGTEYWVQIGGVKGGNWSAPLSAIFTT

SEQ ID NO: 257 - protein, 91 aa, synthetic construct
MSLPAPKNLVVSHVTEDSARLSWTAPDAAFDSFEIGYWENAYKGEAIVLTVPGSERSYDLTGLKPGTEYVVFIGGVKGGVWSVPLSAIFTT

SEQ ID NO: 258 - protein, 91 aa, synthetic construct
MSLPAPKNLVVSRVTEDSARLSWTAPDAAFDSFFIGYLEPQPPGEAIVLGVPGSERSYDLTGLKPGTEYNVTIQGVKGGFPSDPLVASFTT

SEQ ID NO: 259 - protein, 91 aa, synthetic construct
MSLPAPKNLVVSRVTEDSARLSWTAPDAAFDSFYIAYAEPRPDGEAILLEVPGSERSYDLTGLKPGTEYSVLIHGVKGGLLSSPLSATFTT

SEQ ID NO: 260 - protein, 91 aa, synthetic construct
MSLPAPKNLVVSRITEDSARLSWTAPDAAFDSFEIGYFENLYLGEAIVLTVPGSERSYDLTGLKPGTEYWVQIGGVKGGNWSAPLSAIFTT

SEQ ID NO: 261 - protein, 91 aa, synthetic construct
MSLPAPKNLVVSRVTEDSARLSWTAPDAAFDSFYIAYAEPRPDGEAILLWVPGSERSYDLTGLKPGTEYSVLIHGVKGGLLSSPLHAYFTT

SEQ ID NO: 262 - protein, 91 aa, synthetic construct
MSLPAPKNLVVSRVTEDSARLSWTAPDAAFDSFFIGYLEPQPPGEVIHLFVPGSERSYDLTGLKPGTEYNVTIQGVKGGFPSDPLVASFTT

SEQ ID NO: 263 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSSPLSAIFTT

SEQ ID NO: 264 - protein, 91 aa, synthetic construct
MSLPAPKNLVVSRVTEDSARLSWTAPDAAFDSFFIGYLEPQPPGEAIALYVPGSERSYDLTGLKPGTEYNVTIQGVKGGFPSEPLVVHFTT

SEQ ID NO: 265 - protein, 91 aa, synthetic construct
MSLPAPKNLVVSRVTEDSARLSWTAPDAAFDSFFIGYLEPQPPGEAISLYVPGSERSYDLTGLKPGTEYNVTIQGVKGGFPSDPLVAHFTT

SEQ ID NO: 266 - protein, 91 aa, synthetic construct
MSLPAPKNLVVSRVTEDSARLSWTAPDAAFDSFFIGYLEPQPPGEAIYLLVPGSERSYDLTGLKPGTEYNVTIQGVKGGFPSLPLVATFTT

SEQ ID NO: 267 - protein, 91 aa, synthetic construct
MSLPAPKNLVVSRVTEDSARLSWTAPDAAFDSFEISYWEHTFNGEAIILFVPGSERSYDLTGLKPGTEYYVTIGGVKGGAWSPPLWAEFTT

SEQ ID NO: 268 - protein, 91 aa, synthetic construct
MSLPAPKNLVVSRVTEDSARLSWTAPDAAFDSFEIGYFENLYLGEAIVLTVPGSERSYDPTGLKPGTEYWVQIGGVKGGNWSAPLSAIFTT

SEQ ID NO: 269 - protein, 91 aa, synthetic construct
MSLPAPKNLVVSRVTEDSARLSWTAPDAAFDSFEIGYFENDYFGEAIVLTVPGSERSYDLTGLKPGTEYVVFIGGVKGGNWSAPLSAIFTT

SEQ ID NO: 270 - protein, 91 aa, synthetic construct
MSLPAPKNLVVSRVTEDSARLSWTAPDAAFDSFFIGYLEPQPPGEAIHLYVPGSERSYDLTGLKPGTEYNVTIQGVKGGFPSEPLIANSTT

SEQ ID NO: 271 - protein, 91 aa, synthetic construct
MSLPAPKNLVVSRITEDSARLSWTAPDAAFDSFEIGYWENAYKGEAIVLTVPGSERSYDLTGLKPGTEYVVFIGGVKGGVWSVPLSAIFTT

SEQ ID NO: 272 - protein, 91 aa, synthetic construct
MSLPAPKNLVVSRVTEDSARLSWTAPDAAFDSFEIAYGEAAYDGEAIALLVPGSERSYDLTGLKPGTEYWVQIGGVKGGNWSAPLSAIFTT

SEQ ID NO: 273 - protein, 90 aa, synthetic construct
MLPAPKNLVVSRVTEDSARLSWTAPDAAFDSFEISYWEHTFNGEAIILFVPGSERSYDLTGLKPGTEYYVTIGGVKGGAWSPPLWAEFTT

SEQ ID NO: 274 - protein, 91 aa, synthetic construct
MSLPAPKNLVVSRVTEDSARLSWTAPDAAFDSFEIVYWEQVGVGEAIVLTVPGSERSYDLTGLKPGTEYWVQIGGVKGGGFSEPLSAIFTT

SEQ ID NO: 275 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLVAEFTT

SEQ ID NO: 276 - protein, 91 aa, synthetic construct
MSLPAPKNLVVSRVTEDSARLSWTAPDAAFDSFEIAYFELDYVGEAIVLTVPGSERSYDLTGLKPGTEYWVQIGGVKGGTWSGPLSAIFTI

SEQ ID NO: 277 - protein, 91 aa, synthetic construct
MSLPAPKNLVVSRVTEDSARLSWTAPDAAFDSFEIGYFELTYYGEAIVLTVPGSERSYDLTGLKPGTEYWVQIGGVKGGGFSEPLSAIFTT

SEQ ID NO: 278 - protein, 91 aa, synthetic construct
MSLPAPKNLVVSRVTEDSARLSWTAPDAAFDSFEIGYFENLYLGEAIVLTVPGSERSHDLTGLKPGTEYWVQIGGVKGGNWSAPLSAIFTT

SEQ ID NO: 279 - protein, 91 aa, synthetic construct
MSLPAPKNLVVSRVTEDSARLSWTAPDAAFDSFFIGYLEPQPPGEAIHLYVPGSERSYDLTGLKPGTEYNVTIQGVKGGNWSAPLSAIFTT

SEQ ID NO: 280 - protein, 91 aa, synthetic construct
MSLPAPKNLVVSRVTEDSARLSWTALDAAFDSFEIGYFENLYLGEAIVLTVPGSERSYDLTGLKPGTEYWVQIGGVKGGNWSAPLSAIFTT

SEQ ID NO: 281 - protein, 91 aa, synthetic construct
MSLPAPKNLVVSRVTEDSARLSWTAPDAAFDSFFIGYLEPQPPGEAIALYVPGSERSYDLTGLKPGTEYNVTIQGVKGGFPSEPLIANFTT

SEQ ID NO: 282 - protein, 91 aa, synthetic construct
MSLPAPKNLVVSRVTEDSARLSWTAPDAAFDSFDIGYFELTWYGEAIVLTVPGSERSYDLTGLKPGTEYWVSIGGVKGGRFSEPLYARFTT

SEQ ID NO: 283 - protein, 91 aa, synthetic construct
MSLPAPKNLVVSRVTEDSARLSWTAPDAAFDSFEIAYQELSLWGEAIVLTVPGSERSYDLTGLKPGTEYYVHIGGVKGGIWSSPLSAYSTT

SEQ ID NO: 284 - protein, 91 aa, synthetic construct
MSLPAPKNLVVSRVTEDSARLSWTAPDAAFDSFEIGYWENAYKGEAIVLTVPGSERSYDLTGLKPGTEYMVFIGGVKGGVWSVPLSAIFTT

SEQ ID NO: 285 - protein, 91 aa, synthetic construct
MSLPAPKNLVVSRVTEDSARLSWTATDAAFDSFEIGYWENAYKGEAIVLTVPGSERSYDLTGLKPGTEYVVFIGGVKGGVWSVPLSAIFTT

SEQ ID NO: 286 - protein, 91 aa, synthetic construct
MSLPAPKNLVVSRVTEDSARLSWTAPDAAFDSFEIVYAEPVRFGEAIVLTVPGSERSYDLTGLKPGTEYWVQIGGVKGGEWSSPLSAIFTT

SEQ ID NO: 287 - protein, 91 aa, synthetic construct; identical to SEQ ID NO: 154

SEQ ID NO: 288 - protein, 91 aa, synthetic construct; identical to SEQ ID NO: 260

SEQ ID NO: 289 - protein, 91 aa, synthetic construct
MSLPAPKNLVVSRVTEDSARLSWTAPDAAFDSFEIAYFEIAWLGEAIVLTVPGSERSYDLTGLKPGTEYWVQIGGVKGGAYSTPLSAIFTT

SEQ ID NO: 290 - protein, 91 aa, synthetic construct
MSLPAPKNLVVSRVTEDSARLSWTAPDAAFDSFFIGYLEPQPPGEAIHLYVPGSERSYDLTGLKPGTEYNVTIQGVKGGFPSEPLVATFTT

SEQ ID NO: 291 - protein, 91 aa, synthetic construct; identical to SEQ ID NO: 260

SEQ ID NO: 292 - protein, 91 aa, synthetic construct
MSLPAPKNLVVSRVTEDSARLSWTAPDAAFDSFEIGYWENAYKGEAIVLTVPGSERSYDLTGLKPGTEYVVFIGGVKGGVWSVSLSAIFTT

SEQ ID NO: 293 - protein, 91 aa, synthetic construct
MSLPAPKNLVVSRVTEDSARLSWTAPDAAFDSFFIGYLEPQPPGEAIDLYVPGSERSYDLTGLKPGTEYNVTIQGVKGGFPSHPLVATFTT

SEQ ID NO: 294 - protein, 91 aa, synthetic construct
MSLPAPKNLVVSRVTEDSARLSWTAPDAAFDSFFIGYLEPQPLGEAIHLFVPGSERSYDLTGLKPGTEYNVTIQGVKGGFPSEPLIASFTT

SEQ ID NO: 295 - protein, 91 aa, synthetic construct
MSLPAPKNLVVSRVTEDSARLSWTAPDAAFDSFFIGYLEPQPPGEAIDLYVPGSERSYDLTGLKPGTEYNVTIQGVKGGFPSEPLVATFTT

SEQ ID NO: 296 - protein, 91 aa, synthetic construct
MSLPAPKNLVVSRVTEDSARLSWTAPDAAFDSFDIGYFELTWYGEAIVLTVPGSERSYDLTGLKPGTEYWVSIGGVKGGIWSSPLSAIFTT

SEQ ID NO: 297 - protein, 91 aa, synthetic construct
MSLPAPKNLVVSRVTEDSARLSWTAPDAAFDSFEISYFENTWYGEAIVLTVPGSERSYDLTGLKPGTEYSVRIFGVKGGNFSFPLSAIFTT

SEQ ID NO: 298 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGDNWSAPLSAIFTT

SEQ ID NO: 299 - protein, 91 aa, synthetic construct
MSLPAPKNLVVSRVTEDSARLSWTAPDAAFDSFEIAYFENLYLGEAIVLTVPGSERSYDLTGLKPGTEYWVQIGGVKGGNWSAPLSAIFTT

SEQ ID NO: 300 - protein, 91 aa, synthetic construct
MSLPAPKNLVVSRVTEDSARLSWTAPDAAFDSFEIGYFEATPNGEAIVLTVPGSERSYDLTGLKPGTEYKVFIGGVKGGRWSKPLSAIFTT

SEQ ID NO: 301 - protein, 91 aa, synthetic construct
MSLPAPKNLVVSRVTEDSARLSWTTPDAAFDSFEIGYFENLYLGEAIVLTVPGSERSYDLTGLKPGTEYWVQIGGVKGGNWSAPLSAIFTT

SEQ ID NO: 302 - skipped

SEQ ID NO: 303 - protein, 91 aa, synthetic construct
MSLPAPKNLVVSRVTEDSARLSWTAPDAAFDSFWIEYWEGWKSGEAIVLTVPGSERSYDLTGLKPGTEYRVHIWGVKGGIVSWPLSAIFTT

SEQ ID NO: 304 - protein, 91 aa, synthetic construct
MSLPAPKNLVVSRVTEDSARLSWTAPDAAFDSFEIGYFENLYLGEAIVLTVPGSERSYDLTGLKPGTEYVVFIGGVKGGVWSVPLSAIFTT

SEQ ID NO: 305 - protein, 91 aa, synthetic construct
MSLPAPKNLVVSRVTEDSARLSWTAPDAAFDSFEIAYFEQTYTGEAIVLTVPGSERSYDLTGLKPGTEYWVQIGGVKGGNWSAPLSAIFTT

SEQ ID NO: 306 - protein, 91 aa, synthetic construct
MSLPAPKNLVVSRVTEDSARLSWTAPDAAFDSFEIGHFENLYLGEAIVLTVPGSERSYDLTGLKPGIEYWVQIGGVKGGVWSVSLSAIFTT

SEQ ID NO: 307 - protein, 91 aa, synthetic construct
MSLPAPKNLVVSRVTEDSARLSWTAPDAAFNSFEIGYFENLYLGEAIVLTVPGSERSYDLTGLKPGTEYWVQIGGVKGGTFSAPLSAIFTT

SEQ ID NO: 308 - protein, 91 aa, synthetic construct
MSLPAPKNLVVSRVTEDSARMSWTTPDAAFDSFEIGYFENLYLGEAIVLTVPGSERSYDLTGLKPGTEYWVQIGGVKGGNWSAPLSAIFTT

SEQ ID NO: 309 - protein, 91 aa, synthetic construct
MSLPAPENLVVSRVTEDSARLSWTAPDAAFDSFEIGYFENLYLGEAIVLTVPGSERSYDLTGLKPGTEYWVQIGGVKGGNWSAPLSAIFTT

SEQ ID NO: 310 - protein, 91 aa, synthetic construct
MSLPAPKNLVVSRVTEDSARLSWTAPDAAFDSFEIDYGEYSQAGEAIGLLVPGSERSYDLTGLKPGTEYAVWIGGVKGGFFSTPLEADFTT

SEQ ID NO: 311 - protein, 91 aa, synthetic construct
MSLPAPKNLVVSRVTEDSARLSWTAPDAAFDSFEIGYWENAYKGEAIVLTVPGSERSYDLTGLKPGTEYVVFIGGVKGGSWSNPLSAIFTT

SEQ ID NO: 312 - protein, 91 aa, synthetic construct
MSLPAPKNLVVSRVTEDSARLSWTAPDAAFDSFEIGYFENLYLGEAIVLTIPGSERSYDLTGLKPGTEYWVQIGGVKGGNWSAPLSAIFTT

SEQ ID NO: 313 - protein, 91 aa, synthetic construct
MSLPAPKNLVVSRVTEDSARLSWTAPDAAFDSFEIAYFENDWAGEAIVLTIPGSERSYDLTGLKPGTEYWVQIGGVKGGNWSAPLSAIFTT

SEQ ID NO: 314 - protein, 92 aa, synthetic construct
MGSLPAPKNLVVSRVTEDSARLSWTAPDAAFDSFEIGYFENLYLGEAIVLTVPGSERSYDLTGLKPGTEYWVQIGGVKGGNWSAPLSAIFTT

SEQ ID NO: 315 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLSAISTT

SEQ ID NO: 316 - protein, 91 aa, synthetic construct
MSLPAPKNLVISRVTEDSARLSWTAPDAAFDSFEIGYFENLYLGEAIVLTVPGSERSYDLTGLKPGTEYWVQIGGVKGGNWSAPLSAIFTT

SEQ ID NO: 317 - protein, 91 aa, synthetic construct; identical to SEQ ID NO: 154

SEQ ID NO: 318 - skipped

SEQ ID NO: 319 - protein, 91 aa, synthetic construct
MSLPAPKNLVVSRVTEDSARLSWTAPGAAFDSFEIGYFENLYLGEAIVLTVPGSERSYDLTGLKPGTEYWVQIGGVKGGVWSVPLSAIFTT

SEQ ID NO: 320 - protein, 91 aa, synthetic construct
MSLPVPKNLVVSRVTEDSARLSWTAPDAAFDSFEIGYWENAYKGEAIVLTVPGSERSYDLTGLKPGTEYWVQIGGVKGGNWSAPLSAIFTT

SEQ ID NO: 321 - protein, 91 aa, synthetic construct; identical to SEQ ID NO: 154

SEQ ID NO: 322 - protein, 91 aa, synthetic construct
MSLPAPKNLVVSRVTEDSARLSWTAPDAAFDSFEIGYFEITRYGEAIILFVPGSERSYDLTGLKPGTEYWVQIGGVKGGNWSAPLSAIFTT

SEQ ID NO: 323 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSGPLSAIFTT

SEQ ID NO: 324 - protein, 91 aa, synthetic construct
MSLPAPKNLVVSRITEDSARLSWTAPDAAFDSFEIGYFENLYLGEAIVLTVPGSERSYDLTGLKPGTEYWVQIGGVKGGNWSAPLSAISTT

SEQ ID NO: 325 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLSEIFTT

SEQ ID NO: 326 - protein, 91 aa, synthetic construct
MSLPAPKNLVVSRVTEDSARLSWTAPDAAFDSFEIGYWENAYKGEAIVLTVPGSERSYDLTGLKPGTEYWVQIGGVKGGVWSTPLSAIFTT

SEQ ID NO: 327 - protein, 91 aa, synthetic construct
MSLPAPKNLVVSRVTEDSARLSWTAPDAAFDSFEIGYWENAYKGEAIVLTVPGSERSYDLTGLKPGTEYWVQIGGVKGGNWSAPLSAIFTT

SEQ ID NO: 328 - protein, 91 aa, synthetic construct
MSLPAPKNLVVSRVTEDSARLSWTAPDAAFDSFEIGYFGNLYLGEAIVLTVPGSERSYDLTGLKPGTEYWVQIGGVKGGNWSAPLSAIFTT

SEQ ID NO: 329 - protein, 91 aa, synthetic construct
MSLPAPKNLVVSRVTEDSARLSWTAPDAAFDSFDIGYFEATYYGEAIVLTVPGSERSYDLTGLKPGTEYRVWIGGVKGGYYSNPLSAIFTT

SEQ ID NO: 330 - protein, 91 aa, synthetic construct
MSLPAPKNLVVSRVTEDSARLSWTAPDAAFDSFEIGYFENLYLGEAIVLTVPGSERSYDLTGLKPGTEYWVQIGGVKGGNWSAPLSAIVTT

SEQ ID NO: 331 - protein, 91 aa, synthetic construct
MSLPAPKNLVVSRVTEDSARLSWTAPDAAFDSFFIGYLEPQPPGEAIHLYVPGSERSYDLTGLKPGTEYNVTIQGVKVGFPSEPLIANFTT

SEQ ID NO: 332 - protein, 91 aa, synthetic construct
MSLPAPKNLVVSRVTEDSARLSWTAPDAAFDSFEIGYWEGPGGGEAIILVVPGSERSYDLTGLKPGTEYYVQIGGVKGGDWSTPLWATFTT

SEQ ID NO: 333 - protein, 91 aa, synthetic construct
MSLPAPENLVVSRVTEDSARLSWTAPDAAFDSFFIGYLEPQPPGEAIALYVPGSERSYDLTGLKPGTEYNVTIQGVKGGFPSLPLVATFTT

SEQ ID NO: 334 - protein, 91 aa, synthetic construct
MSLPAPKNLVVSRVTEDSARLSWTAPDAAFDSFEIAYQELSLWGEAIVLTVPGSERSYDLTGLKPGTEYWVQIGGVKGGIWSSPLVASFTT

SEQ ID NO: 335 - protein, 91 aa, synthetic construct
MSLPAPKNLVVSRVTEDSARLSWTAPDAAFDSFFIGYLEPQPPGEAIDLYVPGSERSYDLTGLKPGTEYNVTIQGVKGGFPSLPLVATFTT

SEQ ID NO: 336 - protein, 91 aa, synthetic construct
MSLPAPKNLVVSRVTEDSARLSWTAPDAAFDSFNIMYHEHYDNGEAIVLTVPGSERSYDLTGLKPGTEYSVVINGVKGGPHSAPLSAIFTT

SEQ ID NO: 337 - protein, 91 aa, synthetic construct
MSLPAPKNLVVSRVTEDSARLSWTAPDAAFDSFFIGYLEPQPPGEAIHLYVPGSERSYDLTGLKPGTEYNVTIQGVKGGFPSIPLIASFTT

SEQ ID NO: 338 - protein, 91 aa, synthetic construct
MSLPAPKNLVVSRVTEDSARLSWTAPDAAFDSFEIGYFENLYLGEAIVLTVPGSERSYDLTGLKPGTEYCVYICGVKGGRDSMPLSAIFTT

SEQ ID NO: 339 - protein, 91 aa, synthetic construct
MSLPAPKNLVVSRVTEDSARLSWTAPDAAFDSFEIGYWEMSYYGEAIVLTVPGSERSYDLTGLKPGTEYGVSIGGVKGGRWSLPLSAIFTT

SEQ ID NO: 340 - protein, 91 aa, synthetic construct
MSLPAPKNLVVSRVTEDSSRLSWTAPDAAFDSFEIGYFENLYLGEAIVLTVPGSERSYDLTGLKPGTEYWVQIGGVKGGNWSAPLSAIFTT

SEQ ID NO: 341 - protein, 91 aa, synthetic construct
MSLPAPKNLVVSRVTEDSARLSWTAPDAAFDSFFIGYLEPQPPGEAIELYVPGSERSYDLTGLKPGTEYNVTIQGVKGGFPSLPLVAIFTT

SEQ ID NO: 342 - protein, 91 aa, synthetic construct
MSLPAPKNLVVSRVTEDSARLSWTAPDAAFDSFFIGYLEPQPPGEAIVLTVPGSERSYDLTGLKPGTEYNVTIQGVKGGFPSEPLIANFTT

SEQ ID NO: 343 - protein, 91 aa, synthetic construct
MSLPAPKNLVVSRVTEDSARLSWTAPDAAFDSFFIGYLEPQPPGEAIALWVPGSERSYDLTGLKPGTEYNVTIQGVKGGFPSEPLVATFTT

SEQ ID NO: 344 - protein, 91 aa, synthetic construct
MSLPAPKNLFVSRVTEDSARLSWTAPDAAFDSFEIGYWENAYKGEAIVLTVPGSERSYDLTGLKPGTEYWVQIGGVKGGNWSAPLSAIFTT

SEQ ID NO: 345 - protein, 91 aa, synthetic construct
MSLPAPKNLVVSRVTEDSAHLSWTAPDAAFDSFEIGYFENDYFGEAIVLTVPGSERSYDLTGLKPGTEYVVFIGGVKGGKWSAPLSAIFTT

SEQ ID NO: 346 - protein, 90 aa, synthetic construct
MLPAPKNLVVSRVTEDSARLSWTAPDAAFDSFFIGYLEPQPPGEAISLFVPGSERSYDLTGLKPGTEYNVTIQGVKGGFPSDPLNATFNT

SEQ ID NO: 347 - protein, 90 aa, synthetic construct
MLPAPKNLVVSRVTEDSARLSWTAPDAAFDSFFIGYLEPQPPGEAIGLFVPGSERSYDLTGLKPGTEYNVTIQGVKGGFPSEPLKAAFTT

SEQ ID NO: 348 - protein, 90 aa, synthetic construct
MLPAPKNLVVSRVTEDSARLSWTAPDAAFDSFEISYFENTWYGEAIVLTVPGSERSYDLTGLKPGTEYVVWIGGVKGGLWSKPLSAIFTT

SEQ ID NO: 349 - protein, 90 aa, synthetic construct
MLPAPKNLVVSRVTEDSARLSWTAPDAAFDSFFIGYLEPQPPGEAIELYVPGSERSYDLTGLKPGTEYNVTIQGVKGGFPSEPLIAHFTT

SEQ ID NO: 350 - protein, 90 aa, synthetic construct
MLPAPKNLVVSRVTEDSARLSWTAPDAAFDSFFIGYLEPQPPGEAISLFVPGSERSYDLTGLKPGTEYNVTIQGVKGGFPSMPLVAQFTT

SEQ ID NO: 351 - protein, 90 aa, synthetic construct
MLPAPKNLVVSRVTEDSARLSWTAPDAAFDSFFIGYLEPQPPGEAITLAVPGSERSYDLTGLKPGTEYNVTIQGVKGGFPSNPLLAAFTT

SEQ ID NO: 352 - protein, 90 aa, synthetic construct
MLPAPKNLVVSRVTEDSARLSWTAPDAAFDSFEIAYQELSLWGEAIVLTVPGSERSYDLTGLKPGTEYYVHIGGVKGGIWSSPLSAIFTT

SEQ ID NO: 353 - protein, 90 aa, synthetic construct
MLPAPKNLVVSRVTEDSARLSWTAPDAAFDSFFIGYLEPQPPGEAIGLFVPGSERSYDLTGLKPGTEYNVTIQGVKGGFPSNPLEARFTT

SEQ ID NO: 354 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSLPLVATFTT

SEQ ID NO: 355 - protein, 90 aa, synthetic construct
MLPAPKNLVVSRVTEDSARLSWTAPDAAFDSFFIGYLEPQPPGEAIHLYVPGSERSYDLTGLKPGTEYNVTIQGVKGGFPSHPLYADFTT

SEQ ID NO: 356 - protein, 90 aa, synthetic construct
MLPAPKNLVVSRITEDSARLSWTAPDAAFDSFFIGYLEPQPPGEAITLLVPASERSYDLTGLKPGTEYNVTIQGVKGGFPSHPLQAQFTT

SEQ ID NO: 357 - protein, 90 aa, synthetic construct
MLPAPKNLVVSRVTEDSARLSWTAPDAAFDSFFIGYLEPQPPGEAIGLFVPGSERSYDLTGLKPGTEYNVTIQGVKGGFPSDPLVAYFTT

SEQ ID NO: 358 - protein, 90 aa, synthetic construct
MLPAPKNLVVSRVTEDSARLSWTAPDAAFDSFEIGYWENAYKGEAIVLTVPGSERSYDLTGLKPGTEYVVFIGGVKGGVWSVPLSAIFTT

SEQ ID NO: 359 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGDYSSPLSAIFTT

SEQ ID NO: 360 - protein, 90 aa, synthetic construct
MLPAPKNLVVSRVTEDSARLSWTAPDAAFDSFFIGYLEPQPPGEAISLYVPGSERSYDLTGLKPGTEYNVTIQGVKGGFPSHPLLAVFTT

SEQ ID NO: 361 - protein, 90 aa, synthetic construct
MLPAPKNLVVSRVTEDSARLSWTAPDAAFDSFFIGYLEPQPPGEAIQLFVPGSERSYDLTGLKPGTEYNVTIQGVKGGFPSIPLSALFTT

SEQ ID NO: 362 - protein, 90 aa, synthetic construct
MLPAPKNLVVSRVTEDSARLSWTAPDAAFDSFEIAYWENNFNGEAIILIVPGSERSYDLTGLKPGTEYVVFISGVKGGTWSYPLVAQFTT

SEQ ID NO: 363 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSLPLSAIFTT

SEQ ID NO: 364 - protein, 90 aa, synthetic construct
MLPAPNNLVVSRVTEDSARLSWTAPDAAFDSFFIGYLEPQPPGEAIHLFVPGSERSYDLTGLKPGTEYNVTIQGVKGGFPSGPLHATFTT

SEQ ID NO: 365 - protein, 90 aa, synthetic construct
MLPAPKNLVVSRVTEDSARLSWTAPDAAFDSFFIGYLEPQPPGEAIVLWVPGSERSYDLTGLKPGTEYNVTIQGVKGGFPSHPLVAKFTT

SEQ ID NO: 366 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSNPLKAQFTT

SEQ ID NO: 367 - protein, 90 aa, synthetic construct
MLPAPKNLVVSRVTEDSARLSWTAPDAAFDSFYIAYAEPRPDGEAILLVVPGSERSYDLTGLKPGTEYSVLIHGVKGGLLSSPLVAFFTT

SEQ ID NO: 368 - protein, 90 aa, synthetic construct
MLPAPKNLVVSRVTEDSARLSWTAPDAAFDSFFIGYLEPQPPGEAITLTVPGSERSYDLTGLKPGTEYNVTIQGVKGGFPSGPLIASFTT

SEQ ID NO: 369 - protein, 90 aa, synthetic construct
MLPAPKNLVVSRVTEDSARLSWTAPDAAFDSFFIGYLEPQPPGEAIVLWVPGSERSYDLTGLKPGTEYNVTIQGVKGGFPSHPLVAKSTT

SEQ ID NO: 370 - protein, 90 aa, synthetic construct
MLPAPKNLVVSRVTEDSARLSWTAPDAAFDSFYIAYAEPRPDGEAIVLVVPGSERSYDLTGLKPGTEYSVLIHGVKGGLLSSPLSAHFTT

SEQ ID NO: 371 - protein, 90 aa, synthetic construct
MLPAPKNLVVSRVTEDSARLSWTAPDAAFDSFDIDYVEFGWTGEAIALLVPGSERSYDLTGLKPGTEYWVWIGGVKGGDYSPPLNAYFTT

SEQ ID NO: 372 - protein, 90 aa, synthetic construct
MLPAPKNLVVSRVTEDSARLSWTAPDAAFDSFFIGYLEPQPPGEAINLLVPGSERSYDLTGLKPGTEYNVTIQGVKGGFPSLPLLAEFTT

SEQ ID NO: 373 - protein, 90 aa, synthetic construct
MLPAPKNLVVSRVTEDSARLSWTAPDAAFDSFFIGYLEPQPPGEAISLFVPGSERSYDLTGLKPGTEYNVTIQGVKGGFPSDPLVASFTT

SEQ ID NO: 374 - protein, 90 aa, synthetic construct
MLPAPKNLVVSRVTEDSARLSWTAPDAAFDSFYIAYAEPRPDGEAIQLSVPGSERSYDLTGLKPGTEYSVLIHGVKGGLLSSPLHASFTT

SEQ ID NO: 375 - protein, 90 aa, synthetic construct
MLPAPKNLVVSRVTEDSARLSWTAPDAAFDSFFIGYLEPQPPGEAIDLTVPGSERSYDLTGLKPGTEYNVTIQGVKGGFPSMPLSAIFTT

SEQ ID NO: 376 - protein, 90 aa, synthetic construct
MLPAPKNLVVSRVTEDSARLSWTAPDAAFDSFFIGYLEPQPPGEAIALLVPGSERSYDLTGLKPGTEYNVTIQGVKGGFPSNPLEAKFTT

SEQ ID NO: 377 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSTPLQALFTT

SEQ ID NO: 378 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSTPLIATVTT

SEQ ID NO: 379 - protein, 90 aa, synthetic construct
MLPAPKNLVVSRVTEDSARLSWTAPDAAFDSFYIEYWEGYRSGEAIVLTVPGSERSYDLTGLKPGTEYRVHIWGVKGGAVSYPLSAIFTT

SEQ ID NO: 380 - protein, 90 aa, synthetic construct
MLPAPKNLVVSRVTEDSARLSWTAPDAAFDSFFIGYLEPQPPGEAIGLFVPGSERSYDLTGLKPGTEYNVTIQGVKGGFPSMPLSAIFTT

SEQ ID NO: 381 - protein, 90 aa, synthetic construct
MLPAPKNLVVSRVTEDSARLSWTAPDAAFDSFEIAYQELSLWGEAIVLTVPGSERSYDLTGLKPGTEYYVHIGGVKGGIWSRPLSAIFTT

SEQ ID NO: 382 - protein, 90 aa, synthetic construct
MLPAPKNLVVSRVTEDSARLSWTAPDAAFDSFFIGYLEPQPPGEAIELYVPGSERSYDLTGLKPGTEYNVTIQGVKGGFPSDPLEADFTT

SEQ ID NO: 383 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSLPLDAVFTT

SEQ ID NO: 384 - protein, 90 aa, synthetic construct
MLPAPKNLVVSRVTEDSARLSWTAPDAAFDSFEIDYAENRHHGEAIALLVPGSERSYDLTGLKPGTEYIVFIGGVKGGRWSQPLVASFTT

SEQ ID NO: 385 - protein, 90 aa, synthetic construct
MLPAPKNLVVSRVTEDSARLSWTAPDAAFDSFFIGYLEPQPPGEAIDLYVPGSERSYDLTGLKPGTEYNVTIQGVKGGFPSHPLQAHFTT

SEQ ID NO: 386 - protein, 90 aa, synthetic construct
MLPAPKNLVVSRVTEDSARLSWTAPDAAFDSFYIAYAEPRPDGEAITLLVPGSERSYDLTGLKPGTEYSVLIHGVKGGLLSSPLSAIFTT

SEQ ID NO: 387 - protein, 90 aa, synthetic construct
MLPAPKNLVVSRVTEDSARLSWTAPDAAFDSFFIGYLEPQPPGEAIHLVVPGSERSYDLTGLKPGTEYNVTIQGVKGGFPSDPLYAWFTT

SEQ ID NO: 388 - protein, 90 aa, synthetic construct
MLPAPKNLVVSRVTEDSARLSWTAPDAAFDSFEIAYFEYCLNGEAIVLTVPGSERSYDLTGLKPGTEYWVQIGGVKGGTWSWPLSAIFTT

SEQ ID NO: 389 - protein, 90 aa, synthetic construct
MLPAPKNLVVSRVTEDSARLSWTAPDAAFDSFFIGYLEPQPPGEAIHLLVPGSERSYDLTGLKPGTEYNVTIQGVKGGFPSMPLSAIVTT

SEQ ID NO: 390 - protein, 90 aa, synthetic construct
MLPAPKNLVVSRVTEDSARLSWTAPDAAFDSFEIGYFERAWFGEAIVLTVPGSERSYDLTGLKPGTEYAVFIGGVKGGSYSYPLSAIFTT

SEQ ID NO: 391 - protein, 90 aa, synthetic construct
MLPAPKNLIVSRVTEDSARLSWTAPDAAFDSFFIGYLEPQPPGEAISLYVPGSERSYDLTGLKPGTEYNVTIQGVKGGFPSDPLQAIFTT

SEQ ID NO: 392 - protein, 90 aa, synthetic construct
MLPAPKNLVVSRVTEDSARLSWTAPDAAFDSFFIGYLEPQPPGEAIHLGVPGSERSYDLTGLKPGTEYNVTIQGVKGGFPSLPLVATFTT

SEQ ID NO: 393 - protein, 90 aa, synthetic construct
MLPAPKNLVVSRVTEDSARLSWTAPDAAFDSFFIGYLEPQPPGEAIGLFVPGSERSYDLTGLKPGTEYNVTIQGVKGGFPSLPLQAHFTT

SEQ ID NO: 394 - protein, 90 aa, synthetic construct
MLPAPKNLVVSRVTEDSARLSWTAPDAAFDSFFIGYLEPQPPGEAIYLLVPGSERSYDLTGLKPGTEYNVTIQGVKGGFPSTPLEASFTT

SEQ ID NO: 395 - protein, 90 aa, synthetic construct
MLPAPKNLVVSRVTEDSARLSWTAPDTAFDSFFIGYLEPQPPGEAIGLFVPGSERSYDLTGLKPGTEYNVTIQGVKGGFPSDPLSAFFTT

SEQ ID NO: 396 - protein, 90 aa, synthetic construct
MLPAPKNLVVSRVTEDSARLSWTAPDAAFDSFFIGYLEPQPPGEAIYLTVPGSERSYDLTGLKPGTEYNVTIQGVKGGFPSMPLSAIFTT

SEQ ID NO: 397 - protein, 90 aa, synthetic construct
MLPAPKNLVVSRVTEDSARLSWTAPDAAFDSFIILYGEAQYDGEAIVLTVPGSERSYDLTGLKPGTEYPVDIYGVKGGPYSWPLSAIFTT

SEQ ID NO: 398 - protein, 90 aa, synthetic construct
MLPAPKNLVVSRVTEDSARLSWTAPDAAFDSFFIGYLEPQPPGEAIYLTVPGSERSYDLTGLKPGTEYNVTIQGVKGGFPSKPLTANFTT

SEQ ID NO: 399 - protein, 90 aa, synthetic construct
MLPAPKNLVVSRVTEDSARLSWTAPDAAFDSFFIGYLEPQPPGEAITLYVPGSERSYDLTGLKPGTEYNVTIQGVKGGFPSTPLTATFTT

SEQ ID NO: 400 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSGPLHATFTT

SEQ ID NO: 401 - protein, 90 aa, synthetic construct
MLPAPKNLVVSRVTEDSARLSWTAPDAAFDSFFIGYLEPQPPGEAIALWVPGSERSYDLTGLKPGTEYNITIQGVKGGFPSMPLVANFTT

SEQ ID NO: 402 - protein, 89 aa, synthetic construct
MLPAPKNLVVSRVTEDSARLSWTAPDAAFDSFGIYYWEYTGGEAIVLTVPGSERSYDLTGLKPGTEYVVRILGVKGGAYSTPLSAIFTT

SEQ ID NO: 403 - protein, 90 aa, synthetic construct
MLPAPKNLVVSRVTEDSARLSWTAPDAAFDSFYIAYAEPRPDGEAIVLDVPGSERSYDLTGLKPGTEYSVLIHGVKGGLLSSPLVADFTT

SEQ ID NO: 404 - protein, 90 aa, synthetic construct
MLPAPKNLVVSRVTEDSARLSWTAPDAAFDSFFIGYLEPQPPGEAIHLLVPGSERSYDLTGLKPGTEYNVTIQGVKGGFPSEPLLAIVTT

SEQ ID NO: 405 - protein, 90 aa, synthetic construct
MLPAPKNLVVSRVTEDSARLSWTAPDAAFDSFFIGYLEPQPPGEAIHLWVPGSERSYDLTGLKPGTEYNVTIQGVKGGFPSLPLSAFFTT

SEQ ID NO: 406 - protein, 90 aa, synthetic construct
MLPAPKNLVVSRVTEDSARLSWTAPDAAFDSFFIDYDESLDSGEAIVLTVPGSERSYDLTGLKPGTEYNVTIQGVKGGFPSSPLEAAFTT

SEQ ID NO: 407 - protein, 90 aa, synthetic construct
MLPAPKNLVVSRVTEDSARLSWTAPDAAFDSFEIAYFETCRTGEAIVLTVPGSERSYDLTGLKPGTEYRVWIGGVKGGVWSRPLSAIFTT

SEQ ID NO: 408 - protein, 90 aa, synthetic construct
MLPAPKNLVVSRVTEDSARLSWTAPDAAFDSFFIGYLEPQPPGEAIGLWVPGSERSYDLTGLKPGTEYNVTIQGVKGGFPSMPLSAIFTT

SEQ ID NO: 409 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSNPLSAQFTT

SEQ ID NO: 410 - protein, 90 aa, synthetic construct
MLPAPKNLVVSRVTEDSARLSWTAPDAAFDSFFIGYLEPQPPGEAIALWVPGSERSYDLTGLKPGTEYNVTIQGVKGGFPSQPLHAHFTT

SEQ ID NO: 411 - protein, 90 aa, synthetic construct
MLPAPKNLVVSRVTEDSARLSWTAPDAAFDSFFIGYLEPQPPGEAISLFVPGSERSYDLTGLKPGTEYNVTIQGVKGGFPSVPLSATFTT

SEQ ID NO: 412 - protein, 90 aa, synthetic construct
MLPAPKNLVVSRVTEDSARLSWTAPDAAFDSFFIGYLEPQPPGEAIGLLVPGSERSYDLTGLKPGTEYNVTIQGVKGGFPSHPLLAVFTT

SEQ ID NO: 413 - protein, 90 aa, synthetic construct
MLPAPKNLVVSRVTEDSARLSWTAPDAAFDSFFIGYLEPQPPGEAIYLRVPGSERSYDLTGLKPGTEYNVTIQGVKGGFPSAPLHAHFTT

SEQ ID NO: 414 - protein, 90 aa, synthetic construct
MLPAPKNLVVSRVTEDSARLSWTAPDAAFDSFFIGYLEPQPPGEAIHLYVPGSERSYDLTGLKPGTEYNVTIQGVKGGFPSHPLIANFTT

SEQ ID NO: 415 - protein, 90 aa, synthetic construct
MLPAPKNLVVSRVTEDSARLSWTAPDAAFDSFFIGYLEPQPPGEAIHLQVPGSERSYDLTGLKPGTEYNVTIQGVKGGFPSMPLSAIFTT

SEQ ID NO: 416 - protein, 90 aa, synthetic construct
MLPAPKNLVVSRVTEDSARLSWTAPDAAFDSFFIGYLEPQPPGEAIALYVPGSERSYDLTGLKPGTEYNVTIQGVKGGFPSLPLVATFTT

SEQ ID NO: 417 - protein, 90 aa, synthetic construct
MLPAPKNLVVSRVTEDSARLSWTAPDAAFDSFFIGYLEPQPPGEAIQLFVPGSERSYDLTGLKPGTEYNVTIQGVKGGFPSDPLVASFTT

SEQ ID NO: 418 - protein, 90 aa, synthetic construct
MLPAPKNLVVSRVTEDSARLSWTAPDAAFDSFEIAYFENSYYGEAIVLTVPGSERSYDLTGLKPGTEYAVYIGGVKGGSWSNPLSAISTT

SEQ ID NO: 419 - protein, 90 aa, synthetic construct
MLPAPKNLVVSRVTEDSARLSWTAPDAAFDSFFIGYLEPQPPGEAIALLVPGSERSYDLTGLKPGTEYNVTIQGVKGGFPSSPLNAYFTT

SEQ ID NO: 420 - protein, 90 aa, synthetic construct
MLPAPKNLVVSRVTEDSARLSWTAPDAAFDSFFIGYLEPQPPGEAIHLQVPGSERSYDLTGLKPGTEYNVTIHGVKGGIPSMPLSAKFTT

SEQ ID NO: 421 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSMPLEAVFTT

SEQ ID NO: 422 - protein, 90 aa, synthetic construct
MLPAPKNLVVSRVTEDSARLSWTAPDAAFDSFFIGYLEPQPPGEAISLLVPGSERSYDLTGLKPGTEYNVTIQGVKGGFPSHPLDASFTT

SEQ ID NO: 423 - protein, 90 aa, synthetic construct
MLPAPKNLVVSRVTEDSARLSWTAPDAAFDSFFIGYLEPQPPGEAIALYVPGSERSYDLTGLKPGTEYNVTIQGVKGGFPSEPLVATFTT

SEQ ID NO: 424 - protein, 90 aa, synthetic construct
MLPAPKNLVVSRVTEDSARLSWTAPDAAFDSFFIGYLEPQPPGEAIHLIVPGSERSYDLTGLKPGTEYNVTIQGVKGGFPSDPLDASFTT

SEQ ID NO: 425 - protein, 90 aa, synthetic construct
MLPAPKNLVVSRVTEDSARLSWTAPDAAFDSFFIGYLEPQPPGEAIGLLVPGSERSYDLTGLKPGTEYNVTIQGVKGGFPSNPLSAIFTT

SEQ ID NO: 426 - protein, 90 aa, synthetic construct
MLPAPKNLVVSRVTEDSARLSWTAPDAAFDSFFIGYLEPQPPGEAIFLLVPGSERSYDLTGLKPGTEYNVTIQGVKGGFPSMPLSAIFTT

SEQ ID NO: 427 - protein, 90 aa, synthetic construct
MLPAPKNLVVSRVTEDSARLSWTAPDAAFDSFFIGYLEPQPPGEAIHLRVPGSERSYDLTGLKPGTEYNVTIQGVKGGFPSDPLIAVFTT

SEQ ID NO: 428 - protein, 90 aa, synthetic construct
MLPAPKNLVVSRVTEDSARLSWTAPDAAFDSFFIGYLEPQPPGEAISLYVPGSERSYDLTGLKPGTEYNVTIQGVKGGFPSNPLIAQFTT

SEQ ID NO: 429 - protein, 90 aa, synthetic construct
MLPAPKNLVVSRVTEDSARLSWTAPDAAFDSFEIGYFERSFYGEAIILFVPGSERSYDLTGLKPGTEYAVWIGGVKGGVWSRPLSAIFTT

SEQ ID NO: 430 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSYPLSASFTT

SEQ ID NO: 431 - protein, 90 aa, synthetic construct
MLPAPKNLVVSRVTEDSARLSWTAPDAAFDSFFIGYLEPQPPGEAIVLFVPGSERSYDLTGLKPGTEYNVTIQGVKGGFPSNPLSAISTT

SEQ ID NO: 432 - protein, 90 aa, synthetic construct
MLPAPKNLVVSRVTEDSARLSWTAPDAAFDSFFIGYLEPQPPGEAIALYVPGSERSYDLTGLKPGTEYNVTIQGVKGGFPSGPLEAYFTT

SEQ ID NO: 433 - protein, 90 aa, synthetic construct
MLPAPKNLVVSRVTEDSARLSWTAPDAAFDSFFIGYLEPQPPGEAIALYVPGSERSYDLTGLKPGTEYNVTIQGVKGGFPSLPLEANFTT

SEQ ID NO: 434 - protein, 90 aa, synthetic construct
MLPAPKNLVVSRVTEDSARLSWTAPDAAFDSFFIGYLEPQPPGEAIGLQVPGSERSYDLTGLKPGTEYNVTIQGVKGGFPSDPLQAIFTT

SEQ ID NO: 435 - protein, 90 aa, synthetic construct
MLPAPKNLVVSRVTEDSARLSWTAPDAAFDSFFIGYLEPQPPGEAIALWVPGSERSYDLTGLKPGTEYNVTIQGVKGGFPSHPLAAVFTT

SEQ ID NO: 436 - protein, 90 aa, synthetic construct
MLPAPKNLVVSRVTEDSARLSWTAPDAAFDSFFIGYLEPQPPGEAIHLLVPGSERSYDLTGLKPGTEYNVTIQGVKGGFPSEPLWAKFTT

SEQ ID NO: 437 - protein, 90 aa, synthetic construct
MLPAPKNLVVSRVTEDSARLSWTAPDAAFDSFFIGYLEPQPPGEAISLWVPGSERSYDLTGLKPGTEYNVTIQGVKGGFPSTPLSAKFTT

SEQ ID NO: 438 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSYPLLAIFTT

SEQ ID NO: 439 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSVPLNAYFTT

SEQ ID NO: 440 - protein, 90 aa, synthetic construct
MLPAPKNLVVSRVTEDSARLSWTAPDAAFDSFEIVYAEWTQHGEAIVLTVPGSERSYDLTGLKPGTEYYVHIGGVKGGKWSPPLYAIFTT

SEQ ID NO: 441 - protein, 90 aa, synthetic construct
MLPAPKNLVVSRVTEDSARLSWTAPDAAFDSFFIGYLEPQPPGEAILLWVPGSERSYDLTGLKPGTEYNVTIQGVKGGFPSDPLIANFTT

SEQ ID NO: 442 - protein, 90 aa, synthetic construct
MLPAPKNLVVSRVTEDSARLSWTAPDAAFDSFEIWYAEWRQVGEAIVLTVPGSERSYDLTGLKPGTEYNVDIHGVKGGKVSWPLSAISTT

SEQ ID NO: 443 - protein, 90 aa, synthetic construct
MLPAPKNLVVSRVTEDSARLSWTAPDAAFDSFFIGYLEPQPPGEAIELYVPGSERSYDLTGLKPGTEYNVTIQGVKGGFPSDPLHAAFTT

SEQ ID NO: 444 - protein, 90 aa, synthetic construct
MLPAPKNLVVSRVTEDSARLSWTAPDAAFDSFEIDYVEFIFTGEAIGLIVPGSERSYDLTGLKPGTKYWVTIAGVKGGEWSTPLQAFFTT

SEQ ID NO: 445 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSDPLVASFTT

SEQ ID NO: 446 - protein, 90 aa, synthetic construct
MLPAPKNLVVSRVTEDSARLSWTAPDAAFDSFFIGYLEPQPPGEAIQLFVPGSERSYDLTGLKPGTEYNVTIQGVKGGFPSLPLIAYFTT

SEQ ID NO: 447 - protein, 90 aa, synthetic construct
MLPAPKNLVVSRVTEDSARLSWTAPDAAFDSFFIGYLEPQPPGEAIYLYVPGSERSYDLTGLKPGTEYNVTIQGVKGGFPSYPLTAQFTT

SEQ ID NO: 448 - protein, 90 aa, synthetic construct
MLPAPKNLVVSRVTEDSARLSWTAPDAAFDSFFIGYLEPQPPGEAIDLYVPGSERSYDLTGLKPGTEYNVTIQGVKGGFPSMPLYARFTT

SEQ ID NO: 449 - protein, 90 aa, synthetic construct
MLPAPKNLVVSRVTEDSARLSWTAPDAAFDSFYIAYQELSLWGEAIVLTVPGSERSYDLTGLKPGTEYNVTIQGVKGGFPSLPLAAQFTT

SEQ ID NO: 450 - protein, 90 aa, synthetic construct
MLPAPKNLVVSRVTEDSARLSWTAPDAAFDSFFIGYLEPQPPGEAISLFVPGSERSYDLTGLKPGTEYNVTIQGVKGGFPSEPLAAKFTT

SEQ ID NO: 451 - protein, 90 aa, synthetic construct
MLPAPKNLVVSRVTEDSARLSWTAPDAAFDSFFIGYLEPQPPGEAIGLLVPGSERSYDLTGLKPGTEYNVTIQGVKGGFPSIPLVADFTT

SEQ ID NO: 452 - protein, 90 aa, synthetic construct
MLPAPKNLVVSRVTEDSARLSWTAPDAAFDSFFIGYLEPQPPGEAIHLQVPGSERSYDLTGLKPGTEYNVTIQGVKGGFPSIPLVAQFTT

SEQ ID NO: 453 - protein, 90 aa, synthetic construct
MLPAPKNLVVSRVTEDSARLSWTAQDAAFDSFFIGYLEPQPPGEAIALLVPGSERSYDLTGLKPGTEYNVTIQGVKGGFPSHPLSASFTT

SEQ ID NO: 454 - protein, 90 aa, synthetic construct
MLPAPKNLVVSRVTEDSARLSWTAPDAAFDSFFIGYLEPQPPGEAINLTVPGSERSYDLTGLKPGTEYNVTIQGVKGGFPSYPLQASFTT

SEQ ID NO: 455 - protein, 90 aa, synthetic construct
MLPAPKNLVVSRVTEDSARLSWTAPDAAFDSFTISYPEEAKHGEAIVLTVPGSERSYDLTGLKPGTEYGVPINGVKGGVSSLPLSAIFTT

SEQ ID NO: 456 - protein, 90 aa, synthetic construct
MLPAPKNLVVSRVTEDSARLSWTAPDAAFDSFFIGYLEPQPPGEAIALYVPGSERSYDLTGLKPGTEYNVTIQGVKGGFPSHPLQAHFTT

SEQ ID NO: 457 - protein, 90 aa, synthetic construct
MLPAPKNLVVSRVTEDSARLSWTAPDAAFDSFFIGYLEPQPPGEAIVLLVPGSERSYDLTGLKPGTEYNVTIQGVKGGFPSAPLVATFTT

SEQ ID NO: 458 - protein, 91 aa, synthetic construct
MLPAPKNLVVSRVTEDSARLSWTAPDAAFDSFLDLLPSNWDIAGEAIVLTVPGSERSYDLTGLKPGTEYHVNILGVKGGKESLPLVANFTT

SEQ ID NO: 459 - protein, 90 aa, synthetic construct
MLPAPKNLVVSRVTEDSARLSWTAPDAAFDSFFIRYLEPQPPGEAIHLSVPGSERSYDLTGLKPGTEYNVTIQGVKGGFPSMPLYAIFTT

SEQ ID NO: 460 - protein, 90 aa, synthetic construct
MLPAPKNLVVSRVTEDSARLSWTAPDAAFDSFFIGYLEPQPPGEAISLYVPGSERSYDQTGLKPGTEYNVTIQGVKGGFPSDPLSARFTT

SEQ ID NO: 461 - protein, 90 aa, synthetic construct
MLPAPKNLVVSRVTEDSARLSWTAPDAAFDSFFIGYLEPQPPGEAIGLFVPGSERSYDLTGLKPGTEYNVTIQGVKGGFPSMPLSPIFTT

SEQ ID NO: 462 - protein, 90 aa, synthetic construct
MLPAPKNLVVSRVTEDSARLSWTAPDAAFDSFFIGYLEPQPPGEAIHLQVPGSERSYDLTGLKPGTEYNVTIQGVKGGFPSVPLHARFTT

SEQ ID NO: 463 - protein, 90 aa, synthetic construct
MLPAPKNLVVSRVTEDSARLSWTAPDAAFDSFLILYIEQDHRGEAIVLTVPGSERSYDLTGLKPGTEYWVHITGVKGGYYSAPLSAIFTT

SEQ ID NO: 464 - protein, 90 aa, synthetic construct
MLPAPKNLVVSRVTEDSARLSWTAPDAAFDSFFIGYLEPQPPGEAIQLFVPGSERSYDLTGLKPGTEYNVTIQGVKGGFPSTPLVASFTT

SEQ ID NO: 465 - protein, 90 aa, synthetic construct
MLPAPKNLVVSRVTEDSARLSWTAPDAAFDSFFIGYLEPQPPGEAIDLYVPGSERSYDLTGLKPGTEYNVTIQGVKGGFPSEPLWADFTT

SEQ ID NO: 466 - protein, 90 aa, synthetic construct
MLPAPKNLVVSRVTEDSARLSWTAPDAAFDSFYIAYAEPRPDGEAIWLLVPGSERSYDLTGLKPGTEYSVLIHGVKGGLLSSPLSADFTT

SEQ ID NO: 467 - protein, 90 aa, synthetic construct
MLPAPKNLVVSRVTEDSARLSWTAPDAAFDSFFIGYLEPQPPGEAIQLLVPGSERSYDLTGLKPGTEYNVTIQGVKGGFPSDPLVAYSTT

SEQ ID NO: 468 - protein, 90 aa, synthetic construct
MLPAPKNLVVSRVTEDSARLSWTAPDAAFDSFLIQYDEEGVWGEAIVLTVPGSERSYDLTGLKPGTEYSVGIGGVKGGWSSVPLSAIFTT

SEQ ID NO: 469 - protein, 90 aa, synthetic construct
MLPAPKNLVVSRVTEDSARLSWTAPDAAFDSFFIGYLEPQPPGEAIHLYVPGSERSYDLTGLKPGTEYNVTIQGVKGGFPSLPLVATFTT

SEQ ID NO: 470 - protein, 90 aa, synthetic construct
MLPAPKNLVVSRVTEDSARLSWTAPDAAFDSFFIGYLEPQPPGEAITLLVPGSERSYDLTGLKPGTEYNVTIQGVKGGFPSMPLSAIFTT

SEQ ID NO: 471 - protein, 90 aa, synthetic construct
MLPAPKNLVVSRVTEDSARLSWTAPDAAFDSFTIWYYESPTGGEAIVLTVPGSERSYDLTGLKPGTEYMVFIQGVKGGCFSTPLYAIFTT

SEQ ID NO: 472 - protein, 90 aa, synthetic construct
MLPAPKNLVVSRVTEDSARLSWTAPDAAFDSFFIGYLEPQPPGEAIELYVPGSERSYDLTGLKPGTEYNVTIQGVKGGFPSLPLVATFTT

SEQ ID NO: 473 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSLPLSASFTT

SEQ ID NO: 474 - protein, 90 aa, synthetic construct
MLPAPKNLVVSRVTEDSARLSWTAPDAAFDSFFIGYLEPQPPGEAIGLAVPGSERSYDLTGLKPGTEYNVTIQGVKGGFPSEPLIADFTT

SEQ ID NO: 475 - protein, 90 aa, synthetic construct
MLPAPKNLVVSRVTEDSARLSWTAPDAAFDSFFIGYLEPQPPGEAITLYVPGSERSYDLTGLKPGTEYNVTIQGVKGGFPSIPLSAEFTT

SEQ ID NO: 476 - protein, 90 aa, synthetic construct
MLPAPKNLVVSRVTEDSARLSWTAPDAAFDSFFIGYLEPQPPGEAIHLQVPGSERSYDLTGLKPGTEYNVTIQGVKGGFPSIPLIAVFTT

SEQ ID NO: 477 - protein, 90 aa, synthetic construct
MLPAPKNLVVSRVTEDSARLSWTAPDAAFDSFEIAYFETCRTGEAIVLTVPGSERSYDLTGLKPGTEYGVSIYGVKGGAHSGPLSAIFTT

SEQ ID NO: 478 - protein, 90 aa, synthetic construct
MLPAPKNLVVSRVTEDSARLSWTAPDAAFDSFFIGYLEPQPPGEAIHLYVPGSERSYDLTGLKPGTEYNVTIQGVKGGFPSVPLIAKFTT

SEQ ID NO: 479 - protein, 90 aa, synthetic construct
MLPAPKNLVVSRVTEDSARLSWTAPDAAFDSFFIGYLEPQPPGEAINLWVPGSERSYDLTGLKPGTEYNVTIQGVKGGFPSTPLFAGFTT

SEQ ID NO: 480 - protein, 90 aa, synthetic construct
MLPAPKNLVVSRVTEDSARLSWTAPDAAFDSFEIDYAEDIREGEAIALVVPGSERSYDLTGLKPGTEYWVSIGGVKGGTWSRPLFAPFTT

SEQ ID NO: 481 - protein, 90 aa, synthetic construct
MLPAPKNLVVSRVTEDSARLSWTAPDAAFDSFFIGYLEPQPPGEAITLYVPGSERSYDMTGLKPGTEYNVTIQGVKGGFPSLPLQAHFTT

SEQ ID NO: 482 - protein, 90 aa, synthetic construct
MLPAPKNLIVSRVTEDSARLSWTAPDAAFDSFFIGYLEPQPPGEAIHLQVPGSERSYDLTGLKPGTEYNVTIQGVKGGFPSNPLHAEFTT

SEQ ID NO: 483 - protein, 90 aa, synthetic construct
MLPAPKNLVVSRVTEDSARLSWTAPDAAFDSFFIGYLEPQPPGEAITLVVPGSERSYDLTGLKPGTEYNVTIQGVKGGFPSDPLVASFTT

SEQ ID NO: 484 - protein, 90 aa, synthetic construct
MLPAPKNLVVSRVTEDSARLSWTAPDAAFDSFEIAYQELSLWGEAIVLTVPGSERSYDLTGLKPGTEYYVHIGGVKGGIWSSPLSAISTT

SEQ ID NO: 485 - protein, 90 aa, synthetic construct
MLPAPKNLVVSRVTEDSARLSWTAPDAAFDSFFIGYLEPQPPGEAIHLWVPGSERSYDLTGLKPGTEYNVTIQGVKGGFPSLPLLAKFTT

SEQ ID NO: 486 - protein, 90 aa, synthetic construct
MLPAPENLVVSRVTEDSARLSWTALDAAFDSFFIGYLEPQPPGEAISLLVPGSERSYDLTGLKPGTEYNVTIQGVKGGIASEPLSAAFTT

SEQ ID NO: 487 - protein, 90 aa, synthetic construct
MLPAPKNLVVSRVTEDSARLSWTAPDAAFDSFFIGYLEPQPPGEAIGLRVPGSERSYDLTGLKPGTEYNVTIQGVKGGFPSEPLIADFTT

SEQ ID NO: 488 - protein, 90 aa, synthetic construct
MLPAPKNLVVSRVTEDSARLSWTAPDAAFDSFIIQYGEYLRWGEAIVLTVPGSERSYDLTGLKPGTEYQVEIYGVKGGPLSKPLSAIFTT

SEQ ID NO: 489 - protein, 89 aa, synthetic construct
MLAPKNLVVSRVTEDSARLSWTAPDAAFDSFFIGYLEPQPPGEAISLLVPGSERSYDLTGLKPGTEYNVTIQGVKGGFPSSPLLAGFTT

SEQ ID NO: 490 - protein, 90 aa, synthetic construct
MLPAPKNLVVSRVTEDSARLSWTAPDAAFDSFFIGYLEPQPPGEAISLQVPGSERSYDLTGLKPGTEYNVTIQGVKGGFPSAPLVANFTT

SEQ ID NO: 491 - protein, 90 aa, synthetic construct
MLPAPKNLVVSRVTEDSARLSWTAPDAAFDSFFIGYLEPQPSGEAIHLIVPGSERSYDLTGLKPGTEYNVTIQGVKGGFPSDPLVASFTT

SEQ ID NO: 492 - protein, 90 aa, synthetic construct; identical to SEQ ID NO: 365

SEQ ID NO: 493 - protein, 90 aa, synthetic construct
MLPAPKNLVVSRVTEDSARLSWTAPDAAFDSFFIGYLEPQPPGEAILLAVPGSERSYDLTGLKPGTEYNVTIQGVKGGFPSLPLVAHFTT

SEQ ID NO: 494 - protein, 90 aa, synthetic construct
MLPAPKNLVVSRVTEDSARLSWTTPDAAFDSFFIGYLEPQPPGEAISLYVPGSERSYDLTGLKPGTEYNVTIQGVKGGFPSMPLSAIFTT

SEQ ID NO: 495 - protein, 90 aa, synthetic construct
MLPAPKNLVVSRVTEDSARLSWTAPDAAFDSFFINYREDKWIGEAIVLTVPGSERSYDLTGLKPGTEYSVPIDGVKGGAASPPLSAIFTT

SEQ ID NO: 496 - protein, 90 aa, synthetic construct
MLPAPKNLVVSRVTEDSARLSWTAPDAAFDSFFIGYLEPQPPGEAIILWVPGSERSYDLTGLKPGTEYNVTIQGVKGGFPSVPLIAQFTT

SEQ ID NO: 497 - protein, 90 aa, synthetic construct
MLPAPKNLVVSRVTEDSARLSWTAPDAAFDSFFIGYLEPQPPGEAITLLVPGSERSYDLTGLKPGTEYNVTIQGVKGGFPSKPLYAYFTT

SEQ ID NO: 498 - protein, 90 aa, synthetic construct
MLPAPKNLVVSRVTEDSARLSWTAPDAAFDSFFIGYLEPQPPGEAIILWVPGSERSYDLTGLKPGTEYNVTIQGVKGGFPSIPLIAVFTT

SEQ ID NO: 499 - protein, 90 aa, synthetic construct
MLPAPKNLVVSRVTEDSARLSWTAPDAAFDSFFIGYLEPQPPGEAISLYVPGSERSYDLTGLKPGTEYNVTIQGVKGGFPSSPLFADFTT

SEQ ID NO: 500 - protein, 90 aa, synthetic construct
MLPAPKNLVVSRVTEDSARLSWTAPDAAFDSFFIGYLEPQPPGEAIDLLVPGSERSYDLTGLKPGTEYNVTIQGVKGGFPSMPLSAIFTT

SEQ ID NO: 501 - protein, 90 aa, synthetic construct
MLPAPKNLVVSRVTEDSARLSWTAPDAAFDSFFIGYLEPQPPGEAISLLVPGSERSYDLTGLKPGTEYNVTIQGVKGGFPSVPLSAIFTT

SEQ ID NO: 502 - protein, 90 aa, synthetic construct
MLPAPKNLVVSRVTEDSARLSWTAPDAAFDSFFIGYLEPQPPGEAIGLHVPGSERSYDLTGLKPGTEYNVTIQGVKGGFPSIPLEAKFTT

SEQ ID NO: 503 - protein, 90 aa, synthetic construct
MLPAPKNLVVSRVTEDSARLSWTAPDAAFDSFEISYFETSWHGEAIVLTVLGSERSYDLTGLKPGTEYRVYIGGVKGGSWSQPLSAIFTT

SEQ ID NO: 504 - protein, 90 aa, synthetic construct
MLPAPKNLVVSRVTEDSARLSWTAPDAAFDSFFIGYLEPQPPGEAIGLVVPGSERSYDLTGLKPGTEYNVTIQGVKGGFPSHPLGAIFTT

SEQ ID NO: 505 - protein, 90 aa, synthetic construct
MLPAPKNLVVSRVTEDSARLSWTALDAAFDSFFIGYLEPQPPGEAIHLFVPGSERSYDLTGLKPGTEYNVTIQGVKGGFPSEPLIANFTT

SEQ ID NO: 506 - protein, 90 aa, synthetic construct
MLPAPKNLVVSRVTEDSARLSWTAPDAAFDSFEIGYWENAYKGEAIVLTVPGSERSYDLTGLKPGTEYVVFIGGVKGGVWSVPLSAISTT

SEQ ID NO: 507 - protein, 90 aa, synthetic construct
MLPAPKNLVVSRVTEDSARLSWTAPDAAFDSFFIGYLEPQPPGEAIHLLVPGSERSYDLTGLKPGTEYNVTIQGVKGGFPSMPLSAIFTT

SEQ ID NO: 508 - protein, 90 aa, synthetic construct
MLPAPKNLVVSRVTEDSARLSWTAPDAAFDSFFIGYLEPQPPGEAITLSVPGSERSYDLTGLKPGTEYNVTIQGVKGGFPSEPLIAIFTT

SEQ ID NO: 509 - protein, 90 aa, synthetic construct
MLPAPKNLVVSRVTEDSARLSWTAPDAAFDSFEIGYWENAYKGEAIVLTVPGSERSYDLTGLKPGTEYEVFIGGVKGGVWSVPLSAIFTT

SEQ ID NO: 510 - protein, 90 aa, synthetic construct
MLPAPKNLVVSRVTEDSARLSWTAPDAAFDSFFIAYGESPESGEAIVLTVPGSERSYDLTGLKPGTEYLVWIAGVKGGYYSDPLSAIFTT

SEQ ID NO: 511 - protein, 90 aa, synthetic construct
MLPAPKNLVVSRVTEDSARLSWTAPDAAFDSFFIGYLEPQPPGEAIDLFVPGSERSYDLTGLKPGTEYNVTIQGVKGGFPSLPLVASFTT

SEQ ID NO: 512 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSDPLDAHFTT

SEQ ID NO: 513 - protein, 90 aa, synthetic construct
MLPAPKNLVVSRVTEDSARLSWTAPDAAFDSFFIGYLEPQPPGEAISLYVPGSERSYDLTGLKPGTEYNVTIQGVKGGFPSLPLAAQFTT

SEQ ID NO: 514 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSEPLIANFTT

SEQ ID NO: 515 - protein, 90 aa, synthetic construct
MLPAPKNLVVSRVTEDSARLSWTAPDAAFDSFFIGYLEPQPPGEAIALWVPGSERSYDLTGLKPGTEYNVTIQGVKGGFPSTPLIAQFTT

SEQ ID NO: 516 - protein, 90 aa, synthetic construct
MLPAPKNLVVSRVTEDSARLSWTAPDAAFDSFFIGYLEPQPPGEAISLWVPGSERSYDLTGLKPGTEYNVTIQGVKGGFPSSPLVAYFTT

SEQ ID NO: 517 - protein, 90 aa, synthetic construct
MLPAPKNLVVSRVTEDSARLSWTAPDAAFDSFFIGYLEPQPPGEAIALVVPGSERSYDLTGLKPGTEYNVTIQGVKGGFPSMPLFAYFTT

SEQ ID NO: 518 - protein, 90 aa, synthetic construct
MLPAPKNLVVSRVTEDSARLSWTAPDAAFDSFFIGYLEPQPPGEAISLWVPGSERSYDLTGLKPGTEYNVTIQGVKGGFPSDPLGAHFTT

SEQ ID NO: 519 - protein, 90 aa, synthetic construct
MLPAPKNLVVSRVTEDSARLSWTAPDAAFDSFPIAYPEHLPPGEAIVLTVPGSERSYDLTGLKPGTEYPVNIRGVKGGFVSFPLSAIFTT

SEQ ID NO: 520 - protein, 90 aa, synthetic construct; identical to SEQ ID NO: 365

SEQ ID NO: 521 - protein, 90 aa, synthetic construct
MLPAPKNLVVSRVTEDSARLSWTAPDAAFDSFFIGYLEPQPPGEAIVLWVPGSERSYDLTGLKPGTEYNVTIQGVKGGFPSHPLVAHFTT

SEQ ID NO: 522 - protein, 90 aa, synthetic construct
MLPAPKNLVVSRVTEDSARLSWTAPDAAFDSFFIGYLEPQPPGEAITLYVPGSERSYDLNGLKPGTEYNVTIQGVKGGFPSMPLSAIFTT

SEQ ID NO: 523 - protein, 90 aa, synthetic construct
MLPAPKNLVVSRVTEDSARLSWTAPDAAFDSFFIGYLEPQPPGEAIDLLVPGSERSYDLTGLKPGTEYNVTIQGVKGGFPSEPLTAEFTT

SEQ ID NO: 524 - protein, 90 aa, synthetic construct
MLPAPKNLVVSRVTEDSARLSWTAPDAAFDSFFIGYLEPQPPGEAIHLWVPGSERSYDLTGLKPGTEYNVTIQGVKGGFPSMPLSAIFTT

SEQ ID NO: 525 - protein, 90 aa, synthetic construct; identical to SEQ ID NO: 423

SEQ ID NO: 526 - protein, 90 aa, synthetic construct
MLPAPKNLVVSRVTEDSARLSWTAPDAAFDSFFIGYLEPQPPGEAISLIVPGSERSYDLTGLKPGTEYNVTIQGVKGGFPSDPLIAIFTT

SEQ ID NO: 527 - protein, 90 aa, synthetic construct
MLPAPKNLVVSRVTEDSARLSWTAPDAAFDSFFIGYLEPQPPGEAIHLYVPGSERSYDLTGLKPGTEYNVTIQGVKGGFPSEPLIANFTT

SEQ ID NO: 528 - protein, 90 aa, synthetic construct
MLPAPKNLVVSRVTEDSARLSWTAPDAAFDSFFIGYLEPQPPGEAITLLVPGSERSYDLTGLKPGTEYNVTIQGVKGGFPSIPLIAIFTT

SEQ ID NO: 529 - protein, 90 aa, synthetic construct
MLPAPKNLVVSRVTEDSARLSWTAPDAAFDSFFIGYLEPQPPGEAIDLYVPGSERSYDLTGLKPGTEYNVTIQGVKGGFPSLPLQAHFTT

SEQ ID NO: 530 - protein, 90 aa, synthetic construct
MLPAPKNLVVSRVTEDSARLSWTAPDAAFDSFFIGYLEPQPPGEAIDLLVPGSERSYDLTGLKPGTEYNVTIQGVKGGFPSEPLFAHFTT

SEQ ID NO: 531 - protein, 90 aa, synthetic construct
MLPAPKNLVVSRVTEDSARLSWTAPDAAFDSFEIGYFEYSYYGEAIVLTVPGSERSYDLTGLKPGTEYRVYIGGVKGGGWSRPLSAIFTT

SEQ ID NO: 532 - protein, 90 aa, synthetic construct
MLPAPKNLVVSRVTEDSARLSWTAPDAAFDSFDINYDELSGEGEAIALFVPGSERSYDLTGLKPGTEYWVSIGGVKGGRFSEPLYARFTT

SEQ ID NO: 533 - protein, 90 aa, synthetic construct
MLPAPKNLVVSRVTEDSARLSWTAPDAAFDSFFIGYLEPQPPGEAINLFVPGSERSYDLTGLKPGTEYNVTIQGVKGGFPSMPLSAIFTT

SEQ ID NO: 534 - protein, 90 aa, synthetic construct
MLPAPKNLVVSRVTEDSARLSWTAPDAAFDSFFIGYLEPQPPGEAIHLLVPGSERSYDLTGLKPGTEYNVTIQGVKGGFPSGPLQASFTT

SEQ ID NO: 535 - protein, 90 aa, synthetic construct
MLPAPKNLVVSRVTEDSARLSWTAPDAAFDSFFIGYLEPQPPGEAIHLQVPGSERSYDLTGLKPGTEYNVTIQGVKGGFPSDPLVAKFTT

SEQ ID NO: 536 - protein, 90 aa, synthetic construct
MLPAPKNLVVSRVTEDSARLSWTAPDAAFDSFDIGYTETVSFGEAIVLTVPGSERSYDLTGLKPGTEYIVKILGVKGGFASFPLSAIFTT

SEQ ID NO: 537 - protein, 90 aa, synthetic construct
MLPAPKNLVVSRVTEDSARLSWTAPDAAFDSFEIGYFENLYLGEAIVLTVPGSERSYDLTGLKPGTEYWVQIGGVKGGNWSAPLSAIFTT

SEQ ID NO: 538 - protein, 90 aa, synthetic construct
MLPAPKNLVVSRVTEDSARLSWTAPDAAFDSFFIGYLEPQPPGEAIDLYVPGSERSYDLTGLKPGTEYNVTIQGVKGGFPSHPLVAVFTT

SEQ ID NO: 539 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSDPLHAAFTT

SEQ ID NO: 540 - protein, 90 aa, synthetic construct
MLPAPKNLVVSRVTEDSARLSWTAPDAAFDSFYIAYAEPRPDGEAIILVVPGSERSYDLTGLKPGTEYSVLIHGVKGGLLSSPLDAGFTT

SEQ ID NO: 541 - protein, 90 aa, synthetic construct
MLPAPKNLVVSRVTEDSARLSWTAPDAAFDSFFIGYLEPQPPGEAIDLWVPGSERSYDLTGLKPGTEYNVTIQGVKGGFPSMPLDAYFTT

SEQ ID NO: 542 - protein, 90 aa, synthetic construct
MLPAPKNLVVSRVTEDSARLSWTAPDAAFDSFFIGYLEPQPPGEAIELFVPGSERSYDLTGLKPGTEYNVTIQGVKGGFPSMPLSAIFTT

SEQ ID NO: 543 - protein, 90 aa, synthetic construct
MLPAPKNLVVSRVTEDSARLSWTAPDAAFDSFFIGYLEPQPPGEAISLYVPGSERSYDLTGLKPGTEYNVTIQGVKGGFPSIPLVAVFTT

SEQ ID NO: 544 - protein, 90 aa, synthetic construct
MLPAPKNLVVSRVTEDSARLSWTAPDAAFDSFFIGYLEPQPPGEAIALLVPGSERSYDLTGLKPGTEYNVTIQGVKGGFPSHPLSASFTT

SEQ ID NO: 545 - protein, 90 aa, synthetic construct
MLPAPKNLVVSRVTEDSARLSWTAPDAAFDSFFIGYLEPQPPGEAISLVVPGSERSYDLTGLKPGTEYNVTIQGVKGGFPSNPLIATFTT

SEQ ID NO: 546 - protein, 90 aa, synthetic construct
MLPAPKNLVVSRVTEDSARLSWTAPDAAFDSFEIAYIEVNIQGEAIVLTVPGSERSYDLTGLKPGTEYYVHIGGVKGGPSSSPLSAIFTT

SEQ ID NO: 547 - protein, 90 aa, synthetic construct
MLPAPKNLVVSRVTEDSARLSWTAPDAAFDSFFIGYLEPQPPGEAIHLIVPGSERSYDLTGLKPGTEYNVTIQGVKGGFPSDPLVASFTT

SEQ ID NO: 548 - protein, 90 aa, synthetic construct
MLPAPKNLVVSRVTEDSARLSWTAPDAAFDSFFIGYLEPQPPGEAIYLFVPGSERSYDLTGLKPGTEYNVTIQGVKGGFPSMPLSAIFTT

SEQ ID NO: 549 - protein, 90 aa, synthetic construct
MLPAPKNLVVSRVTEDSARLSWTAPDAAFDSFFIGYLEPQPPGEAISLWVPGSERSYDLTGLKPGTEYNVTIQGVKGGFPSMPLSAIFTT

SEQ ID NO: 550 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSIPLYARFTT

SEQ ID NO: 551 - protein, 90 aa, synthetic construct
MLPAPKNLVVSRVTEDSARLSWTAPDAAFDSFEIAYFETCRTGEAIVLTVPGSERSYDLTGLKPGTEYWVQIGGVKGGNWSAPLSAIFTT

SEQ ID NO: 552 - protein, 90 aa, synthetic construct
MLPAPKNLVVSRVTEDSARLSWTAPDAAFDSFFIGYLEPQPPGEAIHLFVPGSERSYDLTGLKPGTEYNVTIQGVKGGFPSMPLSAIFTT

SEQ ID NO: 553-599 - skipped (47 entries)

SEQ ID NO: 600 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
ctgggaggatgaattcgaat

SEQ ID NO: 601 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
caaggtgggtggcatctaat

SEQ ID NO: 602 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
tccgcagcctggatgattat

SEQ ID NO: 603 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gcgactgcctgtagcaacat

SEQ ID NO: 604 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
tttatgggcatctggactat

SEQ ID NO: 605 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gcgcggaccaacaatttcat

SEQ ID NO: 606 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ggaccaacaatttcaacgat

SEQ ID NO: 607 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
accaacaatttcaacgtgat

SEQ ID NO: 608 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gcgcaaacagctttgggaat

SEQ ID NO: 609 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
agagccatctttgcaacgat

SEQ ID NO: 610 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcaacgcat

SEQ ID NO: 611 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
agccatctttgcaacgcaat

SEQ ID NO: 612 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ccatctttgcaacgcagcat

SEQ ID NO: 613 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
acgcagcggcagtctttcat

SEQ ID NO: 614 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
tgaccaccatccgccgaaat

SEQ ID NO: 615 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
cgaatcggcctcttcaatat

SEQ ID NO: 616 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
cttcaatagcagtgccgaat

SEQ ID NO: 617 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
cagtatctccaccaatctat

SEQ ID NO: 618 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ccaccaatctctccggctat

SEQ ID NO: 619 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caccaatctctccggcttat

SEQ ID NO: 620 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
cccctcagcttacggtatat

SEQ ID NO: 621 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ccctcagcttacggtatcat

SEQ ID NO: 622 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacattctat

SEQ ID NO: 623 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ggaaccgcacggagcgccat

SEQ ID NO: 624 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
acggagcgcctctccgacat

SEQ ID NO: 625 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
agcaagcgcaactctgtgat

SEQ ID NO: 626 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
cagatggtcctccatttcat

SEQ ID NO: 627 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
tctagatctggaaccttaat

SEQ ID NO: 628 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
cccacatttacctcgaggat

SEQ ID NO: 629 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
accgcttggtttgcagttat

SEQ ID NO: 630 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
ctgggaggatgaattcggat

SEQ ID NO: 631 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
ctgggaggatgaattaggat

SEQ ID NO: 632 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
ctgggaggatgaatgaggat

SEQ ID NO: 633 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
ctgggaggatgaattcgaat

SEQ ID NO: 634 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
  misc_feature 1..19: note = RNA
  misc_feature 20: note = DNA
ctgggaggatgaattcgaat

SEQ ID NO: 635 - DNA, 15 bp, synthetic construct
  source 1..15: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..13: mod_base = OTHER; note = 2'-O-methyl ribonucleotide
  modified_base 14: mod_base = OTHER; note = 2'-fluoro ribonucleotide
  modified_base 15: mod_base = OTHER; note = inverted deoxythymidine
  modified_base 1..3: mod_base = OTHER; note = phosphorothioate linkage
  modified_base 13..14: mod_base = OTHER; note = phosphorothioate linkage
  misc_feature 1..14: note = RNA
  misc_feature 15: note = DNA
ctgggaggatgaatt

SEQ ID NO: 636 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcatctaat

SEQ ID NO: 637 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcatctagt

SEQ ID NO: 638 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcatctgat

SEQ ID NO: 639 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcatcgaat

SEQ ID NO: 640 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcatataat

SEQ ID NO: 641 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcagctaat

SEQ ID NO: 642 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcagctagt

SEQ ID NO: 643 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcaacgcat

SEQ ID NO: 644 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcaacgcgt

SEQ ID NO: 645 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcaacgaat

SEQ ID NO: 646 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcaacacat

SEQ ID NO: 647 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcaaagcat

SEQ ID NO: 648 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcagcgcat

SEQ ID NO: 649 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
gagccatctttgcagcgcgt

SEQ ID NO: 650 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacattctat

SEQ ID NO: 651 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacattctgt

SEQ ID NO: 652 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacattcgat

SEQ ID NO: 653 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacattgtat

SEQ ID NO: 654 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacatgctat

SEQ ID NO: 655 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacatgctat

SEQ ID NO: 656 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacagtctat

SEQ ID NO: 657 - DNA, 20 bp, synthetic construct
  source 1..20: mol_type = other DNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
ttacggtatctacagtctgt

SEQ ID NO: 658 - DNA, 20 bp, synthetic construct
  source 1..20: mol_type = other DNA
  modified_base 1: mod_base = OTHER; note = 2'-fluoro ribonucleotide
  modified_base 2: mod_base = OTHER; note = 2'-O-methyl ribonucleotide
  modified_base 3: mod_base = OTHER; note = 2'-fluoro ribonucleotide
  modified_base 4: mod_base = OTHER; note = 2'-O-methyl ribonucleotide
  modified_base 5: mod_base = OTHER; note = 2'-fluoro ribonucleotide
  modified_base 6: mod_base = OTHER; note = 2'-O-methyl ribonucleotide
  modified_base 7..9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-fluoro ribonucleotide
  modified_base 12..14: mod_base = OTHER; note = 2'-O-methyl ribonucleotide
  modified_base 15: mod_base = OTHER; note = 2'-fluoro ribonucleotide
  modified_base 16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 20: mod_base = OTHER; note = inverted deoxythymidine
  modified_base 1..3: mod_base = OTHER; note = phosphorothioate linkage
  misc_feature 1..19: note = RNA
  misc_feature 20: note = DNA
caaggtgggtggcatctagt

SEQ ID NO: 659 - RNA, 19 nt, synthetic construct
  source 1..19: mol_type = other RNA
  modified_base 1..2: mod_base = OTHER; note = 2'-O-methyl ribonucleotide
  modified_base 3: mod_base = OTHER; note = 2'-fluoro ribonucleotide
  modified_base 4..6: mod_base = OTHER; note = 2'-O-methyl ribonucleotide
  modified_base 7..9: mod_base = OTHER; note = 2'-fluoro ribonucleotide
  modified_base 10..11: mod_base = OTHER; note = 2'-O-methyl ribonucleotide
  modified_base 12: mod_base = OTHER; note = 2'-fluoro ribonucleotide
  modified_base 13..16: mod_base = OTHER; note = 2'-O-methyl ribonucleotide
  modified_base 17: mod_base = OTHER; note = 2'-fluoro ribonucleotide
  modified_base 18..19: mod_base = OTHER; note = 2'-O-methyl ribonucleotide
  modified_base 1..3: mod_base = OTHER; note = phosphorothioate linkage
ttacggtatctacattcta

SEQ ID NO: 660 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaattcatcctcccagtt

SEQ ID NO: 661 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 662 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taatcatccaggctgcggatt

SEQ ID NO: 663 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgttgctacaggcagtcgctt

SEQ ID NO: 664 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagtccagatgcccataaatt

SEQ ID NO: 665 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgaaattgttggtccgcgctt

SEQ ID NO: 666 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tcgttgaaattgttggtcctt

SEQ ID NO: 667 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tcacgttgaaattgttggttt

SEQ ID NO: 668 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcccaaagctgtttgcgctt

SEQ ID NO: 669 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tcgttgcaaagatggctcttt

SEQ ID NO: 670 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 671 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttgcgttgcaaagatggcttt

SEQ ID NO: 672 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgctgcgttgcaaagatggtt

SEQ ID NO: 673 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgaaagactgccgctgcgttt

SEQ ID NO: 674 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tttcggcggatggtggtcatt

SEQ ID NO: 675 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tattgaagaggccgattcgtt

SEQ ID NO: 676 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcggcactgctattgaagtt

SEQ ID NO: 677 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagattggtggagatactgtt

SEQ ID NO: 678 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagccggagagattggtggtt

SEQ ID NO: 679 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taagccggagagattggtgtt

SEQ ID NO: 680 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tataccgtaagctgaggggtt

SEQ ID NO: 681 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgataccgtaagctgagggtt

SEQ ID NO: 682 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 683 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tggcgctccgtgcggttcctt

SEQ ID NO: 684 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgtcggagaggcgctccgttt

SEQ ID NO: 685 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tcacagagttgcgcttgcttt

SEQ ID NO: 686 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgaaatggaggaccatctgtt

SEQ ID NO: 687 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttaaggttccagatctagatt

SEQ ID NO: 688 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tcctcgaggtaaatgtgggtt

SEQ ID NO: 689 - RNA, 21 nt, synthetic construct
  source 1..21: mol_type = other RNA
  modified_base 1..2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taactgcaaaccaagcggttt

SEQ ID NO: 690 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaattcatcctcccagtt

SEQ ID NO: 691 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaattcatcctcccagtt

SEQ ID NO: 692 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaattcatcctcccagtt

SEQ ID NO: 693 - RNA, 21 nt, synthetic construct
  source 1..21: mol_type = other RNA
  modified_base 1..21: mod_base = OTHER; note = 2'-fluoro ribonucleotide
  modified_base 1..3: mod_base = OTHER; note = phosphorothioate linkage
  modified_base 19..21: mod_base = OTHER; note = phosphorothioate linkage
ttcgaattcatcctcccagtt

SEQ ID NO: 694 - RNA, 21 nt, synthetic construct
  source 1..21: mol_type = other RNA
  modified_base 1: mod_base = OTHER; note = 2'-O-methyl ribonucleotide
  modified_base 2..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11: mod_base = OTHER; note = 2'-O-methyl ribonucleotide
  modified_base 12..20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaattcatcctcccagtt

SEQ ID NO: 695 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 18..21: mod_base = OTHER; note = phosphorothioate linkage
ttcgaattcatcctcccagtt

SEQ ID NO: 696 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 697 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 698 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 699 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 700 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 701 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 702 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 703 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 704 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 705 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 706 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 707 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 708 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 709 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tgcgttgcaaagatggctctt

SEQ ID NO: 710 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 711 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 712 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 713 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 714 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 715 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 716 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 717 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 718 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-fluoro ribonucleotide
  modified_base 5: mod_base = OTHER; note = 2'-O-methyl ribonucleotide
  modified_base 6..8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-fluoro ribonucleotide
  modified_base 11..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-fluoro ribonucleotide
  modified_base 19: mod_base = OTHER; note = 2'-O-methyl ribonucleotide
  modified_base 20: mod_base = OTHER; note = 2'-fluoro ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttagatgccacccaccttgtt

SEQ ID NO: 719 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..13: mod_base = OTHER; note = 2'-O-methyl ribonucleotide
  modified_base 14: mod_base = OTHER; note = 2'-fluoro ribonucleotide
  modified_base 15..21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
tagaatgtagataccgtaatt

SEQ ID NO: 720 - RNA, 19 nt, synthetic construct
  source 1..19: mol_type = other RNA
caggggtgcggtcttgcaa

SEQ ID NO: 721 - RNA, 19 nt, synthetic construct
  source 1..19: mol_type = other RNA
ggtgcggtcttgcaatagg

SEQ ID NO: 722 - RNA, 19 nt, synthetic construct
  source 1..19: mol_type = other RNA
gtgcggtcttgcaatagga

SEQ ID NO: 723 - RNA, 19 nt, synthetic construct
  source 1..19: mol_type = other RNA
agccatgcctttaaaccgc

SEQ ID NO: 724 - RNA, 19 nt, synthetic construct
  source 1..19: mol_type = other RNA
atgcctttaaaccgcactt

SEQ ID NO: 725 - RNA, 19 nt, synthetic construct
  source 1..19: mol_type = other RNA
tgggaggatgaattcgacc

SEQ ID NO: 726 - RNA, 19 nt, synthetic construct
  source 1..19: mol_type = other RNA
aacgcagtgctcttcgaag

SEQ ID NO: 727 - RNA, 19 nt, synthetic construct
  source 1..19: mol_type = other RNA
ggacgaatggggcgacaac

SEQ ID NO: 728 - RNA, 19 nt, synthetic construct
  source 1..19: mol_type = other RNA
ggcgactgcctgtagcaac

SEQ ID NO: 729 - RNA, 19 nt, synthetic construct
  source 1..19: mol_type = other RNA
cacgctgctggggcgctac

SEQ ID NO: 730 - RNA, 19 nt, synthetic construct
  source 1..19: mol_type = other RNA
tgcgctcacgtcttcacta

SEQ ID NO: 731 - RNA, 19 nt, synthetic construct
  source 1..19: mol_type = other RNA
ccttatacttctttatcgc

SEQ ID NO: 732 - RNA, 19 nt, synthetic construct
  source 1..19: mol_type = other RNA
ctttatcgccggccgctat

SEQ ID NO: 733 - RNA, 19 nt, synthetic construct
  source 1..19: mol_type = other RNA
tcgccggccgctatgagtt

SEQ ID NO: 734 - RNA, 19 nt, synthetic construct
  source 1..19: mol_type = other RNA
gccgctatgagttctccaa

SEQ ID NO: 735 - RNA, 19 nt, synthetic construct
  source 1..19: mol_type = other RNA
ccgctatgagttctccaac

SEQ ID NO: 736 - RNA, 19 nt, synthetic construct
  source 1..19: mol_type = other RNA
ggctcggctcaactatctg

SEQ ID NO: 737 - RNA, 19 nt, synthetic construct
  source 1..19: mol_type = other RNA
gctcggctcaactatctgc

SEQ ID NO: 738 - RNA, 19 nt, synthetic construct
  source 1..19: mol_type = other RNA
tcggctcaactatctgctc

SEQ ID NO: 739 - RNA, 19 nt, synthetic construct
  source 1..19: mol_type = other RNA
ccagcgcggaccaacaatt

SEQ ID NO: 740 - RNA, 19 nt, synthetic construct
  source 1..19: mol_type = other RNA
agcgcggaccaacaatttc

SEQ ID NO: 741 - RNA, 19 nt, synthetic construct
  source 1..19: mol_type = other RNA
tcgggaggaagctttatga

SEQ ID NO: 742 - RNA, 19 nt, synthetic construct
  source 1..19: mol_type = other RNA
acgcagcggcagtctttcc

SEQ ID NO: 743 - RNA, 19 nt, synthetic construct
  source 1..19: mol_type = other RNA
gaccaccatccgccgaatc

SEQ ID NO: 744 - RNA, 19 nt, synthetic construct
  source 1..19: mol_type = other RNA
ccgaatcggcctcttcaat

SEQ ID NO: 745 - RNA, 19 nt, synthetic construct
  source 1..19: mol_type = other RNA
aatcggcctcttcaatagc

SEQ ID NO: 746 - RNA, 19 nt, synthetic construct
  source 1..19: mol_type = other RNA
caatagcagtgccgacagg

SEQ ID NO: 747 - RNA, 19 nt, synthetic construct
  source 1..19: mol_type = other RNA
aggagtttgtccgtggctg

SEQ ID NO: 748 - RNA, 19 nt, synthetic construct
  source 1..19: mol_type = other RNA
cggctgagtgcacggttat

SEQ ID NO: 749 - RNA, 19 nt, synthetic construct
  source 1..19: mol_type = other RNA
tgggaatccccagtatctc

SEQ ID NO: 750 - RNA, 19 nt, synthetic construct
  source 1..19: mol_type = other RNA
cagacccctcagcttacgg

SEQ ID NO: 751 - RNA, 19 nt, synthetic construct
  source 1..19: mol_type = other RNA
cccctcagcttacggtatc

SEQ ID NO: 752 - RNA, 19 nt, synthetic construct
  source 1..19: mol_type = other RNA
ctcagcttacggtatctac

SEQ ID NO: 753 - RNA, 19 nt, synthetic construct
  source 1..19: mol_type = other RNA
gcttacggtatctacattc

SEQ ID NO: 754 - RNA, 19 nt, synthetic construct
  source 1..19: mol_type = other RNA
ccggcggcagcgtatcatc

SEQ ID NO: 755 - RNA, 19 nt, synthetic construct
  source 1..19: mol_type = other RNA
agcgtatcatccagcggaa

SEQ ID NO: 756 - RNA, 19 nt, synthetic construct
  source 1..19: mol_type = other RNA
gcgtatcatccagcggaac

SEQ ID NO: 757 - RNA, 19 nt, synthetic construct
  source 1..19: mol_type = other RNA
tacctaggccggtactata

SEQ ID NO: 758 - RNA, 19 nt, synthetic construct
  source 1..19: mol_type = other RNA
ctaggccggtactatatgt

SEQ ID NO: 759 - RNA, 19 nt, synthetic construct
  source 1..19: mol_type = other RNA
aggccggtactatatgtct

SEQ ID NO: 760 - RNA, 19 nt, synthetic construct
  source 1..19: mol_type = other RNA
ggccggtactatatgtctg

SEQ ID NO: 761 - RNA, 19 nt, synthetic construct
  source 1..19: mol_type = other RNA
gccggtactatatgtctgc

SEQ ID NO: 762 - RNA, 19 nt, synthetic construct
  source 1..19: mol_type = other RNA
cggtactatatgtctgcgc

SEQ ID NO: 763 - RNA, 19 nt, synthetic construct
  source 1..19: mol_type = other RNA
ggtactatatgtctgcgcg

SEQ ID NO: 764 - RNA, 19 nt, synthetic construct
  source 1..19: mol_type = other RNA
ctatatgtctgcgcgccac

SEQ ID NO: 765 - RNA, 19 nt, synthetic construct
  source 1..19: mol_type = other RNA
tatgtctgcgcgccacatg

SEQ ID NO: 766 - RNA, 19 nt, synthetic construct
  source 1..19: mol_type = other RNA
atgtctgcgcgccacatgg

SEQ ID NO: 767 - RNA, 19 nt, synthetic construct
  source 1..19: mol_type = other RNA
tcgccctcgctgtcacgac

SEQ ID NO: 768 - RNA, 19 nt, synthetic construct
  source 1..19: mol_type = other RNA
cggcgagcgctacgatgag

SEQ ID NO: 769 - RNA, 19 nt, synthetic construct
  source 1..19: mol_type = other RNA
caaggaccggcgcaacatc

SEQ ID NO: 770 - RNA, 19 nt, synthetic construct
  source 1..19: mol_type = other RNA
acatccgtgcaccagagtg

SEQ ID NO: 771 - RNA, 19 nt, synthetic construct
  source 1..19: mol_type = other RNA
ctgggcgaggagcgtaact

SEQ ID NO: 772 - RNA, 19 nt, synthetic construct
  source 1..19: mol_type = other RNA
gggcgaggagcgtaactaa

SEQ ID NO: 773 - RNA, 19 nt, synthetic construct
  source 1..19: mol_type = other RNA
gcgaggagcgtaactaagt

SEQ ID NO: 774 - RNA, 19 nt, synthetic construct
  source 1..19: mol_type = other RNA
gaggagcgtaactaagtcc

SEQ ID NO: 775 - RNA, 19 nt, synthetic construct
  source 1..19: mol_type = other RNA
agtccgccaaacactccac

SEQ ID NO: 776 - RNA, 19 nt, synthetic construct
  source 1..19: mol_type = other RNA
ggcgatcaagtccagagcc

SEQ ID NO: 777 - RNA, 19 nt, synthetic construct
  source 1..19: mol_type = other RNA
ccctaacctggcttattcc

SEQ ID NO: 778 - RNA, 19 nt, synthetic construct
  source 1..19: mol_type = other RNA
tgtgaaaccactaggttct

SEQ ID NO: 779 - RNA, 19 nt, synthetic construct
  source 1..19: mol_type = other RNA
accactaggttctaggtcc

SEQ ID NO: 780 - RNA, 19 nt, synthetic construct
  source 1..19: mol_type = other RNA
caggggtgcggtcttgcaa

SEQ ID NO: 781 - RNA, 19 nt, synthetic construct
  source 1..19: mol_type = other RNA
ggtgcggtcttgcaataga

SEQ ID NO: 782 - RNA, 19 nt, synthetic construct
  source 1..19: mol_type = other RNA
gtgcggtcttgcaatagga

SEQ ID NO: 783 - RNA, 19 nt, synthetic construct
  source 1..19: mol_type = other RNA
agccatgcctttaaaccga

SEQ ID NO: 784 - RNA, 19 nt, synthetic construct
  source 1..19: mol_type = other RNA
atgcctttaaaccgcacta

SEQ ID NO: 785 - RNA, 19 nt, synthetic construct
  source 1..19: mol_type = other RNA
tgggaggatgaattcgaca

SEQ ID NO: 786 - RNA, 19 nt, synthetic construct
  source 1..19: mol_type = other RNA
aacgcagtgctcttcgaaa

SEQ ID NO: 787 - RNA, 19 nt, synthetic construct
  source 1..19: mol_type = other RNA
ggacgaatggggcgacaaa

SEQ ID NO: 788 - RNA, 19 nt, synthetic construct
  source 1..19: mol_type = other RNA
ggcgactgcctgtagcaaa

SEQ ID NO: 789 - RNA, 19 nt, synthetic construct
  source 1..19: mol_type = other RNA
cacgctgctggggcgctaa

SEQ ID NO: 790 - RNA, 19 nt, synthetic construct
  source 1..19: mol_type = other RNA
tgcgctcacgtcttcacta

SEQ ID NO: 791 - RNA, 19 nt, synthetic construct
  source 1..19: mol_type = other RNA
ccttatacttctttatcga

SEQ ID NO: 792 - RNA, 19 nt, synthetic construct
  source 1..19: mol_type = other RNA
ctttatcgccggccgctaa

SEQ ID NO: 793 - RNA, 19 nt, synthetic construct
  source 1..19: mol_type = other RNA
tcgccggccgctatgagta

SEQ ID NO: 794 - RNA, 19 nt, synthetic construct
  source 1..19: mol_type = other RNA
gccgctatgagttctccaa

SEQ ID NO: 795 - RNA, 19 nt, synthetic construct
  source 1..19: mol_type = other RNA
ccgctatgagttctccaaa

SEQ ID NO: 796 - RNA, 19 nt, synthetic construct
  source 1..19: mol_type = other RNA
ggctcggctcaactatcta

SEQ ID NO: 797 - RNA, 19 nt, synthetic construct
  source 1..19: mol_type = other RNA
gctcggctcaactatctga

SEQ ID NO: 798 - RNA, 19 nt, synthetic construct
  source 1..19: mol_type = other RNA
tcggctcaactatctgcta

SEQ ID NO: 799 - RNA, 19 nt, synthetic construct
  source 1..19: mol_type = other RNA
ccagcgcggaccaacaata

SEQ ID NO: 800 - RNA, 19 nt, synthetic construct
  source 1..19: mol_type = other RNA
agcgcggaccaacaattta

SEQ ID NO: 801 - RNA, 19 nt, synthetic construct
  source 1..19: mol_type = other RNA
tcgggaggaagctttatga

SEQ ID NO: 802 - RNA, 19 nt, synthetic construct
  source 1..19: mol_type = other RNA
acgcagcggcagtctttca

SEQ ID NO: 803 - RNA, 19 nt, synthetic construct
  source 1..19: mol_type = other RNA
gaccaccatccgccgaata

SEQ ID NO: 804 - RNA, 19 nt, synthetic construct
  source 1..19: mol_type = other RNA
ccgaatcggcctcttcaaa

SEQ ID NO: 805 - RNA, 19 nt, synthetic construct
  source 1..19: mol_type = other RNA
aatcggcctcttcaataga

SEQ ID NO: 806 - RNA, 19 nt, synthetic construct
  source 1..19: mol_type = other RNA
caatagcagtgccgacaga

SEQ ID NO: 807 - RNA, 19 nt, synthetic construct
  source 1..19: mol_type = other RNA
aggagtttgtccgtggcta

SEQ ID NO: 808 - RNA, 19 nt, synthetic construct
  source 1..19: mol_type = other RNA
cggctgagtgcacggttaa

SEQ ID NO: 809 - RNA, 19 nt, synthetic construct
  source 1..19: mol_type = other RNA
tgggaatccccagtatcta

SEQ ID NO: 810 - RNA, 19 nt, synthetic construct
  source 1..19: mol_type = other RNA
cagacccctcagcttacga

SEQ ID NO: 811 - RNA, 19 nt, synthetic construct
  source 1..19: mol_type = other RNA
cccctcagcttacggtata

SEQ ID NO: 812 - RNA, 19 nt, synthetic construct
  source 1..19: mol_type = other RNA
ctcagcttacggtatctaa

SEQ ID NO: 813 - RNA, 19 nt, synthetic construct
  source 1..19: mol_type = other RNA
gcttacggtatctacatta

SEQ ID NO: 814 - RNA, 19 nt, synthetic construct
  source 1..19: mol_type = other RNA
ccggcggcagcgtatcata

SEQ ID NO: 815 - RNA, 19 nt, synthetic construct
  source 1..19: mol_type = other RNA
agcgtatcatccagcggaa

SEQ ID NO: 816 - RNA, 19 nt, synthetic construct
  source 1..19: mol_type = other RNA
gcgtatcatccagcggaaa

SEQ ID NO: 817 - RNA, 19 nt, synthetic construct
  source 1..19: mol_type = other RNA
tacctaggccggtactata

SEQ ID NO: 818 - RNA, 19 nt, synthetic construct
  source 1..19: mol_type = other RNA
ctaggccggtactatatga

SEQ ID NO: 819 - RNA, 19 nt, synthetic construct
  source 1..19: mol_type = other RNA
aggccggtactatatgtca

SEQ ID NO: 820 - RNA, 19 nt, synthetic construct
  source 1..19: mol_type = other RNA
ggccggtactatatgtcta

SEQ ID NO: 821 - RNA, 19 nt, synthetic construct
  source 1..19: mol_type = other RNA
gccggtactatatgtctga

SEQ ID NO: 822 - RNA, 19 nt, synthetic construct
  source 1..19: mol_type = other RNA
cggtactatatgtctgcga

SEQ ID NO: 823 - RNA, 19 nt, synthetic construct
  source 1..19: mol_type = other RNA
ggtactatatgtctgcgca

SEQ ID NO: 824 - RNA, 19 nt, synthetic construct
  source 1..19: mol_type = other RNA
ctatatgtctgcgcgccaa

SEQ ID NO: 825 - RNA, 19 nt, synthetic construct
  source 1..19: mol_type = other RNA
tatgtctgcgcgccacata

SEQ ID NO: 826 - RNA, 19 nt, synthetic construct
  source 1..19: mol_type = other RNA
atgtctgcgcgccacatga

SEQ ID NO: 827 - RNA, 19 nt, synthetic construct
  source 1..19: mol_type = other RNA
tcgccctcgctgtcacgaa

SEQ ID NO: 828 - RNA, 19 nt, synthetic construct
  source 1..19: mol_type = other RNA
cggcgagcgctacgatgaa

SEQ ID NO: 829 - RNA, 19 nt, synthetic construct
  source 1..19: mol_type = other RNA
caaggaccggcgcaacata

SEQ ID NO: 830 - RNA, 19 nt, synthetic construct
  source 1..19: mol_type = other RNA
acatccgtgcaccagagta

SEQ ID NO: 831 - RNA, 19 nt, synthetic construct
  source 1..19: mol_type = other RNA
ctgggcgaggagcgtaaca

SEQ ID NO: 832 - RNA, 19 nt, synthetic construct
  source 1..19: mol_type = other RNA
gggcgaggagcgtaactaa

SEQ ID NO: 833 - RNA, 19 nt, synthetic construct
  source 1..19: mol_type = other RNA
gcgaggagcgtaactaaga

SEQ ID NO: 834 - RNA, 19 nt, synthetic construct
  source 1..19: mol_type = other RNA
gaggagcgtaactaagtca

SEQ ID NO: 835 - RNA, 19 nt, synthetic construct
  source 1..19: mol_type = other RNA
agtccgccaaacactccaa

SEQ ID NO: 836 - RNA, 19 nt, synthetic construct
  source 1..19: mol_type = other RNA
ggcgatcaagtccagagca

SEQ ID NO: 837 - RNA, 19 nt, synthetic construct
  source 1..19: mol_type = other RNA
ccctaacctggcttattca

SEQ ID NO: 838 - RNA, 19 nt, synthetic construct
  source 1..19: mol_type = other RNA
tgtgaaaccactaggttca

SEQ ID NO: 839 - RNA, 19 nt, synthetic construct
  source 1..19: mol_type = other RNA
accactaggttctaggtca

SEQ ID NO: 840 - RNA, 19 nt, synthetic construct
  source 1..19: mol_type = other RNA
tggacttcaacctagacaa

SEQ ID NO: 841 - RNA, 19 nt, synthetic construct
  source 1..19: mol_type = other RNA
ctgggaggatgaattcgaa

SEQ ID NO: 842 - RNA, 19 nt, synthetic construct
  source 1..19: mol_type = other RNA
gggtgacaactactatcta

SEQ ID NO: 843 - RNA, 19 nt, synthetic construct
  source 1..19: mol_type = other RNA
ctgggaggatgaattcgaa

SEQ ID NO: 844 - RNA, 19 nt, synthetic construct
  source 1..19: mol_type = other RNA
caaggtgggtggcatctaa

SEQ ID NO: 845 - RNA, 19 nt, synthetic construct
  source 1..19: mol_type = other RNA
tccgcagcctggatgatta

SEQ ID NO: 846 - RNA, 19 nt, synthetic construct
  source 1..19: mol_type = other RNA
caggggtgcggtcttgcaa

SEQ ID NO: 847 - RNA, 19 nt, synthetic construct
  source 1..19: mol_type = other RNA
ggtgcggtcttgcaatagg

SEQ ID NO: 848 - RNA, 19 nt, synthetic construct
  source 1..19: mol_type = other RNA
gtgcggtcttgcaatagga

SEQ ID NO: 849 - RNA, 19 nt, synthetic construct
  source 1..19: mol_type = other RNA
agccatgcctttaaaccgc

SEQ ID NO: 850 - RNA, 19 nt, synthetic construct
  source 1..19: mol_type = other RNA
atgcctttaaaccgcactt

SEQ ID NO: 851 - RNA, 19 nt, synthetic construct
  source 1..19: mol_type = other RNA
tgggaggatgaattcgacc

SEQ ID NO: 852 - RNA, 19 nt, synthetic construct
  source 1..19: mol_type = other RNA
aacgcagtgctcttcgaag

SEQ ID NO: 853 - RNA, 19 nt, synthetic construct
  source 1..19: mol_type = other RNA
ggacgaatggggcgacaac

SEQ ID NO: 854 - RNA, 19 nt, synthetic construct
  source 1..19: mol_type = other RNA
ggcgactgcctgtagcaac

SEQ ID NO: 855 - RNA, 19 nt, synthetic construct
  source 1..19: mol_type = other RNA
cacgctgctggggcgctac

SEQ ID NO: 856 - RNA, 19 nt, synthetic construct
  source 1..19: mol_type = other RNA
tgcgctcacgtcttcacta

SEQ ID NO: 857 - RNA, 19 nt, synthetic construct
  source 1..19: mol_type = other RNA
ccttatacttctttatcgc

SEQ ID NO: 858 - RNA, 19 nt, synthetic construct
  source 1..19: mol_type = other RNA
ctttatcgccggccgctat

SEQ ID NO: 859 - RNA, 19 nt, synthetic construct
  source 1..19: mol_type = other RNA
tcgccggccgctatgagtt

SEQ ID NO: 860 - RNA, 19 nt, synthetic construct
  source 1..19: mol_type = other RNA
gccgctatgagttctccaa

SEQ ID NO: 861 - RNA, 19 nt, synthetic construct
  source 1..19: mol_type = other RNA
ccgctatgagttctccaac

SEQ ID NO: 862 - RNA, 19 nt, synthetic construct
  source 1..19: mol_type = other RNA
ggctcggctcaactatctg

SEQ ID NO: 863 - RNA, 19 nt, synthetic construct
  source 1..19: mol_type = other RNA
gctcggctcaactatctgc

SEQ ID NO: 864 - RNA, 19 nt, synthetic construct
  source 1..19: mol_type = other RNA
tcggctcaactatctgctc

SEQ ID NO: 865 - RNA, 19 nt, synthetic construct
  source 1..19: mol_type = other RNA
ccagcgcggaccaacaatt

SEQ ID NO: 866 - RNA, 19 nt, synthetic construct
  source 1..19: mol_type = other RNA
agcgcggaccaacaatttc

SEQ ID NO: 867 - RNA, 19 nt, synthetic construct
  source 1..19: mol_type = other RNA
tcgggaggaagctttatga

SEQ ID NO: 868 - RNA, 19 nt, synthetic construct
  source 1..19: mol_type = other RNA
acgcagcggcagtctttcc

SEQ ID NO: 869 - RNA, 19 nt, synthetic construct
  source 1..19: mol_type = other RNA
gaccaccatccgccgaatc

SEQ ID NO: 870 - RNA, 19 nt, synthetic construct
  source 1..19: mol_type = other RNA
ccgaatcggcctcttcaat

SEQ ID NO: 871 - RNA, 19 nt, synthetic construct
  source 1..19: mol_type = other RNA
aatcggcctcttcaatagc

SEQ ID NO: 872 - RNA, 19 nt, synthetic construct
  source 1..19: mol_type = other RNA
caatagcagtgccgacagg

SEQ ID NO: 873 - RNA, 19 nt, synthetic construct
  source 1..19: mol_type = other RNA
aggagtttgtccgtggctg

SEQ ID NO: 874 - RNA, 19 nt, synthetic construct
  source 1..19: mol_type = other RNA
cggctgagtgcacggttat

SEQ ID NO: 875 - RNA, 19 nt, synthetic construct
  source 1..19: mol_type = other RNA
tgggaatccccagtatctc

SEQ ID NO: 876 - RNA, 19 nt, synthetic construct
  source 1..19: mol_type = other RNA
cagacccctcagcttacgg

SEQ ID NO: 877 - RNA, 19 nt, synthetic construct
  source 1..19: mol_type = other RNA
cccctcagcttacggtatc

SEQ ID NO: 878 - RNA, 19 nt, synthetic construct
  source 1..19: mol_type = other RNA
ctcagcttacggtatctac

SEQ ID NO: 879 - RNA, 19 nt, synthetic construct
  source 1..19: mol_type = other RNA
gcttacggtatctacattc

SEQ ID NO: 880 - RNA, 19 nt, synthetic construct
  source 1..19: mol_type = other RNA
ccggcggcagcgtatcatc

SEQ ID NO: 881 - RNA, 19 nt, synthetic construct
  source 1..19: mol_type = other RNA
agcgtatcatccagcggaa

SEQ ID NO: 882 - RNA, 19 nt, synthetic construct
  source 1..19: mol_type = other RNA
gcgtatcatccagcggaac

SEQ ID NO: 883 - RNA, 19 nt, synthetic construct
  source 1..19: mol_type = other RNA
tacctaggccggtactata

SEQ ID NO: 884 - RNA, 19 nt, synthetic construct
  source 1..19: mol_type = other RNA
ctaggccggtactatatgt

SEQ ID NO: 885 - RNA, 19 nt, synthetic construct
  source 1..19: mol_type = other RNA
aggccggtactatatgtct

SEQ ID NO: 886 - RNA, 19 nt, synthetic construct
  source 1..19: mol_type = other RNA
ggccggtactatatgtctg

SEQ ID NO: 887 - RNA, 19 nt, synthetic construct
  source 1..19: mol_type = other RNA
gccggtactatatgtctgc

SEQ ID NO: 888 - RNA, 19 nt, synthetic construct
  source 1..19: mol_type = other RNA
cggtactatatgtctgcgc

SEQ ID NO: 889 - RNA, 19 nt, synthetic construct
  source 1..19: mol_type = other RNA
ggtactatatgtctgcgcg

SEQ ID NO: 890 - RNA, 19 nt, synthetic construct
  source 1..19: mol_type = other RNA
ctatatgtctgcgcgccac

SEQ ID NO: 891 - RNA, 19 nt, synthetic construct
  source 1..19: mol_type = other RNA
tatgtctgcgcgccacatg

SEQ ID NO: 892 - RNA, 19 nt, synthetic construct
  source 1..19: mol_type = other RNA
atgtctgcgcgccacatgg

SEQ ID NO: 893 - RNA, 19 nt, synthetic construct
  source 1..19: mol_type = other RNA
tcgccctcgctgtcacgac

SEQ ID NO: 894 - RNA, 19 nt, synthetic construct
  source 1..19: mol_type = other RNA
cggcgagcgctacgatgag

SEQ ID NO: 895 - RNA, 19 nt, synthetic construct
  source 1..19: mol_type = other RNA
caaggaccggcgcaacatc

SEQ ID NO: 896 - RNA, 19 nt, synthetic construct
  source 1..19: mol_type = other RNA
acatccgtgcaccagagtg

SEQ ID NO: 897 - RNA, 19 nt, synthetic construct
  source 1..19: mol_type = other RNA
ctgggcgaggagcgtaact

SEQ ID NO: 898 - RNA, 19 nt, synthetic construct
  source 1..19: mol_type = other RNA
gggcgaggagcgtaactaa

SEQ ID NO: 899 - RNA, 19 nt, synthetic construct
  source 1..19: mol_type = other RNA
gcgaggagcgtaactaagt

SEQ ID NO: 900 - RNA, 19 nt, synthetic construct
  source 1..19: mol_type = other RNA
gaggagcgtaactaagtcc

SEQ ID NO: 901 - RNA, 19 nt, synthetic construct
  source 1..19: mol_type = other RNA
agtccgccaaacactccac

SEQ ID NO: 902 - RNA, 19 nt, synthetic construct
  source 1..19: mol_type = other RNA
ggcgatcaagtccagagcc

SEQ ID NO: 903 - RNA, 19 nt, synthetic construct
  source 1..19: mol_type = other RNA
ccctaacctggcttattcc

SEQ ID NO: 904 - RNA, 19 nt, synthetic construct
  source 1..19: mol_type = other RNA
tgtgaaaccactaggttct

SEQ ID NO: 905 - RNA, 19 nt, synthetic construct
  source 1..19: mol_type = other RNA
accactaggttctaggtcc

SEQ ID NO: 906 - RNA, 19 nt, synthetic construct
  source 1..19: mol_type = other RNA
caggggtgcggtcttgcaa

SEQ ID NO: 907 - RNA, 19 nt, synthetic construct
  source 1..19: mol_type = other RNA
ggtgcggtcttgcaataga

SEQ ID NO: 908 - RNA, 19 nt, synthetic construct
  source 1..19: mol_type = other RNA
gtgcggtcttgcaatagga

SEQ ID NO: 909 - RNA, 19 nt, synthetic construct
  source 1..19: mol_type = other RNA
agccatgcctttaaaccga

SEQ ID NO: 910 - RNA, 19 nt, synthetic construct
  source 1..19: mol_type = other RNA
atgcctttaaaccgcacta

SEQ ID NO: 911 - RNA, 19 nt, synthetic construct
  source 1..19: mol_type = other RNA
tgggaggatgaattcgaca

SEQ ID NO: 912 - RNA, 19 nt, synthetic construct
  source 1..19: mol_type = other RNA
aacgcagtgctcttcgaaa

SEQ ID NO: 913 - RNA, 19 nt, synthetic construct
  source 1..19: mol_type = other RNA
ggacgaatggggcgacaaa

SEQ ID NO: 914 - RNA, 19 nt, synthetic construct
  source 1..19: mol_type = other RNA
ggcgactgcctgtagcaaa

SEQ ID NO: 915 - RNA, 19 nt, synthetic construct
  source 1..19: mol_type = other RNA
cacgctgctggggcgctaa

SEQ ID NO: 916 - RNA, 19 nt, synthetic construct
  source 1..19: mol_type = other RNA
tgcgctcacgtcttcacta

SEQ ID NO: 917 - RNA, 19 nt, synthetic construct
  source 1..19: mol_type = other RNA
ccttatacttctttatcga

SEQ ID NO: 918 - RNA, 19 nt, synthetic construct
  source 1..19: mol_type = other RNA
ctttatcgccggccgctaa

SEQ ID NO: 919 - RNA, 19 nt, synthetic construct
  source 1..19: mol_type = other RNA
tcgccggccgctatgagta

SEQ ID NO: 920 - RNA, 19 nt, synthetic construct
  source 1..19: mol_type = other RNA
gccgctatgagttctccaa

SEQ ID NO: 921 - RNA, 19 nt, synthetic construct
  source 1..19: mol_type = other RNA
ccgctatgagttctccaaa

SEQ ID NO: 922 - RNA, 19 nt, synthetic construct
  source 1..19: mol_type = other RNA
ggctcggctcaactatcta

SEQ ID NO: 923 - RNA, 19 nt, synthetic construct
  source 1..19: mol_type = other RNA
gctcggctcaactatctga

SEQ ID NO: 924 - RNA, 19 nt, synthetic construct
  source 1..19: mol_type = other RNA
tcggctcaactatctgcta

SEQ ID NO: 925 - RNA, 19 nt, synthetic construct
  source 1..19: mol_type = other RNA
ccagcgcggaccaacaata

SEQ ID NO: 926 - RNA, 19 nt, synthetic construct
  source 1..19: mol_type = other RNA
agcgcggaccaacaattta

SEQ ID NO: 927 - RNA, 19 nt, synthetic construct
  source 1..19: mol_type = other RNA
tcgggaggaagctttatga

SEQ ID NO: 928 - RNA, 19 nt, synthetic construct
  source 1..19: mol_type = other RNA
acgcagcggcagtctttca

SEQ ID NO: 929 - RNA, 19 nt, synthetic construct
  source 1..19: mol_type = other RNA
gaccaccatccgccgaata

SEQ ID NO: 930 - RNA, 19 nt, synthetic construct
  source 1..19: mol_type = other RNA
ccgaatcggcctcttcaaa

SEQ ID NO: 931 - RNA, 19 nt, synthetic construct
  source 1..19: mol_type = other RNA
aatcggcctcttcaataga

SEQ ID NO: 932 - RNA, 19 nt, synthetic construct
  source 1..19: mol_type = other RNA
caatagcagtgccgacaga

SEQ ID NO: 933 - RNA, 19 nt, synthetic construct
  source 1..19: mol_type = other RNA
aggagtttgtccgtggcta

SEQ ID NO: 934 - RNA, 19 nt, synthetic construct
  source 1..19: mol_type = other RNA
cggctgagtgcacggttaa

SEQ ID NO: 935 - RNA, 19 nt, synthetic construct
  source 1..19: mol_type = other RNA
tgggaatccccagtatcta

SEQ ID NO: 936 - RNA, 19 nt, synthetic construct
  source 1..19: mol_type = other RNA
cagacccctcagcttacga

SEQ ID NO: 937 - RNA, 19 nt, synthetic construct
  source 1..19: mol_type = other RNA
cccctcagcttacggtata

SEQ ID NO: 938 - RNA, 19 nt, synthetic construct
  source 1..19: mol_type = other RNA
ctcagcttacggtatctaa

SEQ ID NO: 939 - RNA, 19 nt, synthetic construct
  source 1..19: mol_type = other RNA
gcttacggtatctacatta

SEQ ID NO: 940 - RNA, 19 nt, synthetic construct
  source 1..19: mol_type = other RNA
ccggcggcagcgtatcata

SEQ ID NO: 941 - RNA, 19 nt, synthetic construct
  source 1..19: mol_type = other RNA
agcgtatcatccagcggaa

SEQ ID NO: 942 - RNA, 19 nt, synthetic construct
  source 1..19: mol_type = other RNA
gcgtatcatccagcggaaa

SEQ ID NO: 943 - RNA, 19 nt, synthetic construct
  source 1..19: mol_type = other RNA
tacctaggccggtactata

SEQ ID NO: 944 - RNA, 19 nt, synthetic construct
  source 1..19: mol_type = other RNA
ctaggccggtactatatga

SEQ ID NO: 945 - RNA, 19 nt, synthetic construct
  source 1..19: mol_type = other RNA
aggccggtactatatgtca

SEQ ID NO: 946 - RNA, 19 nt, synthetic construct
  source 1..19: mol_type = other RNA
ggccggtactatatgtcta

SEQ ID NO: 947 - RNA, 19 nt, synthetic construct
  source 1..19: mol_type = other RNA
gccggtactatatgtctga

SEQ ID NO: 948 - RNA, 19 nt, synthetic construct
  source 1..19: mol_type = other RNA
cggtactatatgtctgcga

SEQ ID NO: 949 - RNA, 19 nt, synthetic construct
  source 1..19: mol_type = other RNA
ggtactatatgtctgcgca

SEQ ID NO: 950 - RNA, 19 nt, synthetic construct
  source 1..19: mol_type = other RNA
ctatatgtctgcgcgccaa

SEQ ID NO: 951 - RNA, 19 nt, synthetic construct
  source 1..19: mol_type = other RNA
tatgtctgcgcgccacata

SEQ ID NO: 952 - RNA, 19 nt, synthetic construct
  source 1..19: mol_type = other RNA
atgtctgcgcgccacatga

SEQ ID NO: 953 - RNA, 19 nt, synthetic construct
  source 1..19: mol_type = other RNA
tcgccctcgctgtcacgaa

SEQ ID NO: 954 - RNA, 19 nt, synthetic construct
  source 1..19: mol_type = other RNA
cggcgagcgctacgatgaa

SEQ ID NO: 955 - RNA, 19 nt, synthetic construct
  source 1..19: mol_type = other RNA
caaggaccggcgcaacata

SEQ ID NO: 956 - RNA, 19 nt, synthetic construct
  source 1..19: mol_type = other RNA
acatccgtgcaccagagta

SEQ ID NO: 957 - RNA, 19 nt, synthetic construct
  source 1..19: mol_type = other RNA
ctgggcgaggagcgtaaca

SEQ ID NO: 958 - RNA, 19 nt, synthetic construct
  source 1..19: mol_type = other RNA
gggcgaggagcgtaactaa

SEQ ID NO: 959 - RNA, 19 nt, synthetic construct
  source 1..19: mol_type = other RNA
gcgaggagcgtaactaaga

SEQ ID NO: 960 - RNA, 19 nt, synthetic construct
  source 1..19: mol_type = other RNA
gaggagcgtaactaagtca

SEQ ID NO: 961 - RNA, 19 nt, synthetic construct
  source 1..19: mol_type = other RNA
agtccgccaaacactccaa

SEQ ID NO: 962 - RNA, 19 nt, synthetic construct
  source 1..19: mol_type = other RNA
ggcgatcaagtccagagca

SEQ ID NO: 963 - RNA, 19 nt, synthetic construct
  source 1..19: mol_type = other RNA
ccctaacctggcttattca

SEQ ID NO: 964 - RNA, 19 nt, synthetic construct
  source 1..19: mol_type = other RNA
tgtgaaaccactaggttca

SEQ ID NO: 965 - RNA, 19 nt, synthetic construct
  source 1..19: mol_type = other RNA
accactaggttctaggtca

SEQ ID NO: 966 - RNA, 19 nt, synthetic construct
  source 1..19: mol_type = other RNA
tggacttcaacctagacaa

SEQ ID NO: 967 - RNA, 19 nt, synthetic construct
  source 1..19: mol_type = other RNA
ctgggaggatgaattcgaa

SEQ ID NO: 968 - RNA, 19 nt, synthetic construct
  source 1..19: mol_type = other RNA
gggtgacaactactatcta

SEQ ID NO: 969 - RNA, 19 nt, synthetic construct
  source 1..19: mol_type = other RNA
ctgggaggatgaattcgaa

SEQ ID NO: 970 - RNA, 19 nt, synthetic construct
  source 1..19: mol_type = other RNA
caaggtgggtggcatctaa

SEQ ID NO: 971 - RNA, 19 nt, synthetic construct
  source 1..19: mol_type = other RNA
tccgcagcctggatgatta

SEQ ID NO: 972 - protein, 91 aa, synthetic construct
MSLPAPKNLVVSRVTEDSARLSWTAPDAAFDSFFIGYLEPQPPGEAIALYVPGSERSYDLTGLKPGTEYNVTIQGAKGGFPSEPLVVHFTT

SEQ ID NO: 973 - protein, 91 aa, synthetic construct
MSLPAPKNLVVSRVTEDSARLSWTAPDAAFDSFFIGYLEPQPPGEAIHLYVPGSERSYDLTGLKPGTEYNGTIQGVKVGFPSEPLIANFTT

SEQ ID NO: 974 - protein, 91 aa, synthetic construct
MSLPAPKNLVVSRVTEDSARLSWTAPDAAFDSFFIGYLEPQPPGEAIALYVPGSERSYDLTGLKPGTEYNVTIQGVKGGFPSLPLVATFTT

SEQ ID NO: 975 - protein, 90 aa, synthetic construct
MLPAPKNLVVSRVTEDSARLSWTAPDAAFDSFFIGYLEPQPPGEAIELYVPGSCRSYDLTGLKPGTEYNVTIQGVKGGFPSLPLVATFTT

SEQ ID NO: 976 - protein, 90 aa, synthetic construct
MLPAPKNLVVSRVTEDSARLSWTAPDAAFDSFFIGYLEPQPPGEAIHLYVPGSCRSYDLTGLKPGTEYNVTIQGVKGGFPSVPLIAKFTT

SEQ ID NO: 977 - RNA, 1682 nt, Homo sapiens
  source 1..1682: mol_type = mRNA
agtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctgggggagggttggttaaagggagatttggctttcccataatgcttcatcatttttcccaaaaggagagtgaattcacataatgcttatgtaattaaaaaatcatcaaacatgtaaaaa

SEQ ID NO: 978 - RNA, 3377 nt, Homo sapiens
  source 1..3377: mol_type = mRNA
agtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaaggtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccagatattgtgacccccaatgggctgaatgtgaagaagttttctgccatgcatgagttccagaacctccatgctcagagcaaggctcgaatccaggagtttgtgcggggccatttttatgggcatctggacttcaacttggacaagaccttatacttctttatcgccggccgctatgagttctccaacaagggtgctgacgtcttcctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccatccgccgaatcggcctcttcaatagcagtgccgacagggtgaaggtgattttccacccggagttcctctcctccacaagccccctgctccctgtggactatgaggagtttgtccgtggctgtcaccttggagtcttcccctcctactatgagccttggggctacacaccggctgagtgcacggttatgggaatccccagtatctccaccaatctctccggcttcggctgcttcatggaggaacacatcgcagacccctcagcttacggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccggtactatatgtctgcgcgccacatggcgctgtccaaggcctttccagagcacttcacctacgagcccaacgaggcggatgcggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaa